(19) United States
(12) Patent Application Publication
(10) Pub. No.: US 2015/0239842 A1
(43) Pub. Date: Aug. 27, 2015

(54) BENZAMIDE AND HETEROBENZAMIDE COMPOUNDS

(71) Applicant: PFIZER INC., New York, NY (US)

(72) Inventors: Martin Paul Edwards, San Diego, CA (US); Robert Arnold Kumpf, San Diego, CA (US); Pei-Pei Kung, San Diego, CA (US); Indrawan James Mcalpine, San Diego, CA (US); Eugene Yuanjin Rui, San Diego, CA (US); Scott Channing Sutton, San Diego, CA (US); John Howard Tatlock, San Diego, CA (US); Martin James Wythes, San Diego, CA (US)

(73) Assignee: PFIZER INC., New York, NY (US)

(21) Appl. No.: 14/430,799

(22) PCT Filed: Sep. 16, 2013
(86) PCT No.: PCT/IB2013/058580

§ 371 Date: Mar. 24, 2015

Related U.S. Application Data
(60) Provisional application No. 61/707,447, filed on Sep. 28, 2012.

(51) Int. Cl. C07D 213/64; C07D 487/08; C07D 491/08; C07D 405/14; C07D 413/14; C07D 401/12; C07D 401/14

(52) U.S. Cl. CPC C07D 213/64; C07D 401/12; C07D 487/08; C07D 401/14; C07D 405/14; C07D 413/14; C07D 491/08

(57) ABSTRACT

This invention relates to compounds of general formula (I), in which R¹, R², R⁶, U, V, W, X, Y and Z are as defined herein, and the pharmaceutically acceptable salts thereof, pharmaceutical compositions containing such compounds and salts, and to methods of using such compounds, salts and compositions.

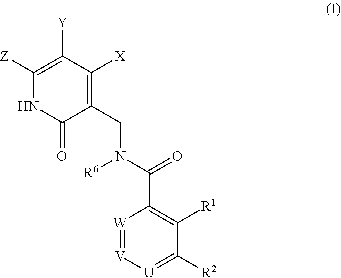

### FIELD OF THE INVENTION

The present invention relates to compounds of formulae (I)-(VII) and their pharmaceutically acceptable salts, to pharmaceutical compositions comprising such compounds and salts, and to the use of such compounds, salts and compositions. The compounds and salts of the present invention are useful for treating or ameliorating abnormal cell proliferative disorders, such as cancer.

### BACKGROUND

Epigenetic alterations play an important role in the regulation of cellular processes, including cell proliferation, cell differentiation and cell survival. The epigenetic silencing of tumor suppressor genes and activation of oncogenes may occur through alteration of CpG island methylation patterns, histone modification, and dysregulation of DNA binding protein. Polycomb genes are a set of epigenetic effectors. EZH2 (enhancer of zeste homolog 2) is the catalytic component of the Polycomb Repressor Complex 2 (PRC2), a conserved multi-subunit complex that represses gene transcription by methylating lysine 27 on Histone H3 (H3K27). Cardoso et al., Eur. J. Hum. Genet. 2000, 8:174-180. EZH2 plays a key role in regulating gene expression patterns that regulate cell fate decisions, such as differentiation and self-renewal. EZH2 is overexpressed in certain cancer cells, where it has been linked to cell proliferation, cell invasion, chemoresistance and metastasis.

High EZH2 expression has been correlated with poor prognosis, high grade, and high stage in several cancer types, including breast, colorectal, endometrial, gastric, liver, kidney, lung, melanoma, ovarian, pancreatic, prostate, and bladder cancers. See Crea et al., Crit. Rev. Oncol. Hematol. 2012, 83:184-193, and references cited therein; see also Kleer et al., Proc. Natl. Acad. Sci. USA 2003, 100:11606-11; Mimori et al., Eur. J. Surg. Oncol. 2005, 31:376-80; Bachmann et al., J. Clin. Oncol. 2006, 24:268-273; Matsukawa et al., Cancer Sci. 2006, 97:484-491; Sasaki et al. Lab. Invest. 2008, 88:873-882; Sudo et al., Br. J. Cancer 2005, 92(9):1754-1758; Breuer et al., Neoplasia 2004, 6:736-43; Lu et al., Cancer Res. 2007, 67:1757-1768; Ougolkov et al., Clin. Cancer Res. 2008, 14:6790-6796; Varambally et al., Nature 2002, 419:624-629; Wagener et al., Int. J. Cancer 2008, 123:1545-1550; and Weikert et al., Int. J. Mol. Med. 2005, 16:349-353.

Recurring somatic mutations in EZH2 have been identified in diffuse large B-cell lymphoma (DLBCL) and follicular lymphomas (FL). Mutations altering EZH2 tyrosine 641 (e.g., Y641C, Y641F, Y641N, Y641S, and Y641H) were reportedly observed in up to 22% of germinal center B-cell DLBCL and 7% of FL. Morin et al. Nat. Genetics 2010 February; 42(2):181-185. Mutations of alanine 677 (A677) and alanine 687 (A687) have also been reported. McCAbe et al., Proc. Natl. Acad. Sci. USA 2012, 109:2989-2994; Majer et al. FEBS Letters 2012, 586:3448-3451. EZH2 activating mutations have been suggested to alter substrate specificity resulting in elevated levels of trimethylated H3K27 (H3K27me3).

Accordingly, compounds that inhibit the activity of wild type and/or mutant forms of EZH2 are of interest for the treatment of cancer.

### SUMMARY

The present invention provides, in part, novel compounds and pharmaceutically acceptable salts that can modulate the activity of EZH2, thereby effecting biological functions, including but not limited to inhibiting cell proliferation and cell invasiveness, inhibiting metastasis, inducing apoptosis or inhibiting angiogenesis. Also provided are pharmaceutical compositions and medicaments comprising the compounds or salts of the invention, alone or in combination with other therapeutic or palliative agents. The present invention also provides, in part, methods for preparing the novel compounds, salts and compositions thereof, and methods of using the foregoing.

In one aspect, the invention provides a compound of formula (I):

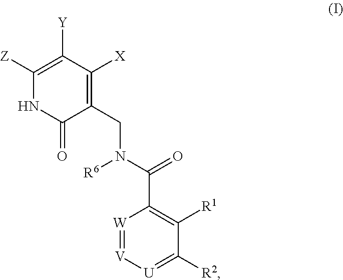

or a pharmaceutically acceptable salt thereof,

wherein:

U is N or CR³;

V is N or CR⁴;

W is N or CR⁵;

R¹ is C₁-C₈ alkyl, C₁-C₈ alkoxy, halo, —OH, —CN or —NR⁷R⁸, where each said C₁-C₈ alkyl or C₁-C₈ alkoxy is optionally substituted by one or more R²¹;

R² is 3-12 membered heterocyclyl, C₆-C₁₂ aryl, 5-12 membered heteroaryl or C₁-C₈ alkoxy, where said C₁-C₈ alkoxy is optionally substituted by one or more R²², and each said heterocyclyl, aryl or heteroaryl is optionally substituted by one or more R³²;

R³ is H, C₁-C₈ alkyl, C₁-C₈ alkoxy, halo, —OH, —CN or —NR⁷R⁸, where each said C₁-C₈ alkyl or C₁-C₈ alkoxy is optionally substituted by one or more R²³;

R⁴ is independently selected from the group consisting of H, C₁-C₈ alkyl, C₁-C₈ alkoxy, C₁-C₈ thioalkoxy, halo, —OH, —CN, C₃-C₈ cycloalkyl, 3-12 membered heterocyclyl, C₆-C₁₂ aryl, 5-12 membered heteroaryl, —OR¹¹ and —NR⁷R⁸, where each said C₁-C₈ alkyl, C₁-C₈ alkoxy, C₁-C₈ thioalkoxy or C₃-C₈ cycloalkyl is optionally substituted by one or more R²⁴, and each said heterocyclyl, aryl, heteroaryl or R¹¹ is optionally substituted by one or more R³⁴;

R⁵ is H, C₁-C₈ alkyl, C₁-C₈ alkoxy, halo, —OH, —CN or —NR⁷R⁸, where each said C₁-C₈ alkyl or C₁-C₈ alkoxy is optionally substituted by one or more R²⁵;

R⁶ is H or C₁-C₄ alkyl;

each R⁷ and R⁸ is independently H or C₁-C₈ alkyl, where said C₁-C₈ alkyl is optionally substituted by one or more R²⁷; or

R⁷ and R⁸ may be taken together with the N atom to which they are attached to form a 3-12 membered heterocyclyl or 5-12 membered heteroaryl, each optionally containing 1, 2 or 3 additional heteroatoms selected from O, N and S, wherein each said heterocyclyl or heteroaryl is optionally substituted by one or more R³⁷;

each R²¹, R²², R²³ and R²⁵ is independently selected from the group consisting of halo, —OH, C₁-C₄ alkoxy, —CN and —NR⁹R¹⁰;

each R²⁴ and R²⁷ is independently selected from the group consisting of halo, —OH, C₁-C₄ alkoxy, —CN, —NR⁹R¹⁰, C₃-C₈ cycloalkyl, 3-12 membered heterocyclyl, C₆-C₁₂ aryl and 5-12 membered heteroaryl, where each said cycloalkyl, heterocyclyl, aryl or heteroaryl is optionally substituted by one or more substituents independently selected from the group consisting of halo, —OH, ═O, C₁-C₄ alkyl, C₁-C₄ alkoxy, —CN, —NH₂, —NH(C₁-C₄ alkyl) and —N(C₁-C₄ alkyl)₂;

each R⁹ and R¹⁰ is independently H or C₁-C₄ alkyl; or

R⁹ and R¹⁰ may be taken together with the N atom to which they are attached to form a 3-12 membered heterocyclyl or 5-12 membered heteroaryl, each optionally containing 1, 2 or 3 additional heteroatoms selected from O, N and S, where each said heterocyclyl or heteroaryl is optionally substituted by one or more substituents independently selected from the group consisting of halo, —OH, ═O, C₁-C₄ alkyl, C₁-C₄ alkoxy, —CN, —NH₂, —NH(C₁-C₄ alkyl) and —N(C₁-C₄ alkyl)₂;

R¹¹ is selected from the group consisting of C₃-C₈ cycloalkyl, 3-12 membered heterocyclyl, C₆-C₁₂ aryl and 5-12 membered heteroaryl;

each R³², R³⁴ and R³⁷ is independently selected from the group consisting of halo, C₁-C₈ alkyl, —CN, ═O, —COR^{c}, —CO₂R^{c}, —CONR^{c}R^{d}, —OR^{c}, —SR^{c}, —SOR^{c}, —SO₂R^{c}, —SO₂NR^{c}R^{d}, —NO₂, —NR^{c}R^{d}, —NR^{c}C(O)R^{d}, —NR^{c}C(O)NR^{c}R^{d}, —NR^{c}C(O)OR^{d}—NR^{c}SO₂R^{d}, —NR^{c}SO₂NR^{c}R^{d}, —OC(O)R^{c}, —OC(O)NR^{c}R^{d}, C₃-C₈ cycloalkyl, 3-12 membered heterocyclyl, C₆-C₁₂ aryl and 5-12 membered heteroaryl;
each R^{c} and R^{d} is independently selected from the group consisting of H, C₁-C₈ alkyl, C₃-C₈ cycloalkyl, 3-12 membered heterocyclyl, C₆-C₁₂ aryl and 5-12 membered heteroaryl; or
R^{c} and R^{d} may be taken together with the N atom to which they are attached to form a 3-12 membered heterocyclyl or 5-12 membered heteroaryl ring, each optionally containing 1, 2 or 3 additional heteroatoms selected from O, N and S;
wherein each said alkyl, cycloalkyl, heterocyclyl, aryl or heteroaryl in R³², R³⁴, R³⁷, R^{c} and R^{d} is optionally substituted by 1 to 3 substituents independently selected from the group consisting of halo, —OH, ═O, C₁-C₄ alkyl, C₁-C₄ alkoxy, C₁-C₆ haloalkyl, C₁-C₆ hydroxyalkyl, C₁-C₄ alkoxy-C₁-C₆ alkyl, —CN, —NH₂, —NH(C₁-C₄ alkyl) and —N(C₁-C₄ alkyl)₂;

X and Z are independently selected from the group consisting of H, C₁-C₈ alkyl, C₂-C₈ alkenyl, C₂-C₈ alkynyl, C₃-C₈ cycloalkyl, 3-12 membered heterocyclyl, C₆-C₁₂ aryl, 5-12 membered heteroaryl, halo, CN, —COR^{a}, —CO₂R^{a}, —CONR^{a}R^{b}, —SR^{a}, —SOR^{a}, —SO₂R^{a}, —SO₂NR^{a}R^{b}, —NO₂, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}—OR^{a}, —OC(O)R^{a} or —OC(O)NR^{a}R^{b};
wherein each said C₁-C₈ alkyl, C₂-C₈ alkenyl, C₂-C₈ alkynyl, C₃-C₈ cycloalkyl, 3-12 membered heterocyclyl, C₆-C₁₂ aryl, or 5-12 membered heteroaryl group is optionally substituted by one or more substituents independently selected from the group consisting of halo, —CN, —COR^{a}, —CO₂R^{a}, —CONR^{a}R^{b}, —SR^{a}, —SOR^{a}, —SO₂R^{a}, —SO₂NR^{a}R^{b}, —NO₂, —NR^{a}R^{b}, —NR^{a}C(O)R^{b}, —NR^{a}C(O)NR^{a}R^{b}, —NR^{a}C(O)OR^{a}—NR^{a}SO₂R^{b}, —NR^{a}SO₂NR^{a}R^{b}, —OR^{a}, —OC(O)R^{a}, —OC(O)NR^{a}R^{b}, C₃-C₈ cycloalkyl, 3-12 membered heterocyclyl, C₆-C₁₂ aryl, and 5-12 membered heteroaryl;
each R^{a} and R^{b} is independently H, C₁-C₈ alkyl, C₂-C₈ alkenyl, C₂-C₈ alkynyl, C₃-C₈ cycloalkyl, 3-12 membered heterocyclyl, C₆-C₁₂ aryl or 5-12 membered heteroaryl, where each said alkyl, alkenyl, alkynyl, cycloalkyl, heterocyclyl, aryl and heteroaryl is optionally substituted by one or more substituents independently selected from the group consisting of halo, C₁-C₄ alkyl, —OR″, —NR″₂, —CO₂R″, —CONR″₂, —SO₂R″ and —SO₂NR″₂, where each R″ is independently H or C₁-C₄ alkyl; or
R^{a} and R^{b} may be taken together with the N atom to which they are attached to form a 3-12 membered heterocyclyl or 5-12 membered heteroaryl, each optionally containing 1, 2 or 3 additional heteroatoms selected from O, N and S, wherein said heterocyclyl or heteroaryl is optionally substituted by one or more substituents independently selected from the group consisting of halo, —OH, ═O, C₁-C₄ alkyl, C₁-C₄ alkoxy, C₁-C₆ haloalkyl, C₁-C₆ hydroxyalkyl, C₁-C₄ alkoxy-C₁-C₆ alkyl, —CN, —NH₂, —NH(C₁-C₄ alkyl) and —N(C₁-C₄ alkyl)₂; and

Y is H, halo, —OH or C₁-C₄ alkoxy.

In another aspect, the invention provides a compound of formula (II):

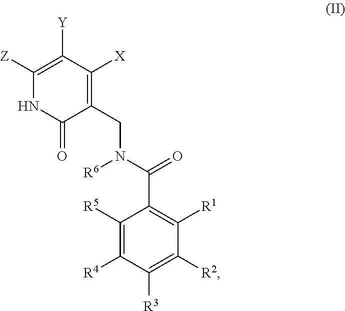

or a pharmaceutically acceptable salt thereof,

wherein R¹, R², R³, R⁴, R⁵, R⁶, X, Y and Z are defined as in formula (I).

In another aspect, the invention provides a compound of formula (III):

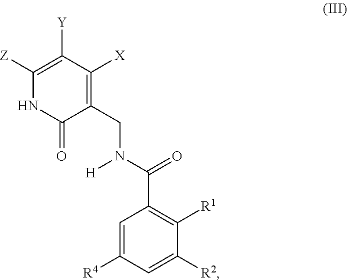

or a pharmaceutically acceptable salt thereof,

wherein R¹, R², R⁴, X, Y and Z are defined as in formula (II).

In a further aspect, the invention provides a compound of formula (IV):

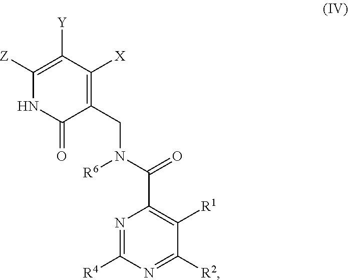

or a pharmaceutically acceptable salt thereof,

wherein R¹, R², R⁴, R⁶, X, Y and Z are defined as in formula (I).

In yet another aspect, the invention provides a compound of formula (V):

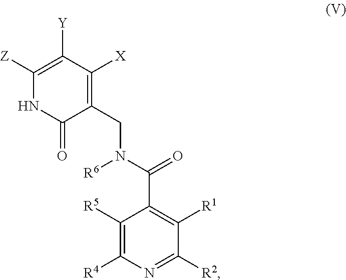

or a pharmaceutically acceptable salt thereof,

wherein R¹, R², R⁴, R⁵, R⁶, X, Y and Z are defined as in formula (I).

In yet another aspect, the invention provides a compound of formula (VI):

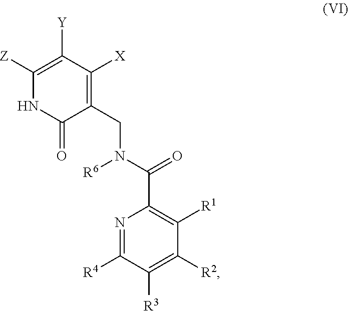

or a pharmaceutically acceptable salt thereof,

wherein R¹, R², R³, R⁴, R⁶, X, Y and Z are defined as in formula (I).

In yet another aspect, the invention provides a compound of formula (VII):

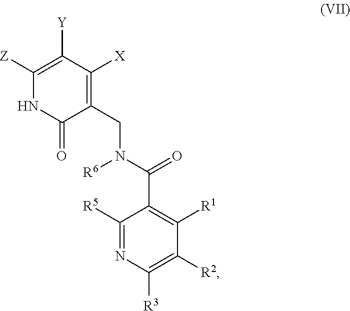

or a pharmaceutically acceptable salt thereof,

wherein R¹, R², R³, R⁵, R⁶, X, Y and Z are defined as in formula (I).

In another aspect, the invention provides a pharmaceutical composition comprising a compound of one of the formulae described herein, or a pharmaceutically acceptable salt thereof, and a pharmaceutically acceptable carrier or excipient. In some embodiments, the pharmaceutical composition comprises two or more pharmaceutically acceptable carriers and/or excipients.

The invention also provides therapeutic methods and uses comprising administering a compound of the invention, or a pharmaceutically acceptable salt thereof.

In one aspect, the invention provides a method for the treatment of abnormal cell growth in a subject comprising administering to the subject a therapeutically effective amount of a compound of the invention, or a pharmaceutically acceptable salt thereof.

In another aspect, the invention provides a method for the treatment of abnormal cell growth in a subject comprising administering to the subject an amount of a compound of the invention, or a pharmaceutically acceptable salt thereof, in combination with an amount of an anti-tumor agent, which amounts are together effective in treating said abnormal cell growth. In some embodiments, the anti-tumor agent is selected from the group consisting of mitotic inhibitors, alkylating agents, anti-metabolites, intercalating antibiotics, growth factor inhibitors, radiation, cell cycle inhibitors, enzyme inhibitors, topoisomerase inhibitors, biological response modifiers, antibodies, cytotoxics, anti-hormones, and anti-androgens.

In frequent embodiments of the methods provided herein, the abnormal cell growth is cancer. In some embodiments, the methods provided result in one or more of the following effects: (1) inhibiting cancer cell proliferation; (2) inhibiting cancer cell invasiveness; (3) inducing apoptosis of cancer cells; (4) inhibiting cancer cell metastasis; or (5) inhibiting angiogenesis.

In another aspect, the invention provides a method for the treatment of a disorder mediated by EZH2 in a subject comprising administering to the subject a compound of the invention, or a pharmaceutically acceptable salt thereof, in an amount that is effective for treating said disorder. The compounds and salts of the present invention inhibit wild-type and certain mutant forms of human histone methyltransferase EZH2. In frequent embodiments the disorder is cancer.

In another aspect, the invention provides a compound of one of the formulae described herein, or pharmaceutically acceptable salt thereof, for use in the treatment of abnormal cell growth. In another aspect, the invention provides a compound of one of the formulae described herein, or pharmaceutically acceptable salt thereof, for use in the treatment of abnormal cell growth in a subject.

In a further aspect, the invention provides the use of a compound of one of the formulae described herein, or pharmaceutically acceptable salt thereof, for the treatment of abnormal cell growth in a subject. In another aspect, the invention provides the use of a compound of one of the formulae described herein, or pharmaceutically acceptable salt thereof, for the treatment of abnormal cell growth.

In yet another aspect, the invention provides the use of a compound of one of the formulae described herein, or pharmaceutically acceptable salt thereof, for the preparation of a medicament for the treatment of abnormal cell growth.

In frequent embodiments, the abnormal cell growth is cancer and the subject is a human.

In some embodiments, the methods described herein further comprise administering to the subject an amount of an anti-cancer therapeutic agent or a palliative agent, which amounts are together effective in treating said abnormal cell growth. In some such embodiments, one or more anti-cancer therapeutic agent are selected from anti-tumor agents, anti-angiogenesis agents, signal transduction inhibitors and antiproliferative agents, which amounts are together effective in treating said abnormal cell growth.

In other embodiments, the uses described herein comprise the use of a compound of one of the formulae described herein or pharmaceutically acceptable salt thereof, in combination with one or more anti-cancer therapeutic agents selected from anti-tumor agents, anti-angiogenesis agents, signal transduction inhibitors and antiproliferative agents.

In some embodiments, the medicaments described herein are adapted for use in combination with one or more anti-cancer therapeutic agents selected from anti-tumor agents, anti-angiogenesis agents, signal transduction inhibitors and antiproliferative agents.

Each of the embodiments of the compounds of the present invention described below can be combined with one or more other embodiments of the compounds of the present invention described herein not inconsistent with the embodiment(s) with which it is combined. In addition, each of the embodiments below describing the invention envisions within its scope the pharmaceutically acceptable salts of the compounds of the invention. Accordingly, the phrase “or a pharmaceutically acceptable salt thereof” is implicit in the description of all compounds described herein.

### DETAILED DESCRIPTION

The present invention may be understood more readily by reference to the following detailed description of the preferred embodiments of the invention and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

As used herein, the singular form “a”, “an”, and “the” include plural references unless indicated otherwise. For example, “a” substituent includes one or more substituents.

“Alkyl” refers to a saturated, monovalent aliphatic hydrocarbon radical including straight chain and branched chain groups having the specified number of carbon atoms. Alkyl substituents typically contain 1 to 20 carbon atoms (“C₁-C₂₀ alkyl”), preferably 1 to 12 carbon atoms (“C₁-C₁₂ alkyl”), more preferably 1 to 8 carbon atoms (“C₁-C₈ alkyl”), or 1 to 6 carbon atoms (“C₁-C₆ alkyl”), or 1 to 4 carbon atoms (“C₁-C₄ alkyl”). Examples of alkyl groups include methyl, ethyl, n-propyl, isopropyl, n-butyl, iso-butyl, tert-butyl, n-pentyl, isopentyl, neopentyl, n-hexyl, n-heptyl, n-octyl and the like. Alkyl groups may be substituted or unsubstituted. In particular, unless otherwise specified, alkyl groups may be substituted by one or more halo groups, up to the total number of hydrogen atoms present on the alkyl moiety. Thus, C₁-C₄ alkyl includes halogenated alkyl groups, e.g., trifluoromethyl or difluoroethyl (i.e., CF₃ and —CH₂CHF₂).

Alkyl groups described herein as optionally substituted by may be substituted by one or more substituent groups, which are selected independently unless otherwise indicated. The total number of substituent groups may equal the total number of hydrogen atoms on the alkyl moiety, to the extent such substitution makes chemical sense. Optionally substituted alkyl groups typically contain from 1 to 6 optional substituents, sometimes 1 to 5 optional substituents, preferably from 1 to 4 optional substituents, or more preferably from 1 to 3 optional substituents.

Optional substituent groups suitable for alkyl include, but are not limited to C₃-C₈ cycloalkyl, 3-12 membered heterocyclyl, C₆-C₁₂ aryl and 5-12 membered heteroaryl, halo, ═O (oxo), ═S (thiono), ═N—CN, ═N—OR^{x}, ═NR^{x}, —CN, —COR^{x}, —CO₂R^{x}, —CONR^{x}R^{y}, —SR^{x}, —SOR^{x}, —SO₂R^{x}, —SO₂NR^{x}R^{y}, —NO₂, —NR^{x}R^{y}, —NR^{x}C(O)R^{y}, —NR^{x}C(O)NR^{x}R^{y}, —NR^{x}C(O)OR^{x}, —NR^{x}SO₂R^{y}, —NR^{x}SO₂NR^{x}R^{y}, —OR^{x}, —OC(O)R^{x} and —OC(O)NR^{x}R^{y}; wherein each R^{x} and R^{y} is independently H, C₁-C₈ alkyl, C₁-C₈ acyl, C₂-C₈ alkenyl, C₂-C₈ alkynyl, C₃-C₈ cycloalkyl, 3-12 membered heterocyclyl, C₆-C₁₂ aryl, or 5-12 membered heteroaryl, or R^{x} and R^{y} may be taken together with the N atom to which they are attached to form a 3-12 membered heterocyclyl or 5-12 membered heteroaryl, each optionally containing 1, 2 or 3 additional heteroatoms selected from O, N and S; each R^{x} and R^{y} is optionally substituted with 1 to 3 substituents independently selected from the group consisting of halo, ═O, ═S, ═N—CN, ═N—OR′, ═NR′, —CN, —COR′, —CO₂R′, —CONR′₂, —SR′, —SOR′, —SO₂R′, —SO₂NR′₂, —NO₂, —NR′₂, —NR′C(O)R′, —NR′C(O)NR′₂, —NR′C(O)OR′, —NR′SO₂R′, —NR′SO₂NR′₂, —OR′, —OC(O)R′ and —OC(O)NR′₂, wherein each R′ is independently H, C₁-C₈ alkyl, C₁-C₈ acyl, C₂-C₈ alkenyl, C₂-C₈ alkynyl, C₃-C₈ cycloalkyl, 3-12 membered heterocyclyl, C₆-C₁₂ aryl, or C₅-C₁₂ heteroaryl; and wherein each said C₃-C₈ cycloalkyl, 3-12 membered heterocyclyl, C₆-C₁₂ aryl and 5-12 membered heteroaryl is optionally substituted as further defined herein.

Typical substituent groups on alkyl include halo, —OH, C₁-C₄ alkoxy, —O—C₆-C₁₂ aryl, —CN, ═O, —COOR^{x}, —OC(O)R^{x}, —CONR^{x}R^{y}, —NR^{x}C(O)R^{y}, —NR^{x}R^{y}, C₃-C₈ cycloalkyl, C₆-C₁₂ aryl, 5-12 membered heteroaryl and 3-12 membered heterocyclyl; where each R^{x} and R^{y} is independently H or C₁-C₄ alkyl, or R^{x} and R^{y} may be taken together with the N to which they are attached form a 3-12 membered heterocyclyl or 5-12 membered heteroaryl ring, each optionally containing 1, 2 or 3 additional heteroatoms selected from O, N and S; wherein each said C₃-C₈ cycloalkyl, C₆-C₁₂ aryl, 5-12 membered heteroaryl and 3-12 membered heterocyclyl is optionally substituted by 1 to 3 substituents independently selected from the group consisting of halo, —OH, ═O, C₁-C₄ alkyl, C₁-C₄ alkoxy, C₁-C₆ haloalkyl, C₁-C₆ hydroxyalkyl, C₁-C₄ alkoxy-C₁-C₆ alkyl, —CN, —NH₂, —NH(C₁-C₄ alkyl), and —N(C₁-C₄ alkyl)₂.

In some embodiments, alkyl is optionally substituted by one or more substituents, and preferably by 1 to 3 substituents, which are independently selected from the group consisting of halo, —OH, C₁-C₄ alkoxy, —O—C₆-C₁₂ aryl, —CN, ═O, —COOR^{x}, —OC(O)R^{x}, —CONR^{x}R^{y}, —NR^{x}C(O)R^{y}, —NR^{x}R^{y}, C₃-C₈ cycloalkyl, C₆-C₁₂ aryl, 5-12 membered heteroaryl and 3-12 membered heterocyclyl; where each R^{x} and R^{y} is independently H or C₁-C₄ alkyl, or R^{x} and R^{y} may be taken together with the N to which they are attached form a 3-12 membered heterocyclyl or 5-12 membered heteroaryl ring, each optionally containing 1, 2 or 3 additional heteroatoms selected from O, N and S; and each said C₃-C₈ cycloalkyl, C₆-C₁₂ aryl, 5-12 membered heteroaryl and 3-12 membered heterocyclyl is optionally substituted by 1 to 3 substituents independently selected from the group consisting of halo, —OH, ═O, C₁-C₄ alkyl, C₁-C₄ alkoxy, C₁-C₆ haloalkyl, C₁-C₆ hydroxyalkyl, C₁-C₄ alkoxy-C₁-C₆ alkyl, —CN, —NH₂, —NH(C₁-C₄ alkyl) and —N(C₁-C₄ alkyl)₂.

In other embodiments, alkyl is optionally substituted by one or more substituent, and preferably by 1 to 3 substituents, independently selected from the group consisting of halo, —OH, C₁-C₄ alkoxy, —CN, —NR^{x}R^{y}, C₃-C₈ cycloalkyl, 3-12 membered heterocyclyl, C₆-C₁₂ aryl and 5-12 membered heteroaryl; where each R^{x} and R^{y} is independently H or C₁-C₄ alkyl, or R^{x} and R^{y} may be taken together with the N to which they are attached form a 3-12 membered heterocyclyl or 5-12 membered heteroaryl ring, each optionally containing 1, 2 or 3 additional heteroatoms selected from O, N and S; and where each said cycloalkyl, heterocyclyl, aryl or heteroaryl is optionally substituted by 1 to 3 substituents independently selected from the group consisting of halo, —OH, ═O, C₁-C₄ alkyl, C₁-C₄ alkoxy, C₁-C₆ haloalkyl, C₁-C₆ hydroxyalkyl, C₁-C₄ alkoxy-C₁-C₆ alkyl, —CN, —NH₂, —NH(C₁-C₄ alkyl) and —N(C₁-C₄ alkyl)₂.

In some instances, substituted alkyl groups may be specifically named with reference to the substituent group. For example, “haloalkyl” refers to an alkyl group having the specified number of carbon atoms that is substituted by one or more halo substituents, and typically contain 1-6 carbon atoms and 1, 2 or 3 halo atoms (i.e., “C₁-C₆ haloalkyl”). Thus, a C₁-C₆ haloalkyl group includes trifluoromethyl (—CF₃) and difluoromethyl (—CF₂H).

Similarly, “hydroxyalkyl” refers to an alkyl group having the specified number of carbon atoms that is substituted by one or more hydroxy substituents, and typically contain 1-6 carbon atoms and 1, 2 or 3 hydroxy (i.e., “C₁-C₆ hydroxyalkyl”). Thus, C₁-C₆ hydroxyalkyl includes hydroxymethyl (—CH₂OH) and 2-hydroxyethyl (—CH₂CH₂OH).

“Alkoxyalkyl” refers to an alkyl group having the specified number of carbon atoms that is substituted by one or more alkoxy substituents. Alkoxyalkyl groups typically contain 1-6 carbon atoms in the alkyl portion and are substituted by 1, 2 or 3 C₁-C₄ alkyoxy substituents. Such groups are sometimes described herein as C₁-C₄ alkyoxy-C₁-C₆ alkyl.

“Aminoalkyl” refers to alkyl group having the specified number of carbon atoms that is substituted by one or more substituted or unsubstituted amino groups, as such groups are further defined herein. Aminoalkyl groups typically contain 1-6 carbon atoms in the alkyl portion and are substituted by 1, 2 or 3 amino substituents. Thus, a C₁-C₆ aminoalkyl group includes, for example, aminomethyl (—CH₂NH₂), N,N-dimethylamino-ethyl (—CH₂CH₂N(CH₃)₂), 3-(N-cyclopropylamino)propyl (—CH₂CH₂CH₂NH-^{c}Pr) and N-pyrrolidinylethyl (—CH₂CH₂—N-pyrrolidinyl).

“Alkenyl” refers to an alkyl group, as defined herein, consisting of at least two carbon atoms and at least one carbon-carbon double bond. Typically, alkenyl groups have 2 to 20 carbon atoms (“C₂-C₂₀ alkenyl”), preferably 2 to 12 carbon atoms (“C₂-C₁₂ alkenyl”), more preferably 2 to 8 carbon atoms (“C₂-C₈ alkenyl”), or 2 to 6 carbon atoms (“C₂-C₆ alkenyl”), or 2 to 4 carbon atoms (“C₂-C₄ alkenyl”). Representative examples include, but are not limited to, ethenyl, 1-propenyl, 2-propenyl, 1-, 2-, or 3-butenyl, and the like. Alkenyl groups may be unsubstituted or substituted by the same groups that are described herein as suitable for alkyl.

“Alkynyl” refers to an alkyl group, as defined herein, consisting of at least two carbon atoms and at least one carbon-carbon triple bond. Alkynyl groups have 2 to 20 carbon atoms (“C₂-C₂₀ alkynyl”), preferably 2 to 12 carbon atoms (“C₂-C₁₂ alkynyl”), more preferably 2 to 8 carbon atoms (“C₂-C₈ alkynyl”), or 2 to 6 carbon atoms (“C₂-C₆ alkynyl”), or 2 to 4 carbon atoms (“C₂-C₄ alkynyl”). Representative examples include, but are not limited to, ethynyl, 1-propynyl, 2-propynyl, 1-, 2-, or 3-butynyl, and the like. Alkynyl groups may be unsubstituted or substituted by the same groups that are described herein as suitable for alkyl.

“Alkylene” as used herein refers to a divalent hydrocarbyl group having the specified number of carbon atoms which can link two other groups together. Sometimes it refers to —(CH₂)ₙ— where n is 1-8, and preferably n is 1-4. Where specified, an alkylene can also be substituted by other groups and may include one or more degrees of unsaturation (i.e., an alkenylene or alkynlene moiety) or rings. The open valences of an alkylene need not be at opposite ends of the chain. Thus —CH(Me)- and —C(Me)₂- are also included within the scope of the term ‘alkylenes’, as are cyclic groups such as cyclopropan-1,1-diyl and unsaturated groups such as ethylene (—CH═CH—) or propylene (—CH₂—CH═CH—). Where an alkylene group is described as optionally substituted, the substituents include those typically present on alkyl groups as described herein.

“Heteroalkylene” refers to an alkylene group as described above, wherein one or more non-contiguous carbon atoms of the alkylene chain are replaced by —N(R)—, —O— or —S(O)_{q}—, where R is H or C₁-C₄ alkyl and q is 0-2. For example, the group —O—(CH₂)₁₋₄— is a ‘C₂-C₅’-heteroalkylene group, where one of the carbon atoms of the corresponding alkylene is replaced by O.

“Alkoxy” refers to a monovalent —O-alkyl group, wherein the alkyl portion has the specified number of carbon atoms. Alkoxy groups typically contain 1 to 8 carbon atoms (“C₁-C₈ alkoxy”), or 1 to 6 carbon atoms (“C₁-C₆ alkoxy”), or 1 to 4 carbon atoms (“C₁-C₄ alkoxy”). For example, C₁-C₄ alkoxy includes —OCH₃, —OCH₂CH₃, —OCH(CH₃)₂, —OC(CH₃)₃, and the like. Such groups may also be referred to herein as methoxy, ethoxy, isopropoxy, tert-butyloxy, etc. Alkoxy groups may be unsubstituted or substituted on the alkyl portion by the same groups that are described herein as suitable for alkyl. In particular, alkoxy groups may be substituted by one or more halo groups, up to the total number of hydrogen atoms present on the alkyl portion. Thus, C₁-C₄ alkoxy includes halogenated alkoxy groups, e.g., trifluoromethoxy and 2,2-difluoroethoxy (i.e., —OCF₃ and —OCH₂CHF₂).

Similarly, “thioalkoxy” refers to a monovalent —S-alkyl group, wherein the alkyl portion has the specified number of carbon atoms, and may be optionally substituted on the alkyl portion by the same groups that are described herein as suitable for alkyl. For example, a C₁-C₄ thioalkoxy includes —SCH₃ and —SCH₂CH₃

“Cycloalkyl” refers to a non-aromatic, saturated or partially unsaturated carbocyclic ring system containing the specified number of carbon atoms, which may be a monocyclic, bridged or fused bicyclic or polycyclic ring system that is connected to the base molecule through a carbon atom of the cycloalkyl ring. Typically, the cycloalkyl groups of the invention contain 3 to 12 carbon atoms (“C₃-C₁₂ cycloalkyl”), preferably 3 to 8 carbon atoms (“C₃-C₈ cycloalkyl”). Representative examples include, e.g., cyclopropane, cyclobutane, cyclopentane, cyclopentene, cyclohexane, cyclohexene, cyclohexadiene, cycloheptane, cycloheptatriene, adamantane, and the like. Cycloalkyl groups may be unsubstituted or substituted by the same groups that are described herein as suitable for alkyl.

Illustrative examples of cycloalkyl rings include, but are not limited to, the following:

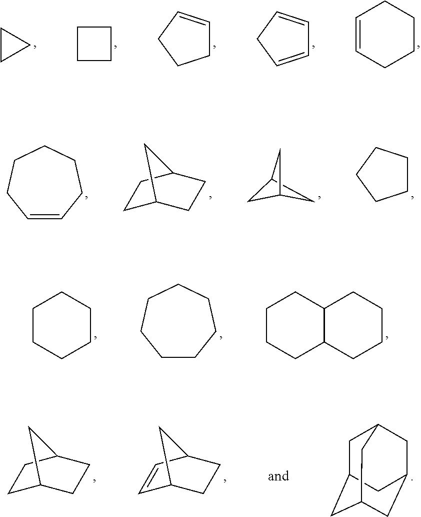

“Cycloalkylalkyl” may be used to describe a cycloalkyl ring, typically a C₃-C₈ cycloalkyl, which is connected to the base molecule through an alkylene linker, typically a C₁-C₄ alkylene. Cycloalkylalkyl groups are described by the total number of carbon atoms in the carbocyclic ring and linker, and typically contain from 4-12 carbon atoms (“C₄-C₁₂ cycloalkylalkyl”). Thus a cyclopropylmethyl group is a C₄-cycloalkylalkyl group and a cyclohexylethyl is a C₈-cycloalkylalkyl. Cycloalkylalkyl groups may be unsubstituted or substituted on the cycloalkyl and/or alkylene portions by the same groups that are described herein as suitable for alkyl groups.

The terms “heterocyclyl”, “heterocyclic” or “heteroalicyclic” may be used interchangeably herein to refer to a non-aromatic, saturated or partially unsaturated ring system containing the specified number of ring atoms, including at least one heteroatom selected from N, O and S as a ring member, wherein the heterocyclic ring is connected to the base molecule via a ring atom, which may be C or N. Heterocyclic rings may be fused to one or more other heterocyclic or carbocyclic rings, which fused rings may be saturated, partially unsaturated or aromatic. Preferably, heterocyclic rings contain 1 to 4 heteroatoms selected from N, O, and S as ring members, and more preferably 1 to 2 ring heteroatoms, provided that such heterocyclic rings do not contain two contiguous oxygen atoms. Heterocyclyl groups may be unsubstituted or substituted by the same groups that are described herein as suitable for alkyl, aryl or heteroaryl moieties. In addition, ring N atoms may be optionally substituted by groups suitable for an amine, e.g., alkyl, acyl, carbamoyl, sulfonyl substituents, etc., and ring S atoms may be optionally substituted by one or two oxo groups (i.e., S(O)_{q}, where q is 0, 1 or 2). Preferred heterocycles include 3-12 membered heterocyclyl groups in accordance with the definition herein. More preferred heterocycles include 4-6 membered heterocyclyl groups in accordance with the definition herein.

Illustrative examples of saturated heterocyclic groups include, but are not limited to:

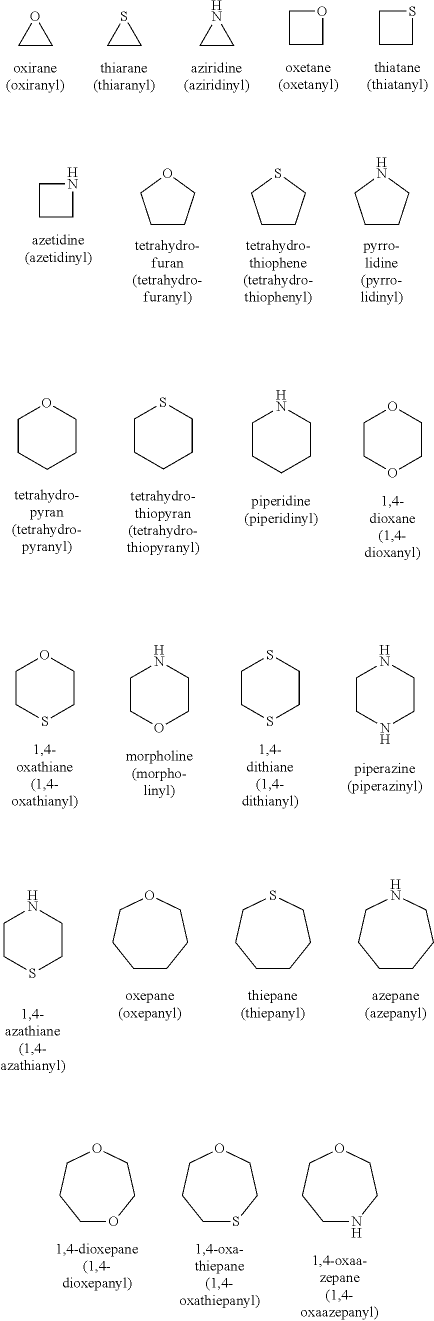

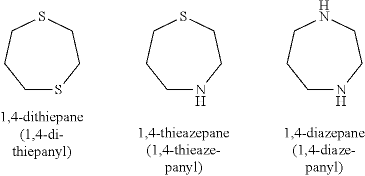 [SMILES: C1CSCCSC1 ; C1CNCCSC1 ; C1CNCCNC1]

Illustrative examples of partially unsaturated heterocyclic groups include, but are not limited to:

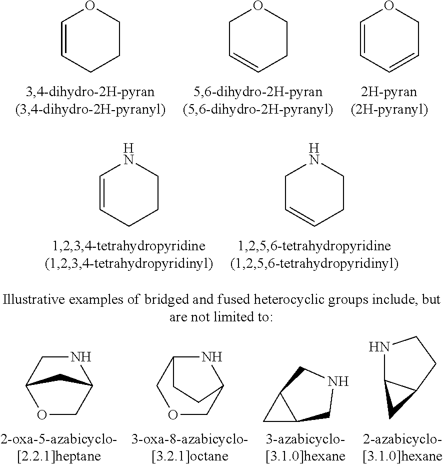

It is understood that no more than two N, O or S atoms are ordinarily connected sequentially, except where an oxo group is attached to N or S to form a nitro or sulfonyl group, or in the case of certain heteroaromatic rings, such as triazine, triazole, tetrazole, oxadiazole, thiadiazole, and the like.

The term “heterocyclylalkyl” may be used to describe a heterocyclic group of the specified size that is connected to the base molecule through an alkylene linker of the specified length. Typically, such groups contain an optionally substituted 3-12 membered heterocycle attached to the base molecule through a C₁-C₄ alkylene linker. Where so indicated, such groups may be optionally substituted on the alkylene portion by the same groups that are described herein as suitable for alkyl groups and on the heterocyclic portion by groups described as suitable for heterocyclic rings.

“Aryl” or “aromatic” refer to an optionally substituted monocyclic or fused bicyclic or polycyclic ring system having the well-known characteristics of aromaticity, wherein at least one ring contains a completely conjugated pi-electron system. Typically, aryl groups contain 6 to 20 carbon atoms (“C₆-C₂₀ aryl”) as ring members, preferably 6 to 14 carbon atoms (“C₆-C₁₄ aryl”) or more preferably, 6 to 12 carbon atoms (“C₆-C₁₂ aryl”). Fused aryl groups may include an aryl ring (e.g., a phenyl ring) fused to another aryl ring, or fused to a saturated or partially unsaturated carbocyclic or heterocyclic ring. The point of attachment to the base molecule on such fused aryl ring systems may be a C atom the aromatic portion or a C or N atom of the non-aromatic portion of the ring system. Examples, without limitation, of aryl groups include phenyl, biphenyl, naphthyl, anthracenyl, phenanthrenyl, indanyl, indenyl, and tetrahydronaphthyl. The aryl group may be unsubstituted or substituted as further described herein.

Similarly, “heteroaryl” or “heteroaromatic” refer to monocyclic or fused bicyclic or polycyclic ring systems having the well-known characteristics of aromaticity that contain the specified number of ring atoms and include at least one heteroatom selected from N, O and S as a ring member in an aromatic ring. The inclusion of a heteroatom permits aromaticity in 5-membered rings as well as 6-membered rings. Typically, heteroaryl groups contain 5 to 20 ring atoms (“5-20 membered heteroaryl”), preferably 5 to 14 ring atoms (“5-14 membered heteroaryl”), and more preferably 5 to 12 ring atoms (“5-12 membered heteroaryl”). Heteroaryl rings are attached to the base molecule via a ring atom of the heteroaromatic ring, such that aromaticity is maintained. Thus, 6-membered heteroaryl rings may be attached to the base molecule via a ring C atom, while 5-membered heteroaryl rings may be attached to the base molecule via a ring C or N atom. Examples of unsubstituted heteroaryl groups include, but are not limited to, pyrrole, furan, thiophene, pyrazole, imidazole, isoxazole, oxazole, isothiazole, thiazole, triazole, oxadiazole, thiadiazole, tetrazole, pyridine, pyridazine, pyrimidine, pyrazine, benzofuran, benzothiophene, indole, benzimidazole, indazole, quinoline, isoquinoline, purine, triazine, naphthryidine and carbazole. The heteroaryl group may be unsubstituted or substituted as further described herein.

Aryl, heteroaryl and heterocyclyl moieties described herein as optionally substituted by may be substituted by one or more substituent groups, which are selected independently unless otherwise indicated. The total number of substituent groups may equal the total number of hydrogen atoms on the aryl, heteroaryl or heterocyclyl moiety, to the extent such substitution makes chemical sense and aromaticity is maintain in the case of aryl and heteroaryl rings. Optionally substituted aryl, heteroaryl or heterocyclyl groups typically contain from 1 to 5 optional substituents, sometimes 1 to 4 optional substituents, preferably 1 to 3 optional substituents, or more preferably from 1-2 optional substituents.

Optional substituent groups suitable for aryl, heteroaryl and heterocyclyl rings include, but are not limited to: C₁-C₈ alkyl, C₂-C₈ alkenyl, C₂-C₈ alkynyl, C₃-C₈ cycloalkyl, 3-12 membered heterocyclyl, C₆-C₁₂ aryl and 5-12 membered heteroaryl; and halo, ═O, —CN, —COR^{x}, —CO₂R^{x}, —CONR^{x}R^{y}, —SR^{x}, —SOR^{x}, —SO₂R^{x}, —SO₂NR^{x}R^{y}, —NO₂, —NR^{x}R^{y}, —NR^{x}C(O)R^{y}, —NR^{x}C(O)NR^{x}R^{y}, —NR^{x}C(O)OR^{x}, —NR^{x}SO₂R^{y}, —NR^{x}SO₂NR^{x}R^{y}, —OR^{x}, —OC(O)R^{x} and —OC(O)NR^{x}R^{y}; where each R^{x} and R^{y} is independently H, C₁-C₈ alkyl, C₁-C₈ acyl, C₂-C₈ alkenyl, C₂-C₈ alkynyl, C₃-C₈ cycloalkyl, 3-12 membered heterocyclyl, C₆-C₁₂ aryl, or 5-12 membered heteroaryl, or R^{x} and R^{y} may be taken together with the N atom to which they are attached to form a 3-12 membered heterocyclyl or 5-12 membered heteroaryl, each optionally containing 1, 2 or 3 additional heteroatoms selected from O, N and S; each R^{x} and R^{y} is optionally substituted with 1 to 3 substituents independently selected from the group consisting of halo, ═O, ═S, ═N—CN, ═N—OR′, ═NR′, —CN, —COR′, —CO₂R′, —CONR′₂, —SR′, —SOR′, —SO₂R′, —SO₂NR′₂, —NO₂, —NR′₂, —NR′C(O)R′, —NR′C(O)NR′₂, —NR′C(O)OR′, —NR′SO₂R′, —NR′SO₂NR′₂, —OR′, —OC(O)R′ and —OC(O)NR′₂, wherein each R′ is independently H, C₁-C₈ alkyl, C₁-C₈ acyl, C₂-C₈ alkenyl, C₂-C₈ alkynyl, C₃-C₈ cycloalkyl, 3-12 membered heterocyclyl, C₆-C₁₂ aryl, or 5-12 membered heteroaryl; and each said C₁-C₈ alkyl, C₂-C₈ alkenyl, C₂-C₈ alkynyl, C₃-C₈ cycloalkyl, 3-12 membered heterocyclyl, C₆-C₁₂ aryl and 5-12 membered heteroaryl is optionally substituted as further defined herein.

In typical embodiments, optional substitution on aryl, heteroaryl and heterocyclyl rings includes one or more substituents, and preferably 1 to 3 substituents, independently selected from the group consisting of halo, C₁-C₈ alkyl, —OH, C₁-C₈ alkoxy, —CN, ═O, —COR^{x}, —COOR^{x}, —OC(O)R^{x}, —CONR^{x}R^{y}, —NR^{x}C(O)R^{y}, —SR^{x}, —SOR^{x}, —SO₂R^{x}, —SO₂NR^{x}R^{y}, —NO₂, —NR^{x}R^{y}, —NR^{x}C(O)R^{y}, —NR^{x}C(O)NR^{x}R^{y}, —NR^{x}C(O)OR^{y}—NR^{x}SO₂R^{y}, —NR^{x}SO₂NR^{x}R^{y}, —OC(O)R^{x}, —OC(O)NR^{x}R^{y}, C₃-C₈ cycloalkyl, 3-12 membered heterocyclyl, C₆-C₁₂ aryl, 5-12 membered heteroaryl, —O—(C₃-C₈ cycloalkyl), —O-(3-12 membered heterocyclyl), —O—(C₆-C₁₂ aryl) and —O—(5-12 membered heteroaryl); where each R^{x} and R^{y} is independently H or C₁-C₄ alkyl, or R^{x} and R^{y} may be taken together with the N to which they are attached form a 3-12 membered heterocyclyl or 5-12 membered heteroaryl ring, each optionally containing 1, 2 or 3 additional heteroatoms selected from O, N and S; and wherein each said C₁-C₈ alkyl, C₁-C₈ alkoxy, C₃-C₈ cycloalkyl, 3-12 membered heterocyclyl, C₆-C₁₂ aryl, 5-12 membered heteroaryl, —O—(C₃-C₈ cycloalkyl), —O-(3-12 membered heterocyclyl), —O—(C₆-C₁₂ aryl) and —O-(5-12 membered heteroaryl) that is described as an optional substituent or is part of R^{x} or R^{y} is optionally substituted by 1 to 3 substituents independently selected from the group consisting of halo, —OH, ═O, C₁-C₄ alkyl, C₁-C₄ alkoxy, C₁-C₆ haloalkyl, C₁-C₆ hydroxyalkyl, C₁-C₄ alkoxy-C₁-C₆ alkyl, —CN, —NH₂, —NH(C₁-C₄ alkyl), —N(C₁-C₄ alkyl)₂ and N-pyrrolidinyl.

Illustrative examples of monocyclic heteroaryl groups include, but are not limited to:

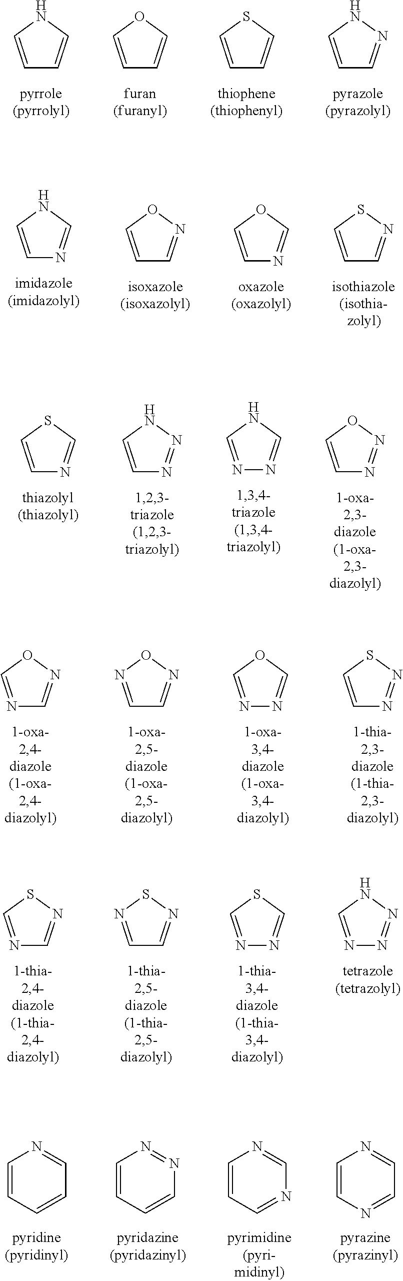

Illustrative examples of fused ring heteroaryl groups include, but are not limited to:

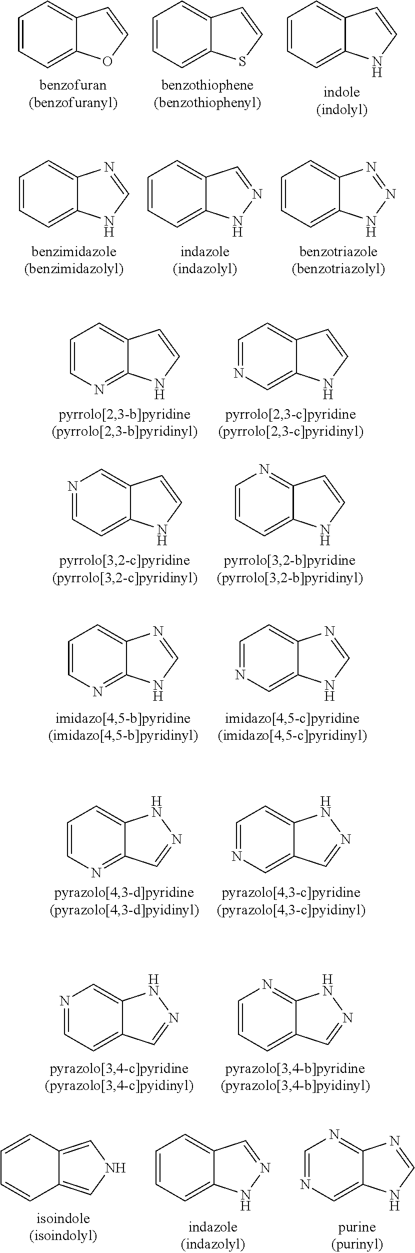

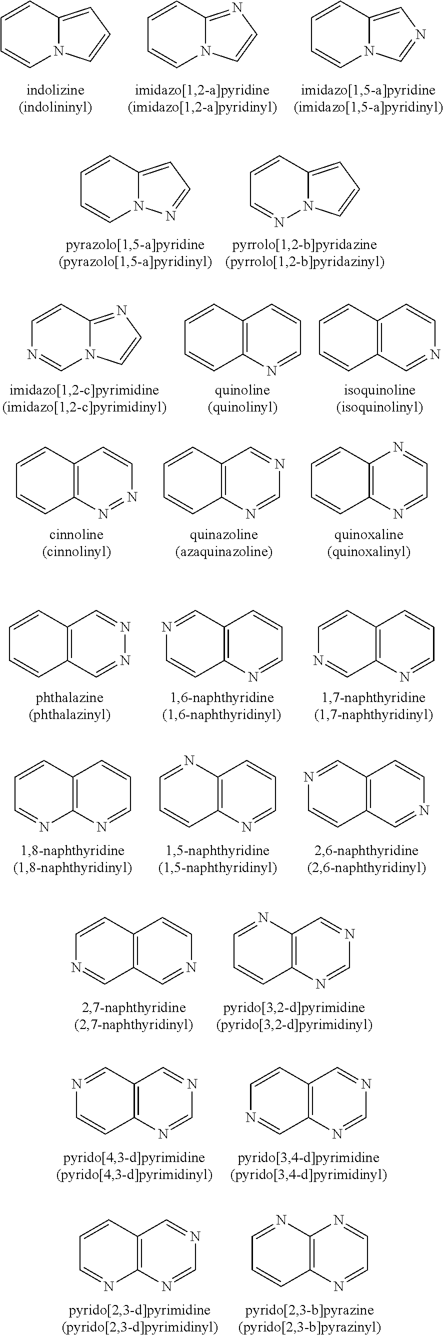

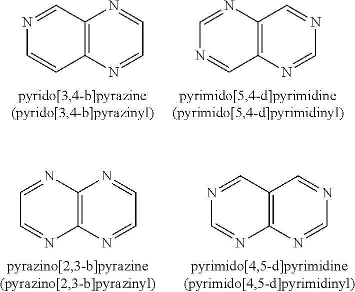 [SMILES: c1cc2nccnc2cn1 ; c1ncc2ncncc2n1 ; c1cnc2nccnc2n1 ; c1ncc2cncnc2n1]

An “arylalkyl” group refers to an aryl group as described herein which is linked to the base molecule through an alkylene or similar linker. Arylalkyl groups are described by the total number of carbon atoms in the ring and linker. Thus a benzyl group is a C₇-arylalkyl group and a phenylethyl is a C₈-arylalkyl. Typically, arylalkyl groups contain 7-16 carbon atoms (“C₇-C₁₆ arylalkyl”), wherein the aryl portion contains 6-12 carbon atoms and the alkylene portion contains 1-4 carbon atoms. Such groups may also be represented as —C₁-C₄ alkylene-C₆-C₁₂ aryl.

“Heteroarylalkyl” refers to a heteroaryl group as described above that is attached to the base molecule through an alkylene linker, and differs from “arylalkyl” in that at least one ring atom of the aromatic moiety is a heteroatom selected from N, O and S. Heteroarylalkyl groups are sometimes described herein according to the total number of non-hydrogen atoms (i.e., C, N, S and O atoms) in the ring and linker combined, excluding substituent groups. Thus, for example, pyridinylmethyl may be referred to as a “C₇”-heteroarylalkyl. Typically, unsubstituted heteroarylalkyl groups contain 6-20 non-hydrogen atoms (including C, N, S and O atoms), wherein the heteroaryl portion typically contains 5-12 atoms and the alkylene portion typically contains 1-4 carbon atoms. Such groups may also be represented as —C₁-C₄ alkylene-5-12 membered heteroaryl.

Similarly, “arylalkoxy” and “heteroarylalkoxy” refer to aryl and heteroaryl groups, attached to the base molecule through a heteroalkylene linker (i.e., —O-alkylene-), wherein the groups are described according to the total number of non-hydrogen atoms (i.e., C, N, S and O atoms) in the ring and linker combined. Thus, —O—CH₂-phenyl and —O—CH₂-pyridinyl groups would be referred to as C₈-arylalkoxy and C₈-heteroarylalkoxy groups, respectively.

Where an arylalkyl, arylalkoxy, heteroarylalkyl or heteroarylalkoxy group is described as optionally substituted, the substituents may be on either the divalent linker portion or on the aryl or heteroaryl portion of the group. The substituents optionally present on the alkylene or heteroalkylene portion are the same as those described above for alkyl or alkoxy groups generally, while the substituents optionally present on the aryl or heteroaryl portion are the same as those described above for aryl or heteroaryl groups generally.

“Hydroxy” refers to an —OH group.

“Acyloxy” refers to a monovalent group —OC(O)alkyl, wherein the alkyl portion has the specified number of carbon atoms (typically C₁-C₈, preferably C₁-C₆ or C₁-C₄) and may be optionally substituted by groups suitable for alkyl. Thus, C₁-C₄ acyloxy includes an —OC(O)C₁-C₄ alkyl substituent, e.g., —OC(O)CH₃.

“Acylamino” refers to a monovalent group, —NHC(O)alkyl or —NRC(O)alkyl, wherein the alkyl portion has the specified number of carbon atoms (typically C₁-C₈, preferably C₁-C₆ or C₁-C₄) and may be optionally substituted by groups suitable for alkyl. Thus, C₁-C₄ acylamino includes an —NHC(O)C₁-C₄ alkyl substituent, e.g., —NHC(O)CH₃.

“Aryloxy” or “heteroaryloxy” refer to optionally substituted —O-aryl or —O-heteroaryl, in each case where aryl and heteroaryl are as further defined herein.

“Arylamino” or “heteroarylamino” refer to optionally substituted —NH-aryl, —NR-aryl, —NH— heteroaryl or —NR-heteroaryl, in each case where aryl and heteroaryl are as further defined herein and R represents a substituent suitable for an amine, e.g., an alkyl, acyl, carbamoyl or sulfonyl group, or the like.

“Cyano” refers to a —C≡N group.

“Unsubstituted amino” refers to a group —NH₂. Where the amino is described as substituted or optionally substituted, the term includes groups of the form —NR^{x}R^{y}, where each or R^{x} and R^{y} is independently H, alkyl, alkenyl, alkynyl, cycloalkyl, heterocyclyl, acyl, thioacyl, aryl, heteroaryl, cycloalkylalkyl, arylalkyl or heteroarylalkyl, in each case having the specified number of atoms and optionally substituted as described herein. For example, “alkylamino” refers to a group —NR^{x}R^{y}, wherein one of R^{x} and R^{y} is an alkyl moiety and the other is H, and “dialkylamino” refers to —NR^{x}R^{y} wherein both of R^{x} and R^{y} are alkyl moieties, where the alkyl moieties having the specified number of carbon atoms (e.g., —NH—C₁-C₄ alkyl or —N(C₁-C₄ alkyl)₂). Typically, alkyl substituents on amines contain 1 to 8 carbon atoms, preferably 1 to 6 carbon atoms, or more preferably 1 to 4 carbon atoms. The term also includes forms wherein R^{x} and R^{y} are taken together with the N atom to which they are attached to form a 3-12 membered heterocyclyl or 5-12 membered heteroaryl ring, each of which may itself be optionally substituted as described herein for heterocyclyl or heteroaryl rings, and which may contain 1 to 3 additional heteroatoms selected from N, O and S as ring members, provided that such rings do not contain two contiguous oxygen atoms.

“Halogen” or “halo” refers to fluoro, chloro, bromo and iodo (F, Cl, Br, I). Preferably, halo refers to fluoro or chloro (F or Cl).

“Heteroform” is sometimes used herein to refer to a derivative of a group such as, e.g., an alkyl, aryl, or acyl, wherein at least one carbon atom of the designated carbocyclic group has been replaced by a heteroatom selected from N, O and S. Thus the heteroforms of alkyl, alkenyl, alkynyl, acyl, aryl, and arylalkyl are heteroalkyl, heteroalkenyl, heteroalkynyl, heteroacyl, heteroaryl, and heteroarylalkyl, respectively. It is understood that no more than two N, O or S atoms are ordinarily connected sequentially, except where an oxo group is attached to N or S to form a nitro or sulfonyl group.

“Optional” or “optionally” means that the subsequently described event or circumstance may but need not occur, and the description includes instances where the event or circumstance occurs and instances in which it does not.

The terms “optionally substituted” and “substituted or unsubstituted” may be used interchangeably to indicate that the particular group being described may have no non-hydrogen substituents (i.e., unsubstituted), or the group may have one or more non-hydrogen substituents (i.e., substituted). If not otherwise specified, the total number of substituents that may be present is equal to the number of H atoms present on the unsubstituted form of the group being described, to the extent that such substitution makes chemical sense. Where an optional substituent is attached via a double bond, such as an oxo (═O) substituent, the group occupies two available valences, so the total number of other substituents that may be included is reduced by two. In the case where optional substituents are selected independently from a list of alternatives, the selected groups may be the same or different.

In one aspect, the invention provides a compound of formula (I):

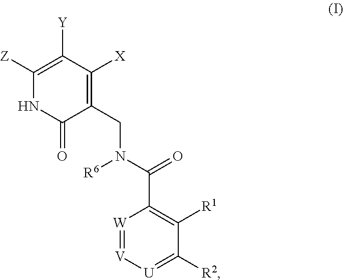

or a pharmaceutically acceptable salt thereof,

wherein:

U is N or CR³;

V is N or CR⁴;

W is N or CR⁵;

R¹ is C₁-C₈ alkyl, C₁-C₈ alkoxy, halo, —OH, —CN or —NR⁷R⁸, where each said C₁-C₈ alkyl or C₁-C₈ alkoxy is optionally substituted by one or more R²¹;

R² is 3-12 membered heterocyclyl, C₆-C₁₂ aryl, 5-12 membered heteroaryl or C₁-C₈ alkoxy, where said C₁-C₈ alkoxy is optionally substituted by one or more R²², and each said heterocyclyl, aryl or heteroaryl is optionally substituted by one or more R³²;

R³ is H, C₁-C₈ alkyl, C₁-C₈ alkoxy, halo, —OH, —CN or —NR⁷R⁸, where each said C₁-C₈ alkyl or C₁-C₈ alkoxy is optionally substituted by one or more R²³;

R⁴ is independently selected from the group consisting of H, C₁-C₈ alkyl, C₁-C₈ alkoxy, C₁-C₈ thioalkoxy, halo, —OH, —CN, C₃-C₈ cycloalkyl, 3-12 membered heterocyclyl, C₆-C₁₂ aryl, 5-12 membered heteroaryl, —OR¹¹ and —NR⁷R⁸, where each said C₁-C₈ alkyl, C₁-C₈ alkoxy, C₁-C₈ thioalkoxy or C₃-C₈ cycloalkyl is optionally substituted by one or more R²⁴, and each said heterocyclyl, aryl, heteroaryl or R¹¹ is optionally substituted by one or more R³⁴;

R⁵ is H, C₁-C₈ alkyl, C₁-C₈ alkoxy, halo, —OH, —CN or —NR⁷R⁸, where each said C₁-C₈ alkyl or C₁-C₈ alkoxy is optionally substituted by one or more R²⁵;

R⁶ is H or C₁-C₄ alkyl;

each R⁷ and R⁸ is independently H or C₁-C₈ alkyl, where said C₁-C₈ alkyl is optionally substituted by one or more R²⁷; or

R⁷ and R⁸ may be taken together with the N atom to which they are attached to form a 3-12 membered heterocyclyl or 5-12 membered heteroaryl, each optionally containing 1, 2 or 3 additional heteroatoms selected from O, N and S, wherein each said heterocyclyl or heteroaryl is optionally substituted by one or more R³⁷;

each R²¹, R²², R²³ and R²⁵ is independently selected from the group consisting of halo, —OH, C₁-C₄ alkoxy, —CN and —NR⁹R¹⁰;

each R²⁴ and R²⁷ is independently selected from the group consisting of halo, —OH, C₁-C₄ alkoxy, —CN, —NR⁹R¹⁰, C₃-C₈ cycloalkyl, 3-12 membered heterocyclyl, C₆-C₁₂ aryl and 5-12 membered heteroaryl, where each said cycloalkyl, heterocyclyl, aryl or heteroaryl is optionally substituted by one or more substituents independently selected from the group consisting of halo, —OH, ═O, C₁-C₄ alkyl, C₁-C₄ alkoxy, —CN, —NH₂, —NH(C₁-C₄ alkyl) and —N(C₁-C₄ alkyl)₂;

each R⁹ and R¹⁰ is independently H or C₁-C₄ alkyl; or

R⁹ and R¹⁰ may be taken together with the N atom to which they are attached to form a 3-12 membered heterocyclyl or 5-12 membered heteroaryl, each optionally containing 1, 2 or 3 additional heteroatoms selected from O, N and S, where each said heterocyclyl or heteroaryl is optionally substituted by one or more substituents independently selected from the group consisting of halo, —OH, ═O, C₁-C₄ alkyl, C₁-C₄ alkoxy, —CN, —NH₂, —NH(C₁-C₄ alkyl) and —N(C₁-C₄ alkyl)₂;

R¹¹ is selected from the group consisting of C₃-C₈ cycloalkyl, 3-12 membered heterocyclyl, C₆-C₁₂ aryl and 5-12 membered heteroaryl;

each R³², R³⁴ and R³⁷ is independently selected from the group consisting of halo, C₁-C₈ alkyl, —CN, ═O, —COR^{c}, —CO₂R^{c}, —CONR^{c}R^{d}, —OR^{c}, —SOR^{c}, —SOR^{c}, —SO₂R^{c}, —SO₂NR^{c}R^{d}, —NO₂, —NR^{c}R^{d}, —NR^{c}C(O)R^{d}, —NR^{c}C(O)NR^{c}R^{d}, —NR^{c}C(O)OR^{d}—NR^{c}SO₂R^{d}, —NR^{c}SO₂NR^{c}R^{d}, —OC(O)R^{c}, —OC(O)NR^{c}R^{d}, C₃-C₈ cycloalkyl, 3-12 membered heterocyclyl, C₆-C₁₂ aryl and 5-12 membered heteroaryl;
each R^{c} and R^{d} is independently selected from the group consisting of H, C₁-C₈ alkyl, C₃-C₈ cycloalkyl, 3-12 membered heterocyclyl, C₆-C₁₂ aryl and 5-12 membered heteroaryl; or
R^{c} and R^{d} may be taken together with the N atom to which they are attached to form a 3-12 membered heterocyclyl or 5-12 membered heteroaryl ring, each optionally containing 1, 2 or 3 additional heteroatoms selected from O, N and S;
wherein each said alkyl, cycloalkyl, heterocyclyl, aryl or heteroaryl in R³², R³⁴, R³⁷, R^{c} and R^{d} is optionally substituted by 1 to 3 substituents independently selected from the group consisting of halo, —OH, ═O, C₁-C₄ alkyl, C₁-C₄ alkoxy, C₁-C₆ haloalkyl, C₁-C₆ hydroxyalkyl, C₁-C₄ alkoxy-C₁-C₆ alkyl, —CN, —NH₂, —NH(C₁-C₄ alkyl) and —N(C₁-C₄ alkyl)₂;

X and Z are independently selected from the group consisting of H, C₁-C₈ alkyl, C₂-C₈ alkenyl, C₂-C₈ alkynyl, C₃-C₈ cycloalkyl, 3-12 membered heterocyclyl, C₆-C₁₂ aryl, 5-12 membered heteroaryl, halo, CN, —COR^{a}, —CO₂R^{a}, —CONR^{a}R^{b}, —SR^{a}, —SOR^{a}, —SO₂R^{a}, —SO₂NR^{a}R^{b}, —NO₂, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}—OR^{a}, —OC(O)R^{a} or —OC(O)NR^{a}R^{b};
wherein each said C₁-C₈ alkyl, C₂-C₈ alkenyl, C₂-C₈ alkynyl, C₃-C₈ cycloalkyl, 3-12 membered heterocyclyl, C₆-C₁₂ aryl, or 5-12 membered heteroaryl group is optionally substituted by one or more substituents independently selected from the group consisting of halo, —CN, —COR^{a}, —CO₂R^{a}, —CONR^{a}R^{b}, —SR^{a}, —SOR^{a}, —SO₂R^{a}, —SO₂NR^{a}R^{b}, —NO₂, —NR^{a}R^{b}, —NR^{a}C(O)R^{b}, —NR^{a}C(O)NR^{a}R^{b}, —NR^{a}C(O)OR^{a}—NR^{a}SO₂R^{b}, —NR^{a}SO₂NR^{a}R^{b}, —OR^{a}, —OC(O)R^{a}, —OC(O)NR^{a}R^{b}, C₃-C₈ cycloalkyl, 3-12 membered heterocyclyl, C₆-C₁₂ aryl, and 5-12 membered heteroaryl;
each R^{a} and R^{b} is independently H, C₁-C₈ alkyl, C₂-C₈ alkenyl, C₂-C₈ alkynyl, C₃-C₈ cycloalkyl, 3-12 membered heterocyclyl, C₆-C₁₂ aryl or 5-12 membered heteroaryl, where each said alkyl, alkenyl, alkynyl, cycloalkyl, heterocyclyl, aryl and heteroaryl is optionally substituted by one or more substituents independently selected from the group consisting of halo, C₁-C₄ alkyl, —OR″, —NR″₂, —CO₂R″, —CONR″₂, —SO₂R″ and —SO₂NR″₂, where each R″ is independently H or C₁-C₄ alkyl; or
R^{a} and R^{b} may be taken together with the N atom to which they are attached to form a 3-12 membered heterocyclyl or 5-12 membered heteroaryl, each optionally containing 1, 2 or 3 additional heteroatoms selected from O, N and S, wherein said heterocyclyl or heteroaryl is optionally substituted by one or more substituents independently selected from the group consisting of halo, —OH, ═O, C₁-C₄ alkyl, C₁-C₄ alkoxy, C₁-C₆ haloalkyl, C₁-C₆ hydroxyalkyl, C₁-C₄ alkoxy-C₁-C₆ alkyl, —CN, —NH₂, —NH(C₁-C₄ alkyl) and —N(C₁-C₄ alkyl)₂; and

Y is H, halo, —OH or C₁-C₄ alkoxy.

In compounds of formula (I), each or U, V and W is independently selected from N and a substituted carbon atom (i.e., CR³, CR⁴ and CR⁵, respectively), such that the core ring containing U, V and W can be variously a phenyl, pyridinyl, pyrimidinyl, pyridazinyl or triazinyl ring. In some embodiments of formula (I), no more than two of U, V and W are N. In other embodiments of formula (I), no more than one of U, V and W is N. In other embodiments of formula (I), two of U, V and W are N. In other embodiments of formula (I), one of U, V and W are N. In still further embodiments, none of U, V and W is N.

In one embodiment of formula (I), U is CR³, V is CR⁴ and W is CR⁵, such that the ring containing U, V and W is a phenyl ring. In some such embodiments, R³ is H or F, preferably H. In other such embodiments, R⁵ is H or F, preferably H. In some embodiments, R³ and R⁵ are H, such that U is CH, V is CR⁴ and W is CH.

In another embodiment of formula (I), U is N, V is CR⁴ and W is CR⁵, such that the ring containing U, V and W is a 4-carboxamide substituted pyridine ring. In some such embodiments, R⁵ is H or F, preferably H.

In another embodiment of formula (I), U is CR³, V is CR⁴ and W is N, such that the ring containing U, V and W is a 2-carboxamide substituted pyridine ring. In some such embodiments, R³ is H or F, preferably H.

In yet another embodiment of formula (I), U is N, V is CR⁴ and W is N, such that the ring containing U, V and W is a 4-carboxamide substituted pyrimidine ring.

In compounds of formula (I), R¹ is C₁-C₈ alkyl, C₁-C₈ alkoxy, halo, —OH, —CN or —NR⁷R⁸, where each said C₁-C₈ alkyl or C₁-C₈ alkoxy is optionally substituted by one or more R²¹ groups. In some such embodiments, said C₁-C₈ alkyl or C₁-C₈ alkoxy is optionally substituted by 1 to 3 R²¹ groups.

In frequent embodiments of formula (I), R¹ is optionally substituted C₁-C₈ alkyl or halo. In some such embodiments, R¹ is optionally substituted C₁-C₄ alkyl or halo. In further embodiments, R¹ is C₁-C₄ alkyl or halo. In other embodiments, R¹ is C₁-C₄ alkyl, preferably methyl or ethyl. In other embodiments, R¹ is halo, preferably chloro or fluoro (Cl or F). In specific embodiments, R¹ is methyl, ethyl, chloro or fluoro.

In compounds of formula (I), each R²¹ is independently selected from the group consisting of halo, —OH, C₁-C₄ alkoxy, —CN and —NR⁹R¹⁰. When R²¹ is —NR⁹R¹⁰, each R⁹ and R¹⁰ is independently H or C₁-C₄ alkyl, or R⁹ and R¹⁰ may be taken together with the N atom to which they are attached to form an optionally substituted 3-12 membered heterocyclyl or an optionally substituted 5-12 membered heteroaryl moiety, optionally containing 1, 2 or 3 additional heteroatoms selected from N, O and S. In some such embodiments, each said 3-12 membered heterocyclyl or 5-12 membered heteroaryl moiety is optionally substituted by 1 to 3 substituents independently selected from the group consisting of halo, —OH, ═O, C₁-C₄ alkyl, C₁-C₄ alkoxy, —CN, —NH₂, —NH(C₁-C₄ alkyl) and —N(C₁-C₄ alkyl)₂.

In certain embodiments of formula (I), each R²¹ is independently selected from the group consisting of —OH, —Cl, —F, —OCH₃, —OC₂H₅, —OCF₃, —CN, —NH₂, —NHCH₃, —N(CH₃)₂ and N-pyrrolidinyl.

In compounds of formula (I), R² is 3-12 membered heterocyclyl, C₆-C₁₂ aryl, 5-12 membered heteroaryl or C₁-C₈ alkoxy, where said C₁-C₈ alkoxy is optionally substituted by one or more R²², and each said heterocyclyl, aryl or heteroaryl is optionally substituted by one or more R³². In some embodiments, said C₁-C₈ alkoxy is optionally substituted by 1 to 3 R²² groups, and each said heterocyclyl, aryl or heteroaryl is optionally substituted by 1 to 3 R³² groups.

In one embodiment, R² is C₁-C₈ alkoxy, where said C₁-C₈ alkoxy is optionally substituted by one or more R²² groups. In some embodiments, R² is C₁-C₈ alkoxy optionally substituted by 1 to 3 R²² groups. In some such embodiments, R² is C₁-C₄ alkoxy optionally substituted by 1 to 3 R²². In specific embodiments, said C₁-C₄ alkoxy is methoxy, ethoxy, propoxy, isopropoxy, n-butoxy, sec-butoxy or tert-butoxy.

Each R²² is independently selected from the group consisting of halo, —OH, C₁-C₄ alkoxy, —CN and —NR⁹R¹⁰. When R²² is —NR⁹R¹⁰, each R⁹ and R¹⁰ is independently H or C₁-C₄ alkyl, or R⁹ and R¹⁰ may be taken together with the N atom to which they are attached to form an optionally substituted 3-12 membered heterocyclyl or an optionally substituted 5-12 membered heteroaryl moiety, optionally containing 1, 2 or 3 additional heteroatoms selected from N, O and S. In some such embodiments, each said 3-12 membered heterocyclyl or 5-12 membered heteroaryl moiety is optionally substituted by 1 to 3 substituents independently selected from the group consisting of halo, —OH, ═O, C₁-C₄ alkyl, C₁-C₄ alkoxy, —CN, —NH₂, —NH(C₁-C₄ alkyl) and —N(C₁-C₄ alkyl)₂.

In certain embodiments, each R²² is independently selected from the group consisting of —OH, Cl, F, —OCH₃, —OC₂H₅, —OCF₃, —CN, —NH₂, —NHCH₃, —N(CH₃)₂, optionally substituted 4-6 membered heterocyclyl and optionally substituted 5-6 membered heteroaryl. In some embodiments, said 4-6 membered heterocyclyl or said heteroaryl 5-6 membered heteroaryl is optionally substituted by 1 to 3 substituents independently selected from the group consisting of halo, —OH, ═O, C₁-C₄ alkyl, C₁-C₄ alkoxy, —CN, —NH₂, —NH(C₁-C₄ alkyl) and —N(C₁-C₄ alkyl)₂.

In specific embodiments each R²² is independently selected from the group consisting of —OH, Cl, F, —OCH₃, —OC₂H₅, —OCF₃, —CN, —NH₂, —NHCH₃, —N(CH₃)₂ and N-pyrrolidinyl.

In another embodiment of formula (I), R² is 5-12 membered heteroaryl, where said heteroaryl is optionally substituted by one or more R³². In some such embodiments, said 5-12 membered heteroaryl is optionally substituted by 1 to 3 R³² groups.

In some such embodiments, R² is a 5-6 membered heteroaryl, optionally substituted by 1 to 3 R³² groups. In some such embodiments, said 5-6 membered heteroaryl is selected from the group consisting of pyrazolyl, imidazolyl, pyrrolyl, triazolyl, tetrazolyl, thienyl, thiazolyl, isothiazolyl, furanyl, oxazoyl, isoxazolyl, oxadiazolyl, thiadiazolyl, pyridinyl, pyrimidinyl, pyrazinyl and pyridazinyl, each of which may be optionally substituted by 1 to 3 R³² groups.

In specific embodiments, R² may be selected from the following 5-6 membered heteroaryl groups, where the asterisk (*) represents the point of attachment to the base molecule and the optional substituent groups R³² may be present on any atom of the heteroaryl ring (N or C) bearing a H atom in its unsubstituted form:

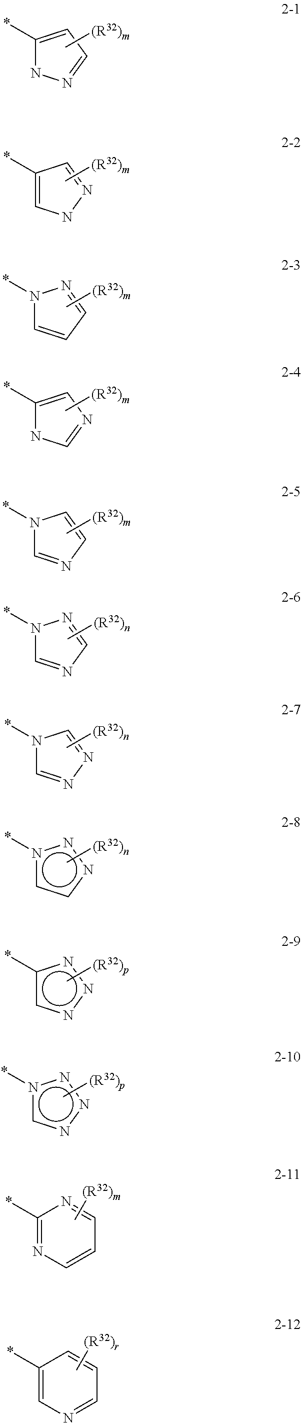

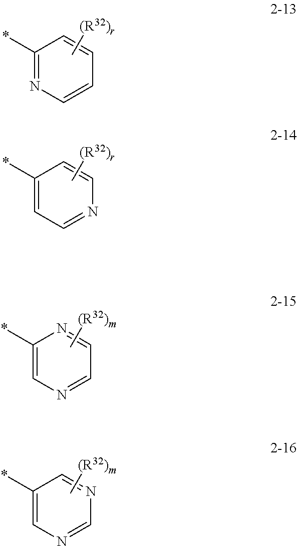

where m is 0, 1, 2 or 3;

n is 0, 1 or 2;

p is 0 or 1; and

r is 0, 1, 2, 3 or 4.

In another embodiment, R² is 3-12 membered heterocyclyl, where said heterocyclyl is optionally substituted by one or more R³² groups. In some embodiments, said heterocyclyl is optionally substituted by 1 to 3 R³² groups. In some such embodiments, said 3-12 membered heterocyclyl is selected from the group consisting of pyrrolidinyl, piperidinyl, morpholinyl, piperazinyl, 2-oxa-5-azabicyclo[2.2.1]heptanyl, 3-oxa-8-azabicyclo[3.2.1]octanyl, dihydropyranyl, tetrahydrofuranyl and tetrahydropyranyl, each optionally substituted by 1 to 3 R³² groups.

In yet another embodiment, R² is C₆-C₁₂ aryl, where said aryl is optionally substituted by one or more R³². In some such embodiments, said aryl is optionally substituted by 1 to 3 R³² groups. In specific embodiments, said aryl is selected from the group consisting of phenyl, biphenyl, naphthyl, indanyl, indenyl and tetrahydronaphthyl, each optionally substituted by 1 to 3 R³² groups.

In compounds of formula (I), when R² is 3-12 membered heterocyclyl, C₆-C₁₂ aryl, or 5-12 membered heteroaryl, each of said heterocyclyl, aryl and heteroaryl is optionally substituted by one or more R³² (preferably 1 to 3 R³²), where each R³² is independently selected from the group consisting of halo, C₁-C₈ alkyl, —CN, ═O, —COR^{c}, —CO₂R^{c}, —CONR^{c}R^{d}, —OR^{c}, —SR^{c}, —SOR^{c}, —SO₂R^{c}, —SO₂NR^{c}R^{d}, —NO₂, —NR^{c}R^{d}, —NR^{c}C(O)R^{d}, —NR^{c}C(O)NR^{c}R^{d}, —NR^{c}C(O)OR^{d}, —NR^{c}SO₂R^{d}, —NR^{c}SO₂NR^{c}R^{d}, —OC(O)R^{c}, —OC(O)NR^{c}R^{d}, C₃-C₈ cycloalkyl, 3-12 membered heterocyclyl, C₆-C₁₂ aryl and 5-12 membered heteroaryl, and R^{c} and R^{d} are defined as in formula (I) above.

In some such embodiments, each R³² is independently halo, C₁-C₈ alkyl, —CN, —CONR^{c}R^{d}, —NR^{c}R^{d}, —NR^{c}C(O)R^{d}, C₃-C₈ cycloalkyl, C₆-C₁₂ aryl and 5-12 membered heteroaryl, where said C₁-C₈ alkyl is optionally substituted by —OH, —C₁-C₄ alkoxy or halo, and each R^{c} and R^{d} is independently H or C₁-C₄ alkyl. In other embodiments, each R³² is independently halo, C₁-C₈ alkyl, —CN, —CONR^{c}R^{d}, —NR^{c}R^{d}, —NR^{c}C(O)R^{d}, C₃-C₈ cycloalkyl, C₆-C₁₂ aryl and 5-12 membered heteroaryl, where said C₁-C₈ alkyl is optionally substituted by —OH, —C₁-C₄ alkoxy or halo; and each R^{c} and R^{d} is independently H or C₁-C₄ alkyl; or R^{c} and R^{d} in —NR^{c}R^{d} may be taken together with the N atom to which they are attached to form a 4-6 membered heterocyclyl optionally containing 1 additional heteroatom selected from O, N and S, where said 4-6 membered heterocyclyl is optionally substituted by 1 to 3 substituents independently selected from the group consisting of halo, —OH, ═O, C₁-C₄ alkyl, C₁-C₄ alkoxy, C₁-C₆ haloalkyl, C₁-C₆ hydroxyalkyl, C₁-C₄ alkoxy-C₁-C₆ alkyl, —CN, —NH₂, —NH(C₁-C₄ alkyl) and —N(C₁-C₄ alkyl)₂.

In specific embodiments, each R³² is independently —Cl, —F, —OH, —CH₃, —CH₂CH₃, —CF₃, —CH₂OH, —CH₂OCH₃, —OCH₃, —OC₂H₅, —OCF₃, —CN, —CONH₂, —CONHCH₃, —CON(CH₃)₂, —NHC(O)CH₃, —NH₂, —NHCH₃, —N(CH₃)₂, cyclopropyl, 4-6 membered heterocyclyl, phenyl or 5-6 membered heteroaryl, where said 4-6 membered heterocyclyl, phenyl or 5-6 membered heteroaryl are optionally substituted by halo, C₁-C₄ alkyl or C₁-C₄ alkoxy.

In compounds of formula (I), R³ is H, C₁-C₈ alkyl, C₁-C₈ alkoxy, halo, —OH, —CN or —NR⁷R⁸, where said C₁-C₈ alkyl or C₁-C₈ alkoxy is optionally substituted by one or more R²³. In some such embodiments, said C₁-C₈ alkyl or C₁-C₈ alkoxy is optionally substituted by 1 to 3 R²³ groups, where R²³ is defined as in formula (I) above. In specific embodiments, R³ is H or halo, preferably H or F.

In compounds of formula (I), R⁴ is independently selected from the group consisting of H, C₁-C₈ alkyl, C₁-C₈ alkoxy, C₁-C₈ thioalkoxy, halo, —OH, —CN, C₃-C₈ cycloalkyl, 3-12 membered heterocyclyl, C₆-C₁₂ aryl, 5-12 membered heteroaryl, —OR¹¹ and —NR⁷R⁸, where each said C₁-C₈ alkyl, C₁-C₈ alkoxy, C₁-C₈ thioalkoxy or C₃-C₈ cycloalkyl is optionally substituted by one or more R²⁴, and each said heterocyclyl, aryl, heteroaryl or R¹¹ is optionally substituted by one or more R³⁴. In some such embodiments, each said C₁-C₈ alkyl, C₁-C₈ alkoxy, C₁-C₈ thioalkoxy or C₃-C₈ cycloalkyl is optionally substituted by 1 to 3 R²⁴, and each said 3-12 membered heterocyclyl, C₆-C₁₂ aryl, 5-12 membered heteroaryl or R¹¹ is optionally substituted by 1 to 3 R³⁴.

In one embodiment, R⁴ is H, halo or —CN. In some such embodiments, R⁴ is H. In other such embodiments, R⁴ is halo, preferably CI or F. In still other such embodiments, R⁴ is —CN.

In another embodiment, R⁴ is C₁-C₈ alkyl, C₁-C₈ alkoxy, C₁-C₈ thioalkoxy or C₃-C₈ cycloalkyl, where each said C₁-C₈ alkyl, C₁-C₈ alkoxy or C₃-C₈ cycloalkyl is optionally substituted by 1 to 3 R²⁴. In other such embodiments, R⁴ is C₁-C₄ alkyl or C₁-C₄ alkoxy optionally substituted by 1 to 3 R²⁴ groups.

In compounds of formula (I), R²⁴ is independently selected from the group consisting of halo, —OH, C₁-C₄ alkoxy, —CN, —NR⁹R¹⁰, C₃-C₈ cycloalkyl, 3-12 membered heterocyclyl, C₆-C₁₂ aryl and 5-12 membered heteroaryl, where each said cycloalkyl, heterocyclyl, aryl or heteroaryl is optionally substituted by 1 to 3 substituents independently selected from the group consisting of halo, —OH, ═O, C₁-C₄ alkyl, C₁-C₄ alkoxy, —CN, —NH₂, —NH(C₁-C₄ alkyl) and —N(C₁-C₄ alkyl)₂.

When R²⁴ is —NR⁹R¹⁰, each R⁹ and R¹⁰ is independently H or C₁-C₄ alkyl, or R⁹ and R¹⁰ may be taken together with the N atom to which they are attached to form an optionally substituted 3-12 membered heterocyclyl or an optionally substituted 5-12 membered heteroaryl moiety, each optionally containing 1, 2 or 3 additional heteroatoms selected from N, O and S. In some such embodiments, each said heterocyclyl or heteroaryl is optionally substituted by 1 to 3 substituents independently selected from the group consisting of halo, —OH, ═O, C₁-C₄ alkyl, C₁-C₄ alkoxy, —CN, —NH₂, —NH(C₁-C₄ alkyl) and —N(C₁-C₄ alkyl)₂.

In certain embodiments, each R²⁴ is independently selected from the group consisting of Cl, F, —OH, —OCH₃, —OC₂H₅, —OCF₃, —CN, —NH₂, —NHCH₃, —N(CH₃)₂, cyclopropyl, optionally substituted 4-6 membered heterocyclyl, optionally substituted phenyl, and optionally substituted 5-6 membered heteroaryl. In some such embodiments, said 4-6 membered heterocyclyl is pyrrolidinyl, morpholinyl, azetidinyl, piperidinyl, piperazinyl, each of which may be optionally substituted as defined in formula (I). In other such embodiments, said 5-6 membered heteroaryl is optionally substituted pyridyl or pyrimidinyl. In some embodiments, said 4-6 membered heterocyclyl or said 5-6 membered heteroaryl is optionally substituted by 1-3 substituents independently selected from halo, —OH, ═O, C₁-C₄ alkyl, C₁-C₄ alkoxy, —CN—NH₂, —NH(C₁-C₄ alkyl) and —N(C₁-C₄ alkyl)₂.

In another embodiment of formula (I), R⁴ is independently selected from the group consisting of 3-12 membered heterocyclyl, C₆-C₁₂ aryl, 5-12 membered heteroaryl, —OR¹¹ and —NR⁷R⁸, where each said heterocyclyl, aryl, heteroaryl or R¹¹ is optionally substituted by one or more R³⁴. In some embodiments, each said heterocyclyl, aryl, heteroaryl or R¹¹ is optionally substituted by 1 to 3 R³⁴ groups.

In one such embodiment of formula (I), R⁴ is 3-12 membered heterocyclyl, where said heterocyclyl is optionally substituted by one or more R³⁴. In some embodiments, said heterocyclyl is optionally substituted by 1 to 3 R³⁴ groups. In some such embodiments, said 3-12 membered heterocyclyl is selected from the group consisting of pyrrolidinyl, piperidinyl, morpholinyl, piperazinyl, 2-oxa-5-azabicyclo[2.2.1]heptanyl, 3-oxa-8-azabicyclo[3.2.1]octanyl, dihydropyranyl, tetrahydrofuranyl and tetrahydropyranyl, each of which is optionally substituted by 1 to 3 R³⁴ groups.

In another embodiment, R⁴ is a 5-12 membered heteroaryl, where said heteroaryl is optionally substituted by one or more R³⁴. In some such embodiments, said 5-12 membered heteroaryl is optionally substituted by 1 to 3 R³⁴ groups. In other embodiments, R⁴ is a 5-6 membered heteroaryl, optionally substituted by 1 to 3 R³⁴ groups. In some such embodiments, said 5-6 membered heteroaryl is selected from the group consisting of pyrazolyl, imidazolyl, pyrrolyl, triazolyl, tetrazolyl, thienyl, thiazolyl, isothiazolyl, furanyl, oxazoyl, isoxazolyl, oxadiazolyl, thiadiazolyl, pyridinyl, pyrimidinyl, pyrazinyl or pyridazinyl ring, each of which is optionally substituted by 1 to 3 R³⁴ groups.

In a further embodiment of formula (I), R⁴ is —OR¹¹, where R¹¹ is selected from the group consisting of C₃-C₈ cycloalkyl, 3-12 membered heterocyclyl, C₆-C₁₂ aryl and 5-12 membered heteroaryl, each of which may be optionally substituted by one or more R³⁴. In some embodiments, R¹¹ is optionally substituted by 1 to 3 R³⁴ groups.

In still other embodiments of formula (I), R⁴ is —NR⁷R⁸, where R⁷ and R⁸ are taken together with the N atom to which they are attached to form a 3-12 membered heterocyclyl or 5-12 membered heteroaryl, each optionally containing 1, 2 or 3 additional heteroatoms selected from O, N and S, wherein each said heterocyclyl or heteroaryl is optionally substituted by one or more R³⁷.

In some embodiments of formula (I), when R⁴ is 3-12 membered heterocyclyl, C₆-C₁₂ aryl, 5-12 membered heteroaryl or —OR¹¹, each said 3-12 membered heterocyclyl, C₆-C₁₂ aryl, 5-12 membered heteroaryl, or R¹¹ is optionally substituted by 1 to 3 R³⁴, wherein each R³⁴ is independently selected from the group consisting of halo, C₁-C₈ alkyl, —CN, ═O, —COR^{c}, —CO₂R^{c}, —CONR^{c}R^{d}, —OR^{c}, —SR^{c}, —SOR^{c}, —SO₂R^{c}, —SO₂NR^{c}R^{d}, —NO₂, —NR^{c}R^{d}, —NR^{c}C(O)R^{d}, —NR^{c}C(O)NR^{c}R^{d}, —NR^{c}C(O)OR^{d}, —NR^{c}SO₂R^{d}, —NR^{c}SO₂NR^{c}R^{d}, —OC(O)R^{c}, —OC(O)NR^{c}R^{d}, C₃-C₈ cycloalkyl, 3-12 membered heterocyclyl, C₆-C₁₂ aryl and 5-12 membered heteroaryl, and where R^{c} and R^{d} are defined as in formula (I) above.

In some such embodiments, each R³⁴ is independently selected from the group consisting of halo, C₁-C₈ alkyl, —CN, —CONR^{c}R^{d}, —NR^{c}R^{d}, —NR^{c}C(O)R^{d}, —OR^{c}, —C₃-C₈ cycloalkyl, C₆-C₁₂ aryl and 5-12 membered heteroaryl, where said C₁-C₈ alkyl is optionally substituted by —OH, —C₁-C₄ alkoxy and halo, and each R^{c} and R^{d} is independently H or C₁-C₄ alkyl.

In other embodiments, each R³⁴ is independently selected from the group consisting of halo, C₁-C₄ alkyl, CN, —OR^{c}, —SR^{c}, —SO₂R^{c} and —NR^{c}R^{d}, where each R^{c} and R^{d} is independently H or C₁-C₄ alkyl; or R^{c} and R^{d} in —NR^{c}R^{d} may be taken together with the N atom to which they are attached to form a 4-6 membered heterocyclyl optionally containing 1 additional heteroatom selected from O, N and S, where said 4-6 membered heterocyclyl is optionally substituted by 1 to 3 substituents independently selected from the group consisting of halo, —OH, ═O, C₁-C₄ alkyl, C₁-C₄ alkoxy, C₁-C₆ haloalkyl, C₁-C₆ hydroxyalkyl, C₁-C₄ alkoxy-C₁-C₆ alkyl, —CN, —NH₂, —NH(C₁-C₄ alkyl) and —N(C₁-C₄ alkyl)₂.

In specific embodiments, each R³⁴ is independently selected from the groups consisting of —Cl, —F, —OH, —CH₃, —CH₂CH₃, —CF₃, —CH₂OH, —CH₂OCH₃, —OCH₃, —OC₂H₅, —OCF₃, —CN, —CONH₂, —CONHCH₃, —CON(CH₃)₂, —NHC(O)CH₃, —NH₂, —NHCH₃, —N(CH₃)₂, cyclopropyl, optionally substituted 4-6 membered heterocyclyl, optionally substituted phenyl and optionally substituted 5-6 membered heteroaryl, where said 4-6 membered heterocyclyl, phenyl or 5-6 membered heteroaryl are optionally substituted by halo, C₁-C₄ alkyl or C₁-C₄ alkoxy.

In specific embodiments, each R³⁴ is independently selected from the groups consisting of —Cl, —F, —OH, —CH₃, —CH₂CH₃, —CF₃, —CH₂OH, —CH₂OCH₃, —OCH₃, —OC₂H₅, —OCF₃, —CN, —CONH₂, —CONHCH₃, —CON(CH₃)₂, —NHC(O)CH₃, —NH₂, —NHCH₃, —N(CH₃)₂, cyclopropyl, optionally substituted 4-6 membered heterocyclyl, optionally substituted phenyl and optionally substituted 5-6 membered heteroaryl, where said 4-6 membered heterocyclyl, phenyl or 5-6 membered heteroaryl are optionally substituted by 1 to 3 halo, C₁-C₄ alkyl or C₁-C₄ alkoxy.

In compounds of formula (I), R⁵ is H, C₁-C₈ alkyl, C₁-C₈ alkoxy, halo, —OH, —CN or —NR⁷R⁸, where said C₁-C₈ alkyl or C₁-C₈ alkoxy is optionally substituted by one or more R²⁵. In some such embodiments, said C₁-C₈ alkyl or C₁-C₈ alkoxy is optionally substituted by 1 to 3 R²⁵ groups, where R²⁵ is defined as in formula (I) above. In specific embodiments, R⁵ is H or halo, preferably H or F.

In compounds of formula (I), R⁶ is H or C₁-C₄ alkyl. In some embodiments of formula (I), R⁶ is H or methyl. In preferred embodiments, R⁶ is H.

In some embodiments of formula (I), each R⁷ and R⁸ is independently H or C₁-C₈ alkyl, where said C₁-C₈ alkyl is optionally substituted by one or more R²⁷. In some such embodiments, said C₁-C₈ alkyl is optionally substituted by 1 to 3 R²⁷ groups.

In compounds of formula (I), R²⁷ is independently selected from the group consisting of halo, —OH, C₁-C₄ alkoxy, —CN, —NR⁹R¹⁰, C₃-C₈ cycloalkyl, 3-12 membered heterocyclyl, C₆-C₁₂ aryl and 5-12 membered heteroaryl, where each said cycloalkyl, heterocyclyl, aryl or heteroaryl is optionally substituted by 1 to 3 substituents independently selected from the group consisting of halo, —OH, ═O, C₁-C₄ alkyl, C₁-C₄ alkoxy, —CN, —NH₂, —NH(C₁-C₄ alkyl) and —N(C₁-C₄ alkyl)₂.

When R²⁷ is —NR⁹R¹⁰, each R⁹ and R¹⁰ is independently H or C₁-C₄ alkyl, or R⁹ and R¹⁰ may be taken together with the N atom to which they are attached to form an optionally substituted 3-12 membered heterocyclyl or an optionally substituted 5-12 membered heteroaryl moiety, optionally containing 1, 2 or 3 additional heteroatoms selected from N, O and S. In some such embodiments, each said 3-12 membered heterocyclyl or 5-12 membered heteroaryl moiety is optionally substituted by 1 to 3 substituents independently selected from the group consisting of halo, —OH, ═O, C₁-C₄ alkyl, C₁-C₄ alkoxy, —CN, —NH₂, —NH(C₁-C₄ alkyl) and —N(C₁-C₄ alkyl)₂.

In certain embodiments, each R²⁷ is independently selected from the group consisting of chloro, fluoro, —OH, —OCH₃, —OC₂H₅, —OCF₃, —CN, —NH₂, —NHCH₃, —N(CH₃)₂ and N-pyrrolidinyl.

In other embodiments, R⁷ and R⁸ in —NR⁷R⁸ may be taken together with the N atom to which they are attached to form a 3-12 membered heterocyclyl or 5-12 membered heteroaryl, each optionally containing 1, 2 or 3 additional heteroatoms selected from O, N and S, wherein each said heterocyclyl or heteroaryl is optionally substituted by one or more R³⁷, preferably by 1 to 3 R³⁷ groups.

In compounds of formula (I), each R³⁷ is independently selected from the group consisting of halo, C₁-C₈ alkyl, —CN, ═O, —COR^{c}, —CO₂R^{c}, —CONR^{c}R^{d}, —OR^{c}, —SR^{c}, —SOR^{c}, —SO₂R^{c}, —SO₂NR^{c}R^{d}, —NO₂, —NR^{c}R^{d}, —NR^{c}C(O)R^{d}, —NR^{c}C(O)NR^{c}R^{d}, —NR^{c}C(O)OR^{d}, —NR^{c}SO₂R^{d}, —NR^{c}SO₂NR^{c}R^{d}, —OC(O)R^{c}, —OC(O)NR^{c}R^{d}, C₃-C₈ cycloalkyl, 3-12 membered heterocyclyl, C₆-C₁₂ aryl and 5-12 membered heteroaryl, and where R^{c} and R^{d} are defined as in formula (I) above.

In some such embodiments, each R³⁷ is independently halo, C₁-C₈ alkyl, —CN, —CONR^{c}R^{d}, —NR^{c}R^{d}, —NR^{c}C(O)R^{d}, —OR^{c}, —C₃-C₈ cycloalkyl, C₆-C₁₂ aryl and 5-12 membered heteroaryl, where said alkyl is optionally substituted by —OH, —C₁-C₄ alkoxy and halo, and each R^{c} and R^{d} is independently H or C₁-C₄ alkyl. In specific embodiments, each R³⁷ is independently selected from the group consisting of —Cl, —F, —OH, —CH₃, —CH₂CH₃, —CF₃, —CH₂OH, —CH₂OCH₃, OCH₃, —OC₂H₅, —OCF₃, —CN, —CONH₂, —CONHCH₃, —NHC(O)CH₃, —NH₂, —NHCH₃, —N(CH₃)₂, cyclopropyl, optionally substituted 4-6 membered heterocyclyl, optionally substituted phenyl and optionally substituted 5-6 membered heteroaryl. In some embodiments, said 4-6 membered heterocyclyl or said heteroaryl 5-6 membered heteroaryl is optionally substituted by 1 to 3 substituents independently selected from halo, —OH, ═O, C₁-C₄ alkyl, C₁-C₄ alkoxy, —CN, —NH₂, —NH(C₁-C₄ alkyl) and —N(C₁-C₄ alkyl)₂.

In some embodiments of formula (I), each R⁹ and R¹⁰ is independently H or C₁-C₄ alkyl. In other embodiments, R⁹ and R¹⁰ are taken together with the N atom to which they are attached to form a 3-12 membered heterocyclyl or 5-12 membered heteroaryl, each optionally containing 1, 2 or 3 additional heteroatoms selected from O, N and S, where each said heterocyclyl or heteroaryl is optionally substituted by 1 to 3 substituents independently selected from the group consisting of halo, —OH, ═O, C₁-C₄ alkyl, C₁-C₄ alkoxy, —CN, —NH₂, —NH(C₁-C₄ alkyl) and —N(C₁-C₄ alkyl)₂.

In compounds of formula (I), R¹¹ is selected from the group consisting of C₃-C₈ cycloalkyl, 3-12 membered heterocyclyl, C₆-C₁₂ aryl and 5-12 membered heteroaryl, where each said cycloalkyl, heterocyclyl, aryl and heteroaryl is optionally substituted by one or more R³⁴. In some such embodiments, each said cycloalkyl, heterocyclyl, aryl and heteroaryl is optionally substituted by 1 to 3 R³⁴

In compounds of formula (I), X and Z are independently selected from the group consisting of H, C₁-C₈ alkyl, C₂-C₈ alkenyl, C₂-C₈ alkynyl, C₃-C₈ cycloalkyl, 3-12 membered heterocyclyl, C₆-C₁₂ aryl, 5-12 membered heteroaryl, halo, —CN, —COR^{a}, —OC₂R^{a}, —CONR^{a}R^{b}, —SR^{a}, —SOR^{a}, —SO₂R^{a}, —SO₂NR^{a}R^{b}, —NO₂, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}—OR^{a}, —OC(O)R^{a} and —OC(O)NR^{a}R^{b}; wherein said alkyl, alkenyl, alkynyl, cycloalkyl, heterocyclyl, aryl, and heteroaryl groups may be optionally substituted by 1 to 3 substituents independently selected from the group consisting of halo, —CN, —COR^{a}, —CO₂R^{a}, —CONR^{a}R^{b}, —SR^{a}, —SOR^{a}, —SO₂R^{a}, —SO₂NR^{a}R^{b}, —NO₂, —NR^{a}R^{b}, —NR^{a}C(O)R^{b}, —NR^{a}C(O)NR^{a}R^{b}, —NR^{a}C(O)OR^{a}—NR^{a}SO₂R^{b}, —NR^{a}SO₂NR^{a}R^{b}, —OR^{a}, —OC(O)R^{a}, —OC(O)NR^{a}R^{b}, C₃-C₈ cycloalkyl, 3-12 membered heterocyclyl, C₆-C₁₂ aryl and 5-12 membered heteroaryl; where R^{a} and R^{b} are defined as in formula (I) above.

In some embodiments, X and Z are independently selected from the group consisting of C₁-C₈ alkyl, C₃-C₈ cycloalkyl, 3-12 membered heterocyclyl, C₆-C₁₂ aryl, 5-12 membered heteroaryl, each of which may be optionally substituted as described in formula (I) above. In other embodiments, X and Z are independently selected from the group consisting of —NR^{a}R^{b} and —OR^{a}, where R^{a} and R^{b} are defined as in formula (I) above. In specific embodiments of formula (I), X and Z are each independently C₁-C₈ alkyl, preferably C₁-C₄ alkyl, where said alkyl is optionally substituted by halo, —OH, C₁-C₄ alkoxy, —CN, —NH₂, —NH(C₁-C₄ alkyl) and —N(C₁-C₄ alkyl)₂. In preferred embodiments, X and Z are each independently C₁-C₄ alkyl.

In compounds of formula (I), Y is H, halo, —OH or C₁-C₄ alkoxy. In specific embodiments, Y is H or F. In some such embodiments, Y is H. In other such embodiments, Y is F. In other embodiments, Y is OH. In still other embodiments, Y is C₁-C₄ alkoxy.

In preferred embodiments of formula (I), X and Z are each independently selected from C₁-C₈ alkyl, and Y is H or F. In more preferred embodiments of formula (I), X and Z are each independently selected from C₁-C₄ alkyl, and Y is H.

Each of the embodiments described herein with respect to compounds of formula (I) is also applicable to the compounds of formulae (II), (Ill), (IV), (V), (VI) and (VII) described herein, provided the particular embodiment of formula (I) and description of formulae (II) to (VII) are not inconsistent with each other. The described embodiments can be combined with one or more other embodiments described herein not inconsistent with the embodiment(s) with which it is combined.

In another aspect, the invention provides compounds of formula (II),

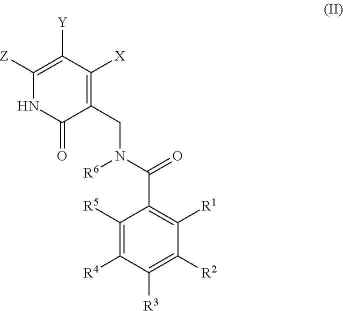

or a pharmaceutically acceptable salt thereof,

wherein:
R¹ is C₁-C₈ alkyl, C₁-C₈ alkoxy, halo, —OH, —CN or —NR⁷R⁸, where each said C₁-C₈ alkyl or C₁-C₈ alkoxy is optionally substituted by one or more R²¹;

R² is 3-12 membered heterocyclyl, C₆-C₁₂ aryl, 5-12 membered heteroaryl or C₁-C₈ alkoxy, where said C₁-C₈ alkoxy is optionally substituted by one or more R²², and each said heterocyclyl, aryl or heteroaryl is optionally substituted by one or more R³²;

R³ is H, C₁-C₈ alkyl, C₁-C₈ alkoxy, halo, —OH, —CN or —NR⁷R⁸, where each said C₁-C₈ alkyl or C₁-C₈ alkoxy is optionally substituted by one or more R²³;

R⁴ is independently selected from the group consisting of H, C₁-C₈ alkyl, C₁-C₈ alkoxy, C₁-C₈ thioalkoxy, halo, —OH, —CN, C₃-C₈ cycloalkyl, 3-12 membered heterocyclyl, C₆-C₁₂ aryl, 5-12 membered heteroaryl, —OR¹¹ and —NR⁷R⁸, where each said C₁-C₈ alkyl, C₁-C₈ alkoxy, C₁-C₈ thioalkoxy or C₃-C₈ cycloalkyl is optionally substituted by one or more R²⁴, and each said heterocyclyl, aryl, heteroaryl or R¹¹ is optionally substituted by one or more R³⁴;

R⁵ is H, C₁-C₈ alkyl, C₁-C₈ alkoxy, halo, —OH, —CN or —NR⁷R⁸, where each said C₁-C₈ alkyl or C₁-C₈ alkoxy is optionally substituted by one or more R²⁵;

R⁶ is H or C₁-C₄ alkyl;

each R⁷ and R⁸ is independently H or C₁-C₈ alkyl, where said C₁-C₈ alkyl is optionally substituted by one or more R²⁷; or

R⁷ and R⁸ may be taken together with the N atom to which they are attached to form a 3-12 membered heterocyclyl or 5-12 membered heteroaryl, each optionally containing 1, 2 or 3 additional heteroatoms selected from O, N and S, wherein each said heterocyclyl or heteroaryl is optionally substituted by one or more R³⁷;

each R²¹, R²², R²³ and R²⁵ is independently selected from the group consisting of halo, —OH, C₁-C₄ alkoxy, —CN and —NR⁹R¹⁰;

each R²⁴ and R²⁷ is independently selected from the group consisting of halo, —OH, C₁-C₄ alkoxy, —CN, —NR⁹R¹⁰, C₃-C₈ cycloalkyl, 3-12 membered heterocyclyl, C₆-C₁₂ aryl and 5-12 membered heteroaryl, where each said cycloalkyl, heterocyclyl, aryl or heteroaryl is optionally substituted by one or more substituents independently selected from the group consisting of halo, —OH, ═O, C₁-C₄ alkyl, C₁-C₄ alkoxy, —CN, —NH₂, —NH(C₁-C₄ alkyl) and —N(C₁-C₄ alkyl)₂;

each R⁹ and R¹⁰ is independently H or C₁-C₄ alkyl; or

R⁹ and R¹⁰ may be taken together with the N atom to which they are attached to form a 3-12 membered heterocyclyl or 5-12 membered heteroaryl, each optionally containing 1, 2 or 3 additional heteroatoms selected from O, N and S, where each said heterocyclyl or heteroaryl is optionally substituted by one or more substituents independently selected from the group consisting of halo, —OH, ═O, C₁-C₄ alkyl, C₁-C₄ alkoxy, —CN, —NH₂, —NH(C₁-C₄ alkyl) and —N(C₁-C₄ alkyl)₂;

R¹¹ is selected from the group consisting of C₃-C₈ cycloalkyl, 3-12 membered heterocyclyl, C₆-C₁₂ aryl and 5-12 membered heteroaryl;
each R³², R³⁴ and R³⁷ is independently selected from the group consisting of halo, C₁-C₈ alkyl, —CN, ═O, —COR^{c}, —CO₂R^{c}, —CONR^{c}R^{d}, —OR^{c}, —SR^{c}, —SOR^{c}, —SO₂R^{c}, —SO₂NR^{c}R^{d}, —NO₂, —NR^{c}R^{d}, —NR^{c}C(O)R^{d}, —NR^{c}C(O)NR^{c}R^{d}, —NR^{c}C(O)OR^{d}—NR^{c}SO₂R^{d}, —NR^{c}SO₂NR^{c}R^{d}, —OC(O)R^{c}, —OC(O)NR^{c}R^{d}, C₃-C₈ cycloalkyl, 3-12 membered heterocyclyl, C₆-C₁₂ aryl and 5-12 membered heteroaryl;
each R^{c} and R^{d} is independently selected from the group consisting of H, C₁-C₈ alkyl, C₃-C₈ cycloalkyl, 3-12 membered heterocyclyl, C₆-C₁₂ aryl and 5-12 membered heteroaryl; or
R^{c} and R^{d} may be taken together with the N atom to which they are attached to form a 3-12 membered heterocyclyl or 5-12 membered heteroaryl ring, each optionally containing 1, 2 or 3 additional heteroatoms selected from O, N and S;
wherein each said alkyl, cycloalkyl, heterocyclyl, aryl or heteroaryl in R³², R³⁴, R³⁷, R^{c} and R^{d} is optionally substituted by 1 to 3 substituents independently selected from the group consisting of halo, —OH, ═O, C₁-C₄ alkyl, C₁-C₄ alkoxy, C₁-C₆ haloalkyl, C₁-C₆ hydroxyalkyl, C₁-C₄ alkoxy-C₁-C₆ alkyl, —CN, —NH₂, —NH(C₁-C₄ alkyl) and —N(C₁-C₄ alkyl)₂;

X and Z are independently selected from the group consisting of H, C₁-C₈ alkyl, C₂-C₈ alkenyl, C₂-C₈ alkynyl, C₃-C₈ cycloalkyl, 3-12 membered heterocyclyl, C₆-C₁₂ aryl, 5-12 membered heteroaryl, halo, CN, —COR^{a}, —CO₂R^{a}, —CONR^{a}R^{b}, —SR^{a}, —SOR^{a}, —SO₂R^{a}, —SO₂NR^{a}R^{b}, —NO₂, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}—OR^{a}, —OC(O)R^{a} or —OC(O)NR^{a}R^{b};
wherein each said C₁-C₈ alkyl, C₂-C₈ alkenyl, C₂-C₈ alkynyl, C₃-C₈ cycloalkyl, 3-12 membered heterocyclyl, C₈-C₁₂ aryl, or 5-12 membered heteroaryl group is optionally substituted by one or more substituents independently selected from the group consisting of halo, —CN, —COR^{a}, —CO₂R^{a}, —CONR^{a}R^{b}, —SR^{a}, —SOR^{a}, —SO₂R^{a}, —SO₂NR^{a}R^{b}, —NO₂, —NR^{a}R^{b}, —NR^{a}C(O)R^{b}, —NR^{a}C(O)NR^{a}R^{b}, —NR^{a}C(O)OR^{a}—NR^{a}SO₂R^{b}, —NR^{a}SO₂NR^{a}R^{b}, —OR^{a}, —OC(O)R^{a}, —OC(O)NR^{a}R^{b}, C₃-C₈ cycloalkyl, 3-12 membered heterocyclyl, C₈-C₁₂ aryl, and 5-12 membered heteroaryl;
each R^{a} and R^{b} is independently H, C₁-C₈ alkyl, C₂-C₈ alkenyl, C₂-C₈ alkynyl, C₃-C₈ cycloalkyl, 3-12 membered heterocyclyl, C₆-C₁₂ aryl or 5-12 membered heteroaryl, where each said alkyl, alkenyl, alkynyl, cycloalkyl, heterocyclyl, aryl and heteroaryl is optionally substituted by one or more substituents independently selected from the group consisting of halo, C₁-C₄ alkyl, —OR″, —NR″₂, —CO₂R″, —CONR″₂, —SO₂R″ and —SO₂NR″₂, where each R″ is independently H or C₁-C₄ alkyl; or
R^{a} and R^{b} may be taken together with the N atom to which they are attached to form a 3-12 membered heterocyclyl or 5-12 membered heteroaryl, each optionally containing 1, 2 or 3 additional heteroatoms selected from O, N and S, wherein said heterocyclyl or heteroaryl is optionally substituted by one or more substituents independently selected from the group consisting of halo, —OH, ═O, C₁-C₄ alkyl, C₁-C₄ alkoxy, C₁-C₆ haloalkyl, C₁-C₆ hydroxyalkyl, C₁-C₄ alkoxy-C₁-C₆ alkyl, —CN, —NH₂, —NH(C₁-C₄ alkyl) and —N(C₁-C₄ alkyl)₂; and

Y is H, halo, —OH or C₁-C₄ alkoxy.

The embodiments described herein with respect to formula (I), and combinations thereof, are also applicable to formula (II).

In one embodiment of formula (II), R¹ is optionally substituted C₁-C₄ alkyl or halo. In some such embodiments, R¹ is C₁-C₄ alkyl optionally substituted by 1 to 3 R²¹, where each R²¹ is independently halo, —OH, C₁-C₄ alkoxy, —CN and —NR⁹R¹⁰, and each R⁹ and R¹⁰ is independently H or C₁-C₄ alkyl.

In another embodiment of formula (II), R¹ is C₁-C₄ alkyl or halo. In some such embodiments, R¹ is methyl, ethyl, n-propyl, isopropyl, n-butyl, sec-butyl or tert-butyl. In other such embodiments, R¹ is chloro or fluoro (Cl or F). In further embodiments, R¹ is methyl, ethyl, chloro or fluoro.

In another embodiment of formula (II), R² is 5-12 membered heteroaryl optionally substituted by one or more R³². In some such embodiments, said 5-12 membered heteroaryl is optionally substituted by 1 to 3 R³² groups. In some such embodiments, said 5-12 membered heteroaryl is selected from the group consisting of pyrazolyl, imidazolyl, triazolyl and pyrrolyl, where said heteroaryl is optionally substituted by one or more R³², preferably by 1 to 3 R³² groups.

In some embodiments of formula (II), each R³² is independently halo, C₁-C₈ alkyl, —CN, —CONR^{c}R^{d}, —NR^{c}R^{d}, —NR^{c}C(O)R^{d}, C₃-C₈ cycloalkyl, C₆-C₁₂ aryl and 5-12 membered heteroaryl, where said C₁-C₈ alkyl is optionally substituted by —OH, —C₁-C₄ alkoxy or halo, and each R^{c} and R^{d} is independently H or C₁-C₄ alkyl, or R^{c} and R^{d} may be taken together with the N atom to which they are attached to form a 4-6 membered heterocyclyl ring optionally containing 1 additional heteroatom selected from O, N and S, where said 4-6 membered heterocyclyl ring is optionally substituted by 1 to 3 substituents independently selected from the group consisting of halo, —OH, ═O, C₁-C₄ alkyl, C₁-C₄ alkoxy, C₁-C₆ haloalkyl, C₁-C₆ hydroxyalkyl, C₁-C₄ alkoxy-C₁-C₆ alkyl, —CN, —NH₂, —NH(C₁-C₄ alkyl) and —N(C₁-C₄ alkyl)₂. In specific embodiments, each R³² is independently —Cl, —F, —OH, —CH₃, —CH₂CH₃, —CF₃, —CH₂OH, —CH₂OCH₃, —OCH₃, —OC₂H₅, —OCF₃, —CN, —CONH₂, —CONHCH₃, —CON(CH₃)₂, —NHC(O)CH₃, —NH₂, —NHCH₃, —N(CH₃)₂, cyclopropyl, 4-6 membered heterocyclyl, phenyl or 5-6 membered heteroaryl, where said 4-6 membered heterocyclyl, phenyl or 5-6 membered heteroaryl are optionally substituted by halo, C₁-C₄ alkyl or C₁-C₄ alkoxy. In preferred embodiments of formula (II), each R³² is independently halo or C₁-C₄ alkyl.

In yet another embodiment of formula (II), R² is C₁-C₈ alkoxy optionally substituted by one or more R²². In some such embodiments, R² is C₁-C₈ alkoxy optionally substituted by 1 to 3 R²² groups. In some such embodiments, R² is C₁-C₄ alkoxy optionally substituted by 1 to 3 R²² groups. In other such embodiments, R² is C₁-C₄ alkoxy. In specific embodiments, said alkoxy is methoxy, ethoxy, propoxy, isopropoxy, n-butoxy, sec-butoxy or tert-butoxy.

In some embodiments, each R²² is independently selected from the group consisting of halo, —OH, C₁-C₄ alkoxy, —CN and —NR⁹R¹⁰, where each R⁹ and R¹⁰ is independently H or C₁-C₄ alkyl, or R⁹ and R¹⁰ may be taken together with the N atom to which they are attached to form an optionally substituted heterocyclyl or heteroaryl moiety, optionally containing 1, 2 or 3 additional heteroatoms selected from N, O and S. In some such embodiments, each R²² is independently selected from the group consisting of Cl, F, —OH, —OCH₃, —OC₂H₅, —OCF₃, —CN, —NH₂, —NHCH₃, —N(CH₃)₂, cyclopropyl, optionally substituted 4-6 membered heterocyclyl and optionally substituted 5-6 membered heteroaryl. In some embodiments, said 4-6 membered heterocyclyl or said 5-6 membered heteroaryl is optionally substituted by halo, —OH, ═O, C₁-C₄ alkyl, C₁-C₄ alkoxy, —CN, —NH₂, —NH(C₁-C₄ alkyl) or —N(C₁-C₄ alkyl)₂.

In other embodiments, each R²² is independently selected from the group consisting of halo, —OH, C₁-C₄ alkoxy, —CN and —NR⁹R¹⁰, where each R⁹ and R¹⁰ is independently H or C₁-C₄ alkyl, or R⁹ and R¹⁰ in —NR⁹R¹⁰ may be taken together with the N atom to which they are attached to form an optionally substituted 3-12 membered heterocyclyl or 5-12 membered heteroaryl moiety, optionally containing 1, 2 or 3 additional heteroatoms selected from N, O and S. In some such embodiments, each R²² is independently selected from the group consisting of Cl, F, —OH, —OCH₃, —OC₂H₅, —OCF₃, —CN, —NH₂, —NHCH₃, —N(CH₃)₂, cyclopropyl, optionally substituted 4-6 membered heterocyclyl and optionally substituted 5-6 membered heteroaryl. In some embodiments, said 4-6 membered heterocyclyl or said 5-6 membered heteroaryl is optionally substituted by 1 to 3 halo, —OH, ═O, C₁-C₄ alkyl, C₁-C₄ alkoxy, —CN, —NH₂, —NH(C₁-C₄ alkyl) or —N(C₁-C₄ alkyl)₂.

In frequent embodiments of formula (II), R³ and R⁵ are independently H or halo, preferably R³ and R⁵ are independently H or F, and more preferably R³ and R⁵ are H.

In compounds of formula (II), R⁴ is independently selected from the group consisting of H, C₁-C₈ alkyl, C₁-C₈ alkoxy, C₁-C₈ thioalkoxy, halo, —OH, —CN, C₃-C₈ cycloalkyl, 3-12 membered heterocyclyl, C₆-C₁₂ aryl, 5-12 membered heteroaryl, —OR¹¹ and —NR⁷R⁸, where each said C₁-C₈ alkyl, C₁-C₈ alkoxy, C₁-C₈ thioalkoxy or C₃-C₈ cycloalkyl is optionally substituted by one or more R²⁴, and each said heterocyclyl, aryl, heteroaryl or R¹¹ is optionally substituted by one or more R³⁴.

In one embodiment of formula (II), R⁴ is H, halo, —CN or 5-12 membered heteroaryl, where said heteroaryl is optionally substituted by one or more R³⁴, and R³⁴ is defined as for formula (I).

In another embodiment of formula (II), R⁴ is 5-12 membered heteroaryl, where said heteroaryl is optionally substituted by one or more R³⁴, preferably 1 to 3 R³⁴. In some such embodiments, R⁴ is selected from the group consisting of pyridyl, pyrimidinyl, pyrazinyl, pyrazolyl, imidazolyl, triazolyl and pyrrolyl, where said heteroaryl is optionally substituted by one or more R³⁴. Preferably said heteroaryl is optionally substituted by 1 to 3 R³⁴. In another embodiment of formula (II), R⁴ is 5-6 membered heteroaryl, where said heteroaryl is optionally substituted by 1 to 3 R³⁴. In some such embodiments, said 5-6 membered heteroaryl is selected from the group consisting of pyridyl, pyrimidinyl, pyrazinyl, pyrazolyl, imidazolyl, triazolyl and pyrrolyl, where said heteroaryl is optionally substituted by 1 to 3 R³⁴.

In some embodiments of formula (II), when R⁴ is 3-12 membered heterocyclyl, C₆-C₁₂ aryl, 5-12 membered heteroaryl or —OR¹¹, each said 3-12 membered heterocyclyl, C₆-C₁₂ aryl, 5-12 membered heteroaryl, or R¹¹ is optionally substituted by 1 to 3 R³⁴, wherein each R³⁴ is independently selected from the group consisting of halo, C₁-C₈ alkyl, —CN, ═O, —COR^{c}, —CO₂R^{c}, —CONR^{c}R^{d}, —SR^{c}, —SOR^{c}, —SO₂R^{c}, —SO₂NR^{c}R^{d}, —NO₂, —NR^{c}R^{d}, —NR^{c}C(O)R^{d}, —NR^{c}C(O)NR^{c}R^{d}, —NR^{c}C(O)OR^{d}, —NR^{c}SO₂R^{d}, —NR^{c}SO₂NR^{c}R^{d}, —OC(O)R^{c}, —OC(O)NR^{c}R^{d}, C₃-C₈ cycloalkyl, 3-12 membered heterocyclyl, C₆-C₁₂ aryl and 5-12 membered heteroaryl, and where R^{c} and R^{d} are defined as in formula (I) above.

In some such embodiments, each R³⁴ is independently halo, C₁-C₈ alkyl, —CN, —CONR^{c}R^{d}, —NR^{c}R^{d}, —NR^{c}C(O)R^{d}, —OR^{c}, —C₃-C₈ cycloalkyl, C₈-C₁₂ aryl and 5-12 membered heteroaryl, where said C₁-C₈ alkyl is optionally substituted by —OH, —C₁-C₄ alkoxy and halo, and each R^{c} and R^{d} is independently H or C₁-C₄ alkyl. In specific embodiments, each R³⁴ is independently selected from the groups consisting of —Cl, —F, —OH, —CH₃, —CH₂CH₃, —CF₃, —CH₂OH, —CH₂OCH₃, —OCH₃, —OC₂H₅, —OCF₃, —CN, —CONH₂, —CONHCH₃, —CON(CH₃)₂, —NHC(O)CH₃, —NH₂, —NHCH₃, —N(CH₃)₂, cyclopropyl, optionally substituted 4-6 membered heterocyclyl, optionally substituted phenyl and optionally substituted 5-6 membered heteroaryl, where said 4-6 membered heterocyclyl, phenyl or 5-6 membered heteroaryl are optionally substituted by halo, C₁-C₄ alkyl or C₁-C₄ alkoxy.

In specific embodiments of formula (II), each R³⁴ is independently selected from the group consisting of halo, C₁-C₄ alkyl, CN, —OR^{c}, —SR^{c}, —SO₂R^{c} and —NR^{c}R^{d}, where each R^{c} and R^{d} is independently H or C₁-C₄ alkyl. In other embodiments, each R³⁴ is independently selected from the group consisting of halo, C₁-C₄ alkyl, CN, —OR^{c}, —SR^{c}, —SO₂R^{c} and —NR^{c}R^{d}, where each R^{c} and R^{d} is independently H or C₁-C₄ alkyl, or R^{c} and R^{d} in —NR^{c}R^{d} may be taken together with the N atom to which they are attached to form a 4-6 membered heterocyclyl optionally containing 1 additional heteroatom selected from O, N and S, where said 4-6 membered heterocyclyl is optionally substituted by 1 to 3 substituents independently selected from the group consisting of halo, —OH, ═O, C₁-C₄ alkyl, C₁-C₄ alkoxy, C₁-C₆ haloalkyl, C₁-C₆ hydroxyalkyl, C₁-C₄ alkoxy-C₁-C₆ alkyl, —CN, —NH₂, —NH(C₁-C₄ alkyl) and —N(C₁-C₄ alkyl)₂.

In yet another embodiment of formula (II), R⁶ is H or methyl, preferably H.

In preferred embodiments of formula (II), X and Z are independently C₁-C₈ alkyl and Y is H or fluoro, preferably H. In some such embodiments, X and Z are independently C₁-C₄ alkyl and Y is H or fluoro, preferably H.

In one preferred embodiment of formula (II), the compounds have a combination of two or more of the following preferred features: R¹ is C₁-C₄ alkyl or halo; R² is optionally substituted 5-6 membered heteroaryl; R³ is H or F; R⁴ is H, halo or optionally substituted heteroaryl; R⁵ is H or F; R⁶ is H; X and Z are independently C₁-C₄ alkyl; and Y is H or F.

In another preferred embodiment of formula (II), the compounds have a combination of two or more of the following preferred features: R¹ is C₁-C₄ alkyl or halo; R² is optionally substituted C₁-C₄ alkoxy; R³ is H or F; R⁴ is H, halo or optionally substituted heteroaryl; R⁵ is H or F; R⁶ is H; X and Z are independently C₁-C₄ alkyl; and Y is H or F.

In another preferred embodiment of formula (II), the compounds have a combination of two or more of the following preferred features: R¹ is C₁-C₄ alkyl or halo; R² is optionally substituted 3-12 membered heterocyclyl; R³ is H or F; R⁴ is H, halo or optionally substituted heteroaryl; R⁵ is H or F; R⁶ is H; X and Z are independently C₁-C₄ alkyl; and Y is H or F.

In yet another preferred embodiment of formula (II), the compounds have a combination of two or more of the following preferred features: R¹ is C₁-C₄ alkyl or halo; R² is optionally substituted 5-6 membered heteroaryl; R³ is H or F; R⁴ is H, halo or optionally substituted 5-6 membered heteroaryl; R⁵ is H or F; R⁶ is H; X and Z are independently C₁-C₄ alkyl; and Y is H or F.

In still another preferred embodiment of formula (II), the compounds have a combination of two or more of the following preferred features: R¹ is C₁-C₄ alkyl or halo; R² is optionally substituted C₁-C₄ alkoxy; R³ is H or F; R⁴ is H, halo or optionally substituted 5-6 membered heteroaryl; R⁵ is H or F; R⁶ is H; X and Z are independently C₁-C₄ alkyl; and Y is H or F.

In a further preferred embodiment of formula (II), the compounds have a combination of two or more of the following preferred features: R¹ is C₁-C₄ alkyl or halo; R² is optionally substituted 3-12 membered heterocyclyl; R³ is H or F; R⁴ is H, halo or optionally substituted 5-6 membered heteroaryl; R⁵ is H or F; R⁶ is H; X and Z are independently C₁-C₄ alkyl; and Y is H or F.

In another preferred embodiment of formula (II), the compounds have a combination of two or more of the following preferred features: R¹ is C₁-C₄ alkyl or halo; R² is 5-6 membered heteroaryl optionally substituted by 1 to 3 R³²; R³ is H; R⁴ is H, halo or 5-6 membered heteroaryl optionally substituted by 1 to 3 R³⁴; R⁵ is H; R⁶ is H; X and Z are independently C₁-C₄ alkyl; and Y is H.

In another preferred embodiment of formula (II), the compounds have a combination of two or more of the following preferred features: R¹ is C₁-C₄ alkyl or halo; R² is C₁-C₄ alkoxy optionally substituted by 1 to 3 R²²; R³ is H; R⁴ is H, halo or 5-6 membered heteroaryl optionally substituted by 1 to 3 R³⁴; R⁵ is H; R⁶ is H; X and Z are independently C₁-C₄ alkyl; and Y is H.

In another preferred embodiment of formula (II), the compounds have a combination of two or more of the following preferred features: R¹ is C₁-C₄ alkyl or halo; R² is 3-12 membered heterocyclyl optionally substituted by 1 to 3 R³²; R³ is H; R⁴ is H, halo or 5-6 membered heteroaryl optionally substituted by 1 to 3 R³⁴; R⁵ is H; R⁶ is H; X and Z are independently C₁-C₄ alkyl; and Y is H.

In some particularly preferred embodiments of formula (II), the compounds have a combination of three, four, five, six, seven or eight of the preferred features in each of the preferred sets described above.

In another aspect, the invention provides compounds of formula (III),

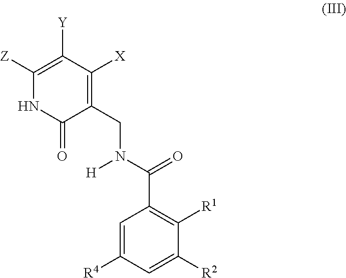

or a pharmaceutically acceptable salt thereof,

wherein:

R¹ is C₁-C₈ alkyl, C₁-C₈ alkoxy, halo, —OH, —CN or —NR⁷R⁸, where each said C₁-C₈ alkyl or C₁-C₈ alkoxy is optionally substituted by one or more R²¹;

R² is 3-12 membered heterocyclyl, C₆-C₁₂ aryl, 5-12 membered heteroaryl or C₁-C₈ alkoxy, where said C₁-C₈ alkoxy is optionally substituted by one or more R²², and each said heterocyclyl, aryl or heteroaryl is optionally substituted by one or more R³²;

R⁴ is independently selected from the group consisting of H, C₁-C₈ alkyl, C₁-C₈ alkoxy, C₁-C₈ thioalkoxy, halo, —OH, —CN, C₃-C₈ cycloalkyl, 3-12 membered heterocyclyl, C₆-C₁₂ aryl, 5-12 membered heteroaryl, —OR¹¹ and —NR⁷R⁸, where each said C₁-C₈ alkyl, C₁-C₈ alkoxy, C₁-C₈ thioalkoxy or C₃-C₈ cycloalkyl is optionally substituted by one or more R²⁴, and each said heterocyclyl, aryl, heteroaryl or R¹¹ is optionally substituted by one or more R³⁴;

each R⁷ and R⁸ is independently H or C₁-C₈ alkyl, where said C₁-C₈ alkyl is optionally substituted by one or more R²⁷; or

R⁷ and R⁸ may be taken together with the N atom to which they are attached to form a 3-12 membered heterocyclyl or 5-12 membered heteroaryl, each optionally containing 1, 2 or 3 additional heteroatoms selected from O, N and S, wherein each said heterocyclyl or heteroaryl is optionally substituted by one or more R³⁷;

each R²¹, and R²² is independently selected from the group consisting of halo, —OH, C₁-C₄ alkoxy, —CN and —NR⁹R¹⁰;

each R²⁴ and R²⁷ is independently selected from the group consisting of halo, —OH, C₁-C₄ alkoxy, —CN, —NR⁹R¹⁰, C₃-C₈ cycloalkyl, 3-12 membered heterocyclyl, C₆-C₁₂ aryl and 5-12 membered heteroaryl, where each said cycloalkyl, heterocyclyl, aryl or heteroaryl is optionally substituted by one or more substituents independently selected from the group consisting of halo, —OH, ═O, C₁-C₄ alkyl, C₁-C₄ alkoxy, —CN, —NH₂, —NH(C₁-C₄ alkyl) and —N(C₁-C₄ alkyl)₂;

each R⁹ and R¹⁰ is independently H or C₁-C₄ alkyl; or

R⁹ and R¹⁰ may be taken together with the N atom to which they are attached to form a 3-12 membered heterocyclyl or 5-12 membered heteroaryl, each optionally containing 1, 2 or 3 additional heteroatoms selected from O, N and S, where each said heterocyclyl or heteroaryl is optionally substituted by one or more substituents independently selected from the group consisting of halo, —OH, ═O, C₁-C₄ alkyl, C₁-C₄ alkoxy, —CN, —NH₂, —NH(C₁-C₄ alkyl) and —N(C₁-C₄ alkyl)₂;

R¹¹ is selected from the group consisting of C₃-C₈ cycloalkyl, 3-12 membered heterocyclyl, C₆-C₁₂ aryl and 5-12 membered heteroaryl;

each R³², R³⁴ and R³⁷ is independently selected from the group consisting of halo, C₁-C₈ alkyl, —CN, ═O, —COR^{c}, —CO₂R^{c}, —CONR^{c}R^{d}, —OR^{c}, —SR^{c}, —SOR^{c}, —SO₂R^{c}, —SO₂NR^{c}R^{d}, —NO₂, —NR^{c}R^{d}, —NR^{c}C(O)R^{d}, —NR^{c}C(O)NR^{c}R^{d}, —NR^{c}C(O)OR^{d}—NR^{c}SO₂R^{d}, —NR^{c}SO₂NR^{c}R^{d}, —OC(O)R^{c}, —OC(O)NR^{c}R^{d}, C₃-C₈ cycloalkyl, 3-12 membered heterocyclyl, C₆-C₁₂ aryl and 5-12 membered heteroaryl;
each R^{c} and R^{d} is independently selected from the group consisting of H, C₁-C₈ alkyl, C₃-C₈ cycloalkyl, 3-12 membered heterocyclyl, C₆-C₁₂ aryl and 5-12 membered heteroaryl; or
R^{c} and R^{d} may be taken together with the N atom to which they are attached to form a 3-12 membered heterocyclyl or 5-12 membered heteroaryl ring, each optionally containing 1, 2 or 3 additional heteroatoms selected from O, N and S;
wherein each said alkyl, cycloalkyl, heterocyclyl, aryl or heteroaryl in R³², R³⁴, R³⁷, R^{c} and R^{d} is optionally substituted by 1 to 3 substituents independently selected from the group consisting of halo, —OH, ═O, C₁-C₄ alkyl, C₁-C₄ alkoxy, C₁-C₆ haloalkyl, C₁-C₆ hydroxyalkyl, C₁-C₄ alkoxy-C₁-C₆ alkyl, —CN, —NH₂, —NH(C₁-C₄ alkyl) and —N(C₁-C₄ alkyl)₂;

X and Z are independently selected from the group consisting of H, C₁-C₈ alkyl, C₂-C₈ alkenyl, C₂-C₈ alkynyl, C₃-C₈ cycloalkyl, 3-12 membered heterocyclyl, C₆-C₁₂ aryl, 5-12 membered heteroaryl, halo, CN, —COR^{a}, —CO₂R^{a}, —CONR^{a}R^{b}, —SR^{a}, —SOR^{a}, —SO₂R^{a}, —SO₂NR^{a}R^{b}, —NO₂, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}—OR^{a}, —OC(O)R^{a} or —OC(O)NR^{a}R^{b};
wherein each said C₁-C₈ alkyl, C₂-C₈ alkenyl, C₂-C₈ alkynyl, C₃-C₈ cycloalkyl, 3-12 membered heterocyclyl, C₆-C₁₂ aryl, or 5-12 membered heteroaryl group is optionally substituted by one or more substituents independently selected from the group consisting of halo, —CN, —COR^{a}, —CO₂R^{a}, —CONR^{a}R^{b}, —SR^{a}, —SOR^{a}, —SO₂R^{a}, —SO₂NR^{a}R^{b}, —NO₂, —NR^{a}R^{b}, —NR^{a}C(O)R^{b}, —NR^{a}C(O)NR^{a}R^{b}, —NR^{a}C(O)OR^{a}—NR^{a}SO₂R^{b}, —NR^{a}SO₂NR^{a}R^{b}, —OR^{a}, —OC(O)R^{a}, —OC(O)NR^{a}R^{b}, C₃-C₈ cycloalkyl, 3-12 membered heterocyclyl, C₆-C₁₂ aryl, and 5-12 membered heteroaryl;
each R^{a} and R^{b} is independently H, C₁-C₈ alkyl, C₂-C₈ alkenyl, C₂-C₈ alkynyl, C₃-C₈ cycloalkyl, 3-12 membered heterocyclyl, C₆-C₁₂ aryl or 5-12 membered heteroaryl, where each said alkyl, alkenyl, alkynyl, cycloalkyl, heterocyclyl, aryl and heteroaryl is optionally substituted by one or more substituents independently selected from the group consisting of halo, C₁-C₄ alkyl, —OR″, —NR″₂, —CO₂R″, —CONR″₂, —SO₂R″ and —SO₂NR″₂, where each R″ is independently H or C₁-C₄ alkyl; or
R^{a} and R^{b} may be taken together with the N atom to which they are attached to form a 3-12 membered heterocyclyl or 5-12 membered heteroaryl, each optionally containing 1, 2 or 3 additional heteroatoms selected from O, N and S, wherein said heterocyclyl or heteroaryl is optionally substituted by one or more substituents independently selected from the group consisting of halo, —OH, ═O, C₁-C₄ alkyl, C₁-C₄ alkoxy, C₁-C₆ haloalkyl, C₁-C₆ hydroxyalkyl, C₁-C₄ alkoxy-C₁-C₆ alkyl, —CN, —NH₂, —NH(C₁-C₄ alkyl) and —N(C₁-C₄ alkyl)₂; and

Y is H, halo, —OH or C₁-C₄ alkoxy.

The embodiments described herein with respect to formula (I) and formula (II), and combinations thereof, are also applicable to formula (III).

In one embodiment of formula (III), R¹ is optionally substituted C₁-C₄ alkyl or halo. In some such embodiments, R¹ is C₁-C₄ alkyl optionally substituted by 1 to 3 R²¹, where each R²¹ is independently halo, —OH, C₁-C₄ alkoxy, —CN and —NR⁹R¹⁰, and each R⁹ and R¹⁰ is independently H or C₁-C₄ alkyl.

In another embodiment of formula (III), R¹ is C₁-C₄ alkyl or halo. In some such embodiments, R¹ is methyl, ethyl, n-propyl, isopropyl, n-butyl, sec-butyl or tert-butyl. In other such embodiments, R¹ is CI or F. In further embodiments, R¹ is methyl, ethyl, chloro or fluoro.

In another embodiment of formula (III), R² is 5-12 membered heteroaryl optionally substituted by 1 to 3 R³². In some such embodiments, said 5-12 membered heteroaryl is selected from the group consisting of pyrazolyl, imidazolyl, triazolyl and pyrrolyl, where said heteroaryl is optionally substituted by 1 to 3 R³² groups. In preferred embodiments of formula (III), each R³² is independently halo or C₁-C₄ alkyl.

In another embodiment of formula (III), R² is C₁-C₈ alkoxy optionally substituted by one or more R²². In some such embodiments, R² is C₁-C₈ alkoxy optionally substituted by 1 to 3 R²² groups. In some such embodiments, R² is C₁-C₄ alkoxy optionally substituted by 1 to 3 R²² groups.

In such embodiments, each R²² is independently selected from the group consisting of halo, —OH, C₁-C₄ alkoxy, —CN and —NR⁹R¹⁰, where each R⁹ and R¹⁰ is independently H or C₁-C₄ alkyl, or R⁹ and R¹⁰ may be taken together with the N atom to which they are attached to form an optionally substituted heterocyclyl or heteroaryl moiety, optionally containing 1, 2 or 3 additional heteroatoms selected from N, O and S. In some such embodiments, R⁹ and R¹⁰ may be taken together with the N atom to which they are attached to form a 3-12 membered heterocyclyl or 5-12 membered heteroaryl, each optionally containing 1, 2 or 3 additional heteroatoms selected from O, N and S, where each said heterocyclyl or heteroaryl is optionally substituted by 1 to 3 substituents independently selected from the group consisting of halo, —OH, ═O, C₁-C₄ alkyl, C₁-C₄ alkoxy, —CN, —NH₂, —NH(C₁-C₄ alkyl) and —N(C₁-C₄ alkyl)₂.

In other such embodiments, each R²² is independently selected from the group consisting of Cl, F, —OH, —OCH₃, —OC₂H₅, —OCF₃, —CN, —NH₂, —NHCH₃, —N(CH₃)₂, cyclopropyl, optionally substituted 4-6 membered heterocyclyl and optionally substituted 5-6 membered heteroaryl. In some embodiments, said 4-6 membered heterocyclyl or said 5-6 membered heteroaryl is optionally substituted by halo, —OH, ═O, C₁-C₄ alkyl, C₁-C₄ alkoxy, —CN—NH₂, —NH(C₁-C₄ alkyl) or —N(C₁-C₄ alkyl)₂.

In further embodiments, R² is C₁-C₄ alkoxy. In specific embodiments, said alkoxy is methoxy, ethoxy, propoxy, isopropoxy, n-butoxy, sec-butoxy or tert-butoxy. In specific embodiments, R² is isopropoxy.

In compounds of formula (III), R⁴ is independently selected from the group consisting of H, C₁-C₈ alkyl, C₁-C₈ alkoxy, C₁-C₈ thioalkoxy, halo, —OH, —CN, C₃-C₈ cycloalkyl, 3-12 membered heterocyclyl, C₆-C₁₂ aryl, 5-12 membered heteroaryl, —OR¹¹ and —NR⁷R⁸, where each said C₁-C₈ alkyl, C₁-C₈ alkoxy, C₁-C₈ thioalkoxy or C₃-C₈ cycloalkyl is optionally substituted by one or more R²⁴, and each said heterocyclyl, aryl, heteroaryl or R¹¹ is optionally substituted by one or more R³⁴.

In one embodiment of formula (III), R⁴ is H, halo, —CN or 5-12 membered heteroaryl, where said heteroaryl is optionally substituted by one or more R³⁴. Preferably, said heteroaryl is optionally substituted by 1 to 3 R³⁴. In some such embodiments, said 5-12 membered heteroaryl is selected from the group consisting of pyridyl, pyrimidinyl, pyrazinyl, pyrazolyl, imidazolyl, triazolyl and pyrrolyl, where said heteroaryl is optionally substituted by one or more R³⁴, preferably, by 1 to 3 R³⁴.

In some such embodiments, each R³⁴ is independently selected from the group consisting of halo, C₁-C₄ alkyl, —OR^{c}, —SR^{c}, —SO₂R^{c} and —NR^{c}R^{d}, where each R^{c} and R^{d} is independently H or C₁-C₄ alkyl. In other embodiments, each R³⁴ is independently selected from the group consisting of halo, C₁-C₄ alkyl, CN, —OR^{c}, —SR^{c}, —SO₂R^{c} and —NR^{c}R^{d}, where each R^{c} and R^{d} is independently H or C₁-C₄ alkyl, or R^{c} and R^{d} in —NR^{c}R^{d} may be taken together with the N atom to which they are attached to form a 4-6 membered heterocyclyl optionally containing 1 additional heteroatom selected from O, N and S, where said 4-6 membered heterocyclyl is optionally substituted by 1 to 3 substituents independently selected from the group consisting of halo, —OH, ═O, C₁-C₄ alkyl, C₁-C₄ alkoxy, C₁-C₆ haloalkyl, C₁-C₆ hydroxyalkyl, C₁-C₄ alkoxy-C₁-C₆ alkyl, —CN, —NH₂, —NH(C₁-C₄ alkyl) and —N(C₁-C₄ alkyl)₂.

In specific embodiments, each R³⁴ is independently selected from the group consisting of halo, —OH, —CN, C₁-C₄ alkyl, C₁-C₄ alkoxy, —NH₂, —NH(C₁-C₄ alkyl) and —N(C₁-C₄ alkyl)₂. In particular embodiments, each R³⁴ is independently selected from the group consisting of Cl, F, —OH, —CH₃, —C₂H₅, —CF₃, —OCH₃, —OC₂H₅, —OCF₃, —SCH₃, —CN, —NH₂, —NHCH₃ and —N(CH₃)₂.

In preferred embodiments of formula (III), X and Z are independently C₁-C₄ alkyl.

In one preferred embodiment of formula (III), the compounds have a combination of two or more of the following preferred features: R¹ is C₁-C₄ alkyl or halo; R² is optionally substituted 5-6 membered heteroaryl; R⁴ is H, halo or optionally substituted heteroaryl; X and Z are independently C₁-C₄ alkyl; and Y is H or F.

In another preferred embodiment of formula (III), the compounds have a combination of two or more of the following preferred features: R¹ is C₁-C₄ alkyl or halo; R² is optionally substituted C₁-C₄ alkoxy; R⁴ is H, halo or optionally substituted heteroaryl; X and Z are independently C₁-C₄ alkyl; and Y is H or F.

In another preferred embodiment of formula (III), the compounds have a combination of two or more of the following preferred features: R¹ is C₁-C₄ alkyl or halo; R² is optionally substituted 3-12 membered heterocyclyl; R⁴ is H, halo or optionally substituted heteroaryl; X and Z are independently C₁-C₄ alkyl; and Y is H or F.

In still another preferred embodiment of formula (III), the compounds have a combination of two or more of the following preferred features: R¹ is C₁-C₄ alkyl or halo; R² is optionally substituted 5-6 membered heteroaryl; R⁴ is H, halo or optionally substituted 5-6 membered heteroaryl; X and Z are independently C₁-C₄ alkyl; and Y is H or F.

In yet another preferred embodiment of formula (III), the compounds have a combination of two or more of the following preferred features: R¹ is C₁-C₄ alkyl or halo; R² is optionally substituted C₁-C₄ alkoxy; R⁴ is H, halo or optionally substituted 5-6 membered heteroaryl; X and Z are independently C₁-C₄ alkyl; and Y is H or F.

In a further preferred embodiment of formula (III), the compounds have a combination of two or more of the following preferred features: R¹ is C₁-C₄ alkyl or halo; R² is optionally substituted 3-12 membered heterocyclyl; R⁴ is H, halo or optionally substituted 5-6 membered heteroaryl; X and Z are independently C₁-C₄ alkyl; and Y is H or F.

In another preferred embodiment of formula (III), the compounds have a combination of two or more of the following preferred features: R¹ is C₁-C₄ alkyl or halo; R² is 5-6 membered heteroaryl optionally substituted by 1 to 3 R³²; R⁴ is H, halo or 5-6 membered heteroaryl optionally substituted by 1 to 3 R³⁴; X and Z are independently C₁-C₄ alkyl; and Y is H.

In another preferred embodiment of formula (III), the compounds have a combination of two or more of the following preferred features: R¹ is C₁-C₄ alkyl or halo; R² is C₁-C₄ alkoxy optionally substituted by 1 to 3 R²²; R⁴ is H, halo or 5-6 membered heteroaryl optionally substituted by 1 to 3 R³⁴; X and Z are independently C₁-C₄ alkyl; and Y is H.

In another preferred embodiment of formula (III), the compounds have a combination of two or more of the following preferred features: R¹ is C₁-C₄ alkyl or halo; R² is 3-12 membered heterocyclyl optionally substituted by 1 to 3 R³²; R⁴ is H, halo or 5-6 membered heteroaryl optionally substituted by 1 to 3 R³⁴; X and Z are independently C₁-C₄ alkyl; and Y is H.

In some particularly preferred embodiments of formula (III), the compounds have a combination of three, four or five of the preferred features in each of the preferred sets described above.

In another aspect, the invention provides compounds of formula (IV),

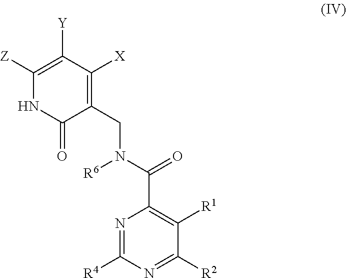

or a pharmaceutically acceptable salt thereof,

wherein R¹, R², R⁴, R⁶, X, Y and Z are defined as in formula (I).

The embodiments described herein with respect to formula (I) and combinations thereof, are also applicable to formula (IV).

In one embodiment of formula (IV), R¹ is optionally substituted C₁-C₄ alkyl or halo. In some such embodiments, R¹ is C₁-C₄ alkyl optionally substituted by 1 to 3 R²¹, where each R²¹ is independently halo, —OH, C₁-C₄ alkoxy, —CN and —NR⁹R¹⁰, where each R⁹ and R¹⁰ is independently H or C₁-C₄ alkyl.

In another embodiment of formula (IV), R¹ is C₁-C₄ alkyl or halo. In some such embodiments, R¹ is methyl, ethyl, n-propyl, isopropyl, n-butyl, sec-butyl or tert-butyl. In other such embodiments, R¹ is CI or F. In further embodiments, R¹ is methyl, ethyl, chloro or fluoro.

In some embodiments of formula (IV), R² is 3-12 membered heterocyclyl, where each said heterocyclyl is optionally substituted by one or more R³². In some such embodiments, each said heterocyclyl is optionally substituted by 1 to 3 R³². In some such embodiments, each R³² is independently selected from the group consisting of halo, C₁-C₄ alkyl, C₃-C₈ cycloalkyl, C₆-C₁₂ aryl and 5-12 membered heteroaryl, where each said aryl or heteroaryl is optionally substituted by halo, —OH, C₁-C₄ alkyl, C₁-C₄ alkoxy, —NH₂, —NH(C₁-C₄ alkyl) or —N(C₁-C₄ alkyl)₂.

In another embodiment of formula (IV), R² is 5-12 membered heteroaryl, where each said heteroaryl is optionally substituted by one or more R³². In some such embodiments, each said heteroaryl is optionally substituted by 1 to 3 R³². In some such embodiments, each R³² is independently selected from the group consisting of halo, C₁-C₄ alkyl, C₁-C₄ alkoxy, where each said alkyl or alkoxy is optionally substituted by halo, —OH, C₁-C₄ alkoxy, —NH₂, —NH(C₁-C₄ alkyl) or —N(C₁-C₄ alkyl)₂.

In compounds of formula (IV), R⁴ is independently selected from the group consisting of H, C₁-C₈ alkyl, C₁-C₈ alkoxy, C₁-C₈ thioalkoxy, halo, —OH, —CN, C₃-C₈ cycloalkyl, 3-12 membered heterocyclyl, C₆-C₁₂ aryl, 5-12 membered heteroaryl, —OR¹¹ and —NR⁷R⁸, where each said C₁-C₈ alkyl, C₁-C₈ alkoxy, C₁-C₈ thioalkoxy or C₃-C₈ cycloalkyl is optionally substituted by one or more R²⁴, and each said heterocyclyl, aryl, heteroaryl or R¹¹ is optionally substituted by one or more R³⁴.

In some embodiments of formula (IV), R⁴ is H, halo, —CN or 5-12 membered heteroaryl, where said heteroaryl is optionally substituted by one or more R³⁴. In some such embodiments, said heteroaryl is optionally substituted by 1 to 3 R³⁴. In some such embodiments, each R³⁴ is independently selected from the group consisting of halo, C₁-C₄ alkyl, —OR^{c}, —SR^{c}, —SO₂R^{c} and —NR^{c}R^{d}, where each R^{c} and R^{d} is independently H or C₁-C₄ alkyl.

In some embodiments of formula (IV), R⁶ is H or methyl, preferably H.

In some embodiments of formula (IV), X and Z are independently C₁-C₈ alkyl. In preferred embodiments, X and Z are independently C₁-C₄ alkyl. In further embodiments, X and Z are independently C₁-C₈ alkyl and Y is H or fluoro, preferably H. In some such embodiments, X and Y are independently C₁-C₄ alkyl and Y is H or fluoro, preferably H.

In one preferred embodiment of formula (IV), the compounds have a combination of two or more of the following preferred features: R¹ is C₁-C₄ alkyl or halo; R² is optionally substituted 5-6 membered heteroaryl; R⁴ is H, halo or optionally substituted heteroaryl; R⁶ is H; X and Z are independently C₁-C₄ alkyl; and Y is H or F.

In another preferred embodiment of formula (IV), the compounds have a combination of two or more of the following preferred features: R¹ is C₁-C₄ alkyl or halo; R² is optionally substituted C₁-C₄ alkoxy; R⁴ is H, halo or optionally substituted heteroaryl; R⁶ is H; X and Z are independently C₁-C₄ alkyl; and Y is H or F.

In another preferred embodiment of formula (IV), the compounds have a combination of two or more of the following preferred features: R¹ is C₁-C₄ alkyl or halo; R² is optionally substituted 3-12 membered heterocyclyl; R⁴ is H, halo or optionally substituted heteroaryl; R⁶ is H; X and Z are independently C₁-C₄ alkyl; and Y is H or F.

In some particularly preferred embodiments of formula (IV), the compounds have a combination of three, four, five or six of the preferred features in each of the preferred sets described above.

In yet another aspect, the invention provides a compound of formula (V):

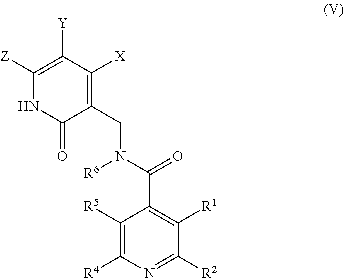

or a pharmaceutically acceptable salt thereof,

wherein R¹, R², R⁴, R⁵, R⁶, X, Y and Z are defined as in formula (I).

The embodiments described herein with respect to formula (I) and combinations thereof, are also applicable to formula (V).

In some embodiments of formula (V), R¹ is C₁-C₄ alkyl or halo. In some such embodiments, R¹ is methyl, ethyl, chloro or fluoro.

In other embodiments of formula (V), R² is 3-12 membered heterocyclyl optionally substituted by one or more R³². In some such embodiments, each R³² is independently selected from the group consisting of halo, C₁-C₄ alkyl, C₃-C₈ cycloalkyl, C₆-C₁₂ aryl and 5-12 membered heteroaryl, where each said aryl or heteroaryl is optionally substituted by halo, —OH, C₁-C₄ alkyl, C₁-C₄ alkoxy, —NH₂, —NH(C₁-C₄ alkyl) or —N(C₁-C₄ alkyl)₂.

In other embodiments of formula (V), R² is C₁-C₈ alkoxy optionally substituted by one or more R²². In some such embodiments, R² is C₁-C₄ alkoxy.

In compounds of formula (V), R⁴ is independently selected from the group consisting of H, C₁-C₈ alkyl, C₁-C₈ alkoxy, C₁-C₈ thioalkoxy, halo, —OH, —CN, C₃-C₈ cycloalkyl, 3-12 membered heterocyclyl, C₆-C₁₂ aryl, 5-12 membered heteroaryl, —OR¹¹ and —NR⁷R⁸, where each said C₁-C₈ alkyl, C₁-C₈ alkoxy, C₁-C₈ thioalkoxy or C₃-C₈ cycloalkyl is optionally substituted by one or more R²⁴, and each said heterocyclyl, aryl, heteroaryl or R¹¹ is optionally substituted by one or more R³⁴.

In one embodiment of formula (V), R⁴ is H, halo, —CN or 5-12 membered heteroaryl, where said heteroaryl is optionally substituted by one or more R³⁴. In some such embodiments, said heteroaryl is substitute by 1 to 3 R³⁴. In some such embodiments, said heteroaryl is selected from the group consisting of pyridyl, pyrimidinyl, pyrazinyl, pyrazolyl, imidazolyl and pyrrolyl, where said heteroaryl is optionally substituted by 1 to 3 R³⁴. In specific embodiments, each R³⁴ is independently selected from the group consisting of halo, —OH, C₁-C₄ alkyl, C₁-C₄ alkoxy, —NH₂, —NH(C₁-C₄ alkyl) and —N(C₁-C₄ alkyl)₂.

In frequent embodiments of formula (V), R⁵ is H or halo, preferably H or F, and more R⁵ is H.

In other embodiments of formula (V), R⁶ is methyl or H. Preferably, R⁶ is H.

In some embodiments of formula (V), X and Z are independently C₁-C₈ alkyl. In preferred embodiments, X and Z are independently C₁-C₄ alkyl. In further embodiments, X and Z are independently C₁-C₈ alkyl and Y is H or fluoro, preferably H. In some such embodiments, X and Y are independently C₁-C₄ alkyl and Y is H or fluoro, preferably H.

In one preferred embodiment of formula (V), the compounds have a combination of two or more of the following preferred features: R¹ is C₁-C₄ alkyl or halo; R² is optionally substituted 5-6 membered heteroaryl; R⁴ is H, halo or optionally substituted heteroaryl; R⁵ is H or F; R⁶ is H; X and Z are independently C₁-C₄alkyl; and Y is H or F.

In another preferred embodiment of formula (V), the compounds have a combination of two or more of the following preferred features: R¹ is C₁-C₄ alkyl or halo; R² is optionally substituted C₁-C₄ alkoxy; R⁴ is H, halo or optionally substituted heteroaryl; R⁵ is H or F; R⁶ is H; X and Z are independently C₁-C₄ alkyl; and Y is H or F.

In another preferred embodiment of formula (V), the compounds have a combination of two or more of the following preferred features: R¹ is C₁-C₄ alkyl or halo; R² is optionally substituted 3-12 membered heterocyclyl; R⁴ is H, halo or optionally substituted heteroaryl; R⁵ is H or F; R⁶ is H; X and Z are independently C₁-C₄ alkyl; and Y is H or F.

In some particularly preferred embodiments of formula (V), the compounds have a combination of three, four, five, six or seven of the preferred features in each of the preferred sets described above.

In yet another aspect, the invention provides a compound of formula (VI):

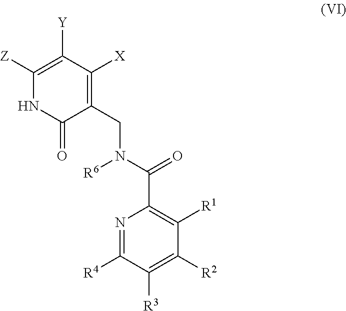

or a pharmaceutically acceptable salt thereof,

wherein R¹, R², R³, R⁴, R⁶, X, Y and Z are defined as in formula (I).

The embodiments described herein with respect to formula (I) and combinations thereof, are also applicable to formula (VI).

In some embodiments of formula (VI), R¹ is C₁-C₄ alkyl or halo. In some such embodiments, R¹ is methyl, ethyl, chloro or fluoro.

In other embodiments of formula (VI), R² is 3-12 membered heterocyclyl or 5-12 membered heteroaryl, each optionally substituted by one or more R³². In some such embodiments, said heterocyclyl or heteroaryl is optionally substituted by 1 to 3 R³². In other such embodiments, each R³² is independently selected from the group consisting of halo, C₁-C₄ alkyl, C₃-C₈ cycloalkyl, C₆-C₁₂ aryl and 5-12 membered heteroaryl, where each said aryl or heteroaryl is optionally substituted by halo, —OH, C₁-C₄ alkyl, C₁-C₄ alkoxy, —NH₂, —NH(C₁-C₄ alkyl) or —N(C₁-C₄ alkyl)₂.

In other embodiments of formula (VI), R² is C₁-C₈ alkoxy optionally substituted by one or more R²². In some such embodiments, R² is C₁-C₄ alkoxy.

In frequent embodiments of formula (VI), R³ is H or halo, preferably H or F, and more R³ is H.

In compounds of formula (VI), R⁴ is independently selected from the group consisting of H, C₁-C₈ alkyl, C₁-C₈ alkoxy, C₁-C₈ thioalkoxy, halo, —OH, —CN, C₃-C₈ cycloalkyl, 3-12 membered heterocyclyl, C₆-C₁₂ aryl, 5-12 membered heteroaryl, —OR¹¹ and —NR⁷R⁸, where each said C₁-C₈ alkyl, C₁-C₈ alkoxy, C₁-C₈ thioalkoxy or C₃-C₈ cycloalkyl is optionally substituted by one or more R²⁴, and each said heterocyclyl, aryl, heteroaryl or R¹¹ is optionally substituted by one or more R³⁴.

In one embodiment of formula (VI), R⁴ is H, halo, —CN or 5-12 membered heteroaryl, where said heteroaryl is optionally substituted by one or more R³⁴. In some such embodiments, said heteroaryl is substituted by 1 to 3 R³⁴. In some such embodiments, said heteroaryl is selected from the group consisting of pyridyl, pyrimidinyl, pyrazinyl, pyrazolyl, imidazolyl and pyrrolyl, where said heteroaryl is optionally substituted by 1 to 3 R³⁴. In specific embodiments, each R³⁴ is independently selected from the group consisting of halo, —OH, C₁-C₄ alkyl, C₁-C₄ alkoxy, —NH₂, —NH(C₁-C₄ alkyl) and —N(C₁-C₄ alkyl)₂.

In other embodiments of formula (VI), R⁶ is methyl or H. Preferably, R⁶ is H.

In some embodiments of formula (VI), X and Z are independently C₁-C₈ alkyl. In preferred embodiments, X and Z are independently C₁-C₄ alkyl. In further embodiments, X and Z are independently C₁-C₈ alkyl and Y is H or fluoro, preferably H. In some such embodiments, X and Y are independently C₁-C₄ alkyl and Y is H or fluoro, preferably H.

In one preferred embodiment of formula (VI), the compounds have a combination of two or more of the following preferred features: R¹ is C₁-C₄ alkyl or halo; R² is optionally substituted 5-6 membered heteroaryl; R³ is H or F; R⁴ is H, halo or optionally substituted heteroaryl; R⁶ is H; X and Z are independently C₁-C₄ alkyl; and Y is H or F.

In another preferred embodiment of formula (VI), the compounds have a combination of two or more of the following preferred features: R¹ is C₁-C₄ alkyl or halo; R² is optionally substituted C₁-C₄ alkoxy; R³ is H or F; R⁴ is H, halo or optionally substituted heteroaryl; R⁶ is H; X and Z are independently C₁-C₄ alkyl; and Y is H or F.

In another preferred embodiment of formula (VI), the compounds have a combination of two or more of the following preferred features: R¹ is C₁-C₄ alkyl or halo; R² is optionally substituted 3-12 membered heterocyclyl; R³ is H or F; R⁴ is H, halo or optionally substituted heteroaryl; R⁶ is H; X and Z are independently C₁-C₄alkyl; and Y is H or F.

In some particularly preferred embodiments of formula (VI), the compounds have a combination of three, four, five, six or seven of the preferred features in each of the preferred sets described above.

In yet another aspect, the invention provides a compound of formula (VII):

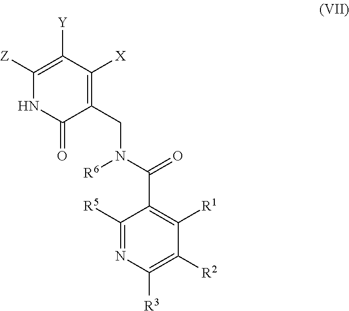

or a pharmaceutically acceptable salt thereof,

wherein R¹, R², R³, R⁵, R⁶, X, Y and Z are defined as in formula (I).

The embodiments described herein with respect to formula (I) and combinations thereof, are also applicable to formula (VII).

In some embodiments of formula (VII), R¹ is C₁-C₄ alkyl or halo. In some such embodiments, R¹ is methyl, ethyl, chloro or fluoro.

In other embodiments of formula (VII), R² is 3-12 membered heterocyclyl or 5-12 membered heteroaryl, each optionally substituted by one or more R³². In some such embodiments, said heterocyclyl or heteroaryl is optionally substituted by 1 to 3 R³². In other such embodiments, each R³² is independently selected from the group consisting of halo, C₁-C₄ alkyl, C₃-C₈ cycloalkyl, C₆-C₁₂ aryl and 5-12 membered heteroaryl, where each said aryl or heteroaryl is optionally substituted by halo, —OH, C₁-C₄ alkyl, C₁-C₄ alkoxy, —NH₂, —NH(C₁-C₄ alkyl) or —N(C₁-C₄ alkyl)₂.

In other embodiments of formula (VII), R² is C₁-C₈ alkoxy optionally substituted by one or more R²². In some such embodiments, R² is C₁-C₄ alkoxy.

In frequent embodiments of formula (VII), R³ and R⁵ are independently H or halo. Preferably R³ and R⁵ are independently H or F, and more preferably, R³ and R⁵ are H.

In other embodiments of formula (VII), R⁶ is methyl or H. Preferably, R⁶ is H.

In some embodiments of formula (VII), X and Z are independently C₁-C₈ alkyl. In preferred embodiments, X and Z are independently C₁-C₄ alkyl. In further embodiments, X and Z are independently C₁-C₈ alkyl and Y is H or fluoro, preferably H. In some such embodiments, X and Y are independently C₁-C₄ alkyl and Y is H or fluoro, preferably H.

In one preferred embodiment of formula (VII), the compounds have a combination of two or more of the following preferred features: R¹ is C₁-C₄ alkyl or halo; R² is optionally substituted 5-6 membered heteroaryl; R³ is H or F; R⁵ is H or F; R⁶ is H; X and Z are independently C₁-C₄alkyl; and Y is H or F.

In another preferred embodiment of formula (VII), the compounds have a combination of two or more of the following preferred features: R¹ is C₁-C₄ alkyl or halo; R² is optionally substituted C₁-C₄ alkoxy; R³ is H or F; R⁵ is H or F; R⁶ is H; X and Z are independently C₁-C₄ alkyl; and Y is H or F.

In another preferred embodiment of formula (VII), the compounds have a combination of two or more of the following preferred features: R¹ is C₁-C₄ alkyl or halo; R² is optionally substituted 3-12 membered heterocyclyl; R³ is H or F; R⁵ is H or F; R⁶ is H; X and Z are independently C₁-C₄ alkyl; and Y is H or F.

In some particularly preferred embodiments of formula (VII), the compounds have a combination of three, four, five, six or of the preferred features in each of the preferred sets described above.

A “pharmaceutical composition” refers to a mixture of one or more of the compounds described herein, or a pharmaceutically acceptable salt, solvate, hydrate or prodrug thereof as an active ingredient, and at least one pharmaceutically acceptable carrier or excipient. The purpose of a pharmaceutical composition is to facilitate administration of a compound to a subject.

Thus, in another aspect the invention provides a pharmaceutical composition comprising a compound of one of the formulae described herein, or a pharmaceutically acceptable salt thereof, and a pharmaceutically acceptable carrier or excipient. In some embodiments, the pharmaceutical composition comprises two or more pharmaceutically acceptable carriers and/or excipients.

In some embodiments, the pharmaceutical composition further comprises at least one additional an anti-cancer therapeutic agent or a palliative agent. In some such embodiments, the at least one additional medicinal or pharmaceutical agent is an anti-cancer agent as described below. In some such embodiments, the combination provides an additive, greater than additive, or synergistic anti-cancer effect. In some such embodiments, the one or more anti-cancer therapeutic agent is selected from the group consisting of anti-tumor agents, anti-angiogenesis agents, signal transduction inhibitors and antiproliferative agents.

In one aspect, the invention provides a method for the treatment of abnormal cell growth in a subject comprising administering to the subject a therapeutically effective amount of a compound of the invention, or a pharmaceutically acceptable salt thereof.

In another aspect, the invention provides a method for the treatment of abnormal cell growth in a subject comprising administering to the subject an amount of a compound of the invention, or a pharmaceutically acceptable salt thereof, in combination with an amount of an anti-tumor agent, which amounts are together effective in treating said abnormal cell growth. In some embodiments, the anti-tumor agent is selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, and anti-androgens.

In frequent embodiments of the methods provided herein, the abnormal cell growth is cancer. In some embodiments, the methods provided result in one or more of the following effects: (1) inhibiting cancer cell proliferation; (2) inhibiting cancer cell invasiveness; (3) inducing apoptosis of cancer cells; (4) inhibiting cancer cell metastasis; or (5) inhibiting angiogenesis.

In another aspect, the invention provides a method for the treatment of a disorder mediated by EZH2 in a subject comprising administering to the subject a compound of the invention, or a pharmaceutically acceptable salt thereof, in an amount that is effective for treating said disorder.

In preferred embodiments of the methods provided herein, the subject is a mammal, in particular a human.

General schemes for synthesizing the compounds of the invention can be found in the Examples section herein.

Unless indicated otherwise, all references herein to the inventive compounds include references to salts, solvates, hydrates and complexes thereof, and to solvates, hydrates and complexes of salts thereof, including polymorphs, stereoisomers, and isotopically labeled versions thereof.

Compounds of the invention may exist in the form of pharmaceutically acceptable salts such as, e.g., acid addition salts and base addition salts of the compounds of one of the formulae provided herein. As used herein, the term “pharmaceutically acceptable salt” refers to those salts which retain the biological effectiveness and properties of the parent compound. The phrase “pharmaceutically acceptable salt(s)”, as used herein, unless otherwise indicated, includes salts of acidic or basic groups which may be present in the compounds of the formulae disclosed herein.

For example, the compounds of the invention that are basic in nature are capable of forming a wide variety of salts with various inorganic and organic acids. Although such salts must be pharmaceutically acceptable for administration to animals, it is often desirable in practice to initially isolate the compound of the present invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention can be prepared by treating the base compound with a substantially equivalent amount of the selected mineral or organic acid in an aqueous solvent medium or in a suitable organic solvent, such as methanol or ethanol. Upon evaporation of the solvent, the desired solid salt is obtained. The desired acid salt can also be precipitated from a solution of the free base in an organic solvent by adding an appropriate mineral or organic acid to the solution.

The acids that may be used to prepare pharmaceutically acceptable acid addition salts of such basic compounds of those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p toluenesulfonate and pamoate [i.e., 1,1′-methylene-bis-(2-hydroxy-3-naphthoate)] salts.

Examples of salts include, but are not limited to, acetate, acrylate, benzenesulfonate, benzoate (such as chlorobenzoate, methylbenzoate, dinitrobenzoate, hydroxybenzoate, and methoxybenzoate), bicarbonate, bisulfate, bisulfite, bitartrate, borate, bromide, butyne-1,4-dioate, calcium edetate, camsylate, carbonate, chloride, caproate, caprylate, clavulanate, citrate, decanoate, dihydrochloride, dihydrogenphosphate, edetate, edislyate, estolate, esylate, ethylsuccinate, formate, fumarate, gluceptate, gluconate, glutamate, glycollate, glycollylarsanilate, heptanoate, hexyne-1,6-dioate, hexylresorcinate, hydrabamine, hydrobromide, hydrochloride, γ-hydroxybutyrate, iodide, isobutyrate, isothionate, lactate, lactobionate, laurate, malate, maleate, malonate, mandelate, mesylate, metaphosphate, methane-sulfonate, methylsulfate, monohydrogenphosphate, mucate, napsylate, naphthalene-1-sulfonate, naphthalene-2-sulfonate, nitrate, oleate, oxalate, pamoate (embonate), palmitate, pantothenate, phenylacetates, phenylbutyrate, phenylpropionate, phthalate, phosphate/diphosphate, polygalacturonate, propanesulfonate, propionate, propiolate, pyrophosphate, pyrosulfate, salicylate, stearate, subacetate, suberate, succinate, sulfate, sulfonate, sulfite, tannate, tartrate, teoclate, tosylate, triethiodode, and valerate salts.

Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

The compounds of the invention that include a basic moiety, such as an amino group, may form pharmaceutically acceptable salts with various amino acids, in addition to the acids mentioned above.

Those compounds of the invention that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds herein. These salts may be prepared by any suitable method, for example, treatment of the free acid with an inorganic or organic base, such as an amine (primary, secondary or tertiary), an alkali metal hydroxide or alkaline earth metal hydroxide, or the like. These salts can also be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

The chemical bases that may be used as reagents to prepare pharmaceutically acceptable base salts of the compounds of the invention that are acidic in nature are those that form non-toxic base salts with such compounds. Such non-toxic base salts include, but are not limited to, those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002). Methods for making pharmaceutically acceptable salts of compounds of the invention are known to one of skill in the art.

Salts of the present invention can be prepared according to methods known to those of skill in the art. A pharmaceutically acceptable salt of the inventive compounds can be readily prepared by mixing together solutions of the compound and the desired acid or base, as appropriate. The salt may precipitate from solution and be collected by filtration or may be recovered by evaporation of the solvent. The degree of ionization in the salt may vary from completely ionized to almost non-ionized.

It will be understood by those of skill in the art that the compounds of the invention in free base form having a basic functionality may be converted to the acid addition salts by treating with a stoichiometric excess of the appropriate acid. The acid addition salts of the compounds of the invention may be reconverted to the corresponding free base by treating with a stoichiometric excess of a suitable base, such as potassium carbonate or sodium hydroxide, typically in the presence of aqueous solvent, and at a temperature of between about 0° C. and 100° C. The free base form may be isolated by conventional means, such as extraction with an organic solvent. In addition, acid addition salts of the compounds of the invention may be interchanged by taking advantage of differential solubilities of the salts, volatilities or acidities of the acids, or by treating with the appropriately loaded ion exchange resin. For example, the interchange may be affected by the reaction of a salt of the compounds of the invention with a slight stoichiometric excess of an acid of a lower pK than the acid component of the starting salt. This conversion is typically carried out at a temperature between about 0° C. and the boiling point of the solvent being used as the medium for the procedure. Similar exchanges are possible with base addition salts, typically via the intermediacy of the free base form.

The compounds of the invention may exist in both unsolvated and solvated forms. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm. The term ‘solvate’ is used herein to describe a molecular complex comprising the compound of the invention and one or more pharmaceutically acceptable solvent molecules, for example, ethanol. The term ‘hydrate’ is employed when the solvent is water. Pharmaceutically acceptable solvates in accordance with the invention include hydrates and solvates wherein the solvent of crystallization may be isotopically substituted, e.g. D₂O, d₆-acetone, d₆-DMSO.

Also included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionized, partially ionized, or non-ionized. For a review of such complexes, see J Pharm Sci, 64 (8), 1269-1288 by Haleblian (August 1975), the disclosure of which is incorporated herein by reference in its entirety.

The invention also relates to prodrugs of the compounds of the formulae provided herein. Thus, certain derivatives of compounds of the invention which may have little or no pharmacological activity themselves can, when administered to a patient, be converted into the inventive compounds, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association), the disclosures of which are incorporated herein by reference in their entireties.

Prodrugs in accordance with the invention can, for example, be produced by replacing appropriate functionalities present in the inventive compounds with certain moieties known to those skilled in the art as ‘pro-moieties’ as described, for example, in “Design of Prodrugs” by H Bundgaard (Elsevier, 1985), the disclosure of which is incorporated herein by reference in its entirety.

Some non-limiting examples of prodrugs in accordance with the invention include:

(i) where the compound contains a carboxylic acid functionality (—COOH), an ester thereof, for example, replacement of the hydrogen with (C₁-C₈)alkyl;

(ii) where the compound contains an alcohol functionality (—OH), an ether thereof, for example, replacement of the hydrogen with (C₁-C₆)alkanoyloxymethyl; and

(iii) where the compound contains a primary or secondary amino functionality (—NH₂ or —NHR where R≠H), an amide thereof, for example, replacement of one or both hydrogens with a suitably metabolically labile group, such as an amide, carbamate, urea, phosphonate, sulfonate, etc.

Further examples of replacement groups in accordance with the foregoing examples and examples of other prodrug types may be found in the aforementioned references.

Finally, certain inventive compounds may themselves act as prodrugs of other of the inventive compounds.

Also included within the scope of the invention are metabolites of compounds of the formulae described herein, i.e., compounds formed in vivo upon administration of the drug.

The compounds of the formulae provided herein may have asymmetric carbon atoms. The carbon-carbon bonds of the compounds of the invention may be depicted herein using a solid line (————), a solid wedge ( [symbol] ) or a dotted wedge ( [symbol] ). The use of a solid line to depict bonds to asymmetric carbon atoms is meant to indicate that all possible stereoisomers (e.g. specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds of the invention may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds of the invention can exist as enantiomers and diastereomers or as racemates and mixtures thereof. The use of a solid line to depict bonds to one or more asymmetric carbon atoms in a compound of the invention and the use of a solid or dotted wedge to depict bonds to other asymmetric carbon atoms in the same compound is meant to indicate that a mixture of diastereomers is present.

Compounds of the invention that have chiral centers may exist as stereoisomers, such as racemates, enantiomers, or diastereomers.

Stereoisomers of the compounds of the formulae herein can include cis and trans isomers, optical isomers such as (R) and (S) enantiomers, diastereomers, geometric isomers, rotational isomers, atropisomers, conformational isomers, and tautomers of the compounds of the invention, including compounds exhibiting more than one type of isomerism; and mixtures thereof (such as racemates and diastereomeric pairs). Also included are acid addition or base addition salts wherein the counterion is optically active, for example, d-lactate or l-lysine, or racemic, for example, dl-tartrate or dl-arginine.

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

The compounds of the invention may exhibit the phenomena of tautomerism and structural isomerism. For example, the compounds may exist in several tautomeric forms, including the enol and imine form, and the keto and enamine form and geometric isomers and mixtures thereof. All such tautomeric forms are included within the scope of compounds of the invention. Tautomers exist as mixtures of a tautomeric set in solution. In solid form, usually one tautomer predominates. Even though one tautomer may be described, the present invention includes all tautomers of the compounds of the formulae provided.

In addition, some of the compounds of the invention may form atropisomers (e.g., substituted biaryls). Atropisomers are conformational stereoisomers which occur when rotation about a single bond in the molecule is prevented, or greatly slowed, as a result of steric interactions with other parts of the molecule and the substituents at both ends of the single bond are unsymmetrical. The interconversion of atropisomers is slow enough to allow separation and isolation under predetermined conditions. The energy barrier to thermal racemization may be determined by the steric hindrance to free rotation of one or more bonds forming a chiral axis. are stereoisomers resulting from restricted rotation about single bonds where the rotation barrier is high enough to permit isolation of the isomeric species.

Where a compound of the invention contains an alkenyl or alkenylene group, geometric cis/trans (or Z/E) isomers are possible. Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to one skilled in the art.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Stereoisomeric conglomerates may be separated by conventional techniques known to those skilled in the art; see, for example, “Stereochemistry of Organic Compounds” by E L Eliel (Wiley, New York, 1994), the disclosure of which is incorporated herein by reference in its entirety.

“Enantiomerically pure” as used herein, describes a compound that is present as a single enantiomer and which is described in terms of enantiomeric excess (e.e.). Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, “diastereomerically pure” as used herein, describes a compound that is present as a diastereomer and which is described in terms of diasteriomeric excess (d.e.). Preferably, wherein the compound is present as a diastereomer, the diastereomer is present at an diastereomeric excess of greater than or equal to about 80%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

The present invention also includes isotopically-labeled compounds, which are identical to those recited in one of the formulae provided, but for the fact that one or more atoms are replaced by an atom having an atomic mass or mass number different from the atomic mass or mass number usually found in nature.

Isotopically-labeled compounds of the invention can generally be prepared by conventional techniques known to those skilled in the art or by processes analogous to those described herein, using an appropriate isotopically-labeled reagent in place of the non-labeled reagent otherwise employed.

Examples of isotopes that may be incorporated into compounds of the invention include isotopes of hydrogen, carbon, nitrogen, oxygen, phosphorus, fluorine and chlorine, such as, but not limited to, ²H, ³H, ¹³C, ¹⁴C, ¹⁵N, ¹⁸O, ¹⁷O, ³¹P, ³²P, ³⁵S, ¹⁸F, and ³⁶Cl. Certain isotopically-labeled compounds of the invention, for example those into which radioactive isotopes such as ³H and ¹⁴Cl are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically-labeled compounds of the invention may generally be prepared by carrying out the procedures disclosed in the Schemes and/or in the Examples and Preparations below, by substituting an isotopically-labeled reagent for a non-isotopically-labeled reagent.

Compounds of the invention intended for pharmaceutical use may be administered as crystalline or amorphous products, or mixtures thereof. They may be obtained, for example, as solid plugs, powders, or films by methods such as precipitation, crystallization, freeze drying, spray drying, or evaporative drying. Microwave or radio frequency drying may be used for this purpose.

### Therapeutic Methods and Uses

The invention further provides therapeutic methods and uses comprising administering the compounds of the invention, or pharmaceutically acceptable salts thereof, alone or in combination with other therapeutic agents or palliative agents.

In one aspect, the invention provides a method for the treatment of abnormal cell growth in a subject comprising administering to the subject a therapeutically effective amount of a compound of the invention, or a pharmaceutically acceptable salt thereof.

In another aspect, the invention provides a method for the treatment of abnormal cell growth in a subject comprising administering to the subject an amount of a compound of the invention, or a pharmaceutically acceptable salt thereof, in combination with an amount of an anti-tumor agent, which amounts are together effective in treating said abnormal cell growth. In some such embodiments, the anti-tumor agent is selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, and anti-androgens.

In another aspect, the invention provides a method for the treatment of abnormal cell growth in a subject comprising administering to the subject an amount of a compound of the invention, or a pharmaceutically acceptable salt thereof, that is effective in treating abnormal cell growth.

In still another aspect, the invention provides a method of inhibiting cancer cell proliferation in a subject, comprising administering to the subject a compound of the invention, or pharmaceutically acceptable salt thereof, in an amount effective to inhibit cell proliferation.

In another aspect, the invention provides a method of inhibiting cancer cell invasiveness in a subject, comprising administering to the subject a compound of the invention, or pharmaceutically acceptable salt thereof, in an amount effective to inhibit cell invasiveness.

In another aspect, the invention provides a method of inducing apoptosis in cancer cells in a subject, comprising administering to the subject a compound of the invention, or pharmaceutically acceptable salt thereof, in an amount effective to induce apoptosis.

In a further aspect, the invention provides a method of inducing apoptosis in a subject, comprising administering to the subject a therapeutically effective amount of a compound of one of the formulae described herein, or pharmaceutically acceptable salt thereof.

In frequent embodiments of the methods provided herein, the abnormal cell growth is cancer, wherein said cancer is selected from the group consisting of basal cell cancer, medulloblastoma cancer, liver cancer, rhabdomyosarcoma, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers.

In specific embodiments, the cancer is selected from the group consisting of breast, colorectal, endometrial, gastric, liver (e.g., HCC), kidney (e.g., RCC), lung (e.g., NSCLC, SCLC), skin (e.g., melanoma), ovarian, pancreatic, prostate and bladder cancers. In other embodiments, the cancer is a lymphoma, (e.g., DLBCL or FL).

The compounds of the invention are useful for the treatment of cancers, including, e.g., tumors such as brain, breast, cervical, colorectal, endometrial, esophageal, gastric/stomach, head and neck, hepatocellular, laryngeal, lung, oral, ovarian, prostate, testicular and thyroid carcinomas and sarcomas. In specific embodiments, the compounds of the invention are useful for the treatment of breast, colorectal, endometrial, gastric, liver, kidney, lung, melanoma, ovarian, pancreatic, prostate or bladder cancers, diffuse large B-cell lymphoma (DLBCL) or follicular lymphomas (FL).

In some embodiments, the compounds of the invention are active against and/or selective for mutant forms of EZH2, such that trimethylation of H3K27, which is associated with certain cancers, is inhibited. In some such embodiments, the EZH2 mutation is selected from a mutation of tyrosine 641 (Y641), alanine 677 (A677) or alanine 687 (A687).

The term “therapeutically effective amount” as used herein refers to that amount of a compound being administered which will relieve to some extent one or more of the symptoms of the disorder being treated. In reference to the treatment of cancer, a therapeutically effective amount refers to that amount which has the effect of (1) reducing the size of the tumor, (2) inhibiting (that is, slowing to some extent, preferably stopping) tumor metastasis, (3) inhibiting to some extent (that is, slowing to some extent, preferably stopping) tumor growth or tumor invasiveness, and/or (4) relieving to some extent (or, preferably, eliminating) one or more signs or symptoms associated with the cancer.

As used herein, “subject” refers to a human or animal subject. In certain preferred embodiments, the subject is a mammal, and more preferably a human.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

The terms “abnormal cell growth” and “hyperproliferative disorder” are used interchangeably in this application.

“Abnormal cell growth”, as used herein, unless otherwise indicated, refers to cell growth that is independent of normal regulatory mechanisms (e.g., loss of contact inhibition). Abnormal cell growth may be benign (not cancerous), or malignant (cancerous). This includes the abnormal growth of: (1) tumor cells (tumors) that show increased expression of EZH2; (2) benign and malignant cells of other proliferative diseases in which EZH2 is over-expressed; (3) tumors that proliferate by aberrant EZH2 activation; and (4) benign and malignant cells of other proliferative diseases in which aberrant EZH2 activation occurs.

As used herein “cancer” refers to any malignant and/or invasive growth or tumor caused by abnormal cell growth. As used herein “cancer” refers to solid tumors named for the type of cells that form them, cancer of blood, bone marrow, or the lymphatic system. Examples of solid tumors include but not limited to sarcomas and carcinomas. Examples of cancers of the blood include but not limited to leukemias, lymphomas and myeloma. The term “cancer” includes but is not limited to a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of different type from latter one. The compounds of the invention inhibit EZH2, and thus are all adapted to therapeutic use as antiproliferative agents (e.g., cancer) or antitumor agent (e.g., effect against solid tumors) in mammals, particularly in humans. In particular, the compounds of the invention are useful in the prevention and treatment of a variety of human hyperproliferative disorders including both malignant and benign abnormal cell growth.

The compounds, compositions and methods provided herein are useful for the treatment of cancers including but not limited to cancers of the:

circulatory system, for example, heart (sarcoma [angiosarcoma, fibrosarcoma, rhabdomyosarcoma, liposarcoma], myxoma, rhabdomyoma, fibroma, lipoma and teratoma), mediastinum and pleura, and other intrathoracic organs, vascular tumors and tumor-associated vascular tissue;

respiratory tract, for example, nasal cavity and middle ear, accessory sinuses, larynx, trachea, bronchus and lung such as small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;

gastrointestinal system, for example, esophagus (squamous cell carcinoma, adenocarcinoma, leiomyosarcoma, lymphoma), stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma);

genitourinary tract, for example, kidney (adenocarcinoma, Wilm's tumor [nephroblastoma], lymphoma, leukemia), bladder and/or urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);

liver, for example, hepatoma (hepatocellular carcinoma), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma);

bone, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;

nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma);

reproductive system, for example, gynecological, uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma) and other sites associated with female genital organs; placenta, penis, prostate, testis, and other sites associated with male genital organs;

hematologic system, for example, blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma];

oral cavity, for example, lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx;

skin, for example, malignant melanoma, cutaneous melanoma, basal cell carcinoma, squamous cell carcinoma, Karposi's sarcoma, moles dysplastic nevi, lipoma, angioma, dermatofibroma, and keloids;

adrenal glands: neuroblastoma; and

other tissues including connective and soft tissue, retroperitoneum and peritoneum, eye, intraocular melanoma, and adnexa, breast, head or/and neck, anal region, thyroid, parathyroid, adrenal gland and other endocrine glands and related structures, secondary and unspecified malignant neoplasm of lymph nodes, secondary malignant neoplasm of respiratory and digestive systems and secondary malignant neoplasm of other sites.

More specifically, examples of “cancer” when used herein in connection with the present invention include cancer selected from lung cancer (NSCLC and SCLC), cancer of the head or neck, ovarian cancer, colon cancer, rectal cancer, cancer of the anal region, stomach cancer, breast cancer, cancer of the kidney or ureter, renal cell carcinoma, carcinoma of the renal pelvis, neoplasms of the central nervous system (CNS), primary CNS lymphoma, non-Hodgkin's lymphoma, spinal axis tumors, or a combination of one or more of the foregoing cancers.

Still more specifically, examples of “cancer” when used herein in connection with the present invention include cancer selected from lung cancer (NSCLC and SCLC), breast cancer, ovarian cancer, colon cancer, rectal cancer, cancer of the anal region, or a combination of one or more of the foregoing cancers.

In further embodiments, examples of “cancer” when used herein in connection with the present invention include cancer selected from lung cancer (NSCLC and SCLC), breast cancer, ovarian cancer, colon cancer, rectal cancer, cancer of the anal region, endometrial cancer, gastric cancer, liver cancer (HCC), kidney cancer (RCC), melanoma, pancreatic cancer, prostate cancer, bladder cancer, or lymphoma (DLBCL or FL), or a combination of one or more of the foregoing cancers.

In one embodiment of the present invention the non-cancerous conditions include such hyperplastic conditions such as benign hyperplasia of the skin (e.g., psoriasis) and benign hyperplasia of the prostate (e.g., BPH).

In another aspect, the invention provides a method for inhibiting cell proliferation, comprising contacting cells with a compound of the invention or a pharmaceutically acceptable salt thereof in an amount effective to inhibit proliferation of the cells.

In another aspect, the invention provides methods for inducing cell apoptosis, comprising contacting cells with a compound described herein in an amount effective to induce apoptosis of the cells.

“Contacting” refers to bringing a compound or pharmaceutically acceptable salt of the invention and a cell expressing EZH2 together in such a manner that the compound can affect the activity of EZH2, either directly or indirectly. Contacting can be accomplished in vitro (i.e., in an artificial environment such as, e.g., without limitation, in a test tube or culture medium) or in vivo (i.e., within a living organism such as, without limitation, a mouse, rat or rabbit.)

In some embodiments, the cells are in a cell line, such as a cancer cell line. In other embodiments, the cells are in a tissue or tumor, and the tissue or tumor may be in a subject, including a human.

### Dosage Forms and Regimens

Administration of the compounds of the invention may be effected by any method that enables delivery of the compounds to the site of action. These methods include oral routes, intraduodenal routes, parenteral injection (including intravenous, subcutaneous, intramuscular, intravascular or infusion), topical, and rectal administration.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

The amount of the compound of the invention administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.1 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

### Formulations and Routes of Administration

As used herein, a “pharmaceutically acceptable carrier” refers to a carrier or diluent that does not cause significant irritation to an organism and does not abrogate the biological activity and properties of the administered compound.

The pharmaceutical acceptable carrier may comprise any conventional pharmaceutical carrier or excipient. The choice of carrier and/or excipient will to a large extent depend on factors such as the particular mode of administration, the effect of the excipient on solubility and stability, and the nature of the dosage form.

Suitable pharmaceutical carriers include inert diluents or fillers, water and various organic solvents (such as hydrates and solvates). The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition may, for example, be in a form suitable for oral administration as a tablet, capsule, pill, powder, sustained release formulations, solution suspension, for parenteral injection as a sterile solution, suspension or emulsion, for topical administration as an ointment or cream or for rectal administration as a suppository.

Exemplary parenteral administration forms include solutions or suspensions of active compounds in sterile aqueous solutions, for example, aqueous propylene glycol or dextrose solutions. Such dosage forms may be suitably buffered, if desired.

The pharmaceutical composition may be in unit dosage forms suitable for single administration of precise dosages.

Pharmaceutical compositions suitable for the delivery of compounds of the invention and methods for their preparation will be readily apparent to those skilled in the art. Such compositions and methods for their preparation can be found, for example, in ‘Remington's Pharmaceutical Sciences’, 19th Edition (Mack Publishing Company, 1995), the disclosure of which is incorporated herein by reference in its entirety.

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001), the disclosure of which is incorporated herein by reference in its entirety.

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally include surface active agents, such as sodium lauryl sulfate and polysorbate 80, and glidants such as silicon dioxide and talc. When present, surface active agents are typically in amounts of from 0.2 wt % to 5 wt % of the tablet, and glidants typically from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, preferably from 0.5 wt % to 3 wt % of the tablet.

Other conventional ingredients include anti-oxidants, colorants, flavoring agents, preservatives and taste-masking agents.

Exemplary tablets contain up to about 80 wt % drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tableting. The final formulation may include one or more layers and may be coated or uncoated; or encapsulated.

The formulation of tablets is discussed in detail in “Pharmaceutical Dosage Forms: Tablets, Vol. 1”, by H. Lieberman and L. Lachman, Marcel Dekker, N.Y., N.Y., 1980 (ISBN 0-8247-6918-X), the disclosure of which is incorporated herein by reference in its entirety.

Solid formulations for oral administration may be formulated to be immediate and/or modified release. Modified release formulations include delayed-, sustained-, pulsed-, controlled-, targeted and programmed release.

Suitable modified release formulations are described in U.S. Pat. No. 6,106,864. Details of other suitable release technologies such as high energy dispersions and osmotic and coated particles can be found in Verma et al, Pharmaceutical Technology On-line, 25(2), 1-14 (2001). The use of chewing gum to achieve controlled release is described in WO 00/35298. The disclosures of these references are incorporated herein by reference in their entireties.

### Parenteral Administration

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including micro needle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilization, may readily be accomplished using standard pharmaceutical techniques well known to those skilled in the art.

The solubility of compounds of the invention used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and PGLA microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated; see, for example, J Pharm Sci, 88 (10), 955-958 by Finnin and Morgan (October 1999). Other means of topical administration include delivery by electroporation, iontophoresis, phonophoresis, sonophoresis and micro needle or needle-free (e.g. Powderject™, Bioject™ etc.) injection. The disclosures of these references are incorporated herein by reference in their entireties.

Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may include a bioadhesive agent, for example, chitosan or cyclodextrin.

The pressurized container, pump, spray, atomizer, or nebulizer contains a solution or suspension of the compound(s) of the invention comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid.

Prior to use in a dry powder or suspension formulation, the drug product is micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying.

Capsules (made, for example, from gelatin or HPMC), blisters and cartridges for use in an inhaler or insufflator may be formulated to contain a powder mix of the compound of the invention, a suitable powder base such as lactose or starch and a performance modifier such as 1-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomizer using electrohydrodynamics to produce a fine mist may contain from 1 μg to 20 mg of the compound of the invention per actuation and the actuation volume may vary from 1 μL to 100 μL. A typical formulation includes a compound of the invention, propylene glycol, sterile water, ethanol and sodium chloride. Alternative solvents which may be used instead of propylene glycol include glycerol and polyethylene glycol.

Suitable flavors, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing a desired mount of the compound of the invention. The overall daily dose may be administered in a single dose or, more usually, as divided doses throughout the day.

Compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

Compounds of the invention may also be administered directly to the eye or ear, typically in the form of drops of a micronized suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

### Other Technologies

Compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubilizer. Most commonly used for these purposes are alpha-, beta- and gamma-cyclodextrins, examples of which may be found in PCT Publication Nos. WO 91/11172, WO 94/02518 and WO 98/55148, the disclosures of which are incorporated herein by reference in their entireties.

### Dosage

The amount of the active compound administered will be dependent on the subject being treated, the severity of the disorder or condition, the rate of administration, the disposition of the compound and the discretion of the prescribing physician. However, an effective dosage is typically in the range of about 0.001 to about 100 mg per kg body weight per day, preferably about 0.01 to about 35 mg/kg/day, in single or divided doses. For a 70 kg human, this would amount to about 0.07 to about 7000 mg/day, preferably about 0.7 to about 2500 mg/day. In some instances, dosage levels below the lower limit of the aforesaid range may be more than adequate, while in other cases still larger doses may be used without causing any harmful side effect, with such larger doses typically divided into several smaller doses for administration throughout the day.

### Kit-of-Parts

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions. Thus the kit of the invention includes two or more separate pharmaceutical compositions, at least one of which contains a compound of the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid.

Combination Therapy

As used herein, the term “combination therapy” refers to the administration of a compound of the invention together with an at least one additional pharmaceutical or medicinal agent (e.g., an anti-cancer agent), either sequentially or simultaneously.

As noted above, the compounds of the invention may be used in combination with one or more additional anti-cancer agents which are described below. When a combination therapy is used, the one or more additional anti-cancer agents may be administered sequentially or simultaneously with the compound of the invention. In one embodiment, the additional anti-cancer agent is administered to a mammal (e.g., a human) prior to administration of the compound of the invention. In another embodiment, the additional anti-cancer agent is administered to the mammal after administration of the compound of the invention. In another embodiment, the additional anti-cancer agent is administered to the mammal (e.g., a human) simultaneously with the administration of the compound of the invention.

The invention also relates to a pharmaceutical composition for the treatment of abnormal cell growth in a mammal, including a human, which comprises an amount of a compound of the invention, as defined above (including hydrates, solvates and polymorphs of said compound or pharmaceutically acceptable salts thereof), in combination with one or more (preferably one to three) anti-cancer agents selected from the group consisting of anti-angiogenesis agents and signal transduction inhibitors and a pharmaceutically acceptable carrier, wherein the amounts of the active agent and the combination anti-cancer agents when taken as a whole is therapeutically effective for treating said abnormal cell growth.

In one embodiment of the present invention the anti-cancer agent used in conjunction with a compound of the invention and pharmaceutical compositions described herein is an anti-angiogenesis agent (e.g., an agent that stops tumors from developing new blood vessels). Examples of anti-angiogenesis agents include for example VEGF inhibitors, VEGFR inhibitors, TIE-2 inhibitors, PDGFR inhibitors, angiopoetin inhibitors, PKCβ inhibitors, COX-2 (cyclooxygenase II) inhibitors, integrins (alpha-v/beta-3), MMP-2 (matrix-metalloproteinase 2) inhibitors, and MMP-9 (matrix-metalloproteinase 9) inhibitors.

Preferred anti-angiogenesis agents include sunitinib (Sutent™), bevacizumab (Avastin™), axitinib (AG 13736), SU 14813 (Pfizer), and AG 13958 (Pfizer).

Additional anti-angiogenesis agents include vatalanib (CGP 79787), Sorafenib (Nexavar™), pegaptanib octasodium (Macugen™), vandetanib (Zactima™), PF-0337210 (Pfizer), SU 14843 (Pfizer), AZD 2171 (AstraZeneca), ranibizumab (Lucentis™), Neovastat™ (AE 941), tetrathiomolybdata (Coprexa™), AMG 706 (Amgen), VEGF Trap (AVE 0005), CEP 7055 (Sanofi-Aventis), XL 880 (Exelixis), telatinib (BAY 57-9352), and CP-868,596 (Pfizer).

Other anti-angiogenesis agents include enzastaurin (LY 317615), midostaurin (CGP 41251), perifosine (KRX 0401), teprenone (Selbex™) and UCN 01 (Kyowa Hakko).

Other examples of anti-angiogenesis agents which can be used in conjunction with a compound of the invention and pharmaceutical compositions described herein include celecoxib (Celebrex™), parecoxib (Dynastat™), deracoxib (SC 59046), lumiracoxib (Preige™), valdecoxib (Bextra™), rofecoxib (Vioxx™), iguratimod (Careram™), IP 751 (Invedus), SC-58125 (Pharmacia) and etoricoxib (Arcoxia™)

Other anti-angiogenesis agents include exisulind (Aptosyn™), salsalate (Amigesic™) diflunisal (Dolobid™), ibuprofen (Motrin™), ketoprofen (Orudis™), nabumetone (Relafen™) piroxicam (Feldene™), naproxen (Aleve™, Naprosyn™), diclofenac (Voltaren™), indomethacin (Indocin™), sulindac (Clinoril™), tolmetin (Tolectin™), etodolac (Lodine™), ketorolac (Toradol™), and oxaprozin (Daypro™)

Other anti-angiogenesis agents include ABT 510 (Abbott), apratastat (TMI 005), AZD 8955 (AstraZeneca), incyclinide (Metastat™), and PCK 3145 (Procyon).

Other anti-angiogenesis agents include acitretin (Neotigason™), plitidepsin (Aplidine™) cilengtide (EMD 121974), combretastatin A4 (CA4P), fenretinide (4 HPR), halofuginone (Tempostatin™), Panzem™ (2-methoxyestradiol), PF-03446962 (Pfizer), rebimastat (BMS 275291), catumaxomab (Removab™), lenalidomide (Revlimid™), squalamine (EVIZON™) thalidomide (Thalomid™), Ukrain™ (NSC 631570), Vitaxin™ (MEDI 522), and zoledronic acid (Zometa™).

In another embodiment the anti-cancer agent is a so called signal transduction inhibitor (e.g., inhibiting the means by which regulatory molecules that govern the fundamental processes of cell growth, differentiation, and survival communicated within the cell). Signal transduction inhibitors include small molecules, antibodies, and antisense molecules. Signal transduction inhibitors include for example kinase inhibitors (e.g., tyrosine kinase inhibitors or serine/threonine kinase inhibitors) and cell cycle inhibitors. More specifically signal transduction inhibitors include, for example, farnesyl protein transferase inhibitors, EGF inhibitor, ErbB-1 (EGFR), ErbB-2, pan erb, IGF1R inhibitors, MEK, c-Kit inhibitors, FLT-3 inhibitors, K-Ras inhibitors, PI3 kinase inhibitors, JAK inhibitors, STAT inhibitors, Raf kinase inhibitors, Akt inhibitors, mTOR inhibitor, P70S6 kinase inhibitors, inhibitors of the WNT pathway and so called multi-targeted kinase inhibitors.

Preferred signal transduction inhibitors include gefitinib (Iressa™), cetuximab (Erbitux™) erlotinib (Tarceva™), trastuzumab (Herceptin™), sunitinib (Sutent™), imatinib (Gleevec™), and PD325901 (Pfizer).

Additional examples of signal transduction inhibitors which may be used in conjunction with a compound of the invention and pharmaceutical compositions described herein include BMS 214662 (Bristol-Myers Squibb), lonafarnib (Sarasar™), pelitrexol (AG 2037), matuzumab (EMD 7200), nimotuzumab (TheraCIM h-R3™), panitumumab (Vectibix™), Vandetanib (Zactima™), pazopanib (SB 786034), ALT 110 (Alteris Therapeutics), BIBW 2992 (Boehringer Ingelheim), and Cervene™ (TP 38).

Other examples of signal transduction inhibitor include PF-2341066 (Pfizer), PF-299804 (Pfizer), canertinib (CI 1033), pertuzumab (Omnitarg™), Lapatinib (Tycerb™), pelitinib (EKB 569), miltefosine (Miltefosin™), BMS 599626 (Bristol-Myers Squibb), Lapuleucel-T (Neuvenge™), NeuVax™ (E75 cancer vaccine), Osidem™ (IDM 1), mubritinib (TAK-165), CP-724,714 (Pfizer), panitumumab (Vectibix™), lapatinib (Tycerb™), PF-299804 (Pfizer), pelitinib (EKB 569), and pertuzumab (Omnitarg™).

Other examples of signal transduction inhibitors include ARRY 142886 (Array Biopharm), everolimus (Certican™), zotarolimus (Endeavor™), temsirolimus (Torisel™), AP 23573 (ARIAD), and VX 680 (Vertex).

Additionally, other signal transduction inhibitors include XL 647 (Exelixis), sorafenib (Nexavar™), LE-AON (Georgetown University), and GI-4000 (Globelmmune).

Other signal transduction inhibitors include ABT 751 (Abbott), alvocidib (flavopiridol), BMS 387032 (Bristol Myers), EM 1421 (Erimos), indisulam (E 7070), seliciclib (CYC 200), BIO 112 (Onc Bio), BMS 387032 (Bristol-Myers Squibb), PD 0332991 (Pfizer), and AG 024322 (Pfizer).

This invention contemplates the use of compounds of the invention together with classical antineoplastic agents. Classical antineoplastic agents include but are not limited to hormonal modulators such as hormonal, anti-hormonal, androgen agonist, androgen antagonist and anti-estrogen therapeutic agents, histone deacetylase (HDAC) inhibitors, gene silencing agents or gene activating agents, ribonucleases, proteosomics, Topoisomerase I inhibitors, Camptothecin derivatives, Topoisomerase II inhibitors, alkylating agents, antimetabolites, poly(ADP-ribose) polymerase-1 (PARP-1) inhibitor, microtubulin inhibitors, antibiotics, plant derived spindle inhibitors, platinum-coordinated compounds, gene therapeutic agents, antisense oligonucleotides, vascular targeting agents (VTAs), and statins

Examples of classical antineoplastic agents used in combination therapy with a compound of the invention, optionally with one or more other agents include, but are not limited to, glucocorticoids, such as dexamethasone, prednisone, prednisolone, methylprednisolone, hydrocortisone, and progestins such as medroxyprogesterone, megestrol acetate (Megace), mifepristone (RU-486), Selective Estrogen Receptor Modulators (SERMs; such as tamoxifen, raloxifene, lasofoxifene, afimoxifene, arzoxifene, bazedoxifene, fispemifene, ormeloxifene, ospemifene, tesmilifene, toremifene, trilostane and CHF 4227 (Cheisi)), Selective Estrogen-Receptor Downregulators (SERD's; such as fulvestrant), exemestane (Aromasin), anastrozole (Arimidex), atamestane, fadrozole, letrozole (Femara), gonadotropin-releasing hormone (GnRH; also commonly referred to as luteinizing hormone-releasing hormone [LHRH]) agonists such as buserelin (Suprefact), goserelin (Zoladex), leuprorelin (Lupron), and triptorelin (Trelstar), abarelix (Plenaxis), bicalutamide (Casodex), cyproterone, flutamide (Eulexin), megestrol, nilutamide (Nilandron), and osaterone, dutasteride, epristeride, finasteride, Serenoa repens, PHL 00801, abarelix, goserelin, leuprorelin, triptorelin, bicalutamide, tamoxifen, exemestane, anastrozole, fadrozole, formestane, letrozole, and combinations thereof.

Other examples of classical antineoplastic agents used in combination with compounds of the invention include but are not limited to suberolanilide hydroxamic acid (SAHA, Merck Inc./Aton Pharmaceuticals), depsipeptide (FR901228 or FK228), G2M-777, MS-275, pivaloyloxymethyl butyrate and PXD-101; Onconase (ranpirnase), PS-341 (MLN-341), Velcade (bortezomib), 9-aminocamptothecin, belotecan, BN-80915 (Roche), camptothecin, diflomotecan, edotecarin, exatecan (Daiichi), gimatecan, 10-hydroxycamptothecin, irinotecan HCl (Camptosar), lurtotecan, Orathecin (rubitecan, Supergen), SN-38, topotecan, camptothecin, 10-hydroxycamptothecin, 9-aminocamptothecin, irinotecan, SN-38, edotecarin, topotecan, aclarubicin, adriamycin, amonafide, amrubicin, annamycin, daunorubicin, doxorubicin, elsamitrucin, epirubicin, etoposide, idarubicin, galarubicin, hydroxycarbamide, nemorubicin, novantrone (mitoxantrone), pirarubicin, pixantrone, procarbazine, rebeccamycin, sobuzoxane, tafluposide, valrubicin, Zinecard (dexrazoxane), nitrogen mustard N-oxide, cyclophosphamide, AMD-473, altretamine, AP-5280, apaziquone, brostallicin, bendamustine, busulfan, carboquone, carmustine, chlorambucil, dacarbazine, estramustine, fotemustine, glufosfamide, ifosfamide, KW-2170, lomustine, mafosfamide, mechlorethamine, melphalan, mitobronitol, mitolactol, mitomycin C, mitoxatrone, nimustine, ranimustine, temozolomide, thiotepa, and platinum-coordinated alkylating compounds such as cisplatin, Paraplatin (carboplatin), eptaplatin, lobaplatin, nedaplatin, Eloxatin (oxaliplatin, Sanofi), streptozocin, satrplatin, and combinations thereof.

The invention also contemplates the use of the compounds of the invention together with dihydrofolate reductase inhibitors (such as methotrexate and NeuTrexin (trimetresate glucuronate)), purine antagonists (such as 6-mercaptopurine riboside, mercaptopurine, 6-thioguanine, cladribine, clofarabine (Clolar), fludarabine, nelarabine, and raltitrexed), pyrimidine antagonists (such as 5-fluorouracil (5-FU), Alimta (premetrexed disodium, LY231514, MTA), capecitabine (Xeloda™), cytosine arabinoside, Gemzar™ (gemcitabine, Eli Lilly), Tegafur (UFT Orzel or Uforal and including TS-1 combination of tegafur, gimestat and otostat), doxifluridine, carmofur, cytarabine (including ocfosfate, phosphate stearate, sustained release and liposomal forms), enocitabine, 5-azacitidine (Vidaza), decitabine, and ethynylcytidine) and other antimetabolites such as eflornithine, hydroxyurea, leucovorin, nolatrexed (Thymitaq), triapine, trimetrexate, N-(5-[N-(3,4-dihydro-2-methyl-4-oxoquinazolin-6-ylmethyl)-N-methylamino]-2-thenoyl)-L-glutamic acid, AG-014699 (Pfizer Inc.), ABT-472 (Abbott Laboratories), INO-1001 (Inotek Pharmaceuticals), KU-0687 (KuDOS Pharmaceuticals) and GPI 18180 (Guilford Pharm Inc) and combinations thereof.

Other examples of classical antineoplastic cytotoxic agents used in combination therapy with a compound of the invention, optionally with one or more other agents include, but are not limited to, Abraxane (Abraxis BioScience, Inc.), Batabulin (Amgen), EPO 906 (Novartis), Vinflunine (Bristol-Myers Squibb Company), actinomycin D, bleomycin, mitomycin C, neocarzinostatin (Zinostatin), vinblastine, vincristine, vindesine, vinorelbine (Navelbine), docetaxel (Taxotere), Ortataxel, paclitaxel (including Taxoprexin a DHA/paciltaxel conjugate), cisplatin, carboplatin, Nedaplatin, oxaliplatin (Eloxatin), Satraplatin, Camptosar, capecitabine (Xeloda), oxaliplatin (Eloxatin), Taxotere alitretinoin, Canfosfamide (Telcyta™), DMXAA (Antisoma), ibandronic acid, L-asparaginase, pegaspargase (Oncaspar™), Efaproxiral (Efaproxyn™—radiation therapy)), bexarotene (Targretin™), Tesmilifene (DPPE—enhances efficacy of cytotoxics)), Theratope™ (Biomira), Tretinoin (Vesanoid™), tirapazamine (Trizaone™), motexafin gadolinium (Xcytrin™) Cotara™ (mAb), and NBI-3001 (Protox Therapeutics), polyglutamate-paclitaxel (Xyotax™) and combinations thereof.

Further examples of classical antineoplastic agents used in combination therapy with a compound of the invention, optionally with one or more other agents include, but are not limited to, as Advexin (ING 201), TNFerade (GeneVec, a compound which express TNFalpha in response to radiotherapy), RB94 (Baylor College of Medicine), Genasense (Oblimersen, Genta), Combretastatin A4P (CA4P), Oxi-4503, AVE-8062, ZD-6126, TZT-1027, Atorvastatin (Lipitor, Pfizer Inc.), Provastatin (Pravachol, Bristol-Myers Squibb), Lovastatin (Mevacor, Merck Inc.), Simvastatin (Zocor, Merck Inc.), Fluvastatin (Lescol, Novartis), Cerivastatin (Baycol, Bayer), Rosuvastatin (Crestor, AstraZeneca), Lovostatin, Niacin (Advicor, Kos Pharmaceuticals), Caduet, Lipitor, torcetrapib, and combinations thereof.

Another embodiment of the present invention of particular interest relates to a method for the treatment of breast cancer in a human in need of such treatment, comprising administering to said human an amount of a compound of the invention, in combination with one or more (preferably one to three) anti-cancer agents selected from the group consisting of trastuzumab, tamoxifen, docetaxel, paclitaxel, capecitabine, gemcitabine, vinorelbine, exemestane, letrozole and anastrozole.

In one embodiment the invention provides a method of treating colorectal cancer in a mammal, such as a human, in need of such treatment, by administering an amount of a compound of the invention, in combination with one or more (preferably one to three) anti-cancer agents. Examples of particular anti-cancer agents include those typically used in adjuvant chemotherapy, such as FOLFOX, a combination of 5-fluorouracil (5-FU) or capecitabine (Xeloda), leucovorin and oxaliplatin (Eloxatin). Further examples of particular anti-cancer agents include those typically used in chemotherapy for metastatic disease, such as FOLFOX or FOLFOX in combination with bevacizumab (Avastin); and FOLFIRI, a combination of 5-FU or capecitabine, leucovorin and irinotecan (Camptosar). Further examples include 17-DMAG, ABX-EFR, AMG-706, AMT-2003, ANX-510 (CoFactor), aplidine (plitidepsin, Aplidin), Aroplatin, axitinib (AG-13736), AZD-0530, AZD-2171, bacillus Calmette-Guerin (BCG), bevacizumab (Avastin), BIO-117, BIO-145, BMS-184476, BMS-275183, BMS-528664, bortezomib (Velcade), C-1311 (Symadex), cantuzumab mertansine, capecitabine (Xeloda), cetuximab (Erbitux), clofarabine (Clofarex), CMD-193, combretastatin, Cotara, CT-2106, CV-247, decitabine (Dacogen), E-7070, E-7820, edotecarin, EMD-273066, enzastaurin (LY-317615) epothilone B (EPO-906), erlotinib (Tarceva), flavopyridol, GCAN-101, gefitinib (Iressa), huA33, huC242-DM4, imatinib (Gleevec), indisulam, ING-1, irinotecan (CPT-11, Camptosar) ISIS 2503, ixabepilone, lapatinib (Tykerb), mapatumumab (HGS-ETR1), MBT-0206, MEDI-522 (Abregrin), Mitomycin, MK-0457 (VX-680), MLN-8054, NB-1011, NGR-TNF, NV-1020, oblimersen (Genasense, G3139), OncoVex, ONYX 015 (CI-1042), oxaliplatin (Eloxatin), panitumumab (ABX-EGF, Vectibix), pelitinib (EKB-569), pemetrexed (Alimta), PD-325901, PF-0337210, PF-2341066, RAD-001 (Everolimus), RAV-12, Resveratrol, Rexin-G, S-1 (TS-1), seliciclib, SN-38 liposome, Sodium stibogluconate (SSG), sorafenib (Nexavar), SU-14813, sunitinib (Sutent), temsirolimus (CCI 779), tetrathiomolybdate, thalomide, TLK-286 (Telcyta), topotecan (Hycamtin), trabectedin (Yondelis), vatalanib (PTK-787), vorinostat (SAHA, Zolinza), WX-UK1, and ZYC300, wherein the amounts of the active agent together with the amounts of the combination anticancer agents are effective in treating colorectal cancer.

Another embodiment of the present invention of particular interest relates to a method for the treatment of renal cell carcinoma in a human in need of such treatment, comprising administering to said human an amount of a compound of the invention, in combination with one or more (preferably one to three) anti-cancer agents selected from the group consisting of axitinib (AG 13736), capecitabine (Xeloda), interferon alpha, interleukin-2, bevacizumab (Avastin), gemcitabine (Gemzar), thalidomide, cetuximab (Erbitux), vatalanib (PTK-787), sunitinib (Sutent™), AG-13736, SU-11248, Tarceva, Iressa, Lapatinib and Gleevec, wherein the amounts of the active agent together with the amounts of the combination anticancer agents is effective in treating renal cell carcinoma.

Another embodiment of the present invention of particular interest relates to a method for the treatment of melanoma in a human in need of such treatment, comprising administering to said human an amount of a compound of the invention, in combination with one or more (preferably one to three) anti-cancer agents selected from the group consisting of interferon alpha, interleukin-2, temozolomide (Temodar), docetaxel (Taxotere), paclitaxel, Dacarbazine (DTIC), carmustine (also known as BCNU), Cisplatin, vinblastine, tamoxifen, PD-325,901, axitinib (AG 13736), bevacizumab (Avastin), thalidomide, sorafanib, vatalanib (PTK-787), sunitinib (Sutent™), CpG-7909, AG-13736, Iressa, Lapatinib and Gleevec, wherein the amounts of the active agent together with the amounts of the combination anticancer agents is effective in treating melanoma.

Another embodiment of the present invention of particular interest relates to a method for the treatment of lung cancer in a human in need of such treatment, comprising administering to said human an amount of a compound of the invention, in combination with one or more (preferably one to three) anti-cancer agents selected from the group consisting of capecitabine (Xeloda), axitinib (AG 13736), bevacizumab (Avastin), gemcitabine (Gemzar), docetaxel (Taxotere), paclitaxel, premetrexed disodium (Alimta), Tarceva, Iressa, Vinorelbine, Irinotecan, Etoposide, Vinblastine, sunitinib (Sutent™), and Paraplatin (carboplatin), wherein the amounts of the active agent together with the amounts of the combination anticancer agents is effective in treating lung cancer.

Compounds of the invention are prepared according to the exemplary procedures provided herein. In frequent embodiments, the compounds of the invention are prepared by sequential amide coupling of a mono- or di-halogenated benzoic acid or heterobenzoic acid compound to a substituted 3-aminomethyl-1H-pyridin-2-one, followed by Suzuki coupling to a boronic acid derivative (e.g., Method A). In some embodiments, the coupling product is subjected to a second cross-coupling reaction, in particular a second Suzuki coupling (e.g., Method C). In further embodiments, the order of the steps is reversed, such that a mono- or di-halogenated benzoic acid or heterobenzoic acid is subjected to one or two Suzuki couplings, followed by amide coupling (e.g., Method B).

In some embodiments, the benzoic or heterobenzoic acid is used in protected form, e.g., as a carboxylate ester, and the method include a step of ester hydrolysis prior to amide formation (e.g., Method D or E).

In further embodiments, a halogenated intermediate is subjected to nucleophilic displacement with an alkoxide to install an alkoxy moiety (e.g., Method E or M)

In other embodiments, a halogenated intermediate is subjected to nucleophilic displacement with an amino substituent to install or an N-linked heterocyclic moiety (e.g., Method H, L or M).

In still other embodiments, the methods involve structural transformations of the core ring system to install one or more of the substituent groups (e.g., Method F, I or J).

In further embodiments, the methods involve chemical modification of a substituent group (e.g., Method N).

These and other methods are exemplified in the preparation of the examples provided herein. Synthetic examples are provided throughout the examples and in Table 1 and Table 2 below. IC₅₀ values (μM) and/or % Effect at 20 μM for exemplary compounds of the invention in wild-type EZH2 and Y641 N EZH2-PCR2 mutant are provided in Table 3.

### Abbreviations

The following abbreviations are used throughout the Examples:

“BOC”, “Boc” or “boc” means N-tert-butoxycarbonyl, “DCM” (CH₂Cl₂) means methylene chloride, “DIPEA” or “DIEA” means diisopropyl ethyl amine, “DBU” means 1,8-diazabicyclo[5.4.0]undec-7-ene, “NMM” means N-methylmorpholine, “DMA” means N,N-dimethylacetamide, “DMF” means N—N-dimethyl formamide, “DMSO” means dimethylsulfoxide, “DPPP” means 1,3-bis(diphenylphosphino)propane, “HOAc” means acetic acid, “IPA” means isopropyl alcohol, “MTBE” means methyl t-butyl ether, “NMP” means 1-methyl 2-pyrrolidinone, “TEA” means triethyl amine, “TFA” means trifluoroacetic acid, “EtOAc” means ethyl acetate, “MgSO₄” means magnesium sulphate, “NaSO₄” means sodium sulphate, “MeOH” means methanol, “EtOH” means ethanol, “THF” means tetrahydrofuran, “Ac” means acetyl, “OAc” means acetoxy, “Et” means ethyl, “Me” means methyl, “Ph” means phenyl, “Bu” means butyl, “tBu” means ter-butyl, “dppf” means (diphenylphosphino)ferrocene, “HATU” means 2-(7-Aza-1H-benzotriazole-1-yl)-1,1,3,3-tetramethyluronium hexafluorophosphate, “mCPBA” means meta-chloroperoxybenzoic acid, “SFC” means supercritical fluid chromatography, “˜” means approximately, “rt” means room temperature, “h” means hours, “min” means minutes, “Tf” means trifluoromethanesulfonate (also commonly called ‘triflate’), “eq.” means equivalents.

### EXAMPLES

### Method A

### Example 1

### N-[(4,6-dimethyl-2-oxo-1,2-dihydropyridin-3-yl)methyl]-3-(1,4-dimethyl-1H-pyrazol-5-yl)-2-methylbenzamide

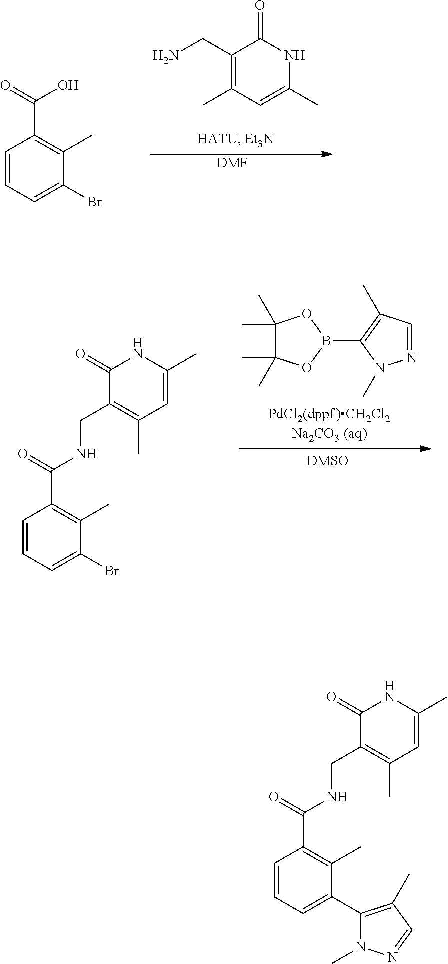

A solution of 3-bromo-2-methylbenzoic acid (213.5 mg, 0.993 mmol), 3-aminomethyl-4,6-dimethyl-1H-pyridin-2-one hydrochloride (267.3 mg, 1.498 mmol; prepared according to published procedure in WO 2011/140324), O-(7-azabenzotriazol-1-yl)-N,N,N′,N′ tetramethyluronium hexafluorophosphate (HATU, 482.0 mg, 1.268 mmol), and triethylamine (0.50 mL, 3.6 mmol) in N,N-dimethylformamide (5.0 mL) was stirred at room temperature for 17 hours. The mixture was diluted with 20 mL deionized water and stirred for 10 minutes, causing a white precipitate to form. The precipitate was collected by suction filtration and air-dried until free-flowing, yielding 3-bromo-N-((4,6-dimethyl-2-oxo-1,2-dihydropyridin-3-yl)methyl)-2-methylbenzamide (292.8 mg, 84% yield) as an off-white solid. ¹H NMR (400 MHz, DMSO-d₆) δ 11.45 (s, 1H), 8.26 (t, J=4.55 Hz, 1H), 7.61 (dd, J=1.14, 7.96 Hz, 1H), 7.21 (dd, J=1.01, 6.57 Hz, 1H), 7.13 (t, J=7.83 Hz, 1H), 5.85 (s, 1H), 4.26 (d, J=5.05 Hz, 2H), 2.30 (s, 3H), 2.19 (s, 3H), 2.11 (s, 3H).

A septum-sealed vial containing 3-bromo-N-((4,6-dimethyl-2-oxo-1,2-dihydropyridin-3-yl)methyl)-2-methylbenzamide (287.0 mg, 0.822 mmol), (1,4-dimethyl-1H-pyrazol-5-yl)boronic acid pinacol ester (213.7 mg, 0.962 mmol), dichloro-1,1′-bis(diphenylphosphino)ferrocene palladium (II)-dichloromethane complex [PdCl₂(dppf).CH₂Cl₂] (65.0 mg, 0.080 mmol) (prepared according to published procedure in WO 2008/98104), and sodium carbonate (300.2 mg, 2.83 mmol) was evacuated and filled with argon. Dimethylsulfoxide (8.0 mL) and deionized water (2.0 mL) were added by syringe. The solution was degassed by evacuation until the solvent began to boil, followed by argon fill 3 cycles, then irradiated in a 100° C. microwave for 20 minutes. After cooling to room temperature, the solution was partitioned between ethyl acetate (30 mL) and pH 4 sodium acetate buffer solution (15 mL). The aqueous layer was further extracted with ethyl acetate (2×20 mL). The combined organic extracts were dried over magnesium sulfate, filtered, concentrated, and purified via reverse-phase HPLC to give the title compound (58.34 mg, 19.5% yield) as a white solid. ¹H NMR (400 MHz, DMSO-d₆) δ 11.46 (br. s., 1H), 8.25 (t, J=4.95 Hz, 1H), 7.28-7.36 (m, 3H), 7.20 (dd, J=1.96, 6.97 Hz, 1H), 5.86 (s, 1H), 4.29 (d, J=5.01 Hz, 2H), 3.47 (s, 3H), 2.20 (s, 3H), 2.11 (s, 3H), 1.98 (s, 3H), 1.79 (s, 3H); MS: 365 [M+1].

### Example 2

### N-[(4,6-dimethyl-2-oxo-1,2-dihydropyridin-3-yl)methyl]-2-methyl-3-(1-methyl-1H-pyrazol-5-yl)benzamide

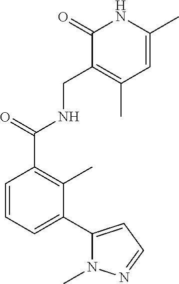 [SMILES: Cc1cc(C)c(CNC(=O)c2cccc(-c3ccnn3C)c2C)c(=O)[nH]1]

The compound of Example 2 was made by the method of Example 1, using 1-methyl-1H-pyrazole-5-boronic acid pinacol ester as the coupling partner and 1,4-dioxane as the solvent in the final Suzuki reaction, to provide the title compound. ¹H NMR (400 MHz, chloroform-d) δ 11.30 (br. s., 1H), 7.53 (d, J=1.77 Hz, 1H), 7.41 (t, J=4.55 Hz, 1H), 7.19-7.26 (m, 3H), 6.17 (d, J=1.77 Hz, 1H), 5.96 (s, 1H), 4.56 (d, J=5.81 Hz, 2H), 3.63 (s, 3H), 2.41 (s, 3H), 2.23 (s, 3H), 2.17 (s, 3H); MS: 351 [M+1].

### Example 3

### N-[(4,6-dimethyl-2-oxo-1,2-dihydropyridin-3-yl)methyl]-3-(3,5-dimethyl-1H-pyrazol-4-yl)-2-methylbenzamide

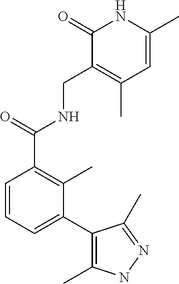 [SMILES: Cc1cc(C)c(CNC(=O)c2cccc(-c3c(C)n[nH]c3C)c2C)c(=O)[nH]1]

The compound of Example 3 was made by the method of Example 1, using 3,5-dimethylpyrazole-4-boronic acid pinacol ester as the coupling partner in the final Suzuki reaction, to provide the title compound. ¹H NMR (400 MHz, DMSO-d₆) δ 11.45 (br. s., 1H), 8.15 (s, 1H), 7.14-7.23 (m, 2H), 7.08 (dd, J=2.9, 6.1 Hz, 1H), 5.85 (s, 1H), 4.28 (d, J=4.9 Hz, 2H), 2.20 (s, 3H), 2.10 (s, 3H), 2.01 (s, 3H), 1.95 (s, 6H); MS: 365 [M+1].

### Example 4

### 3-(1,4-dimethyl-1H-imidazol-5-yl)-N-[(4,6-dimethyl-2-oxo-1,2-dihydropyridin-3-yl)methyl]-2-methylbenzamide

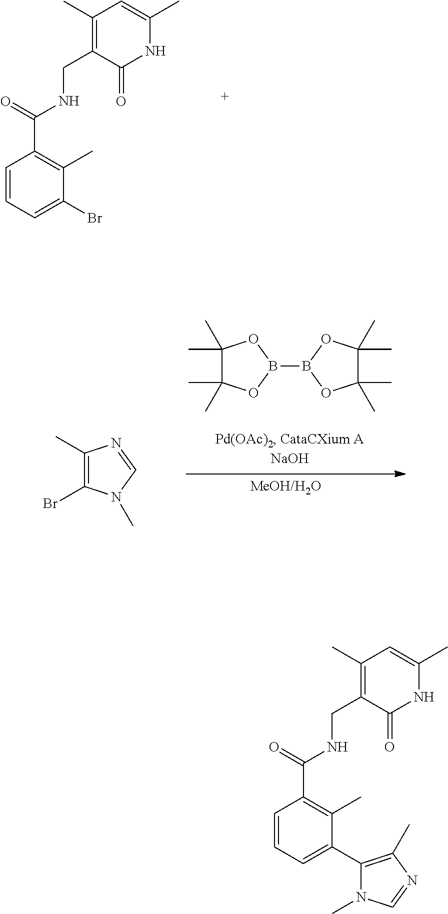

To a solution of 3-bromo-N-((4,6-dimethyl-2-oxo-1,2-dihydropyridin-3-yl)methyl)-2-methylbenzamide (400 mg, 1.12 mmol), bis(pinacolato)diboron (748 mg, 2.96 mmol), and 5-bromo-1,4-dimethyl-1H-imidazole (300 mg, 1.72 mmol) in methanol (80 mL) under nitrogen was added a solution of sodium hydroxide (90 mg, 2.24 mmol) in water (8 mL) and the mixture degassed three times with nitrogen. To this was added di(1-adamantyl)-n-butylphosphine (cataCXium® A, 48 mg, 0.144 mmol) and palladium (II) acetate (30 mg, 0.144 mmol) and the mixture degassed again. After heating at reflux overnight, the mixture was filtered, the filtrate concentrated under vacuum, and the residue purified by preparative HPLC to give the title compound (15 mg, 3.5% yield) as a white solid. ¹H NMR (400 MHz, methanol-d₄): δ 7.71 (s, 1H), 7.43-7.41 (d, 1H), 7.37-7.34 (t, 1H), 7.38-7.26 (d, 1H), 6.13 (s, 1H), 4.50 (s, 2H), 3.39 (s, 3H), 2.40 (s, 3H), 2.26 (s, 3H), 2.10 (s, 3H), 2.00 (s, 3H). MS: 365 [M+1].

### Example 5

### 5-chloro-N-[(4,6-dimethyl-2-oxo-1,2-dihydropyridin-3-yl)methyl]-2-methyl-3-(1-methyl-1H-pyrazol-5-yl)benzamide

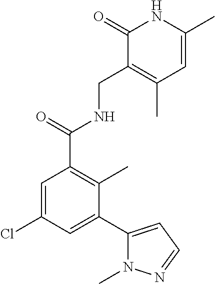 [SMILES: Cc1cc(C)c(CNC(=O)c2cc(Cl)cc(-c3ccnn3C)c2C)c(=O)[nH]1]

The compound of Example 5 was made by the method of Example 1, using 3-bromo-5-chloro-2-methylbenzoic acid in the initial amide coupling reaction, and using 1-methyl-1H-pyrazole-5-boronic acid pinacol ester as the coupling partner in the final Suzuki reaction to provide the title compound. ¹H NMR (400 MHz, DMSO-d₆) δ 11.47 (br. s., 1H), 8.39 (t, J=4.6 Hz, 1H), 7.50 (d, J=1.8 Hz, 1H), 7.36 (s, 2H), 5.86 (s, 1H), 4.27 (d, J=4.8 Hz, 2H), 3.58 (s, 3H), 2.20 (s, 3H), 2.11 (s, 3H), 2.01 (s, 3H); MS: 385 [M+1].

### Example 6

### 5-[9-acetyl-1,2,3,4-tetrahydro-1,4-epiminonaphthalen-6-yl]-N-[(4,6-dimethyl-2-oxo-1,2-dihydropyridin-3-yl)methyl]-2-methyl-3-(1-methyl-1H-pyrazol-5-yl)benzamide

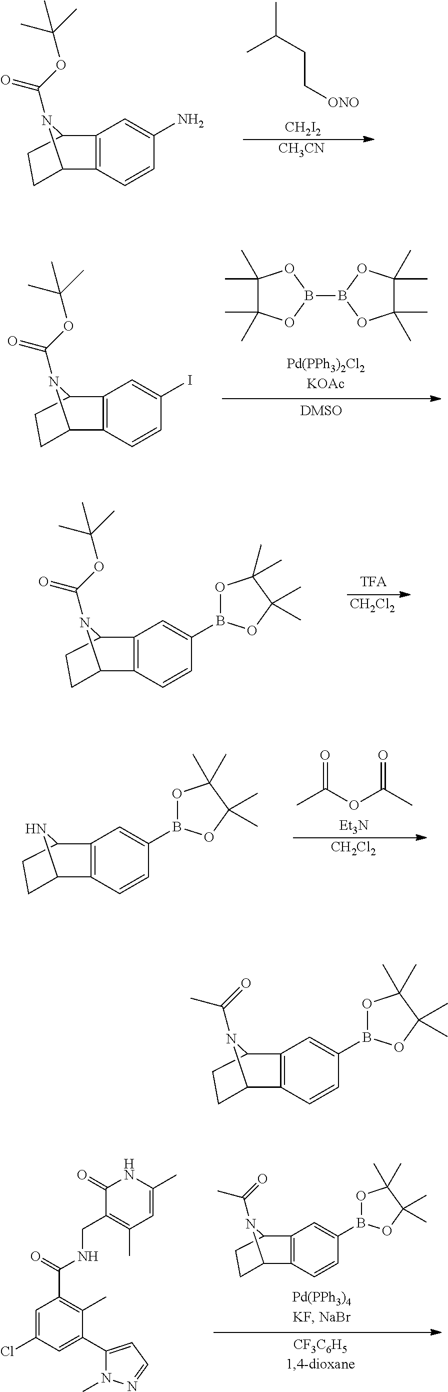

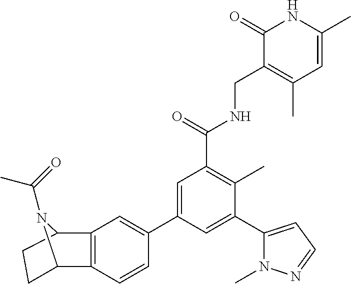 [SMILES: CC(=O)N1C2CCC1c1cc(-c3cc(C(=O)NCc4c(C)cc(C)[nH]c4=O)c(C)c(-c4ccnn4C)c3)ccc12]

To a solution of (+/−)-tert-butyl 6-amino-1,2,3,4-tetrahydro-1,4-epiminonaphthalene-9-carboxylate (5.0 g, 19.23 mmol) and diiodomethane (10.3 g, 38.46 mmol) in acetonitrile (100 mL) was added dropwise isoamyl nitrite (4.5 g, 39.46 mmol) at room temperature, and the resulting mixture was heated at reflux for 4 hours. The solvent was removed in vacuo and the residue was purified by column chromatography (petroleum ether/EtOA_{c}=3/1, Rf ˜0.8) to give (+/−)-tert-butyl 6-iodo-1,2,3,4-tetrahydro-1,4-epiminonaphthalene-9-carboxylate (4.0 g, 56% yield) as yellow oil.

To a solution of (+/−)-tert-butyl 6-iodo-1,2,3,4-tetrahydro-1,4-epiminonaphthalene-9-carboxylate (4.0 g, 10.78 mmol), bis(pinacolato)diboron (2.85 g, 11.86 mmol), and potassium acetate (3.17 g, 32.34 mmol) in dimethylsulfoxide (60 mL) under nitrogen was added bis(triphenylphosphine)palladium(II) dichloride (300 mg, 0.43 mmol). The mixture was degassed with nitrogen three times, and then stirred at 80° C. overnight. Water (50 mL) was added and the solution was extracted with ethyl acetate (3×30 mL). The combined organic phases were washed with brine (100 mL), dried over sodium sulfate, concentrated under vacuum, and the residue purified by column chromatography (silica gel, petroleum ether/EtOA_{c}=10/1, Rf ˜0.4) to yield (+/−)-tert-butyl 6-(4,4,5,5-tetramethyl-1,3,2-dioxaborolan-2-yl)-1,2,3,4-tetrahydro-1,4-epiminonaphthalene-9-carboxylate (3.9 g, 97% yield) as a yellow solid.

A mixture of (+/−)-tert-butyl 6-(4,4,5,5-tetramethyl-1,3,2-dioxaborolan-2-yl)-1,2,3,4-tetrahydro-1,4-epiminonaphthalene-9-carboxylate (0.5 g, 1.35 mmol) and trifluoroacetic acid (5 mL) in dichloromethane (10 mL) was stirred at room temperature overnight. The solvent was removed in vacuum to give crude (+/−)-6-(4,4,5,5-tetramethyl-1,3,2-dioxaborolan-2-yl)-1,2,3,4-tetrahydro-1,4-epiminonaphthalene (0.38 g, 100% yield) as a yellow oil, which was used without any further purification in the next step.

A mixture of crude (+/−)-6-(4,4,5,5-tetramethyl-1,3,2-dioxaborolan-2-yl)-1,2,3,4-tetrahydro-1,4-epiminonaphthalene (0.38 g, 1.4 mmol), acetic anhydride (0.17 g, 1.68 mmol), and triethylamine (2 mL) in dichloromethane (10 mL) was stirred at room temperature overnight.

The solvent was removed under vacuum to give (+/−)-6-(4,4,5,5-tetramethyl-1,3,2-dioxaborolan-2-yl)-1,2,3,4-tetrahydro-1,4-epiminonaphthalen-9-yl)ethanone (0.31 g, 71% yield) as a brown solid.

To a solution of (+/−)-6-(4,4,5,5-tetramethyl-1,3,2-dioxaborolan-2-yl)-1,2,3,4-tetrahydro-1,4-epiminonaphthalen-9-yl)ethanone (0.31 g, 0.98 mmol), 5-chloro-N-[(4,6-dimethyl-2-oxo-1,2-dihydropyridin-3-yl)methyl]-2-methyl-3-(1-methyl-1H-pyrazol-5-yl)benzamide (0.25 g, 0.658 mmol), potassium fluoride (0.145 g, 1.97 mmol), and sodium bromide (0.1 g, 0.98 mmol) in 1,4-dioxane (2 mL)/α,α,α-trifluorotoluene (10 mL) was added tetrakis(triphenylphosphine)-palladium(0) (65 mg, 0.056 mmol) under nitrogen. The resulting mixture was irradiated in a 175° C. microwave reactor for 1 hour. The solvent was removed in vacuum and the residue was purified by preparative HPLC to give the monoformate salt of the title compound (14 mg, 3.9% yield) as a white solid. ¹H NMR (400 MHz, DMSO-d₆): δ 8.377 (s, 2H), 7.667-7.694 (d, 1H), 7.581 (s, 1H), 7.494-7.518 (m, 3H), 7.384-7.403 (d, 1H), 6.292 (s, 1H), 5.877 (d, 1H), 5.358-5.405 (m, 1H), 4.328 (s, 1H), 3.623 (s, 3H), 2.222 (s, 3H), 1.974-2.117 (m, 8H), 1.321-1.343 (m, 3H); MS: 536 [M+1].

### Method B

### Example 7

### 5-chloro-N-[(4,6-dimethyl-2-oxo-1,2-dihydropyridin-3-yl)methyl]-3-(1,4-dimethyl-1H-pyrazol-5-yl)-2-methylbenzamide

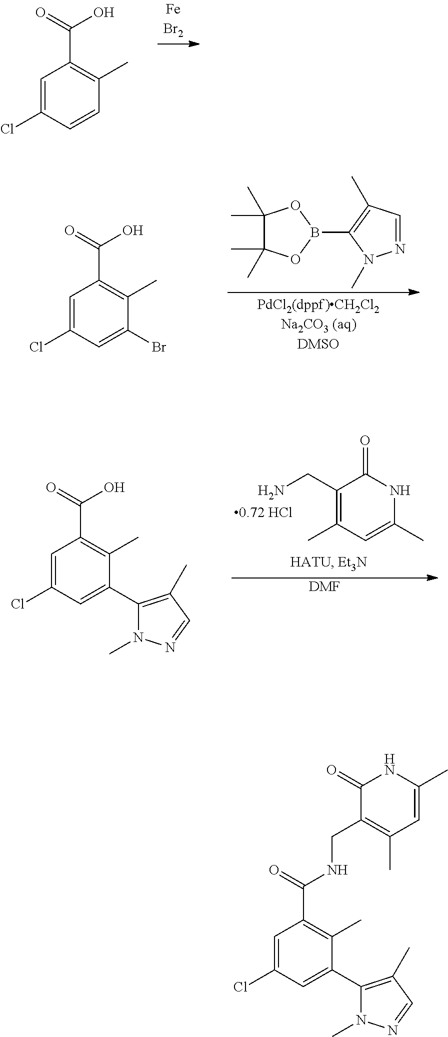

5-Chloro-2-methylbenzoic acid (10.8 g, 63.3 mmol) was added in portions to a mixture of bromine (40 mL) and iron powder (1.8 g, 31.7 mmol). The vial was sealed and the mixture stirred at room temperature for 27 hours. The reaction mixture was poured carefully into an ice bath-cooled solution of sodium thiosulfate (100 g) in deionized water (500 mL), and then extracted with ethyl acetate (300 mL, then 2×150 mL). The combined extracts were washed with saturated aqueous sodium chloride solution (2×100 mL), dried over sodium sulfate, and the solvent evaporated under vacuum. The residue was dissolved in minimum amount of ethyl acetate, packed with silica gel, and purified by column chromatography with 25% [0.05% AcOH in ethyl acetate] in heptane to give 3-bromo-5-chloro-2-methyl-benzoic acid (14.3 g, 60% purity, 55% yield) as a white solid which was contaminated with unreacted 5-chloro-2-methylbenzoic acid, as well as isomeric mono-brominated and bis brominated side products. ¹H NMR (400 MHz, DMSO-d₆) δ 13.56 (br. s., 1H), 7.94 (d, J=2.20 Hz, 2H), 7.74 (d, J=2.20 Hz, 2H), 2.52 (s, 3H).

Aqueous sodium carbonate solution (2.0 M, 0.88 mL, 1.76 mmol) was added via syringe to a septum-sealed vial containing a solution of 3-bromo-5-chloro-2-methyl-benzoic acid (146.2 mg, 0.586 mmol), (1,4-dimethyl-1H-pyrazol-5-yl)boronic acid pinacol ester (136.6 mg, 0.615 mmol), and dichloro-1,1′-bis(diphenylphosphino)ferrocene palladium (II)-dichloromethane complex [PdCl₂(dppf).CH₂Cl₂] (66.0 mg, 0.081 mmol) in N,N-dimethylformamide (3.0 mL). The vial was irradiated in a 120° C. microwave for 20 minutes. After cooling to room temperature, the solution was diluted with ethyl acetate (20 mL) and deionized water (10 mL). The mixture was acidified to pH ˜2 with aqueous hydrochloric acid (1.0 M, ˜5 mL), then suction-filtered to remove a small amount of black precipitate. The layers of the biphasic filtrate were separated, and the aqueous layer extracted further with ethyl acetate (2×20 mL). The combined organic extracts were dried over magnesium sulfate, filtered, and concentrated to minimal volume, affording crude 5-chloro-3-(1,4-dimethyl-1H-pyrazol-5-yl)-2-methylbenzoic acid. This material was dissolved in N,N-dimethylformamide (3.0 mL) and stirred with 3-aminomethyl-4,6-dimethyl-1H-pyridin-2-one hydrochloride (106.5 mg, 0.597 mmol), triethylamine (0.25 mL, 1.8 mmol), and O-(7-azabenzotriazol-1-yl)-N,N,N′,N′-tetramethyluronium hexafluorophosphate (HATU, 482.0 mg, 1.268 mmol) at room temperature for 2 hours. The reaction mixture was then diluted with ethyl acetate (30 mL) and washed with a mixture of deionized water (10 mL), saturated aqueous sodium bicarbonate solution (10 mL), and saturated aqueous sodium chloride solution (10 mL), all in one portion. The aqueous layer was back-extracted with ethyl acetate (2×20 mL). The combined organic extracts were dried over magnesium sulfate, filtered, concentrated, and purified by reverse-phase HPLC to give the title compound (16.56 mg, 6.7% yield) as a white solid. ¹H NMR (400 MHz, DMSO-d₆) δ 11.48 (br. s., 1H), 8.41 (t, J=4.93 Hz, 1H), 7.37 (d, J=2.27 Hz, 1H), 7.34 (s, 1H), 7.31 (d, J=2.27 Hz, 1H), 5.86 (s, 1H), 4.28 (d, J=5.05 Hz, 2H), 3.49 (s, 3H), 2.20 (s, 3H), 2.11 (s, 3H), 1.94 (s, 3H), 1.79 (s, 3H); MS: 399/401 [M+1], CI isotope pattern.

### Example 8

### 5-chloro-2-methyl-N-[(6-methyl-2-oxo-4-propyl-1,2-dihydropyridin-3-yl)methyl]-3-(1-methyl-1H-pyrazol-5-yl)benzamide

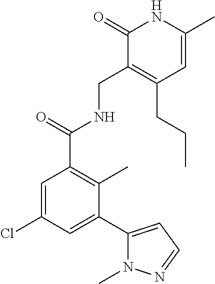 [SMILES: CCCc1cc(C)[nH]c(=O)c1CNC(=O)c1cc(Cl)cc(-c2ccnn2C)c1C]

The compound of Example 8 was made by the method of Example 7, using 1-methyl-1H-pyrazole-5-boronic acid pinacol ester as the coupling partner in the Suzuki reaction with 3-bromo-5-chloro-2-methyl-benzoic acid, and using 3-(aminomethyl)-6-methyl-4-propylpyridin-2(1H)-one hydrochloride in the final amide coupling reaction. ¹H NMR (400 MHz, DMSO-d₆) δ 11.49 (s, 1H), 8.32-8.49 (m, 1H), 7.50 (d, J=1.7 Hz, 1H), 7.37 (d, J=2.2 Hz, 1H), 7.34 (d, J=2.2 Hz, 1H), 6.27 (d, J=1.7 Hz, 1H), 5.89 (s, 1H), 4.28 (d, J=4.9 Hz, 2H), 3.58 (s, 3H), 2.12 (s, 3H), 2.01 (s, 3H), 1.46-1.60 (m, 2H), 1.18-1.31 (m, 2H), 0.92 (t, J=7.3 Hz, 3H); MS: 413 [M+1].

### Example 9

### 2-chloro-N-[(4,6-dimethyl-2-oxo-1,2-dihydropyridin-3-yl)methyl]-3-(1-methyl-1H-pyrazol-5-yl)benzamide

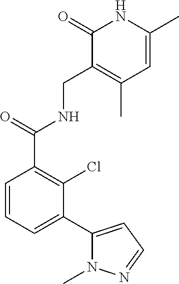 [SMILES: Cc1cc(C)c(CNC(=O)c2cccc(-c3ccnn3C)c2Cl)c(=O)[nH]1]

The compound of Example 9 was made by the method of Example 7, starting with a Suzuki coupling of 3-bromo-2-chlorobenzoic acid and 1-methyl-1H-pyrazole-5-boronic acid pinacol ester, and then using 3-aminomethyl-4,6-dimethyl-1H-pyridin-2-one hydrochloride in the subsequent amide formation. ¹H NMR (400 MHz, DMSO-d₆) δ 8.45 (br. s., 1H) 7.50 (d, J=1.7 Hz, 1H) 7.44 (s, 3H) 6.30 (d, J=1.7 Hz, 1H) 5.86 (s, 1H) 4.29 (d, J=2.9 Hz, 2H) 3.62 (s, 3H) 2.19 (s, 3H) 2.11 (s, 3H); MS: 371 [M+1].

### Example 10

### 3-(4-chloro-1-methyl-1H-pyrazol-5-yl)-N-[(4,6-dimethyl-2-oxo-1,2-dihydropyridin-3-yl)methyl]-2-methylbenzamide

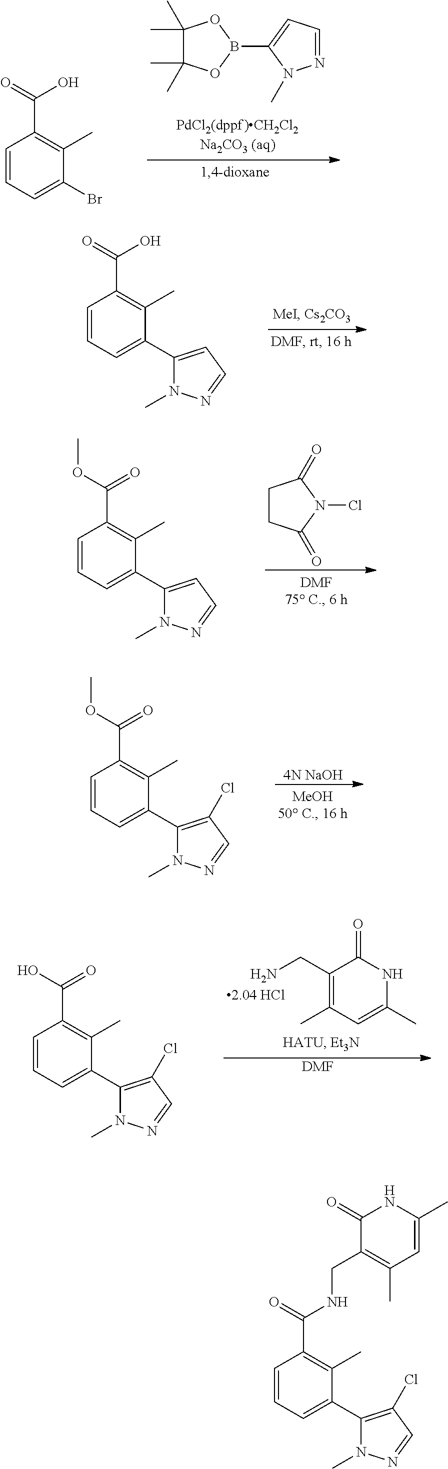

A partial solution of 3-bromo-2-methylbenzoic acid (0.408 g, 1.90 mmol), 1-methyl-1H-pyrazole-5-boronic acid pinacol ester (0.474 g, 2.28 mmol), and 1M aqueous sodium carbonate solution (3.79 mL) in 1,4-dioxane (10 mL) was degassed by bubbling nitrogen through the solution for 10 minutes. The reaction mixture was treated with dichloro-1,1′-bis(diphenylphosphino)ferrocene palladium (II)-dichloromethane complex [PdCl₂(dppf).CH₂Cl₂] (0.107 g, 0.131 mmol) and irradiated in a 130° C. microwave for 30 minutes, then diluted with water and washed with ethyl acetate. The aqueous layer was acidified with 6N hydrochloric acid (3 mL) and extracted with dichloromethane twice. The combined dichloromethane extracts were concentrated to give crude 2-methyl-3-(1-methyl-1H-pyrazol-5-yl)benzoic acid as a purple residue. MS: 217 [M+1].

The crude 2-methyl-3-(1-methyl-1H-pyrazol-5-yl)benzoic acid was dissolved in N,N-dimethylformamide (10 mL) and treated with cesium carbonate (0.725 g, 2.2 mmol), followed by iodomethane (0.200 mL, 3.21 mmol). The reaction was stirred at room temperature overnight. The reaction mixture was poured into ethyl acetate and washed with water twice and saturated aqueous sodium chloride. The combined ethyl acetate extracts were concentrated to give a dark oil which was purified on silica gel (Biotage Flash 40S, 0-50% ethyl acetate in heptane) to give methyl 2-methyl-3-(1-methyl-1H-pyrazol-5-yl)benzoate (0.211 g, 48% yield from 3-bromo-2-methylbenzoic acid) as a tan wax. ¹H NMR (400 MHz, chloroform-d) δ 7.93 (dd, J=1.52, 7.58 Hz, 1H), 7.56 (d, J=1.77 Hz, 1H), 7.30-7.40 (m, 2H), 6.22 (d, J=1.77 Hz, 1H), 3.94 (s, 3H), 3.64 (s, 3H), 2.35 (s, 3H); MS: 231 [M+1].

To a solution of methyl 2-methyl-3-(1-methyl-1H-pyrazol-5-yl)benzoate (0.0801 g, 0.348 mmol) in N,N-dimethylformamide (1.5 mL) was added N-chlorosuccinimide (0.055 mg, 0.41 mmol) and the reaction was heated at 75° C. for 6 hours. The reaction mixture was cooled to room temperature, diluted with ethyl acetate and washed sequentially with water, 1M aqueous sodium hydroxide, water, and then brine. The ethyl acetate layer was concentrated to give methyl 3-(4-chloro-1-methyl-1H-pyrazol-5-yl)-2-methylbenzoate (87 mg, 94% yield) as a thick, clear oil. ¹H NMR (400 MHz, chloroform-d) δ 8.00 (dd, J=2.02, 7.33 Hz, 1H), 7.54 (s, 1H), 7.34-7.42 (m, 2H), 3.94 (s, 3H), 3.63 (s, 3H), 2.36 (s, 3H); MS: 265 [M+1].

A solution of 3-(4-chloro-1-methyl-1H-pyrazol-5-yl)-2-methylbenzoate (0.084 g, 0.32 mmol) in methanol (3 mL) was treated with 4N aqueous sodium hydroxide (0.175 mL, 0.70 mmol) and stirred at 50° C. overnight. The reaction was cooled to room temperature, neutralized with 6N HCl (120 μL) and concentrated to an oily residue. This material was dissolved in N,N-dimethylformamide (2 mL), treated with triethylamine (0.200 mL, 1.44 mmol), 3-aminomethyl-4,6-dimethyl-1H-pyridin-2-one hydrochloride (0.0847 g, 0.374 mmol), and then O-(7-azabenzotriazol-1-yl)-N,N,N′,N′-tetramethyluronium hexafluorophosphate (HATU, 0.149 g, 0.392 mmol) was added. The reaction was stirred at room temperature for 30 minutes. Water (0.5 mL) was added, and the reaction became homogeneous. The reaction mixture was filtered and purified by preparative HPLC to give the title compound (0.043 g, 35% yield) as a white solid. ¹H NMR (400 MHz, DMSO-d₆) δ 11.40 (br. s., 1H), 8.31 (t, J=4.67 Hz, 1H), 7.65 (s, 1H), 7.37-7.41 (m, 1H), 7.32-7.37 (m, 1H), 7.26-7.30 (m, 1H), 5.86 (s, 1H), 4.24-4.34 (m, 2H), 3.56 (s, 3H), 2.20 (s, 3H), 2.11 (s, 3H), 2.03 (s, 3H); MS: 385 [M+1].

### Example 11

### N-[(4,6-dimethyl-2-oxo-1,2-dihydropyridin-3-yl)methyl]-3-(4-fluoro-1-methyl-1H-pyrazol-5-yl)-2-methylbenzamide

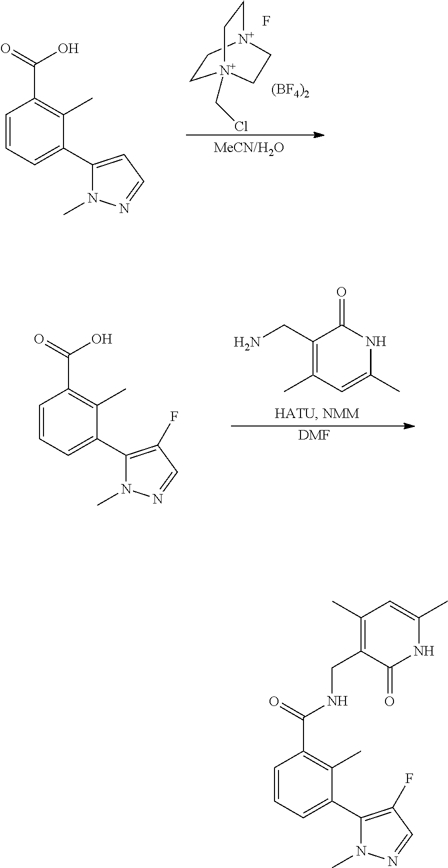

To a solution of 2-methyl-3-(1-methyl-1H-pyrazol-5-yl)benzoic acid (250 mg, 1.16 mmol) in acetonitrile (10 mL), acetic acid (664 μL, 11.6 mmol), and 1-chloromethyl-4-fluoro-1,4-diazoniabicyclo[2.2.2]octane bis(tetrafluoroborate) (Selectfluor®, 1 g, 3 mmol) were added. The reaction was heated at 50° C. for 16 hours. The reaction was diluted with water and extracted with ethyl acetate (3×10 mL). The organics were dried over sodium sulfate and evaporated to dryness. The residue was dissolved in dimethylsulfoxide and purified by HPLC to give 3-(4-fluoro-1-methyl-1H-pyrazol-5-yl)-2-methylbenzoic acid (10 mg, 4% yield) as a light yellow solid. ¹H NMR (400 MHz, DMSO-d₆): δ 7.81 (dd, J=7.3, 1.5 Hz, 1H), 7.59 (d, J=4.5 Hz, 1H), 7.21-7.45 (m, 2H), 3.56 (s, 3H), 2.27 (s, 3H). MS: 235 [M+1].

To a solution of 3-(4-fluoro-1-methyl-1H-pyrazol-5-yl)-2-methylbenzoic acid (97 mg, 0.26 mmol) in N,N-dimethylformamide (5 mL), O-(7-azabenzotriazol-1-yl)-N,N,N′,N′-tetramethyl-uronium hexafluorophosphate (HATU, 99.2 mg, 0.261 mmol) and N-methylmorpholine (43 μL, 0.392 mmol) were added. The solution was stirred at room temperature for 30 min, then 3-(aminomethyl)-4,6-dimethylpyridin-2(1H)-one hydrochloride (74 mg, 0.392 mmol) and N-methylmorpholine (86 μL, 0.78 mmol) were added and the reaction was stirred at room temperature overnight. The reaction was diluted with water and extracted with ethyl acetate (3×10 mL). The organics were dried over sodium sulfate, filtered and evaporated to dryness. The residue was dissolved in dimethylsulfoxide and purified by HPLC, affording the title compound (2.19 mg, 3% yield) as a white solid. ¹H NMR (400 MHz, methanol-d₄): δ 7.44-7.63 (m, 2H), 7.29-7.47 (m, 2H), 6.14 (s, 1H), 4.52 (s, 2H), 3.62 (s, 3H), 2.41 (s, 3H), 2.27 (s, 3H), 2.20 (s, 3H). MS: 369.1/370 [M+1].

### Example 12

### N-[(4,6-dimethyl-2-oxo-1,2-dihydropyridin-3-yl)methyl]-3-(1-ethyl-4-methyl-1H-pyrazol-5-yl)-2-methylbenzamide

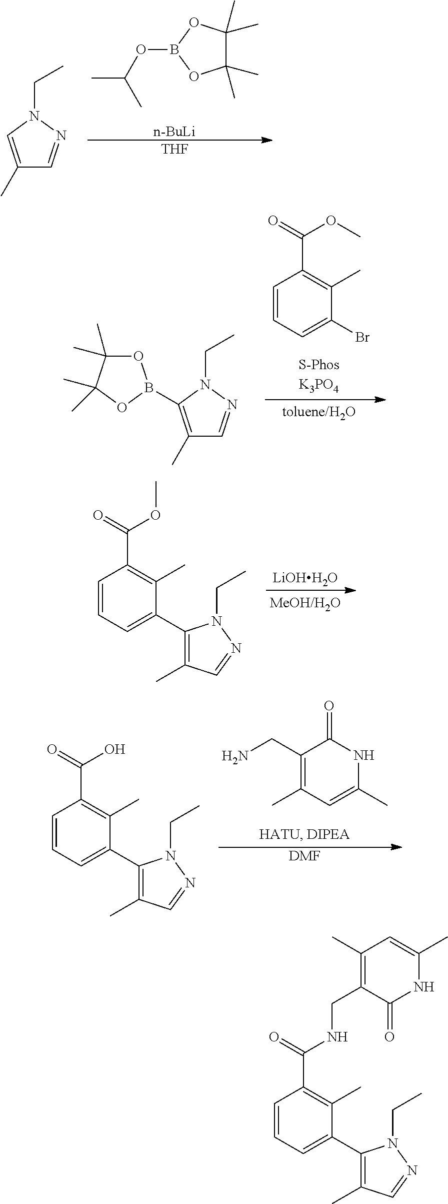

To a cooled (0° C.) suspension of 1-ethyl-4-methyl-1H-pyrazole (1.5 g, 13.6 mmol) in dry tetrahydrofuran (25 mL) was added dropwise n-butyllithium solution (2.5 M in tetrahydrofuran, 6.5 mL, 16.32 mmol). The resulting mixture was stirred at room temperature for 2 hours. The solution was cooled to −65° C. and 2-isopropoxy-4,4,5,5-tetramethyl-1,3,2-dioxaborolane (3.04 g, 16.32 mmol) was added dropwise. When the addition was complete, the mixture was stirred at −65° C. for 15 minutes, at 0° C. for 1 hour, and then at room temperature overnight. The reaction mixture was cooled again to 0° C. and quenched with saturated aqueous ammonium chloride solution. The mixture was extracted with ethyl acetate (3×100 mL). The combined organic layers were washed with brine (50 mL), dried over sodium sulfate and concentrated under vacuum. The residue was purified by column chromatography (silica gel, petroleum ether/EtOAc from 100/1 to 6/1) to give 1-ethyl-4-methyl-5-(4,4,5,5-tetramethyl-1,3,2-dioxaborolan-2-yl)-1H-pyrazole (1.5 g, 47% yield) as yellow oil.

A mixture of methyl 3-bromo-2-methylbenzoate (300 mg, 1.31 mmol), 1-ethyl-4-methyl-5-(4,4,5,5-tetramethyl-1,3,2-dioxaborolan-2-yl)-1H-pyrazole (464 mg, 1.96 mmol), potassium phosphate (556 mg, 2.61 mmol), 2-dicyclohexylphosphino-2′,6′-dimethoxybiphenyl (SPhos, 100 mg, 0.24 mmol) and palladium (II) acetate (44 mg, 0.20 mmol) in toluene (3 mL) and water (0.3 mL) was stirred under nitrogen at 120° C. overnight. The reaction mixture was cooled to room temperature and then diluted with ethyl acetate (50 mL) and water (30 mL). The resulting suspension was filtered, and the layers of the filtrate were separated. The aqueous layer was extracted with ethyl acetate (2×30 mL). The combined organic layers were washed with brine (30 mL), dried over sodium sulfate, concentrated and purified by silica gel chromatography (petroleum ether/EtOAc 10:1) to give methyl 3-(1-ethyl-4-methyl-1H-pyrazol-5-yl)-2-methylbenzoate (200 mg, 59% yield) as colorless oil.

A mixture of methyl 3-(1-ethyl-4-methyl-1H-pyrazol-5-yl)-2-methylbenzoate (200 mg, 0.76 mmol) and lithium hydroxide monohydrate (60 mg, 1.51 mmol) in methanol (10 mL) and water (1 mL) was stirred at room temperature overnight. The reaction mixture was concentrated under vacuum to remove methanol. The residue was dissolved in water (30 mL) and extracted with tert-butyl methyl ether (30 mL). The aqueous layer was acidified with concentrated hydrochloric acid to pH ˜4, and extracted with ethyl acetate (2×50 mL). The combined ethyl acetate layers were washed with brine (50 mL), dried over sodium sulfate, and concentrated under vacuum to give 3-(1-ethyl-4-methyl-1H-pyrazol-5-yl)-2-methylbenzoic acid (180 mg, 95% yield) as a white solid.

To a cooled (0° C.) solution of 3-(1-ethyl-4-methyl-1H-pyrazol-5-yl)-2-methylbenzoic acid (180 mg, 0.73 mmol), 3-aminomethyl-4,6-dimethyl-1H-pyridin-2-one hydrochloride (130 mg, 0.73 mmol) and N,N-diisopropylethyl amine (217 mg, 1.68 mmol) in N,N-dimethylformamide (10 mL) was added O-(7-azabenzotriazol-1-yl)-N,N,N′,N′-tetramethyluronium hexafluorophosphate (HATU, 416 mg, 1.1 mmol). The resulting mixture was stirred at room temperature overnight, then poured into water (50 mL) and extracted with ethyl acetate (4×30 mL). The combined organic layers were washed with brine (5×20 mL), dried over sodium sulfate, and concentrated to dryness under vacuum. The residue was purified via crystallization from ethyl acetate (5 mL) to give the title compound (170 mg, 62% yield) as a white solid. ¹H NMR (400 MHz, methanol-d₄): δ 7.47-7.45 (d, 1H), 7.42-7.36 (t, 1H), 7.28-7.26 (d, 1H), 6.13 (s, 1H), 4.51 (s, 2H), 3.94-3.78 (m, 2H), 2.40 (s, 3H), 2.26 (s, 3H), 2.08 (s, 3H), 1.86 (s, 3H), 1.24-1.20 (t, 3H): MS 401 [M+Na].

### Method C

### Example 13

### 5-[2-(dimethylamino)pyrimidin-5-yl]-N-[(4,6-dimethyl-2-oxo-1,2-dihydropyridin-3-yl)methyl]-2-methyl-3-(1-methyl-1H-pyrazol-5-yl)benzamide

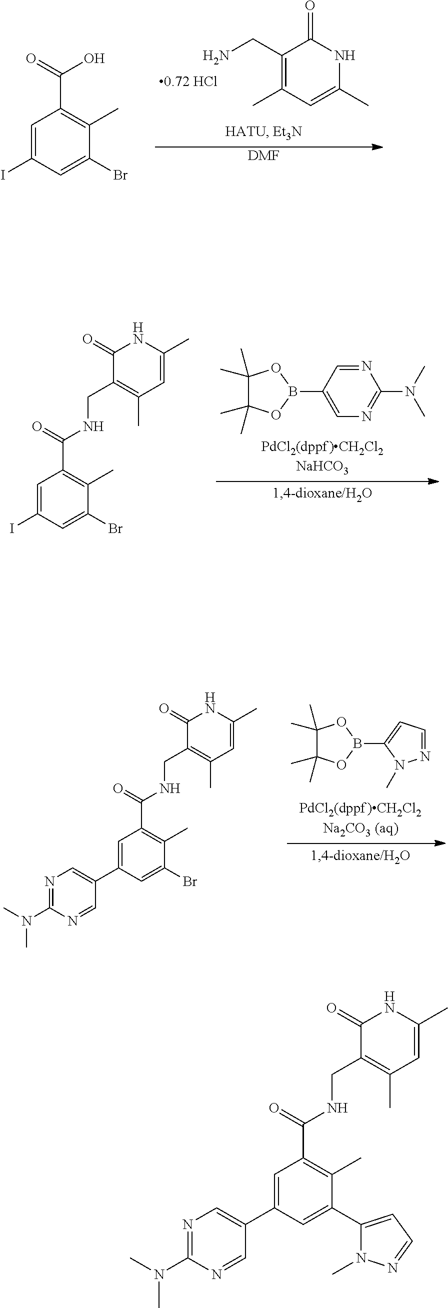

A solution of 3-bromo-5-iodo-2-methylbenzoic acid (1.78 g, 5.23 mmol), 3-aminomethyl-4,6-dimethyl-1H-pyridin-2-one hydrochloride (960.5 mg, 5.38 mmol), O-(7-azabenzotriazol-1-yl)-N,N,N′,N′-tetramethyluronium hexafluorophosphate (HATU, 2.08 g, 5.47 mmol), and triethylamine (2.20 mL, 15.8 mmol) in N,N-dimethylformamide (26 mL) was stirred at room temperature for 1 hour. The mixture was poured into a flask containing ethyl acetate (130 mL) and deionized water (50 mL), and rapidly stirred for 5 minutes. The resulting white precipitate was collected by suction filtration and dried in a 50° C. vacuum oven overnight, yielding 3-bromo-N-((4,6-dimethyl-2-oxo-1,2-dihydropyridin-3-yl)methyl)-5-iodo-2-methylbenzamide (1.65 g, 66% yield) as a white solid. ¹H NMR (400 MHz, DMSO-d₆) δ 11.47 (s, 1H), 8.39 (t, J=4.93 Hz, 1H), 7.97 (d, J=1.52 Hz, 1H), 7.50 (d, J=1.52 Hz, 1H), 5.86 (s, 1H), 4.24 (d, J=5.05 Hz, 2H), 2.24 (s, 3H), 2.18 (s, 3H), 2.11 (s, 3H); MS: 475/477 [M+1], Br isotope pattern.

A suspension of 3-bromo-N-((4,6-dimethyl-2-oxo-1,2-dihydropyridin-3-yl)methyl)-5-iodo-2-methylbenzamide (104.4 mg, 0.22 mmol), 2-(dimethylamino)pyrimidine-5-boronic acid pinacol ester (55.6 mg, 0.22 mmol), solid sodium bicarbonate (72.4 mg, 0.862 mmol), and dichloro-1,1′-bis(diphenylphosphino)ferrocene palladium (II) dichloromethane complex [PdCl₂(dppf).CH₂Cl₂] (10.1 mg, 0.012 mmol) in 1,4-dioxane (3.0 mL) and deionized water (1.0 mL) was sealed in a microwave vial and degassed by evacuation until the solvent begins to boil, followed by argon fill, 3 cycles. The vial was irradiated in a 100° C. microwave for 5 minutes. The vial was then unsealed and 1-methyl-1H-pyrazole-5-boronic acid pinacol ester (100.3 mg, 0.48 mmol), more PdCl₂(dppf).CH₂Cl₂ (27.0 mg; 0.045 mmol), and 2.0 M aqueous sodium carbonate solution (0.44 mL, 0.88 mmol) were added. The vial was resealed and degassed as above, then irradiated in a 120° C. microwave for 20 minutes. The reaction solution was passed through a 0.2 micron filter to remove solids, then purified by SFC to give the title compound (28.55 mg, 27% yield) as a white solid. ¹H NMR (400 MHz, DMSO-d₆) δ 8.72 (s, 2H), 8.32 (t, J=4.65 Hz, 1H), 7.58 (d, J=1.22 Hz, 1H), 7.54 (d, J=0.98 Hz, 1H), 7.50 (d, J=1.71 Hz, 1H), 6.28 (d, J=1.71 Hz, 1H), 5.87 (s, 1H), 4.31 (d, J=4.65 Hz, 2H), 3.62 (s, 3H), 3.16 (s, 6H), 2.21 (s, 3H), 2.11 (s, 3H), 2.06 (s, 3H); MS: 472 [M+1].

### Example 14

### N-[(4,6-dimethyl-2-oxo-1,2-dihydropyridin-3-yl)methyl]-2-methyl-5-[2-(methylamino)pyrimidin-5-yl]-3-(1-methyl-1H-pyrazol-5-yl)benzamide

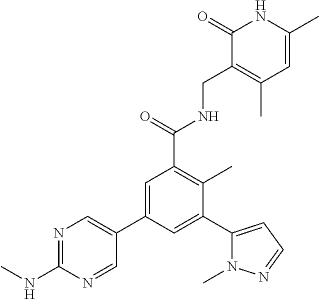

The compound of Example 14 was made using the same method as Example 13, starting with 3-bromo-N-((4,6-dimethyl-2-oxo-1,2-dihydropyridin-3-yl)methyl)-5-iodo-2-methyl-benzamide (108.4 mg, 0.23 mmol) and 2-methylaminopyrimidine-5-boronic acid pinacol ester as the first coupling partner, affording the title compound (26.6 mg, 25% yield) as a light grey solid. ¹H NMR (400 MHz, DMSO-d₆) δ 11.47 (br. s., 1H), 8.66 (s, 2H), 8.32 (t, J=4.77 Hz, 1H), 7.57 (d, J=1.71 Hz, 1H), 7.53 (d, J=1.71 Hz, 1H), 7.50 (d, J=1.71 Hz, 1H), 7.26 (q, J=4.56 Hz, 1H), 6.28 (d, J=1.71 Hz, 1H), 5.86 (s, 1H), 4.31 (d, J=4.89 Hz, 2H), 3.61 (s, 3H), 2.83 (d, J=4.89 Hz, 3H), 2.21 (s, 3H), 2.11 (s, 3H), 2.06 (s, 3H); MS: 458 [M+1].

### Example 15

### N-[(4,6-dimethyl-2-oxo-1,2-dihydropyridin-3-yl)methyl]-3-(1,4-dimethyl-1H-pyrazol-5-yl)-2-methyl-5-[2-(methylamino)pyrimidin-5-yl]benzamide

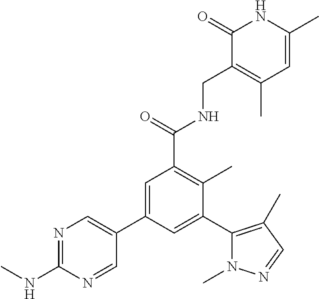

The compound of Example 15 was made using the same method as Example 13, starting with 3-bromo-N-((4,6-dimethyl-2-oxo-1,2-dihydropyridin-3-yl)methyl)-5-iodo-2-methyl-benzamide (330 mg, 0.72 mmol) with 2-methylaminopyrimidine-5-boronic acid pinacol ester as the first coupling partner and (1,4-dimethyl-1H-pyrazol-5-yl)boronic acid pinacol ester as the second coupling partner, affords the title compound (monoformate salt, 10.8 mg, 1.4% yield) as a white solid. ¹H NMR (400 MHz, DMSO-d₆) δ 8.70 (s, 2H), 8.37 (s, 1H), 7.60 (s, 1H), 7.49 (s, 1H), 7.35 (s, 1H), 7.28-7.25 (m, 1H), 5.85 (s, 1H), 4.30 (s, 2H), 3.55 (s, 3H), 2.85 (s, 3H), 2.22 (s, 3H), 2.11 (s, 3H), 1.99 (s, 3H), 1.82 (s, 3H); MS: 472 [M+1].

### Example 16

### 5-(2-aminopyrimidin-5-yl)-N-[(4,6-dimethyl-2-oxo-1,2-dihydropyridin-3-yl)methyl]-2-methyl-3-(1-methyl-1H-pyrazol-5-yl)benzamide

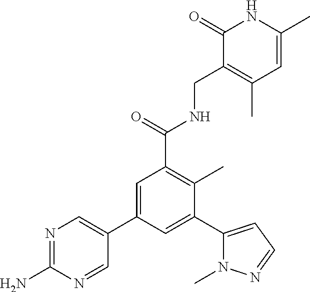 [SMILES: Cc1cc(C)c(CNC(=O)c2cc(-c3cnc(N)nc3)cc(-c3ccnn3C)c2C)c(=O)[nH]1]

The compound of Example 16 was made using the same method as Example 13, starting with 3-bromo-N-((4,6-dimethyl-2-oxo-1,2-dihydropyridin-3-yl)methyl)-5-iodo-2-methyl-benzamide (95.0 mg, 0.20 mmol) and 2-aminopyrimidine-5-boronic acid pinacol ester as the first coupling partner, affords the title compound (14.6 mg, 16% yield) as a white solid. ¹H NMR (400 MHz, DMSO-d₆) δ 11.47 (br. s., 1H), 8.62 (s, 2H), 8.31 (t, J=4.80 Hz, 1H), 7.57 (d, J=1.77 Hz, 1H), 7.53 (d, J=1.77 Hz, 1H), 7.50 (d, J=1.77 Hz, 1H), 6.79 (s, 2H), 6.28 (d, J=1.77 Hz, 1H), 5.86 (s, 1H), 4.31 (d, J=5.05 Hz, 2H), 3.61 (s, 3H), 2.21 (s, 3H), 2.11 (s, 3H), 2.06 (s, 3H); MS: 444 [M+1].

### Example 17

### N-[(4,6-dimethyl-2-oxo-1,2-dihydropyridin-3-yl)methyl]-2-methyl-3-(1-methyl-1H-pyrazol-5-yl)-5-pyrimidin-5-ylbenzamide

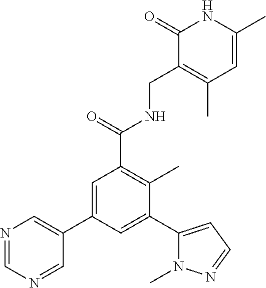 [SMILES: Cc1cc(C)c(CNC(=O)c2cc(-c3cncnc3)cc(-c3ccnn3C)c2C)c(=O)[nH]1]

The compound of Example 17 was made using the same method as Example 13, starting with 3-bromo-N-((4,6-dimethyl-2-oxo-1,2-dihydropyridin-3-yl)methyl)-5-iodo-2-methylbenzamide (94.5 mg, 0.20 mmol) and pyrimidine-5-boronic acid as the first coupling partner, affords the title compound (17.5 mg, 20% yield) as a white solid. ¹H NMR (400 MHz, DMSO-d₆) δ 9.20 (s, 2H), 9.18 (s, 1H), 8.52 (s, 1H), 8.38 (t, J=4.77 Hz, 1H), 7.79 (d, J=1.96 Hz, 1H), 7.75 (d, J=1.96 Hz, 1H), 7.52 (d, J=1.96 Hz, 1H), 6.31 (d, J=1.71 Hz, 1H), 5.87 (s, 1H), 4.32 (d, J=4.89 Hz, 2H), 3.63 (s, 3H), 2.22 (s, 3H), 2.12 (s, 3H), 2.11 (s, 3H); MS: 429 [M+1].

### Example 18

### N-[(4,6-dimethyl-2-oxo-1,2-dihydropyridin-3-yl)methyl]-2-methyl-5-(1-methyl-1H-pyrazol-4-yl)-3-(1-methyl-1H-pyrazol-5-yl)benzamide

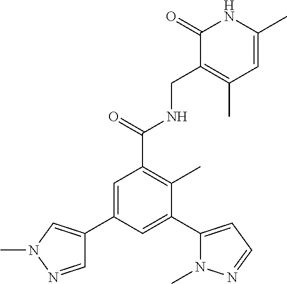

The compound of Example 18 was made using the same method as Example 13, starting with 3-bromo-N-((4,6-dimethyl-2-oxo-1,2-dihydropyridin-3-yl)methyl)-5-iodo-2-methyl-benzamide (284.8 mg, 0.60 mmol) and 1-methylpyrazole-4-boronic acid pinacol ester as the first coupling partner, affords the title compound (22.5 mg, 8.7% yield) as a white solid. ¹H NMR (400 MHz, DMSO-d₆) δ 11.46 (s, 1H), 8.27 (t, J=5.01 Hz, 1H), 8.20 (s, 1H), 7.90 (s, 1H), 7.50 (dd, J=1.71, 4.16 Hz, 2H), 7.47 (d, J=1.71 Hz, 1H), 6.25 (d, J=1.71 Hz, 1H), 5.86 (s, 1H), 4.30 (d, J=4.89 Hz, 2H), 3.83 (s, 3H), 3.60 (s, 3H), 2.21 (s, 3H), 2.11 (s, 3H), 2.01 (s, 3H); MS: 431 [M+1].

### Example 19

### 5-(6-aminopyridin-3-yl)-N-[(4,6-dimethyl-2-oxo-1,2-dihydropyridin-3-yl)methyl]-2-methyl-3-(1-methyl-1H-pyrazol-5-yl)benzamide

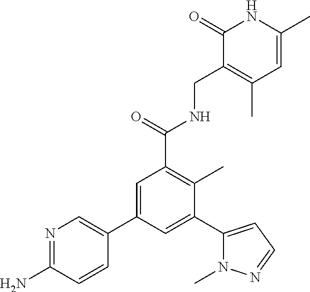 [SMILES: Cc1cc(C)c(CNC(=O)c2cc(-c3ccc(N)nc3)cc(-c3ccnn3C)c2C)c(=O)[nH]1]

The compound of Example 19 was made using the same method as Example 13, starting with 3-bromo-N-((4,6-dimethyl-2-oxo-1,2-dihydropyridin-3-yl)methyl)-5-iodo-2-methyl-benzamide (283.7 mg, 0.60 mmol) and 2-aminopyridine-5-boronic acid pinacol ester as the first coupling partner, affords the title compound (12.9 mg, 4.9% yield) as a white solid. ¹H NMR (400 MHz, DMSO-d₆) δ 11.47 (br. s., 1H), 8.32 (t, J=4.89 Hz, 1H), 8.28 (d, J=2.45 Hz, 1H), 7.74 (dd, J=2.57, 8.68 Hz, 1H), 7.47-7.53 (m, 2H), 7.44 (d, J=1.71 Hz, 1H), 6.50 (d, J=8.80 Hz, 1H), 6.27 (d, J=1.71 Hz, 1H), 6.08 (s, 2H), 5.86 (s, 1H), 4.30 (d, J=4.89 Hz, 2H), 3.61 (s, 3H), 2.21 (s, 3H), 2.11 (s, 3H), 2.05 (s, 3H); MS: 443 [M+1].

### Example 20

### 5-(3,6-dihydro-2H-pyran-4-yl)-N-[(4,6-dimethyl-2-oxo-1,2-dihydropyridin-3-yl)methyl]-2-methyl-3-(1-methyl-1H-pyrazol-5-yl)benzamide

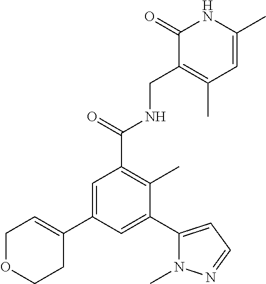 [SMILES: Cc1cc(C)c(CNC(=O)c2cc(C3=CCOCC3)cc(-c3ccnn3C)c2C)c(=O)[nH]1]

The compound of Example 20 was made using the method of Example 13, starting with 3-bromo-N-((4,6-dimethyl-2-oxo-1,2-dihydropyridin-3-yl)methyl)-5-iodo-2-methylbenzamide (100 mg, 0.21 mmol) and 3,6-dihydro-2H-pyran-4-boronic acid pinacol ester as the first coupling partner, affords the title compound (monoacetate salt, 6.9 mg, 7% yield) as a white solid. ¹H NMR (400 MHz, DMSO-d₆) δ 11.46 (br. s., 1H) 8.28 (s, 1H) 7.49 (d, J=1.7 Hz, 1H) 7.38 (d, J=1.7 Hz, 1H) 7.31 (d, J=1.7 Hz, 1H) 6.32 (br. s., 1H) 6.24 (d, J=1.7 Hz, 1H) 5.86 (s, 1H) 4.29 (d, J=4.9 Hz, 2H) 4.20 (d, J=2.7 Hz, 2H) 3.80 (t, J=5.5 Hz, 2H) 3.57 (s, 2H) 2.20 (s, 3H) 2.10 (s, 3H) 2.03 (s, 3H); MS: 433 [M+1].

### Example 21

### N-[(4,6-dimethyl-2-oxo-1,2-dihydropyridin-3-yl)methyl]-3-(1,4-dimethyl-1H-pyrazol-5-yl)-2-methyl-5-(2-morpholin-4-ylpyrimidin-5-yl)benzamide

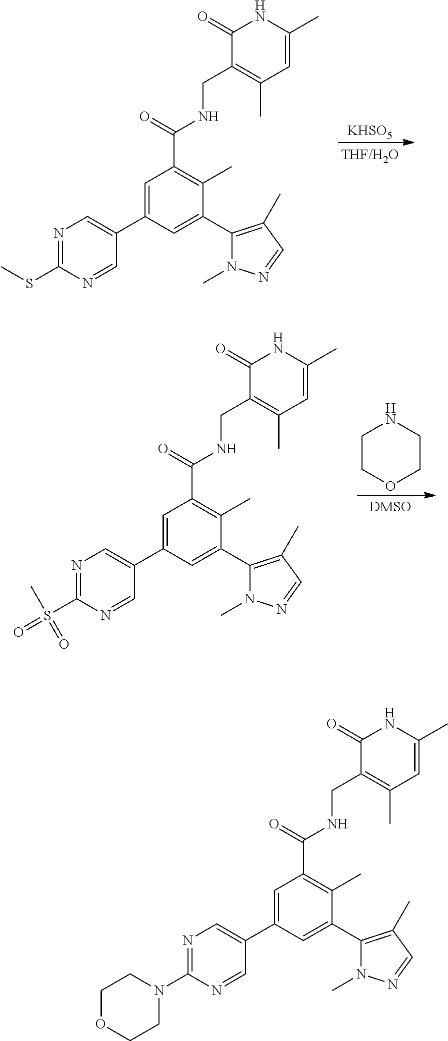

The compound of Example 21 was made following the method of Example 13, starting with 3-bromo-N-((4,6-dimethyl-2-oxo-1,2-dihydropyridin-3-yl)methyl)-5-iodo-2-methylbenzamide (392.0 mg, 0.825 mmol), using 2-(methylthio)pyrimidine-5-boronic acid pinacol ester as the first coupling partner and (1,4-dimethyl-1H-pyrazol-5-yl)boronic acid pinacol ester as the second coupling partner, afforded, after silica gel chromatography, 3-(1,4-dimethyl-1H-pyrazol-5-yl)-N-((4,6-dimethyl-2-oxo-1,2-dihydropyridin-3-yl)methyl)-2-methyl-5-(2-(methylthio)pyrimidin-5-yl)benzamide (49.2 mg, 12% yield) as a light tan solid. ¹H NMR (400 MHz, DMSO-d₆) δ 11.47 (s, 1H), 9.03 (s, 2H), 8.38 (t, J=4.93 Hz, 1H), 7.75 (d, J=2.02 Hz, 1H), 7.65 (d, J=1.77 Hz, 1H), 7.35 (s, 1H), 5.87 (s, 1H), 4.32 (d, J=5.05 Hz, 2H), 3.54 (s, 3H), 2.56 (s, 3H), 2.22 (s, 3H), 2.11 (s, 3H), 2.03 (s, 3H), 1.83 (s, 3H); MS: 489 [M+1].

To a solution of 3-(1,4-dimethyl-1H-pyrazol-5-yl)-N-((4,6-dimethyl-2-oxo-1,2-dihydropyridin-3-yl)methyl)-2-methyl-5-(2-(methylthio)pyrimidin-5-yl)benzamide (44.9 mg, 0.092 mmol) in tetrahydrofuran (1.0 mL) and deionized water (1.0 mL) was added potassium peroxymonosulfate (114.8 mg, 0.187 mmol) at room temperature. After stirring for 3 days, the mixture was partitioned between ethyl acetate (20 mL) and saturated aqueous sodium chloride solution (5 mL). The organic layer was dried over magnesium sulfate, filtered, and concentrated under vacuum, affording crude 3-(1,4-dimethyl-1H-pyrazol-5-yl)-N-((4,6-dimethyl-2-oxo-1,2-dihydropyridin-3-yl)methyl)-2-methyl-5-(2-(methylsulfonyl)pyrimidin-5-yl)benzamide (44.7 mg) as a yellow foam. MS: 521 [M+1].

Morpholine (0.10 mL, 1.1 mmol) was added to a solution of the crude 3-(1,4-dimethyl-1H-pyrazol-5-yl)-N-((4,6-dimethyl-2-oxo-1,2-dihydropyridin-3-yl)methyl)-2-methyl-5-(2-(methylsulfonyl)pyrimidin-5-yl)benzamide in dimethylsulfoxide (1.0 mL), and the mixture irradiated in a 120° C. microwave for 15 minutes. After cooling to room temperature, the mixture was passed through a 0.2 micron filter and purified by reverse-phase HPLC, affording the title compound (6.1 mg, 12.5% yield) as a white solid. ¹H NMR (400 MHz, DMSO-d₆) δ 11.45 (s, 1H), 8.77 (s, 2H), 8.35 (t, J=4.89 Hz, 1H), 7.62 (d, J=1.96 Hz, 1H), 7.51 (d, J=1.96 Hz, 1H), 7.34 (s, 1H), 5.86 (s, 1H), 4.31 (d, J=4.89 Hz, 2H), 3.72-3.77 (m, 4H), 3.64-3.70 (m, 4H), 3.53 (s, 3H), 2.22 (s, 3H), 2.11 (s, 3H), 2.00 (s, 3H), 1.82 (s, 3H); MS: 528 [M+1].

### Method D

### Example 22

### N-[(4,6-dimethyl-2-oxo-1,2-dihydropyridin-3-yl)methyl]-3-(1,4-dimethyl-1H-pyrazol-5-yl)-2-methyl-5-{2-[(1S,4S)-2-oxa-5-azabicyclo[2.2.1]hept-5-yl]pyrimidin-5-yl}benzamide

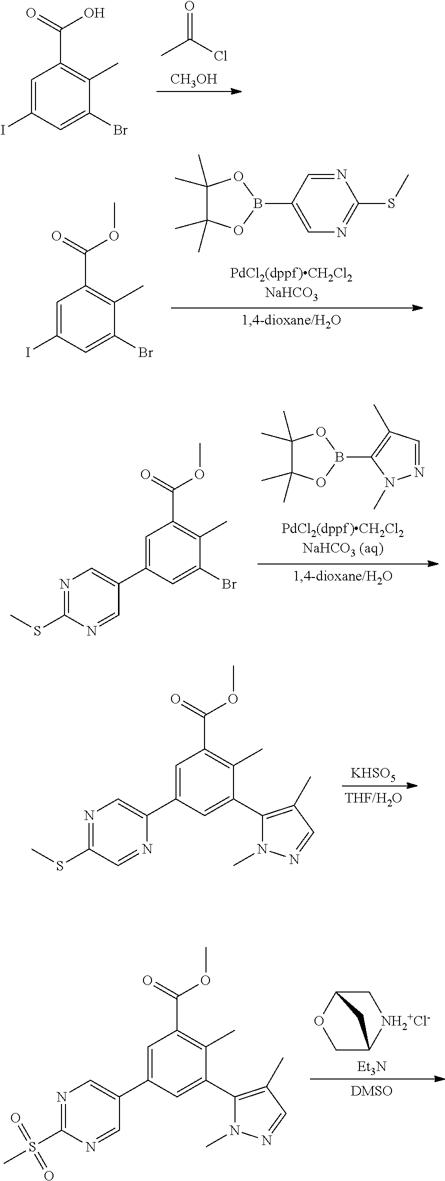

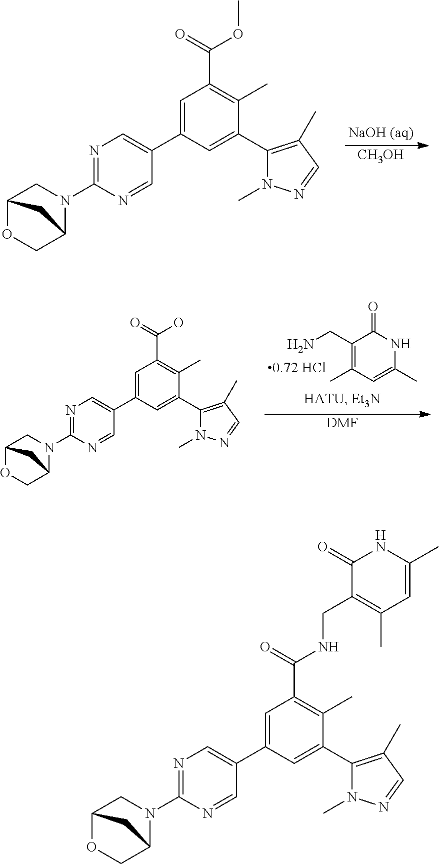

Acetyl chloride (0.70 mL, 9.8 mmol) was added to a suspension of 3-bromo-5-iodo-2-methylbenzoic acid (3.74 g, 11.0 mmol) in methanol (50 mL) at room temperature. The mixture was heated at reflux for 6.5 hours, and then stirred at 55° C. for 17.5 hours. The resulting solution was evaporated to dryness, and the white solid residue triturated from acetonitrile (15 mL). The solids were collected by filtration. The filtrate was concentrated to dryness and the residue triturated from acetonitrile (10 mL) to give a second crop of solids. The combined crops of solids were dried in a 50° C. vacuum oven for 2 hours, 45 minutes. The resulting solid was shown to contain unreacted 3-bromo-5-iodo-2-methylbenzoic acid, so these solids were then partitioned between ethyl acetate (30 mL) and saturated aqueous sodium bicarbonate solution (20 mL). The organic phase was dried over magnesium sulfate, filtered, and concentrated to a sticky white solid. Trituration from acetonitrile as described above and drying overnight in a 50° C. vacuum oven afforded methyl 3-bromo-5-iodo-2-methylbenzoate (1.6751 g, 43% yield) as a free-flowing white solid. ¹H NMR (400 MHz, DMSO-d₆) δ 8.17 (d, J=1.71 Hz, 1H), 8.00 (d, J=1.71 Hz, 1H), 3.84 (s, 3H), 2.45 (s, 3H).

The following reagents were distributed evenly into two 20 mL microwave vials: 3-bromo-5-iodo-2-methylbenzoate (1.5997 g, 4.507 mmol), 2-(methylthio)pyrimidine-5-boronic acid pinacol ester (1.1975 g, 4.749 mmol), solid sodium bicarbonate (1.556 g, 13.76 mmol), and dichloro-1,1′-bis(diphenylphosphino)ferrocene palladium (II)-dichloromethane complex [PdCl₂(dppf).CH₂Cl₂] (187.7 mg, 0.23 mmol). Each vial was sealed with a septum cap, evacuated, and filled with argon. To each vial was added 1,4-dioxane (12 mL) and deionized water (3.0 mL) via syringe. The solution in each vial was degassed by evacuation until the solvent began to boil, followed by argon fill, 3 cycles. Each vial was irradiated in a 100° C. microwave for 5 minutes. After cooling to room temperature the solutions in the two vials were combined and concentrated under vacuum to remove 1,4-dioxane. The residue was partitioned between ethyl acetate (30 mL) and deionized water (15 mL). The organic layer was dried over magnesium sulfate, filtered, concentrated, and purified by silica gel chromatography (eluting with a gradient of 0-100% ethyl acetate in heptanes), to give methyl 3-bromo-2-methyl-5-(2-(methylthio)pyrimidin-5-yl)benzoate (814.4 mg, 51% yield, 89% purity) as a white solid. ¹H NMR (400 MHz, DMSO-d₆) δ 9.01 (s, 2H), 8.24 (d, J=1.83 Hz, 1H), 8.05 (d, J=1.83 Hz, 1H), 3.88 (s, 3H), 2.56 (s, 3H), 2.54 (s, 3H); MS: 353/355 [M+1], Br isotope pattern.

To a solution of methyl 3-bromo-2-methyl-5-(2-(methylthio)pyrimidin-5-yl)benzoate (800.5 mg, 2.266 mmol) in 1,4-dioxane (12 mL) in a 20 mL microwave vial was added (1,4-dimethyl-1H-pyrazol-5-yl)boronic acid pinacol ester (577.1 mg, 2.598 mmol), dichloro-1,1′-bis(diphenylphosphino)ferrocene palladium (II)-dichloromethane complex [PdCl₂(dppf).CH₂Cl₂] (177 mg, 0.22 mmol), and 2.0 M aqueous sodium carbonate solution (3.4 mL, 6.8 mmol). The vial was sealed with a septum cap, and the solution degassed by evacuation until the solvent began to boil, followed by argon fill, 3 cycles. The solution was irradiated in a 120° C. microwave for 20 minutes. After cooling to room temperature, the mixture was partitioned between ethyl acetate (30 mL) and deionized water (20 mL). The organic layer was washed with half-saturated aqueous sodium chloride solution (10 mL), dried over magnesium sulfate, filtered, concentrated, and purified by silica gel chromatography (eluting with a gradient of 0-100% ethyl acetate in heptanes), to give methyl 3-(1,4-dimethyl-1H-pyrazol-5-yl)-2-methyl-5-(2-(methylthio)pyrimidin-5-yl)benzoate (377.1 mg, 45% yield). ¹H NMR (400 MHz, DMSO-d₆) δ 9.05 (s, 2H), 8.21 (d, J=2.02 Hz, 1H), 7.86 (d, J=2.02 Hz, 1H), 7.38 (s, 1H), 3.90 (s, 3H), 3.55 (s, 3H), 2.56 (s, 3H), 2.22 (s, 3H), 1.83 (s, 3H); MS: 369 [M+1].

To a room-temperature solution of methyl 3-(1,4-dimethyl-1H-pyrazol-5-yl)-2-methyl-5-(2-(methylthio)pyrimidin-5-yl)benzoate (377.1 mg, 1.023 mmol) in tetrahydrofuran (5.0 mL) and deionized water (5.0 mL) was added potassium peroxymonosulfate (939.4 mg, 1.528 mmol). After stirring for 25 hours, the mixture was extracted with ethyl acetate (2×20 mL). The combined organic extracts were dried over magnesium sulfate, filtered, and concentrated under vacuum to give crude methyl 3-(1,4-dimethyl-1H-pyrazol-5-yl)-2-methyl-5-(2-(methylsulfonyl)-pyrimidin-5-yl)benzoate (318.2 mg, 78% yield) as a white foam. ¹H NMR (400 MHz, chloroform-d) δ 9.14 (s, 2H), 8.23 (d, J=2.08 Hz, 1H), 7.60 (d, J=2.08 Hz, 1H), 7.46 (s, 1H), 4.00 (s, 3H), 3.64 (s, 3H), 3.41 (s, 3H), 2.40 (s, 3H), 1.92 (s, 3H); MS: 401 [M+1].

Triethylamine (0.50 mL, 3.59 mmol) was added to a suspension of methyl 3-(1,4-dimethyl-1H-pyrazol-5-yl)-2-methyl-5-(2-(methylsulfonyl)pyrimidin-5-yl)benzoate (115.7 mg, 0.289 mmol) and (1S,4S)-2-oxa-5-aza-bicyclo[2.2.1]heptane hydrochloride (120.7 mg, 0.89 mmol) in dimethylsulfoxide (1.50 mL) in a septum-sealed microwave vial. The mixture was sonicated until homogeneous (˜5 minutes), then irradiated in a 150° C. microwave for 30 minutes. After cooling to room temperature, the mixture was partitioned between ethyl acetate (20 mL) and deionized water (5 mL). The organic layer was washed with saturated aqueous sodium chloride solution (5 mL), dried over magnesium sulfate, filtered, and concentrated to dryness. The residue was purified by silica gel chromatography, eluting with a gradient of 0-100% ethyl acetate in heptane, then 0-20%[EtOH+5% NH₄OH] in ethyl acetate, affording methyl 5-(2-((1S,4S)-2-oxa-5-azabicyclo[2.2.1]heptan-5-yl)pyrimidin-5-yl)-3-(1,4-dimethyl-1H-pyrazol-5-yl)-2-methylbenzoate (60.5 mg, 50% yield) as a colorless glass. ¹H NMR (400 MHz, chloroform-d) δ 8.56 (s, 2H), 8.06 (d, J=1.96 Hz, 1H), 7.41-7.44 (m, J=2.20 Hz, 2H), 5.09 (s, 1H), 4.74 (s, 1H), 3.96 (s, 3H), 3.89-3.95 (m, 2H), 3.57-3.69 (m, 5H), 2.32 (s, 3H), 1.99-2.02 (m, 2H), 1.90 (s, 3H); MS: 420 [M+1].

A solution of methyl 5-(2-((1S,4S)-2-oxa-5-azabicyclo[2.2.1]heptan-5-yl)pyrimidin-5-yl)-3-(1,4-dimethyl-1H-pyrazol-5-yl)-2-methylbenzoate (60.5 mg, 0.144 mmol) in methanol (1.44 mL) was stirred with 4.0 M aqueous sodium hydroxide solution (0.36 mL, 1.44 mmol) at room temperature for 19.5 hours. The methanol was removed under vacuum, and the aqueous residue acidified to pH <2 with hydrochloric acid (1.0 N). The acidic solution was lyophilized to give a mixture of 5-(2-((1S,4S)-2-oxa-5-azabicyclo[2.2.1]heptan-5-yl)pyrimidin-5-yl)-3-(1,4-dimethyl-1H-pyrazol-5-yl)-2-methylbenzoic acid and sodium chloride as a pale yellow solid. MS: 406 [M+1].

The acid/salt mixture was taken up in N,N-dimethylformamide (4.0 mL) and sonicated to break up large chunks of solid. The resulting suspension was passed through a 0.2 micron syringe filter to remove inorganic salts. To the filtered solution was added 3-aminomethyl-4,6-dimethyl-1H-pyridin-2-one hydrochloride (28.4 mg, 0.159 mmol), triethylamine (0.10 mL, 0.72 mmol), and O-(7-azabenzotriazol-1-yl)-N,N,N′,N′-tetramethyluronium hexafluorophosphate (HATU, 77.6 mg, 0.204 mmol), and the mixture stirred at room temperature for 19 hours. The reaction solution was diluted with deionized water (5 mL) and extracted with ethyl acetate (3×10 mL). The combined organic extracts were dried over magnesium sulfate, filtered, concentrated, and purified by preparative SFC to give the title compound (20.5 mg, 26% yield) as a white solid. ¹H NMR (400 MHz, DMSO-d₆) δ 11.46 (br. s., 1H), 8.73 (s, 2H), 8.34 (t, J=4.93 Hz, 1H), 7.60 (d, J=1.77 Hz, 1H), 7.49 (d, J=1.77 Hz, 1H), 7.34 (s, 1H), 5.86 (s, 1H), 4.97 (s, 1H), 4.68 (s, 1H), 4.31 (d, J=5.05 Hz, 2H), 3.81 (d, J=6.32 Hz, 1H), 3.67 (d, J=7.33 Hz, 1H), 3.53 (s, 3H), 3.50 (s, 1H), 3.40-3.46 (m, 1H), 2.22 (s, 3H), 2.11 (s, 3H), 2.00 (s, 3H), 1.91-1.96 (m, 1H), 1.85-1.90 (m, 1H), 1.82 (s, 3H); MS: 540 [M+1].

### Example 23

### N-[(4,6-dimethyl-2-oxo-1,2-dihydropyridin-3-yl)methyl]-3-(1,4-dimethyl-1H-pyrazol-5-yl)-2-methyl-5-{2-[3-oxa-8-azabicyclo[3.2.1]oct-8-yl]pyrimidin-5-yl}benzamide

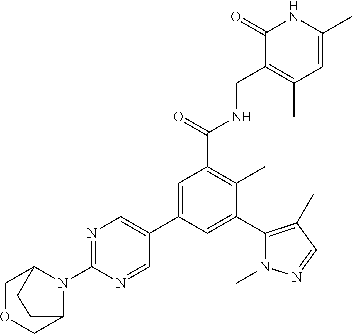 [SMILES: Cc1cc(C)c(CNC(=O)c2cc(-c3cnc(N4C5CCC4COC5)nc3)cc(-c3c(C)cnn3C)c2C)c(=O)[nH]1]

The compound of Example 23 was made by the same method as Example 22, using 3-oxa-8-aza-bicyclo[3.2.1]octane hydrochloride in the reaction with sulfone intermediate, methyl 3-(1,4-dimethyl-1H-pyrazol-5-yl)-2-methyl-5-(2-(methylsulfonyl)pyrimidin-5-yl)benzoate, and following the same procedure for amide formation. ¹H NMR (400 MHz, DMSO-d₆) δ 11.48 (br. s., 1H), 8.77 (s, 2H), 8.35 (t, J=4.93 Hz, 1H), 7.62 (d, J=1.77 Hz, 1H), 7.51 (d, J=1.77 Hz, 1H), 7.34 (s, 1H), 5.87 (s, 1H), 4.64 (br. s., 2H), 4.31 (d, J=5.05 Hz, 2H), 3.57-3.65 (m, 4H), 3.53 (s, 3H), 2.22 (s, 3H), 2.11 (s, 3H), 2.01 (s, 3H), 1.87-2.00 (m, 4H), 1.82 (s, 3H); MS: 554 [M+1].

### Example 24

### N-[(4,6-dimethyl-2-oxo-1,2-dihydropyridin-3-yl)methyl]-3-(1,4-dimethyl-1H-pyrazol-5-yl)-5-[2-(3-fluoroazetidin-1-yl)pyrimidin-5-yl]-2-methylbenzamide

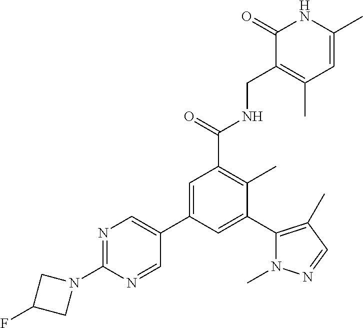 [SMILES: Cc1cc(C)c(CNC(=O)c2cc(-c3cnc(N4CC(F)C4)nc3)cc(-c3c(C)cnn3C)c2C)c(=O)[nH]1]

The compound of Example 24 was made by the same method as Example 22, using 3-fluoro-azetidine hydrochloride in the reaction with sulfone intermediate, methyl 3-(1,4-dimethyl-1H-pyrazol-5-yl)-2-methyl-5-(2-(methylsulfonyl)pyrimidin-5-yl)benzoate, and following the same procedure for amide formation. ¹H NMR (400 MHz, DMSO-d₆) δ 11.44 (br. s., 1H), 8.77 (s, 2H), 8.36 (t, J=4.65 Hz, 1H), 7.62 (d, J=1.96 Hz, 1H), 7.51 (d, J=1.96 Hz, 1H), 7.34 (s, 1H), 5.86 (s, 1H), 5.52 (sptd, J=3.06, 57.34 Hz, 1H), 4.42 (dddd, J=1.34, 5.87, 10.88, 21.52 Hz, 2H), 4.31 (d, J=5.01 Hz, 2H), 4.13 (dddd, J=1.47, 2.81, 10.64, 24.58 Hz, 2H), 3.53 (s, 3H), 2.22 (s, 3H), 2.11 (s, 3H), 2.00 (s, 3H), 1.82 (s, 3H); MS: 516 [M+1].

### Example 25

### N-[(4,6-dimethyl-2-oxo-1,2-dihydropyridin-3-yl)methyl]-3-(1,4-dimethyl-1H-pyrazol-5-yl)-5-(2-methoxypyrimidin-5-yl)-2-methylbenzamide

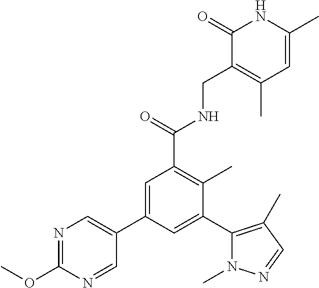

The compound of Example 25 was made by the same method as Example 22, using sodium methoxide in the reaction with sulfone intermediate, methyl 3-(1,4-dimethyl-1H-pyrazol-5-yl)-2-methyl-5-(2-(methylsulfonyl)pyrimidin-5-yl)benzoate, and following the same procedure for amide formation. ¹H NMR (400 MHz, DMSO-d₆) δ 11.49 (br. s., 1H), 8.99 (s, 2H), 8.32-8.47 (m, 1H), 7.69-7.78 (m, 1H), 7.61 (d, J=1.96 Hz, 1H), 7.35 (s, 1H), 5.87 (s, 1H), 4.32 (d, J=5.1 Hz, 2H), 3.96 (s, 3H), 3.53 (s, 3H), 2.22 (s, 3H), 2.11 (s, 3H), 2.03 (s, 3H), 1.77-1.88 (m, 3H), 1.83 (s, 3H); MS: 473 [M+1].

### Method E

### Example 26

### 2-chloro-N-[(4,6-dimethyl-2-oxo-1,2-dihydropyridin-3-yl)methyl]-3-isopropoxy-5-(1-methyl-1H-pyrazol-4-yl)benzamide

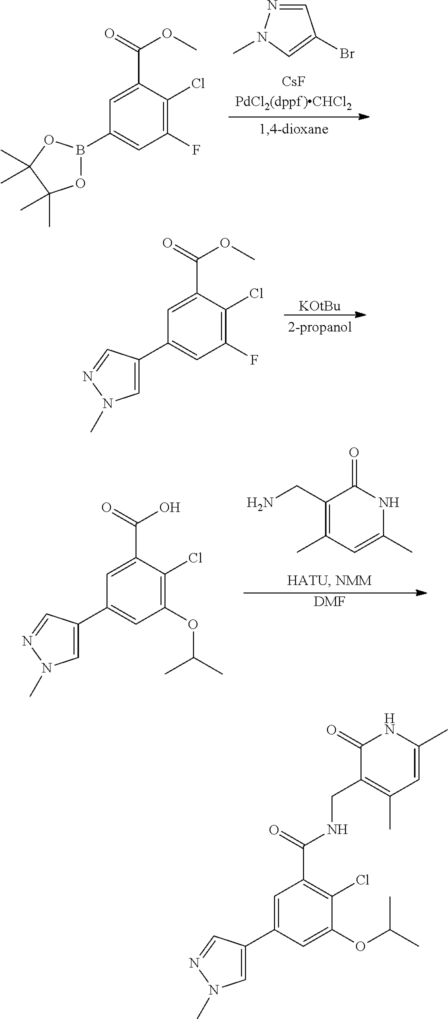

A solution of 4-bromo-1-methyl-1H-pyrazole (563 mg, 3.5 mmol), 2-chloro-3-fluoro-5-(4,4,5,5-tetramethyl-1,3,2-dioxaborolan-2-yl)benzoate (made by a method analogous to that used to make 2-chloro-5-(4,4,5,5-tetramethyl-1,3,2-dioxaborolan-2-yl)-benzoic acid methyl ester in Dombroski, et al., WO2004/99146) (1 g, 3.17 mmol), cesium fluoride (1.44 g, 9.54 mmol) and dichloro-1,1′-bis(diphenylphosphino)ferrocene palladium(II)-dichloromethane complex (130 mg, 0.1595 mmol) in 1,4-dioxane was degassed with nitrogen and heated at 100° C. in a sealed vial in a heating block for 17 hours. The reaction was diluted with water and extracted with ethyl acetate (3×50 mL); the organics were dried over sodium sulfate, evaporated to dryness and purified by silica gel chromatography, eluting with a gradient of 50-100% ethyl acetate in heptanes. The fractions containing the product were combined and evaporated to give methyl 2-chloro-3-fluoro-5-(1-methyl-1H-pyrazol-4-yl)benzoate (162 mg, 19% yield) as a yellow solid. ¹H NMR (400 MHz, acetone-d₆): δ 8.16 (s, 1H), 7.92 (s, 1H), 7.83 (s, 1H), 7.59-7.74 (m, 1H), 3.84-3.96 (m, 6H). MS: 269/270 [M+1], Cl isotope pattern.

To a suspension of methyl 2-chloro-3-fluoro-5-(1-methyl-1H-pyrazol-4-yl)benzoate (162 mg, 0.603 mmol) in 2-propanol (10 mL) was added potassium tert-butoxide (609 mg, 5.43 mmol); and the suspension sonicated until the solids were completely dissolved. The reaction was heated at 150° C. in a microwave for 3 hours. The volatiles were removed, the residue was dissolved in water, and the solution was extracted with ethyl acetate. The aqueous layer was acidified with 3N HCl to pH=3 and extracted with ethyl acetate (3×20 mL). The organics were dried over anhydrous sodium sulfate and evaporated to give 2-chloro-3-isopropoxy-5-(1-methyl-1H-pyrazol-4-yl)benzoic acid (80 mg, 45% yield) as a white solid. ¹H NMR (400 MHz, methanol-d): δ 7.96 (s, 1H), 7.79 (s, 1H), 7.39 (d, J=1.8 Hz, 1H), 7.27 (d, J=1.8 Hz, 1H), 4.68 (dt, J=12.1, 6.0 Hz, 1H), 3.85 (s, 3H), 1.31 (d, J=6.1 Hz, 6H). MS: 295/298 [M+1], Cl isotope pattern.

To a solution of 2-chloro-3-isopropoxy-5-(1-methyl-1H-pyrazol-4-yl)benzoic acid (40 mg, 0.14 mmol) in N,N-dimethylformamide (2 mL), O-(7-azabenzotriazol-1-yl)-N,N,N′,N′-tetramethyluronium hexafluorophosphate (HATU, 52 mg, 0.136 mmol) and N-methylmorpholine (15 μL, 0.136 mmol) were added. The solution was stirred at room temperature for 1 hour, then 3-(aminomethyl)-4,6-dimethyl-1H-pyridin-2-one hydrochloride (27 mg, 0.143 mmol) and N-methylmorpholine (30 μL, 0.27 mmol) were added. The reaction was stirred at room temperature for 16 hours, then diluted with water and the resulting solids were collected by filtration. The precipitate was washed with water, re-crystallized in acetone/water, transferred to a vial with acetone/water, and lyophilized to give the title compound (18 mg, 31% yield) as a white solid. ¹H NMR (400 MHz, acetonitrile-d₃): δ 7.85 (s, 1H), 7.75 (s, 1H), 7.20 (d, J=2.0 Hz, 1H), 7.12 (d, J=1.8 Hz, 1H), 7.08 (br. s., 1H), 5.87 (s, 1H), 4.62-4.77 (m, 1H), 4.39 (d, J=5.6 Hz, 2H), 3.86 (s, 3H), 2.28 (s, 3H), 2.16 (s, 3H), 1.34 (d, J=6.1 Hz, 6H). MS: 429/432 [M+1], Cl isotope pattern.

### Example 27

### 2-chloro-3-isopropoxy-N-[(6-methyl-2-oxo-4-propyl-1,2-dihydropyridin-3-yl)methyl]-5-(1-methyl-1H-pyrazol-4-yl)benzamide

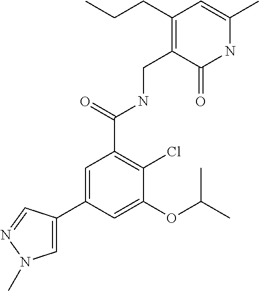 [SMILES: CCCc1cc(C)[nH]c(=O)c1CNC(=O)c1cc(-c2cnn(C)c2)cc(OC(C)C)c1Cl]

The compound of Example 27 was made by the same method as Example 26, using 3-(aminomethyl)-6-methyl-4-propylpyridin-2(1H)-one in the final amide coupling reaction. ¹H NMR (400 MHz, methanol-d₄): δ 8.04 (s, 1H), 7.87 (s, 1H), 7.30 (d, J=2.0 Hz, 1H), 7.21 (d, J=2.0 Hz, 1H), 6.16 (s, 1H), 4.76 (dt, J=12.1, 6.0 Hz, 2H), 4.52 (s, 2H), 3.95 (s, 3H), 2.65-2.80 (m, 2H), 2.29 (s, 3H), 1.92 (s, 1H), 1.60-1.76 (m, 2H), 1.39 (d, J=6.1 Hz, 6H), 1.07 (t, J=7.3 Hz, 3H). MS: 457/458 [M+1], Cl isotope pattern.

### Method F

### Example 28

### 5-chloro-3-(2,5-dimethyl-1H-imidazol-1-yl)-N-[(4,6-dimethyl-2-oxo-1,2-dihydropyridin-3-yl)methyl]-2-methylbenzamide

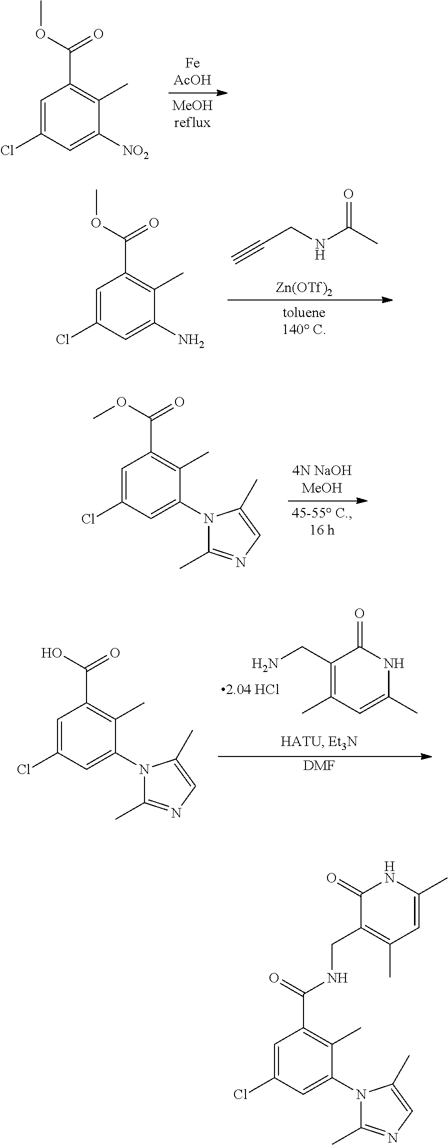

To a suspension of methyl 5-chloro-2-methyl-3-nitrobenzoate (11.4 g, 49.6 mmol) in methanol (50 mL) and glacial acetic acid (50 mL) was added iron metal (10.5 g, 188 mmol) in one portion. A large exotherm brought the methanol to rapid reflux. The reaction was refluxed for an additional 2 hours. The reaction mixture was decanted off of the iron solids and the volatiles were removed under vacuum. The resulting thick suspension was taken up in dichloromethane and shaken with saturated aqueous sodium bicarbonate. The resulting suspension was filtered through a glass frit to remove precipitates, then the layers were separated and the aqueous phase was further extracted with dichloromethane (3×). The combined dichloromethane layers were again filtered, washed with saturated aqueous sodium chloride, dried over sodium sulfate, filtered, and concentrated to give a dark oil. Purification on silica gel gave methyl 3-amino-5-chloro-2-methylbenzoate (7.08 g, 71% yield) as a golden oil. ¹H NMR (400 MHz, DMSO-d₆) δ 6.81-6.84 (m, 2H), 5.44 (s, 2H), 3.79 (s, 3H), 2.13 (s, 3H).

A mixture of methyl 3-amino-5-chloro-2-methylbenzoate (0.385 g, 1.93 mmol), N-(prop-2-yn-1-yl)acetamide (0.250 g, 2.57 mmol), and zinc trifluoromethanesulfonate (0.35 g, 0.096 mmol) in dry toluene (3 mL) was irradiated in a microwave reactor for 1 hour at 140° C. After removal of the solvent, the crude imidazole compound was purified by silica gel chromatography, affording methyl 5-chloro-3-(2,5-dimethyl-1H-imidazol-1-yl)-2-methylbenzoate (128 mg, 36% yield) as a yellow oil. This oil was dissolved in methanol (3 mL), treated with 4N aqueous sodium hydroxide (0.20 mL, 0.80 mmol), and stirred at 55° C. for 3 hours, then at 45° C. overnight. The reaction was cooled to room temperature, neutralized with 6N hydrochloric acid, and concentrated to an oily residue. This residue was dissolved in N,N-dimethylformamide (2.0 mL) and treated with triethylamine (0.20 mL, 1.44 mmol) and O-(7-azabenzotriazol-1-yl)-N,N,N′,N′-tetramethyluronium hexafluorophosphate (HATU, 185 mg, 0.47 mmol). After 5 minutes, 3-aminomethyl-4,6-dimethyl-1H-pyridin-2-one hydrochloride (105 mg, 0.463 mmol) was added and the reaction stirred at room temperature for 30 minutes. The mixture was diluted with water (7 mL), and extracted with ethyl acetate. The organic layer was concentrated and the resulting residue was purified by preparative HPLC to give the acetate salt (1.5 eq.) of the title compound (65.4 mg, 29% yield) as a white powder. ¹H NMR (400 MHz, DMSO-d₆) δ 8.49 (t, J=4.95 Hz, 1H), 7.48 (d, J=2.20 Hz, 1H), 7.44 (d, J=2.20 Hz, 1H), 6.66 (d, J=1.10 Hz, 1H), 5.86 (s, 1H), 4.27 (d, J=4.89 Hz, 2H), 2.20 (s, 3H), 2.11 (s, 3H), 1.98 (s, 3H), 1.84 (s, 3H), 1.77 (s, 3H); MS: 399 [M+1].

### Example 29

### 5-chloro-N-[(4,6-dimethyl-2-oxo-1,2-dihydropyridin-3-yl)methyl]-2-methyl-3-(2-methyl-1H-imidazol-1-yl)benzamide

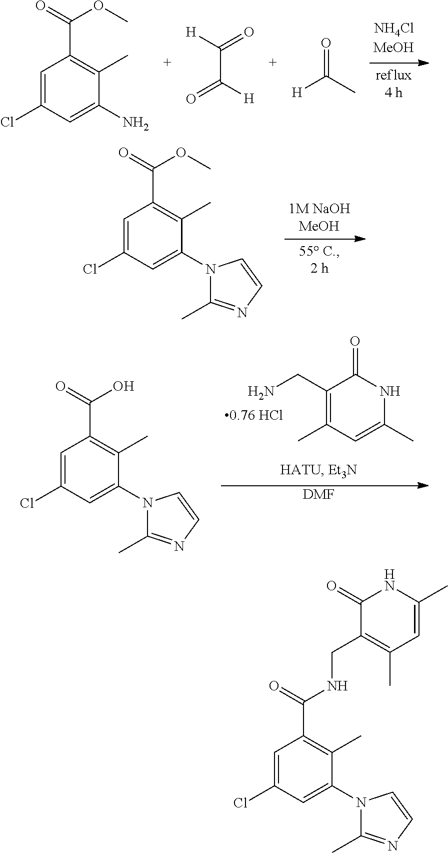

A solution of methyl 3-amino-5-chloro-2-methylbenzoate (0.200 g, 1.00 mmol) in methanol (2 mL) was treated with ammonium acetate (0.154 g, 2.00 mmol), ethanedial (0.229 mL, 2.00 mmol), and then acetaldehyde (0.112 mL, 1.99 mmol). The reaction was heated at reflux for 3 hours. The volatiles were removed under vacuum and the resulting residue was dissolved in dichloromethane and purified on silica gel (Biotage Flash 25S, eluting with a gradient of 0-15% [7N NH₃ in MeOH] in ethyl acetate) to give methyl 5-chloro-2-methyl-3-(2-methyl-1H-imidazol-1-yl)benzoate (0.0399 g, 15% yield) as a brown, sticky solid. ¹H NMR (400 MHz, DMSO-d₆) δ 7.93 (d, J=2.20 Hz, 1H), 7.76 (d, J=2.20 Hz, 1H), 7.20 (d, J=0.98 Hz, 1H), 6.95 (d, J=0.98 Hz, 1H), 3.87 (s, 3H), 2.07 (s, 3H), 2.04 (s, 3H); MS: 265 [M+1].

To a solution of methyl 5-chloro-2-methyl-3-(2-methyl-1H-imidazol-1-yl)benzoate (0.038 g, 0.144 mmol) in methanol (3 mL) was added 1M aqueous sodium hydroxide (0.30 mL, 0.30 mmol) and the reaction was heated at 55° C. for 2 hours. After cooling to room temperature, the mixture was neutralized with 1N HCl (300 μL). The solvents were removed under vacuum to give a brown gum, which contained crude 5-chloro-2-methyl-3-(2-methyl-1H-imidazol-1-yl)benzoic acid. MS: 251 [M+1]. To a solution of this material in N,N-dimethylformamide (1.5 mL) was added triethylamine (0.10 mL, 0.717 mmol) and then O-(7-azabenzotriazol-1-yl)-N,N,N′,N′-tetramethyluronium hexafluorophosphate (HATU, 93 mg, 0.24 mmol). After 5 minutes, 3-aminomethyl-4,6-dimethyl-1H-pyridin-2-one hydrochloride (47 mg, 0.26 mmol) was added and the reaction was stirred at room temperature for 30 minutes. The reaction mixture was made homogeneous with water (1 mL) and purified by reverse phase HPLC to give the title compound (16 mg, 19% yield). ¹H NMR (400 MHz, DMSO-d₆) δ 11.41 (br. s., 1H), 8.38 (t, J=5.01 Hz, 1H), 7.44 (d, J=2.20 Hz, 1H), 7.36 (d, J=2.20 Hz, 1H), 7.06 (d, J=1.22 Hz, 1H), 6.86 (d, J=1.22 Hz, 1H), 5.80 (s, 1H), 4.20 (d, J=4.89 Hz, 2H), 2.13 (s, 3H), 2.04 (s, 3H), 2.00 (s, 3H), 1.79 (s, 3H); MS: 385 [M+1].

### Method G

### Example 30

### 5-cyano-N-[(4,6-dimethyl-2-oxo-1,2-dihydropyridin-3-yl)methyl]-3-(1,4-dimethyl-1H-pyrazol-5-yl)-2-methylbenzamide

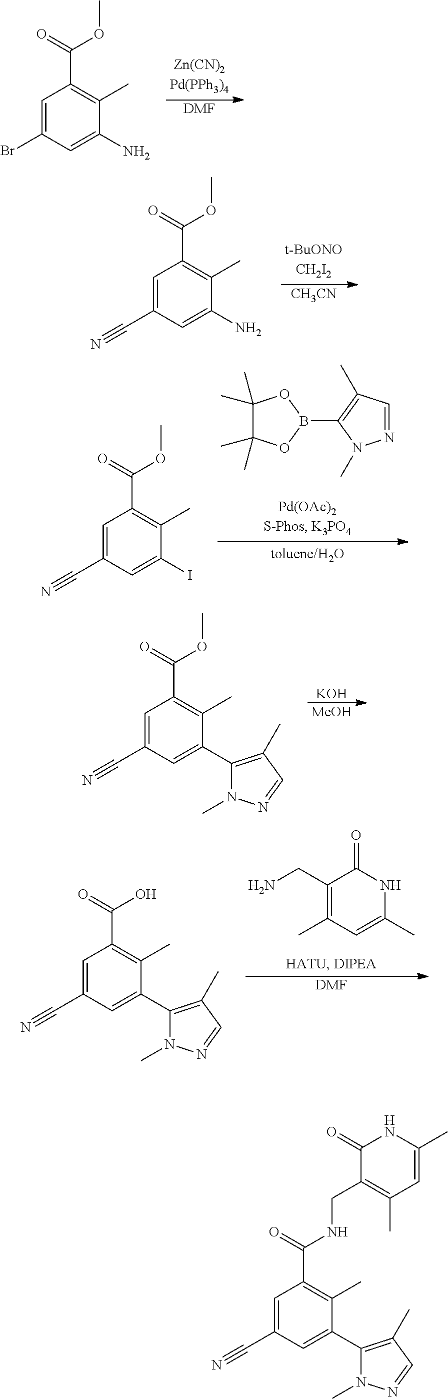

Zinc cyanide (960 mg, 8.2 mmol) and tetrakis(triphenylphosphine) palladium(0) (400 mg, 0.41 mmol) were added to a solution of methyl 3-amino-5-bromo-2-methylbenzoate (2.0 g, 8.2 mmol) in N,N-dimethylformamide (30 mL) under a nitrogen atmosphere. The mixture was stirred at 120° C. overnight, then diluted with water (30 mL) and extracted with ethyl acetate (3×30 mL). The combined organic extracts were washed with water (3×30 mL), followed by saturated aqueous sodium chloride solution (30 mL); then dried over sodium sulfate, concentrated, and purified by silica gel chromatography (eluting with 10% ethyl acetate in petroleum ether) to give methyl 3-amino-5-cyano-2-methylbenzoate (1.1 g, 71% yield) as a white solid.

Diiodomethane (3.06 g, 11.68 mmol) was added dropwise to a solution of methyl 3-amino-5-cyano-2-methylbenzoate (1.1 g, 5.84 mmol) and tert-butyl nitrite (1.34 g, 11.68 mmol) in acetonitrile (40 mL). After the addition was complete, the solution was stirred at 80° C. for 4 hours. The mixture was concentrated under vacuum and purified by silica gel chromatography (eluting with 10% ethyl acetate in petroleum ether), affording methyl 5-cyano-3-iodo-2-methylbenzoate (510 mg, 30% yield) as a white solid.

A solution of methyl 5-cyano-3-iodo-2-methylbenzoate (620 mg, 2.07 mmol) in toluene (10 mL) and water (1.0 mL) was treated with (1,4-dimethyl-1H-pyrazol-5-yl)boronic acid pinacol ester (688 mg, 3.1 mmol), palladium(II) acetate (100 mg, 2.07 mmol), 2-dicyclohexylphosphino-2′,6′-dimethoxybiphenyl (SPhos, 150 mg, 4.13 mmol), and potassium phosphate (876 mg, 4.13 mmol), and the mixture heated at 100° C. overnight. After cooling to room temperature, the solution was diluted with water (20 mL) and extracted with ethyl acetate (3×20 mL). The combined organic extracts were washed with saturated aqueous sodium chloride solution, dried over sodium sulfate, concentrated, and purified by silica gel chromatography (eluting with 10% ethyl acetate in petroleum ether), to give methyl 5-cyano-3-(1,4-dimethyl-1H-pyrazol-5-yl)-2-methylbenzoate (180 mg, 33% yield).

Potassium hydroxide (75 mg, 1.34 mmol) was added to a solution of methyl 5-cyano-3-(1,4-dimethyl-1H-pyrazol-5-yl)-2-methylbenzoate (180 mg, 0.67 mmol) in methanol (10 mL). The mixture was stirred at room temperature for 3 hours, then acidified to pH ˜5 with 1M aqueous hydrochloric acid. The organic solvents were evaporated, and the residue extracted with ethyl acetate (2×30 mL). The combined organic layers were washed with aqueous sodium carbonate solution (20 mL). The resulting basic aqueous layer was extracted with ethyl acetate (30 mL), then re-acidified to pH ˜5 with 1M aqueous hydrochloric acid, and extracted with ethyl acetate (30 mL). The combined organic layers were concentrated under vacuum to give 5-cyano-3-(1,4-dimethyl-1H-pyrazol-5-yl)-2-methylbenzoic acid (120 mg, 75% yield) as a brown oil.

A solution of 5-cyano-3-(1,4-dimethyl-1H-pyrazol-5-yl)-2-methylbenzoic acid (120 mg, 0.47 mmol), 3-aminomethyl-4,6-dimethyl-1H-pyridin-2-one hydrochloride (92 mg, 0.47 mmol), and N,N-diisopropylethylamine (139 mg, 1.08 mmol) in N,N-dimethylformamide (10 mL) was cooled to 0° C., then O-(7-azabenzotriazol-1-yl)-N,N,N′,N′-tetramethyluronium hexafluoro-phosphate (HATU, 253 mg, 0.71 mmol) was added. The mixture was allowed to stir at room temperature overnight, then it was diluted with water (30 mL) and extracted with ethyl acetate (3×30 mL). The combined organic layers were washed with water (2×50 mL), and saturated aqueous sodium chloride solution (50 mL), then concentrated under vacuum and purified by preparative HPLC to give the title compound (51.8 mg, 27% yield) as a white solid. ¹H NMR (400 MHz, methanol-d₄) δ 7.84 (s, 1H), 7.69 (s, 1H), 7.43 (s, 1H), 6.14 (s, 1H), 4.51 (s, 2H), 3.58 (s, 3H), 2.40 (s, 3H), 2.26 (s, 3H), 2.15 (s, 3H), 1.88 (s, 3H). MS: 390 [M+1].

### Method H

### Example 31

### (S)—N-((4,6-dimethyl-2-oxo-1,2-dihydropyridin-3-yl)methyl)-5-methyl-6-(2-methyl-4-(pyrimidin-2-yl)piperazin-1-yl)pyrimidine-4-carboxamide

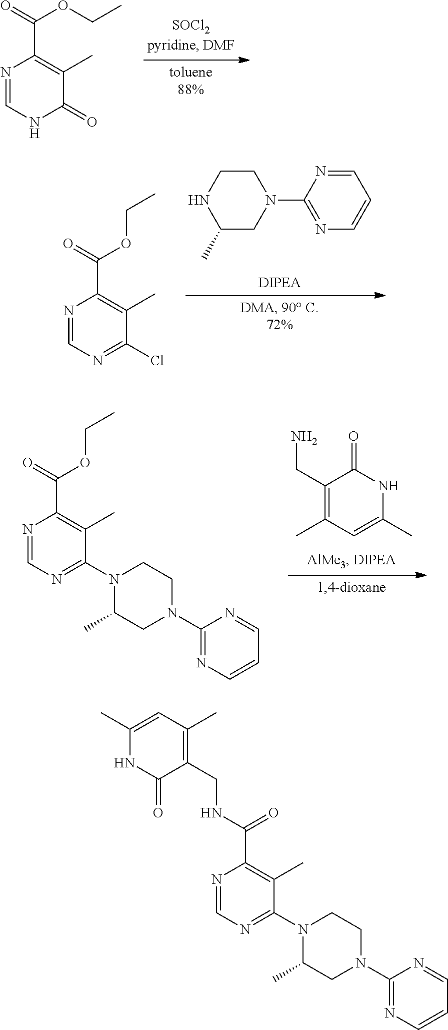

A suspension of ethyl 5-methyl-6-oxo-1,6-dihydropyrimidine-4-carboxylate (378 mg, 2.18 mmol), thionyl chloride (1.96 mL, 26.9 mmol), N,N-dimethylformamide (1.38 μL, 0.178 mmol) and pyridine (34.4 μL, 0.425 mmol) in toluene (2.5 mL) was heated at 90° C. for 1 hour. The mixture was concentrated, diluted with saturated aqueous sodium bicarbonate, and extracted with dichloromethane (2×10 mL). The combined organic extracts were dried (anhydrous magnesium sulfate), filtered, and concentrated to give ethyl 6-chloro-5-methylpyrimidine-4-carboxylate (333 mg, 80% yield). ¹H NMR (400 MHz, methanol-d₄) δ 8.86 (s, 1H), 4.46 (q, J=7.24 Hz, 2H), 2.51 (s, 3H), 1.41 (t, J=7.07 Hz, 3H).

To a solution of ethyl 6-chloro-5-methylpyrimidine-4-carboxylate (75 mg, 0.37 mmol) in N,N-dimethylacetamide (0.5 mL) was added (S)-2-(3-methylpiperazin-1-yl)pyrimidine (67 mg, 0.37 mmol) and N,N-diisopropylethylamine (0.20 mL, 1.1 mmol). The reaction mixture was stirred at 90° C. for 18 hours and at 95° C. for 4 hours, then diluted with water and extracted with methyl tert-butyl ether (2×5 mL). The combined organic extracts were dried (anhydrous magnesium sulfate), filtered, and concentrated to give (S)-ethyl 5-methyl-6-(2-methyl-4-(pyrimidin-2-yl)piperazin-1-yl)pyrimidine-4-carboxylate (92 mg, 72% yield). ¹H NMR (400 MHz, DMSO-d₆) δ 8.58 (1H, s), 8.38 (1H, s), 8.37 (1H, s), 6.64 (1H, t, J=4.80 Hz), 4.54 (1H, d, J=12.38 Hz), 4.39-4.47 (1H, m), 4.35 (2H, q, J=7.16 Hz), 4.21-4.32 (1H, m), 3.73 (1H, d, J=13.39 Hz), 3.34-3.44 (1H, m), 3.19 (1H, td, J=12.25, 3.28 Hz), 2.24 (3H, s), 1.31 (3H, t, J=7.07 Hz), 1.13 (3H, d, J=6.57 Hz); MS: 343 [M+1].

To a white suspension of 3-aminomethyl-4,6-dimethyl-1H-pyridin-2-one hydrochloride (73 mg, 0.32 mmol) in 1,4-dioxane (1 mL) was added N,N-diisopropylethylamine (0.11 mL, 0.65 mmol) followed by trimethylaluminum (2M in heptane, 0.32 mL, 0.65 mmol). The reaction mixture was stirred at room temperature for 30 minutes and became homogenous, then a solution of (S)-ethyl 5-methyl-6-(2-methyl-4-(pyrimidin-2-yl)piperazin-1-yl)pyrimidine-4-carboxylate (92 mg, 0.27 mmol) in 1,4-dioxane (1 mL) was added drop-wise. The reaction was stirred at room temperature for 18 hours, then diluted with a 2M aqueous solution of potassium sodium tartrate and extracted with ethyl acetate (2×10 mL). The combined organic extracts were washed with saturated aqueous sodium chloride, dried (anhydrous magnesium sulfate), filtered, and concentrated. The residue was purified by reverse phase HPLC to provide the title compound (15.2 mg, 13% yield). ¹H NMR (700 MHz, DMSO-d₆) δ 8.57 (1H, t, J=4.95 Hz), 8.53 (1H, s), 8.37 (1H, s), 8.36 (1H, d, J=0.66 Hz), 6.63 (1H, t, J=4.40 Hz), 5.87 (1H, s), 4.52 (1H, d, J=12.76 Hz), 4.37 (1H, d, J=12.98 Hz), 4.25-4.31 (2H, m), 4.16-4.23 (1H, m), 3.62 (1H, d, J=13.42 Hz), 3.16-3.23 (1H, m), 2.31 (3H, s), 2.21 (3H, s), 2.11 (3H, s), 1.10 (3H, d, J=6.38 Hz); MS: 449 [M+1].

### Method I

### Example 32

### N-((4,6-dimethyl-2-oxo-1,2-dihydropyridin-3-yl)methyl)-3-isopropoxy-2-methylbenzamide

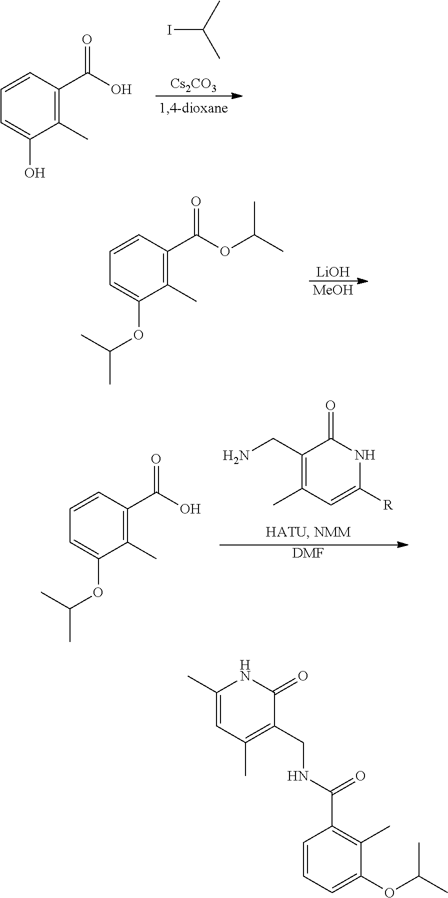

Cesium carbonate (4.71 g, 14.5 mmol) was added to a solution of 3-hydroxy-2-methylbenzoic acid (1 g, 6.573 mmol) in 1,4-dioxane (50 mL). The reaction was stirred at room temperature for 15 minutes, then isopropyl iodide (2.1 mL, 21 mmol) was added portion wise. The resulting mixture was stirred at room temperature for 16 hours. The reaction was diluted with water and extracted with ethyl acetate (3×50 mL), the combined organic layers were washed with water (3×50 mL) and brine (2×50 mL), dried over anhydrous sodium sulfate and evaporated to give isopropyl 3-isopropoxy-2-methylbenzoate (500 mg, 32% yield). ¹H NMR (400 MHz, acetone-d₆): δ 7.30 (d, J=7.6 Hz, 1H), 7.20 (t, J=7.8 Hz, 1H), 7.09-7.16 (m, 1H), 5.18 (ddd, J=12.5, 6.2, 6.1 Hz, 1H), 4.62 (dt, J=11.9, 6.0 Hz, 1H), 2.36 (s, 3H), 1.33 (t, J=6.4 Hz, 12H); GCMS: 236.2 m/z.

To a solution of isopropyl 3-isopropoxy-2-methylbenzoate (500 mg, 2.12 mmol) in methanol, dry lithium hydroxide (507 mg, 21.2 mmol) was added. The reaction was heated at 80° C. in a sealed vial for 8 hours, then the volatiles were removed and the residue was dissolved in water (20 mL) and extracted with ethyl acetate (2×10 mL). The organics extracts were discarded and the aqueous residue was acidified with 3 N HCl (pH=1) and extracted with ethyl acetate (3×50 mL). These extracts were dried over sodium sulfate and evaporated to give 3-isopropoxy-2-methylbenzoic acid (365 mg, 88% yield) as a light yellow oil that crystallized upon standing. ¹H NMR (400 MHz, acetone-d₆): δ 10.97 (br. s., 1H), 7.44 (d, J=7.6 Hz, 1H), 7.18-7.29 (m, 1H), 6.92-7.18 (m, 1H), 4.62 (spt, J=6.0 Hz, 1H), 2.43 (s, 3H), 1.25-1.42 (m, 6H).

To a solution of 3-isopropoxy-2-methylbenzoic acid (100 mg, 0.515 mmol) in N,N-dimethylformamide (6 mL), was added O-(7-azabenzotriazol-1-yl)-N,N,N′,N′-tetramethyluronium hexafluorophosphate (HATU, 196 mg, 0.515 mmol) and N-methylmorpholine (57 μL, 0.515 mmol). The solution was stirred at room temperature for 1 hour, then 3-(aminomethyl)-4,6-dimethyl-1H-pyridin-2-one hydrochloride (97.2 mg, 0.515 mmol) and N-methylmorpholine (114 μL, 1.03 mmol) were added. The reaction was stirred at room temperature for 2 hours, then diluted with water. The resulting precipitate was collected by filtration, washed with water, transferred to a vial with methanol/water, and lyophilized to give the title compound (138 mg, 81% yield) as a white solid. ¹H NMR (400 MHz, methanol-d4): δ 7.10-7.29 (m, 1H), 7.00 (d, J=8.1 Hz, 1H), 6.88 (d, J=7.6 Hz, 1H), 6.13 (s, 1H), 4.61 (dt, J=12.1, 6.1 Hz, 1H), 4.49 (s, 2H), 2.41 (s, 3H), 2.27 (s, 3H), 2.19 (s, 3H), 1.34 (d, J=6.1 Hz, 6H). MS: 329 [M+1].

### Method J

### Example 33

### 3-chloro-N-[(4,6-dimethyl-2-oxo-1,2-dihydropyridin-3-yl)methyl]-2-isopropoxy-6-(1-methyl-1H-pyrazol-4-yl)isonicotinamide

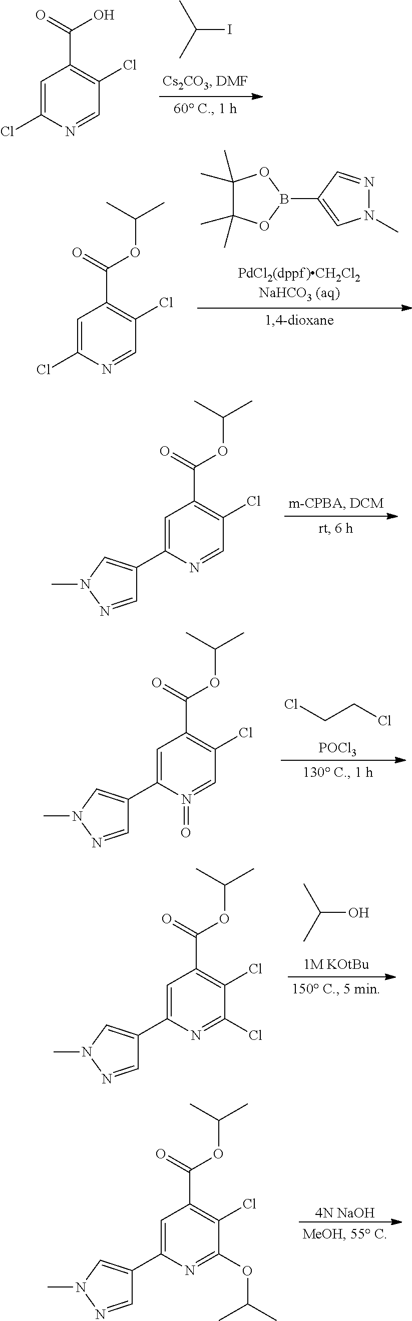

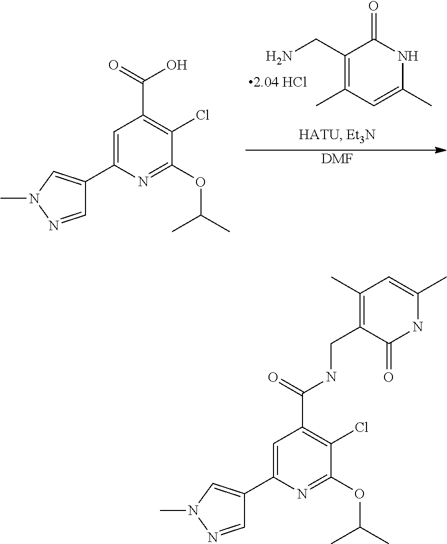

A solution of 2,5-dichloropyridine-4-carboxylic acid (1.36 g, 7.08 mmol) in N,N-dimethylformamide (10 mL) was treated with cesium carbonate (2.45 g, 7.52 mmol) and 2-iodopropane (2.0 mL, 20 mmol) and stirred at 60° C. for 1 hour. The reaction mixture was poured into ethyl acetate and washed with water twice. The ethyl acetate layer was concentrated to give a golden oil containing crude isopropyl 2,5-dichloroisonicotinate. MS: 234/236 [M+1], Cl₂ isotope pattern. To a solution of this oil crude oil in 1,4-dioxane (80 mL) was added 1-methylpyrazole-4-boronic acid pinacol ester (1.96 g, 9.41 mmol), and saturated aqueous sodium bicarbonate (20 mL), and the mixture was degassed by bubbling nitrogen through the solution for 10 minutes. The reaction was treated with dichloro-1,1′-bis(diphenylphosphino)ferrocene palladium (11)-dichloromethane complex [PdCl₂(dppf).CH₂Cl₂] (245 mg, 0.30 mmol), and heated at 100° C. for 1 hour. The reaction mixture was diluted with ethyl acetate and washed with water and saturated aqueous sodium chloride. The ethyl acetate layer was concentrated to give a dark oil which was purified on silica gel (Biotage Flash 40M, 30-80% ethyl acetate gradient in heptanes) to give isopropyl 5-chloro-2-(1-methyl-1H-pyrazol-4-yl)isonicotinate (1.73 g, 87% yield) as a yellow oil. MS: 280 [M+1].

To a solution of isopropyl 5-chloro-2-(1-methyl-1H-pyrazol-4-yl)isonicotinate (701 mg, 2.51 mmol) in dichloromethane (10 mL) was added 3-chloroperbenzoic acid (m-CPBA, 2.1 g, 8.5 mmol) in one portion. The reaction was stirred at room temperature for 6 h. The entire reaction mixture was poured onto silica gel and chromatographed (Biotage Flash 40M, eluting with 70-100% ethyl acetate gradient in heptane) to give 5-chloro-4-(isopropoxycarbonyl)-2-(1-methyl-1H-pyrazol-4-yl)pyridine 1-oxide (0.201 g, 27% yield) as a yellow solid. MS: 296 [M+1].

Phosphorus (V) oxychloride (0.700 mL, 7.51 mmol) was added to a solution of 5-chloro-4-(isopropoxycarbonyl)-2-(1-methyl-1H-pyrazol-4-yl)pyridine 1-oxide (0.280 g, 0.947 mmol) in 1,2-dichloroethane (4 mL), and the mixture irradiated at 130° C. in a microwave reactor for 1 hour. The reaction mixture was diluted with dichloromethane and stirred vigorously with saturated aqueous sodium carbonate solution (25 mL) for 20 minutes. The layers were separated and the dichloromethane layer was concentrated to give crude isopropyl 2,3-dichloro-6-(1-methyl-1H-pyrazol-4-yl)isonicotinate (199 mg, 67% yield). A suspension of crude isopropyl 2,3-dichloro-6-(1-methyl-1H-pyrazol-4-yl)isonicotinate (97 mg, 0.31 mmol) in 2-propanol (3 mL) was treated with potassium t-butoxide (1M in THF, 1.0 mL, 1.0 mmol) and the reaction was heated at 150° C. in a microwave reactor for 5 minutes. The reaction mixture was diluted with water and extracted with ethyl acetate. The ethyl acetate layer was concentrated to give a yellow oil, which was purified by silica gel chromatography (Biotage Flash 25S, 20-80% ethyl acetate gradient in heptane) to give isopropyl 3-chloro-2-isopropoxy-6-(1-methyl-1H-pyrazol-4-yl)isonicotinate (38 mg, 36% yield) as a clear oil. MS: 338 [M+1].

A solution of isopropyl 3-chloro-2-isopropoxy-6-(1-methyl-1H-pyrazol-4-yl)isonicotinate (38 mg, 0.11 mmol) in methanol (4 mL) was treated with 1N aqueous sodium hydroxide solution (0.165 mL, 0.165 mmol) and stirred at 55° C. for 2 hours and then at 35° C. overnight. The reaction was diluted with water (5 mL) and washed with ethyl acetate. The aqueous phase was neutralized with 6N hydrochloric acid, frozen in a dry ice bath, and lyophilized to give a white solid. This solid was dissolved in N,N-dimethylformamide (1.0 mL) and treated with triethylamine (0.040 mL, 0.29 mmol) and O-(7-azabenzotriazol-1-yl)-N,N,N′,N′-tetramethyluronium hexafluorophosphate (HATU, 41 mg, 0.10 mmol). After 5 minutes, 3-aminomethyl-4,6-dimethyl-1H-pyridin-2-one hydrochloride (21 mg, 0.11 mmol) was added in one portion. The reaction was stirred at room temperature for 30 minutes, then diluted with water (6 mL) and was placed in a refrigerator for 1 hour. The resulting precipitate was collected by filtration, washed with water, and dried under vacuum at 80° C. to give the title compound (26 mg, 55% yield) as a tan powder. ¹H NMR (400 MHz, DMSO-d₆) δ 11.46 (s, 1H), 8.50 (t, J=4.93 Hz, 1H), 8.27 (s, 1H), 7.99 (s, 1H), 7.16 (s, 1H), 5.87 (s, 1H), 5.40 (quin, J=6.19 Hz, 1H), 4.28 (d, J=5.05 Hz, 2H), 3.87 (s, 3H), 2.20 (s, 3H), 2.11 (s, 3H), 1.34 (d, J=6.32 Hz, 6H); MS: 430 [M+1].

### Method K

### Example 34

### 2-(6-aminopyridin-3-yl)-N-[(4,6-dimethyl-2-oxo-1,2-dihydropyridin-3-yl)methyl]-5-methyl-6-(1-methyl-1H-pyrazol-5-yl)pyrimidine-4-carboxamide

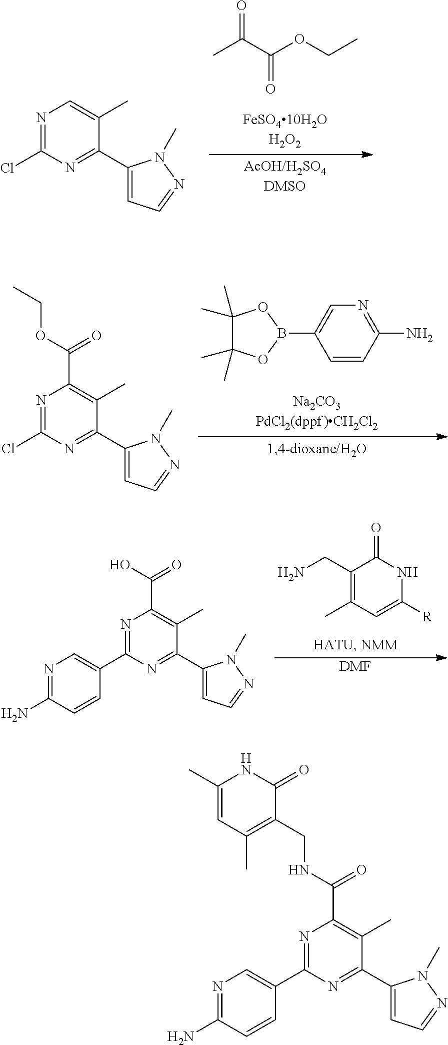

To a solution of 2-chloro-5-methyl-4-(1-methyl-1H-pyrazol-5-yl)pyrimidine (500 mg, 2.4 mmol), ethyl pyruvate (4 mL, 36 mmol), concentrated sulfuric acid (0.4 mL, 7.5 mmol), and iron(II) sulfate heptahydrate (6.66 g, 24 mmol), in water (50 mL), dimethylsulfoxide (50 mL) and acetic acid (6 mL) was added cold hydrogen peroxide (50% aqueous solution) at 1 hour intervals until most of the starting material was consumed (8 additions of 350 μL each). The reaction was exothermic with gas evolution. The reaction was diluted with water and extracted with ethyl acetate (3×20 mL). The organics were combined, dried over anhydrous sodium sulfate and concentrated. The residue was purified by flash chromatography using ethyl acetate/heptane 0-50%. The fractions containing the product plus a co-eluting side product (methylated pyrimidine) were combined and evaporated to give ethyl 2-chloro-5-methyl-6-(1-methyl-1H-pyrazol-5-yl)pyrimidine-4-carboxylate (673 mg, 18% yield, 70% purity) as a yellow solid. ¹H NMR (400 MHz, acetone-d₆): δ 7.51 (d, J=1.8 Hz, 1H), 6.59 (d, J=2.0 Hz, 1H), 4.38-4.57 (m, 2H), 4.03 (s, 3H), 2.40-2.49 (m, 3H), 1.40 (t, J=7.1 Hz, 3H). MS: 281.0/282.0 [M+1].

A solution of ethyl 2-chloro-5-methyl-6-(1-methyl-1H-pyrazol-5-yl)pyrimidine-4-carboxylate (1 g, 4.854 mmol), 5-(4,4,5,5-tetramethyl-1,3,2-dioxaborolan-2-yl)pyridin-2-amine (1.01 g, 4.85 mmol), sodium carbonate (1.54 g, 14.6 mmol) and dichloro-1,1′-bis(diphenylphosphino)ferrocene-palladium(II)-dichloromethane complex (198 mg, 0.243 mmol) in 1,4-dioxane/water 1:1 (30 mL) was degassed with nitrogen and heated at 100° C. in a sealed vial in a microwave for 1 hour. The reaction was diluted with water and extracted with ethyl acetate (10 mL). The organic layer was discarded and the aqueous residue was acidified to pH=3 with citric acid and evaporated. The aqueous residue was cooled in an ice bath and sonicated. The resulting solid was collected by filtration, washed with a minimal amount of cold water, and dried in a vacuum oven to give 2-(6-aminopyridin-3-yl)-5-methyl-6-(1-methyl-1H-pyrazol-5-yl)pyrimidine-4-carboxylic acid (40 mg, 11% yield) as a tan solid. ¹H NMR (400 MHz, DMSO-d₆): δ 8.85 (s, 1H), 8.19 (d, J=8.8 Hz, 1H), 7.51 (d, J=1.8 Hz, 1H), 6.69 (d, J=2.0 Hz, 1H), 6.50 (s, 2H), 6.46 (d, J=8.8 Hz, 1H), 3.92 (s, 3H), 2.26 (s, 3H). MS: 311.2/312.2 [M+1].

To a solution of 2-(6-aminopyridin-3-yl)-5-methyl-6-(1-methyl-1H-pyrazol-5-yl)pyrimidine-4-carboxylic acid (40 mg, 0.13 mmol) in N,N-dimethylformamide (3 mL), O-(7-azabenzotriazol-1-yl)-N,N,N′,N′-tetramethyluronium hexafluorophosphate (HATU, 49 mg, 0.13 mmol) and N-methylmorpholine (14 μL, 0.13 mmol) were added. The solution was stirred at room temperature for 1 hour, then 3-(aminomethyl)-4,6-dimethyl-1H-pyridin-2-one hydrochloride (24 mg, 0.129 mmol) and N-methylmorpholine (28 μL, 0.26 mmol) were added. The reaction was stirred at room temperature for 2 hours, then diluted with water and extracted with ethyl acetate (3×20 mL). The combined organic extracts were dried over sodium sulfate and evaporated. The residue was diluted with water and sonicated; the resulting solid was collected by filtration, then transferred to a vial with acetone/water, and lyophilized to give the title compound (20 mg, 35% yield) as a white solid. ¹H NMR (400 MHz, DMSO-d₆): δ 8.87-8.95 (m, 1H), 8.84 (s, 1H), 8.36 (d, J=13.9 Hz, 1H), 7.50 (d, J=2.0 Hz, 1H), 6.64 (d, J=1.8 Hz, 2H), 5.81 (s, 1H), 4.28 (d, J=5.1 Hz, 2H), 3.88 (s, 3H), 2.31 (s, 3H), 2.16 (s, 3H), 2.04 (s, 3H). MS: 445.2/446.2 [M+1].

### Additional Examples

### Method C

### Example 35

### N-[(4,6-dimethyl-2-oxo-1,2-dihydropyridin-3-yl)methyl]-5-(2,2-dioxido-1,3-dihydro-2-benzothien-5-yl)-2-methyl-3-(1-methyl-1H-pyrazol-5-yl)benzamide

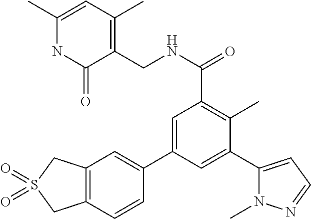 [SMILES: Cc1cc(C)c(CNC(=O)c2cc(-c3ccc4c(c3)CS(=O)(=O)C4)cc(-c3ccnn3C)c2C)c(=O)[nH]1]

The compound of Example 35 was made using the same method as Example 13, starting with 3-bromo-N-((4,6-dimethyl-2-oxo-1,2-dihydropyridin-3-yl)methyl)-5-iodo-2-methyl-benzamide, and 2,2-dioxido-1,3-dihydro-2-benzothiene-5-boronic acid pinacol ester as the first coupling partner, affording the title compound. ¹H NMR (400 MHz, DMSO-d6) δ 11.47 (br. s., 1H), 8.35-8.43 (m, 1H), 7.76-7.79 (m, 1H), 7.72-7.76 (m, 1H), 7.62-7.65 (m, 1H), 7.56-7.59 (m, 1H), 7.52 (d, J=1.76 Hz, 1H), 7.44-7.49 (m, 1H), 6.30 (d, J=1.51 Hz, 1H), 5.88 (s, 1H), 4.53 (s, 4H), 4.33 (d, J=4.52 Hz, 2H), 3.63 (s, 3H), 2.22 (s, 3H) 2.11 (d, J=7.03 Hz, 6H). MS: 517 [M+1].

### Example 149

### N-[(4,6-dimethyl-2-oxo-1,2-dihydropyridin-3-yl)methyl]-2-methyl-5-[6-(4-methyl-piperazin-1-yl)pyridin-3-yl]-3-(1-methyl-1H-pyrazol-5-yl)benzamide

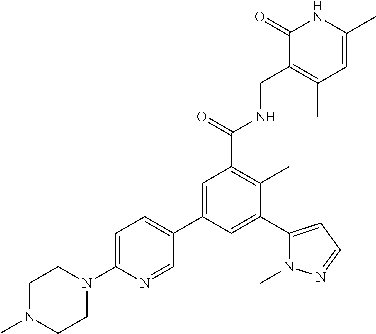

The compound of Example 149 was made using the same method as Example 13, starting with 3-bromo-N-((4,6-dimethyl-2-oxo-1,2-dihydropyridin-3-yl)methyl)-5-iodo-2-methyl-benzamide, and 2-(4-methylpiperazin-1-yl)pyridin-5-boronic acid pinacol ester as the first coupling partner, to afford the title compound. ¹H NMR (400 MHz, methanol-d4) δ 8.42 (d, J=2.32 Hz, 1H), 7.88 (dd, J=8.86, 2.51 Hz, 1H), 7.65 (d, J=1.83 Hz, 1H), 7.57 (d, J=1.83 Hz, 1H), 7.52 (d, J=1.83 Hz, 1H), 6.92 (d, J=8.93 Hz, 1H), 6.31 (d, J=1.96 Hz, 1H), 6.12 (s, 1H), 4.52 (s, 2H); MS: 526.3 [M+1]

### Method L

### Example 36

### 2-chloro-N-[(4,6-dimethyl-2-oxo-1,2-dihydropyridin-3-yl)methyl]-6-[(2S)-2,4-dimethylpiperazin-1-yl]-5-methylpyrimidine-4-carboxamide

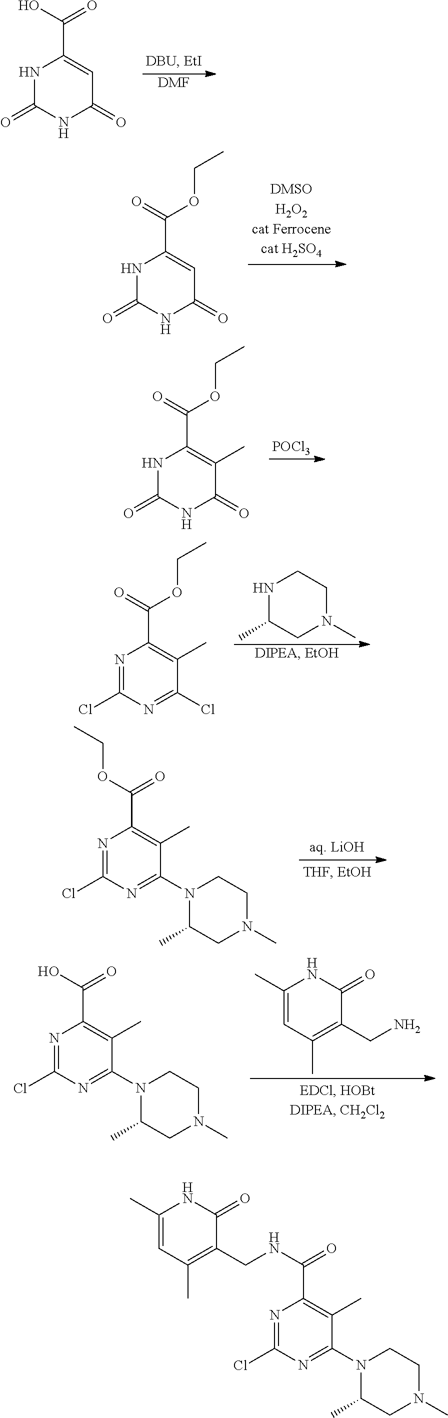

A solution of commercially available orotic acid (10 g, 64 mmol) in DMF (60 mL) was treated with 1,8-diazabicyclo[5.4.0]undec-7-ene (9.75 g, 64 mmol) and stirred at room temperature for 30 minutes. lodoethane (10 g, 64 mmol) was added, and the resulting mixture heated at 60° C. for 2 hours. After cooling to room temperature, deionized water (200 mL) was added, and the resulting white precipitate was collected by filtration. The precipitate was washed with water and dried to give ethyl 2,6-dioxo-1,2,3,6-tetrahydropyrimidine-4-carboxylate (8.1 g, 69% yield) as a white solid.

To a cooled (0° C.) and rapidly stirred solution of ethyl 2,6-dioxo-1,2,3,6-tetrahydropyrimidine-4-carboxylate (8.1 g, 44 mmol), ferrocene (1.64 g, 8.8 mmol), and concentrated sulfuric acid (0.2 mL) in dimethylsulfoxide (50 mL) and deionized water (10 mL) was added 30% aqueous hydrogen peroxide solution (3.23 g, 95 mmol) over 30 minutes. Stirring was continued at 0° C. for 30 minutes, then at room temperature for 3 hours. The reaction mixture was then poured into a flask containing ice and water and sonicated, causing a white precipitate to form. The precipitate was collected by filtration, washed with water, and dried to give ethyl 5-methyl-2,6-dioxo-1,2,3,6-tetrahydropyrimidine-4-carboxylate (7.2 g, 83% yield) as a white solid.

A suspension of ethyl 5-methyl-2,6-dioxo-1,2,3,6-tetrahydropyrimidine-4-carboxylate (54 g, 272 mmol) in phosphorus oxychloride (608.21 g) was stirred at 90° C. for three days. The mixture was evaporated under reduced pressure. The residue was diluted with ethyl acetate and neutralized with aqueous sodium bicarbonate solution. The layers were separated and the aqueous layer extracted with ethyl acetate (2×500 mL). The combined organic layers were dried over sodium sulfate, concentrated, and purified by silica gel chromatography (eluting with ethyl actetate/petroleum ether 1/100) to give ethyl 2,6-dichloro-5-methylpyrimidine-4-carboxylate (41 g, 64% yield) as a colorless oil.

A solution of ethyl 2,6-dichloro-5-methylpyrimidine-4-carboxylate (2.36 g, 10 mmol), in ethanol (100 mL) was treated with (S)-1,3-dimethylpiperazine (1.11 g, 11 mmol) and N,N-diisopropylethylamine (3.88 g, 30 mmol). The mixture was stirred at room temperature overnight, then concentrated under vacuum to give crude (S)-ethyl 2-chloro-6-(2,4-dimethylpiperazin-1-yl)-5-methylpyrimidine-4-carboxylate (2.61 g). This crude material was dissolved in tetrahydrofuran (100 mL) and ethanol (50 mL), then 1M aqueous lithium hydroxide solution (20 mL, 20 mmol) was added. After stirring at room temperature for 4 hours, the mixture was concentrated, adjusted to pH ˜5-6, and extracted with dichloromethane (4×200 mL). The combined dichloromethane extracts were concentrated to afford (S)-ethyl 2-chloro-6-(2,4-dimethylpiperazin-1-yl)-5-methylpyrimidine-4-carboxylate (1.71 g, 63% yield, 2 steps) as a yellow solid.

A solution of (S)-ethyl 2-chloro-6-(2,4-dimethylpiperazin-1-yl)-5-methylpyrimidine-4-carboxylate (5.1 g, 18.8 mmol), 3-aminomethyl-4,6-dimethyl-1H-pyridin-2-one hydrochloride (4.47 g, 20 mmol), O-(7-azabenzotriazol-1-yl)-N,N,N′,N′-tetramethyluronium hexafluoro-phosphate (HATU, 11.41 g, 30 mmol), and N,N-diisopropylethylamine (7.75 g, 60 mmol) in N,N-dimethylformamide (150 mL) was stirred at room temperature overnight. During this time, a precipitate formed. The precipitate was collected by filtration; washed with water, t-butylmethyl ether, and ethyl acetate, and then dried to give 2-chloro-N-[(4,6-dimethyl-2-oxo-1,2-dihydropyridin-3-yl)methyl]-6-[(2S)-2,4-dimethylpiperazin-1-yl]-5-methylpyrimidine-4-carboxamide: (2.94 g, 39% yield) as a white solid. ¹H NMR (400 MHz, DMSO-d6) δ 8.50 (t, J=5.2 Hz, 1H), 8.15 (s, 1H), 5.86 (s, 1H), 4.25 (d, J=5.2 Hz, 2H), 4.05-4.10 (m, J=6.8 Hz, 1H), 3.65 (d, J=11 Hz, 1H), 3.30 (t, J=13 Hz, 1H), 2.85 (d, J=10 Hz, 1H), 2.70 (d, J=10 Hz, 1H), 2.50 (m, 1H), 2.33 (m, 1H), 2.28 (s, 3H), 2.20 (s, 3H), 2.16 (s, 3H), 2.11 (s, 3H), 1.22 (d, J=6.7 Hz, 3H); MS: 419 [M+1].

### Example 45

### 2-(6-aminopyridin-3-yl)-N-[(4,6-dimethyl-2-oxo-1,2-dihydropyridin-3-yl)methyl]-5-methyl-6-[(2S)-2-methylpyrrolidin-1-yl]pyrimidine-4-carboxamide

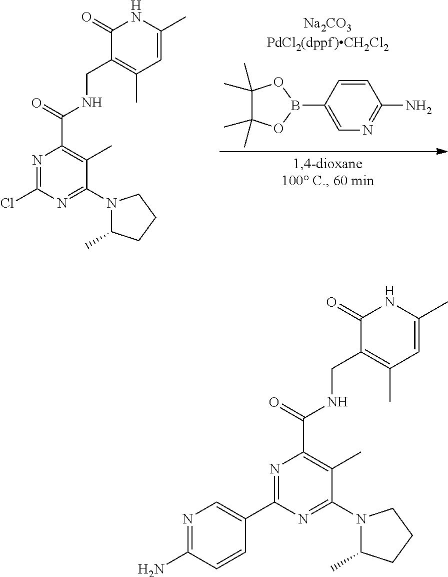 [SMILES: Cc1cc(C)c(CNC(=O)c2nc(Cl)nc(N3CCC[C@@H]3C)c2C)c(=O)[nH]1 ; CC1(C)OB(c2ccc(N)nc2)OC1(C)C ; Cc1cc(C)c(CNC(=O)c2nc(-c3ccc(N)nc3)nc(N3CCC[C@@H]3C)c2C)c(=O)[nH]1]

To a solution of (S)-2-chloro-N-((4,6-dimethyl-2-oxo-1,2-dihydropyridin-3-yl)methyl)-5-methyl-6-(2-methylpyrrolidin-1-yl)pyrimidine-4-carboxamide (50 mg, 0.13 mmol, prepared by the same method as Example 36, using (S)-2-methylpyrrolidine instead of (S)-1,3-dimethylpiperazine) in 1,4-dioxane (4 mL), were added 5-(4,4,5,5-tetramethyl-1,3,2-dioxaborolan-2-yl) pyridin-2-amine (28 mg, 0.13 mmol), sodium carbonate (41 mg, 0.38 mmol), PdCl₂(dppf).CH₂Cl₂ (5 mg, 0.006 mmol), and water (0.5 mL). The reaction vial was sealed, sonicated to dissolve the contents, and degassed with nitrogen, and heated at 100° C. for 60 minutes. The mixture was filtered through a pad of CELITE™ and evaporated to dryness. The residue was loaded with MeOH in a pre-washed SCX (strong cation exchange) column and washed with MeOH (20 mL). The product was cleaved with NH₃/MeOH and the volatiles evaporated. The residue was transferred with MeOH to a vial, diluted with water and lyophilized to give the title product as a tan solid (36 mg, 63% yield). ¹H NMR (400 MHz, DMSO-d6) δ 8.78 (d, J=2.3 Hz, 1H), 7.98-8.28 (m, OH), 6.39 (d, J=8.6 Hz, 1H), 6.25 (s, 1H), 5.59-5.88 (m, 1H), 4.28-4.47 (m, 1H), 4.13-4.28 (m, 2H), 3.62-3.80 (m, 1H), 3.44 (t, J=10.0 Hz, 1H), 2.54-2.62 (m, 1H), 2.23-2.26 (m, J=1.5 Hz, 1H), 2.23 (s, 3H), 2.14 (s, 3H), 2.04-2.09 (m, J=5.3 Hz, 1H), 2.03 (s, 3H), 1.75-1.91 (m, 1H), 1.57-1.71 (m, 1H), 1.42-1.57 (m, 1H), 1.15 (d, J=6.1 Hz, 3H). MS: 448 [M+1].

### Method M (Amine Version)

### Example 43

### 2-(4-aminopiperidin-1-yl)-N-[(4,6-dimethyl-2-oxo-1,2-dihydropyridin-3-yl)methyl]-5-methyl-6-(1-methyl-1H-pyrazol-5-pyrimidine-4-carboxamide

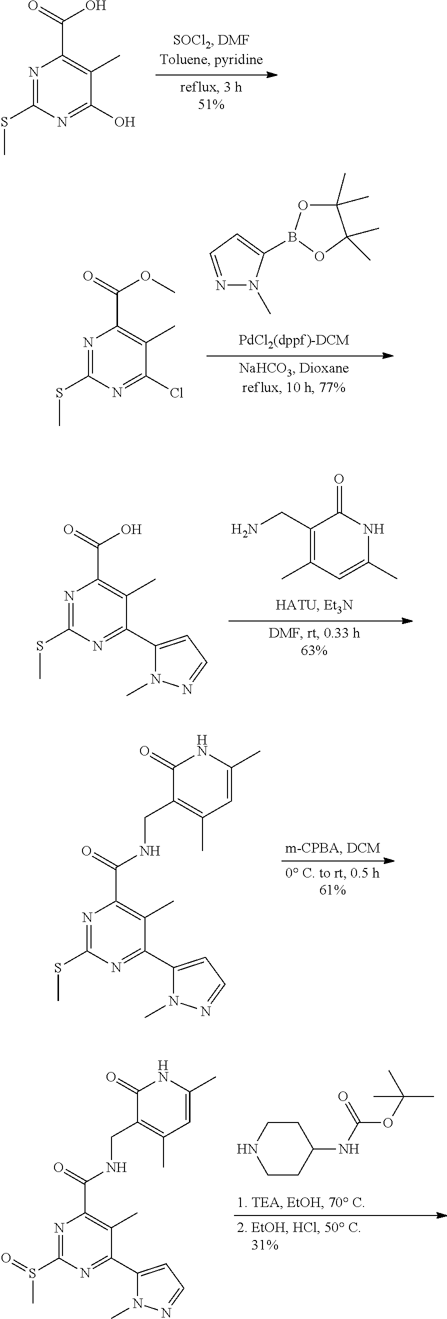

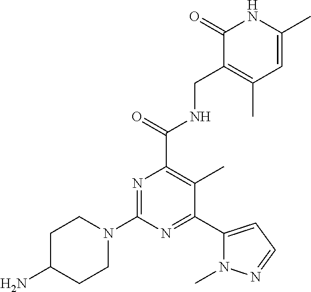 [SMILES: Cc1cc(C)c(CNC(=O)c2nc(N3CCC(N)CC3)nc(-c3ccnn3C)c2C)c(=O)[nH]1]

A suspension of 6-hydroxy-5-methyl-2-(methylsulfanyl)pyrimidine-4-carboxylic acid (3.616 g, 18.06 mmol) in toluene (18 mL) was treated with thionyl chloride (17.1 mL, 234 mmol), DMF (0.120 mL, 1.55 mmol), and pyridine (0.300 mL, 3.71 mmol). The reaction was brought to reflux and stirred for 3 h. The mixture was concentrated in vacuum. The resulting mix of solids and oil was cooled to 0° C. and MeOH (20 mL) was added. After stirring for 5 minutes, the MeOH was removed under vacuum, and the resulting oil was partitioned between saturated aqueous NaHCO₃ and DCM. The DCM layer was concentrated to give an oil which was purified on silica gel (Biotage Flash 40M, eluting with a gradient of 5% to 20% EtOAc in heptanes) to give methyl 6-chloro-5-methyl-2-(methylthio)pyrimidine-4-carboxylate as a light yellow oil, which solidified upon standing (2.144 g, 51% yield). ¹H NMR (400 MHz, DMSO-d₆) δ 3.90-3.95 (m, 3H), 2.53 (s, 3H), 2.32 (s, 3H).

A partial solution of methyl 6-chloro-5-methyl-2-(methylthio)pyrimidine-4-carobxylate (1.832 g, 7.873 mmol), 1-methyl-1H-pyrazole-5-boronic acid pinacol ester (1.97 g, 9.45 mmol), and saturated aqueous NaHCO₃ (24.5 mL) in dioxane (100 mL) was degassed by bubbling N₂ through the solution for 10 minutes. The reaction was treated with PdCl₂(dppf)-DCM (617 mg, 0.756 mmol) and heated at reflux for 2 h. All of the starting chloride was consumed, and about 50% of the desired product was hydrolyzed to the carboxylic acid. Heating for an additional 8 h hydrolyzed the remaining ester. The reaction mixture was cooled to rt, diluted with water (30 mL) and washed with EtOAc. The aqueous layer was brought to pH-2 with 6N HCl and extracted with DCM. The DCM was concentrated to give 5-methyl-6-(1-methyl-1H-pyrazol-5-yl)-2-(methylthio)pyrimidine-4-carboxylic acid as a brown powder (1.602 g, 77%) which was used without further purification. ¹H NMR (400 MHz, DMSO-d₆) δ 7.58 (d, J=2.20 Hz, 1H), 6.77 (d, J=1.96 Hz, 1H), 3.94 (s, 3H), 2.55 (s, 3H), 2.29 (s, 3H).

To a solution of the above pyrimidine-4-carboxylic acid (1.554 g, 5.880 mmol) in DMF (20 mL) was added triethylamine (2.50 mL, 17.9 mmol) and then HATU (2.46 g, 6.3 mmol). After 5 minutes, 3-aminomethyl-4,6-dimethyl-1H-pyridin-2-one hydrochloride was added in one portion. The reaction was stirred at rt for 20 minutes. Water (100 mL) was added, and the precipitate was collected by filtration and dried overnight under high vacuum. The resulting N-((4,6-dimethyl-2-oxo-1,2-dihydropyridin-3-yl)methyl)-5-methyl-6-(1-methyl-1H-pyrazol-5-yl)-2-(methylthio)pyrimidine-4-carboxamide was provided as a powder (1.475 g, 63%), which was used without further purification. ¹H NMR (400 MHz, DMSO-d₆) δ 11.57 (br. s., 1H), 8.77 (t, J=5.26 Hz, 1H), 7.58 (d, J=1.96 Hz, 1H), 6.71 (d, J=1.96 Hz, 1H), 5.89 (s, 1H), 4.32 (d, J=5.38 Hz, 2H), 3.91 (s, 3H), 2.56 (s, 3H), 2.30 (s, 3H), 2.22 (s, 3H), 2.13 (s, 3H).

To a solution of the carboxamide intermediate (0.613 g, 1.54 mmol) in dichloromethane (20 mL) in an ice bath was added m-CPBA (0.384 g, 1.7 mmol, 75% pure) in one portion. The reaction was allowed to warm to rt. The starting material was consumed after 15 minutes. The entire reaction mixture was poured onto silica gel (Biotage Flash 40S, eluting with a gradient of 0-20% MeOH in DCM w/0.5% triethylamine) to give N-((4,6-dimethyl-2-oxo-1,2-dihydropyridin-3-yl)methyl)-5-methyl-6-(1-methyl-1H-pyrazol-5-yl)-2-(methylsulfinyl)pyrimidine-4-carboxamide as a white powder (0.389 g, 61%). ¹H NMR (400 MHz, DMSO-d₆) δ 11.56 (br. s., 1H), 8.80 (t, J=5.50 Hz, 1H), 7.62 (d, J=1.96 Hz, 1H), 6.83 (d, J=2.20 Hz, 1H), 5.89 (s, 1H), 4.35 (d, J=5.38 Hz, 2H), 3.95 (s, 3H), 2.92 (s, 3H), 2.42 (s, 3H), 2.22 (s, 3H), 2.12 (s, 3H).

A fine suspension of N-((4,6-dimethyl-2-oxo-1,2-dihydropyridin-3-yl)methyl)-5-methyl-6-(1-methyl-1H-pyrazol-5-yl)-2-(methylsulfinyl)pyrimidine-4-carboxamide (0.021 g, 0.051 mmol) in EtOH (2 mL) was treated with 4-(N-tert-butoxycarbonylamino)piperidine (0.015 g, 0.075 mmol) and triethylamine (0.010 mL, 0.072 mmol) and heated at 70° C. overnight. The reaction was then treated with concentrated HCl (6 drops) and heated at 50° C. for 6 h. The volatiles were blown off with nitrogen, and the resulting dark residue was dissolved in MeOH and purified by preparative HPLC to give 2-(4-aminopiperidin-1-yl)-N-[(4,6-dimethyl-2-oxo-1,2-dihydropyridin-3-yl)methyl]-5-methyl-6-(1-methyl-1H-pyrazol-5-yl)pyrimidine-4-carboxamide (0.00711 g, 31%). MS: 451 [M+1].

### Method M (Ether Version)

### Example 102

### N-[(4,6-dimethyl-2-oxo-1,2-dihydropyridin-3-yl)methyl]-2-ethoxy-5-methyl-6-(1-methyl-1H-pyrazol-5-yl)pyrimidine-4-carboxamide

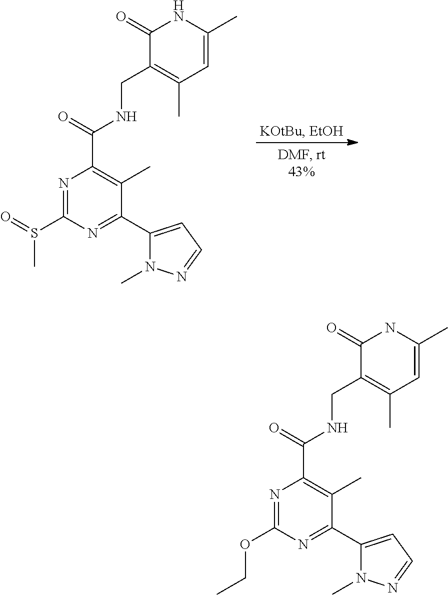

A fine suspension of N-((4,6-dimethyl-2-oxo-1,2-dihydropyridin-3-yl)methyl)-5-methyl-6-(1-methyl-1H-pyrazol-5-yl)-2-(methylsulfinyl)pyrimidine-4-carboxamide (0.051 g, 0.12 mmol), prepared as in Example 43 above, in EtOH (1 mL) and DMF (1 mL) was treated with potassium tert-butoxide (0.40 mL, 0.40 mmol, 1M in THF) at rt. After 5 minutes, the reaction was neutralized with acetic acid (0.016 mL). The resulting mixture was filtered and purified by preparative HPLC to give N-[(4,6-dimethyl-2-oxo-1,2-dihydropyridin-3-yl)methyl]-2-ethoxy-5-methyl-6-(1-methyl-1H-pyrazol-5-yl)pyrimidine-4-carboxamide (0.021 g, 43%). ¹H NMR (400 MHz, DMSO-d₆) δ 8.68 (t, J=5.31 Hz, 1H), 7.56 (d, J=2.02 Hz, 1H), 6.67 (d, J=1.77 Hz, 1H), 5.88 (s, 1H), 4.37 (q, J=6.99 Hz, 2H), 4.31 (d, J=5.31 Hz, 2H), 3.91 (s, 3H), 2.28 (s, 3H), 2.21 (s, 3H), 2.12 (s, 3H), 1.34 (t, J=7.07 Hz, 3H); MS: 397 [M+1].

### Method N

### Example 147

### N-[(5-bromo-4,6-dimethyl-2-oxo-1,2-dihydropyridin-3-yl)methyl]-3-(1-ethyl-4-methyl-1H-pyrazol-5-yl)-2-methylbenzamide

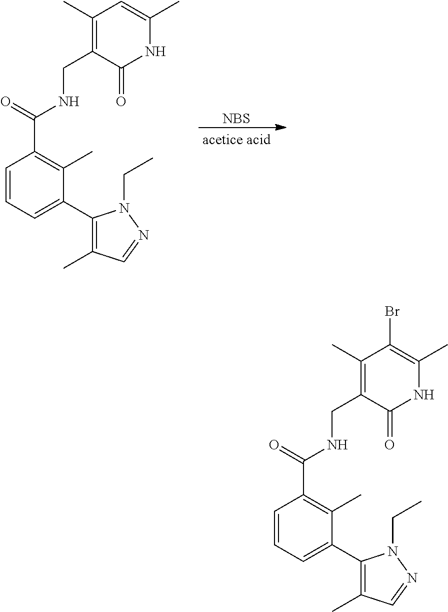 [SMILES: CCn1ncc(C)c1-c1cccc(C(=O)NCc2c(C)cc(C)[nH]c2=O)c1C ; "NBS" ; CCn1ncc(C)c1-c1cccc(C(=O)NCc2c(C)c(Br)c(C)[nH]c2=O)c1C]

N-[(4,6-dimethyl-2-oxo-1,2-dihydropyridin-3-yl)methyl]-3-(1-ethyl-4-methyl-1H-pyrazol-5-yl)-2-methylbenzamide (0.05 g, 0.13 mmol) and N-bromosuccinimide (24 mg, 0.13 mmol) were dissolved in glacial acetic acid (5 mL) to give a clear colorless solution. The mixture was heated at 80° C. for 3 h. Evaporate all solvent and get a white solid. Dichloromethane (10 mL) was added and a white precipitate formed. The precipitate was collected and dried to give N-[(5-bromo-4,6-dimethyl-2-oxo-1,2-dihydropyridin-3-yl)methyl]-3-(1-ethyl-4-methyl-1H-pyrazol-5-yl)-2-methylbenzamide (0.038 g, 64% yield).

The compounds in Table 1 were prepared as described in Examples 1-36.

TABLE 1

| Ex. | Structure | Name |
|---|---|---|
| 1 | 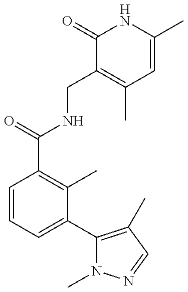 [SMILES: Cc1cc(C)c(CNC(=O)c2cccc(-c3c(C)cnn3C)c2C)c(=O)[nH]1] | N-[(4,6-dimethyl-2-oxo-1,2-dihydropyridin- 3-yl)methyl]-3-(1,4-dimethyl-1H-pyrazol-5- yl)-2-methylbenzamide |
| 2 | 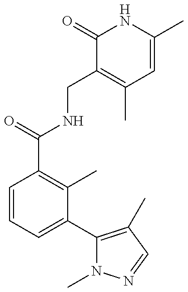 [SMILES: Cc1cc(C)c(CNC(=O)c2cccc(-c3c(C)cnn3C)c2C)c(=O)[nH]1] | N-[(4,6-dimethyl-2-oxo-1,2-dihydropyridin- 3-yl)methyl]-2-methyl-3-(1-methyl-1H- pyrazol-5-yl)benzamide |
| 3 | 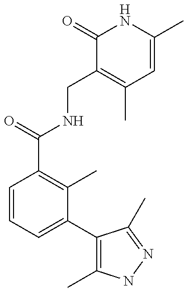 [SMILES: Cc1cc(C)c(CNC(=O)c2cccc(-c3c(C)n[nH]c3C)c2C)c(=O)[nH]1] | N-[(4,6-dimethyl-2-oxo-1,2-dihydropyridin- 3-yl)methyl]-3-(3,5-dimethyl-1H-pyrazol-4- yl)-2-methylbenzamide |
| 4 | 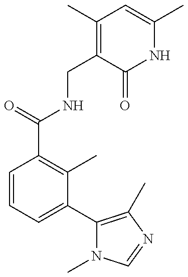 [SMILES: Cc1cc(C)c(CNC(=O)c2cccc(-c3c(C)ncn3C)c2C)c(=O)[nH]1] | 3-(1,4-dimethyl-1H-imidazol-5-yl)-N-[(4,6- dimethyl-2-oxo-1,2-dihydropyridin-3- yl)methyl]-2-methylbenzamide |
| 5 | 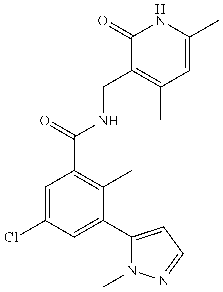 [SMILES: Cc1cc(C)c(CNC(=O)c2cc(Cl)cc(-c3ccnn3C)c2C)c(=O)[nH]1] | 5-chloro-N-[(4,6-dimethyl-2-oxo-1,2- dihydropyridin-3-yl)methyl]-2-methyl-3-(1- methyl-1H-pyrazol-5-yl)benzamide |
| 6 | 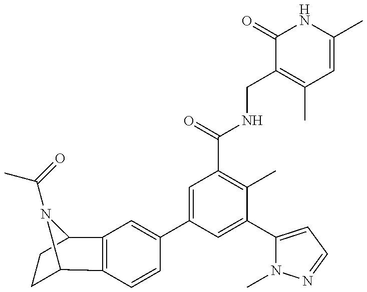 [SMILES: CC(=O)N1C2CCC1c1cc(-c3cc(C(=O)NCc4c(C)cc(C)[nH]c4=O)c(C)c(-c4ccnn4C)c3)ccc12] | 5-[9-acetyl-1,2,3,4-tetrahydro-1,4- epiminonaphthalen-6-yl]-N-[(4,6-dimethyl-2- oxo-1,2-dihydropyridin-3-yl)methyl]-2- methyl-3-(1-methyl-1H-pyrazol-5- yl)benzamide |
| 7 | 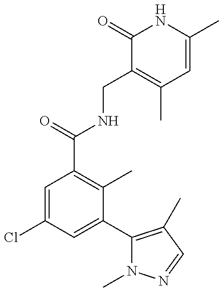 [SMILES: Cc1cc(C)c(CNC(=O)c2cc(Cl)cc(-c3c(C)cnn3C)c2C)c(=O)[nH]1] | 5-chloro-N-[(4,6-dimethyl-2-oxo-1,2- dihydropyridin-3-yl)methyl]-3-(1,4-dimethyl- 1H-pyrazol-5-yl)-2-methylbenzamide |
| 8 | 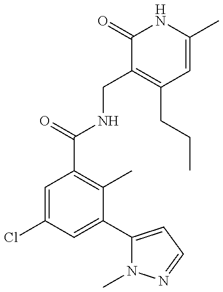 [SMILES: CCCc1cc(C)[nH]c(=O)c1CNC(=O)c1cc(Cl)cc(-c2ccnn2C)c1C] | 5-chloro-2-methyl-N-[(6-methyl-2-oxo-4- propyl-1,2-dihydropyridin-3-yl)methyl]-3-(1- methyl-1H-pyrazol-5-yl)benzamide |
| 9 | 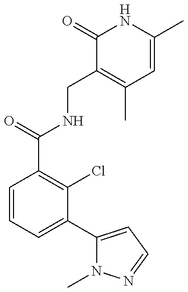 [SMILES: Cc1cc(C)c(CNC(=O)c2cccc(-c3ccnn3C)c2Cl)c(=O)[nH]1] | 2-chloro-N-[(4,6-dimethyl-2-oxo-1,2- dihydropyridin-3-yl)methyl]-3-(1-methyl-1H- pyrazol-5-yl)benzamide |
| 10 | 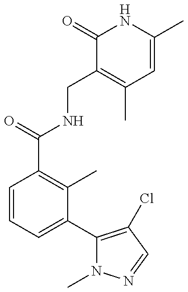 [SMILES: Cc1cc(C)c(CNC(=O)c2cccc(-c3c(Cl)cnn3C)c2C)c(=O)[nH]1] | 3-(4-chloro-1-methyl-1H-pyrazol-5-yl)-N- [(4,6-dimethyl-2-oxo-1,2-dihydropyridin-3- yl)methyl]-2-methylbenzamide |
| 11 | 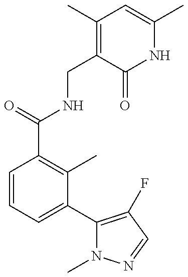 [SMILES: Cc1cc(C)c(CNC(=O)c2cccc(-c3c(F)cnn3C)c2C)c(=O)[nH]1] | N-[(4,6-dimethyl-2-oxo-1,2-dihydropyridin- 3-yl)methyl]-3-(4-fluoro-1-methyl-1H- pyrazol-5-yl)-2-methylbenzamide |
| 12 | 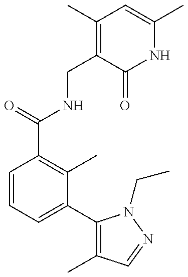 [SMILES: CCn1ncc(C)c1-c1cccc(C(=O)NCc2c(C)cc(C)[nH]c2=O)c1C] | N-[(4,6-dimethyl-2-oxo-1,2-dihydropyridin- 3-yl)methyl]-3-(1-ethyl-4-methyl-1H-pyrazol- 5-yl)-2-methylbenzamide |
| 13 | 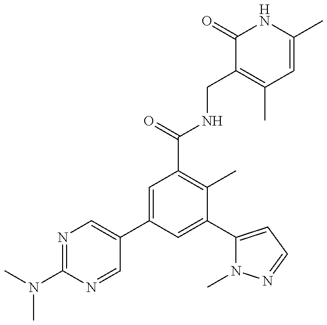 | 5-[2-(dimethylamino)pyrimidin-5-yl]-N-[(4,6- dimethyl-2-oxo-1,2-dihydropyridin-3- yl)methyl]-2-methyl-3-(1-methyl-1H-pyrazol- 5-yl)benzamide |
| 14 | 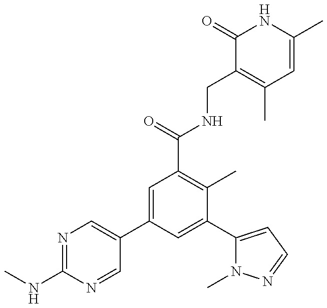 | N-[(4,6-dimethyl-2-oxo-1,2-dihydropyridin- 3-yl)methyl]-2-methyl-5-[2- (methylamino)pyrimidin-5-yl]-3-(1-methyl- 1H-pyrazol-5-yl)benzamide |
| 15 | 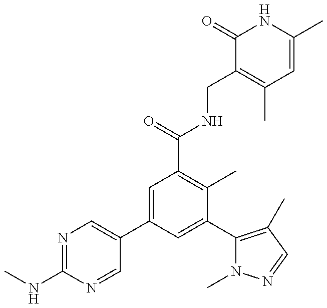 | N-[(4,6-dimethyl-2-oxo-1,2-dihydropyridin- 3-yl)methyl]-3-(1,4-dimethyl-1H-pyrazol-5- yl)-2-methyl-5-[2-(methylamino)pyrimidin-5- yl]benzamide |
| 16 | 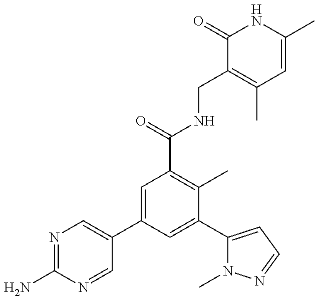 [SMILES: Cc1cc(C)c(CNC(=O)c2cc(-c3cnc(N)nc3)cc(-c3ccnn3C)c2C)c(=O)[nH]1] | 5-(2-aminopyrimidin-5-yl)-N-[(4,6-dimethyl- 2-oxo-1,2-dihydropyridin-3-yl)methyl]-2- methyl-3-(1-methyl-1H-pyrazol-5- yl)benzamide |
| 17 | 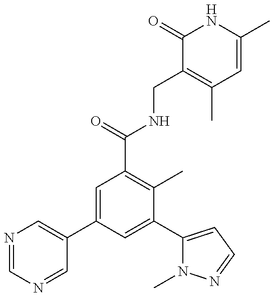 [SMILES: Cc1cc(C)c(CNC(=O)c2cc(-c3cncnc3)cc(-c3ccnn3C)c2C)c(=O)[nH]1] | N-[(4,6-dimethyl-2-oxo-1,2-dihydropyridin- 3-yl)methyl]-2-methyl-3-(1-methyl-1H- pyrazol-5-yl)-5-pyrimidin-5-ylbenzamide |
| 18 | 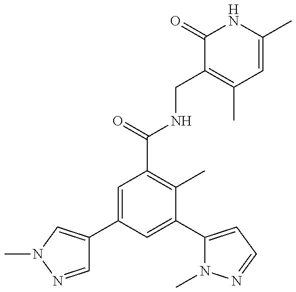 | N-[(4,6-dimethyl-2-oxo-1,2-dihydropyridin- 3-yl)methyl]-2-methyl-5-(1-methyl-1H- pyrazol-4-yl)-3-(1-methyl-1H-pyrazol-5- yl)benzamide |
| 19 | 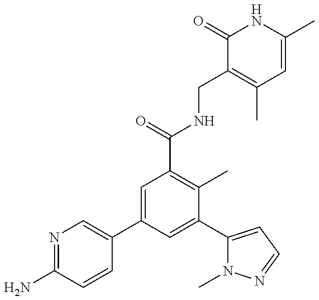 [SMILES: Cc1cc(C)c(CNC(=O)c2cc(-c3ccc(N)nc3)cc(-c3ccnn3C)c2C)c(=O)[nH]1] | 5-(6-aminopyridin-3-yl)-N-[(4,6-dimethyl-2- oxo-1,2-dihydropyridin-3-yl)methyl]-2- methyl-3-(1-methyl-1H-pyrazol-5- yl)benzamide |
| 20 | 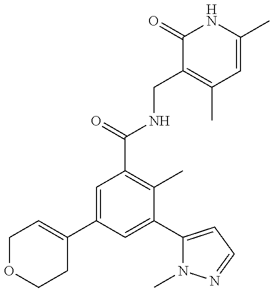 [SMILES: Cc1cc(C)c(CNC(=O)c2cc(C3=CCOCC3)cc(-c3ccnn3C)c2C)c(=O)[nH]1] | 5-(3,6-dihydro-2H-pyran-4-yl)-N-[(4,6- dimethyl-2-oxo-1,2-dihydropyridin-3- yl)methyl]-2-methyl-3-(1-methyl-1H-pyrazol- 5-yl)benzamide |
| 21 | 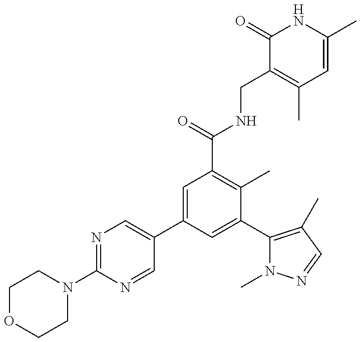 [SMILES: Cc1cc(C)c(CNC(=O)c2cc(-c3cnc(N4CCOCC4)nc3)cc(-c3c(C)cnn3C)c2C)c(=O)[nH]1] | N-[(4,6-dimethyl-2-oxo-1,2-dihydropyridin- 3-yl)methyl]-3-(1,4-dimethyl-1H-pyrazol-5- yl)-2-methyl-5-(2-morpholin-4-ylpyrimidin-5- yl)benzamide |
| 22 | 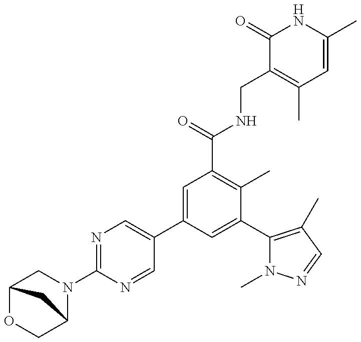 [SMILES: Cc1cc(C)c(CNC(=O)c2cc(-c3cnc(N4C[C@@H]5C[C@H]4CO5)nc3)cc(-c3c(C)cnn3C)c2C)c(=O)[nH]1] | N-[(4,6-dimethyl-2-oxo-1,2-dihydropyridin- 3-yl)methyl]-3-(1,4-dimethyl-1H-pyrazol-5- yl)-2-methyl-5-{2-[(1S,4S)-2-oxa-5- azabicyclo[2.2.1]hept-5-yl]pyrimidin-5- yl}benzamide |
| 23 | 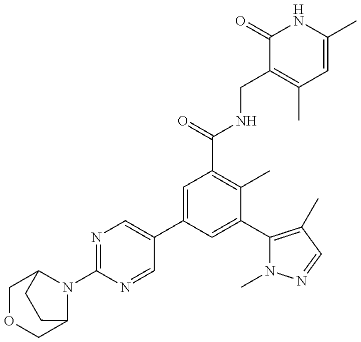 [SMILES: Cc1cc(C)c(CNC(=O)c2cc(-c3cnc(N4C5CCC4COC5)nc3)cc(-c3c(C)cnn3C)c2C)c(=O)[nH]1] | N-[(4,6-dimethyl-2-oxo-1,2-dihydropyridin- 3-yl)methyl]-3-(1,4-dimethyl-1H-pyrazol-5- yl)-2-methyl-5-{2-[3-oxa-8- azabicyclo[3.2.1]oct-8-yl]pyrimidin-5- yl}benzamide |
| 24 | 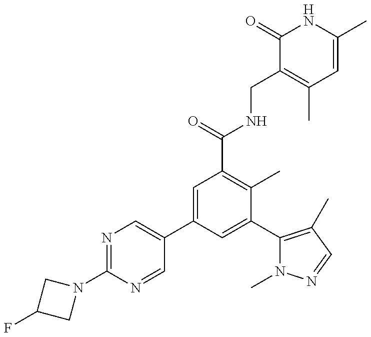 [SMILES: Cc1cc(C)c(CNC(=O)c2cc(-c3cnc(N4CC(F)C4)nc3)cc(-c3c(C)cnn3C)c2C)c(=O)[nH]1] | N-[(4,6-dimethyl-2-oxo-1,2-dihydropyridin- 3-yl)methyl]-3-(1,4-dimethyl-1H-pyrazol-5- yl)-5-[2-(3-fluoroazetidin-1-yl)pyrimidin-5- yl]-2-methylbenzamide |
| 25 | 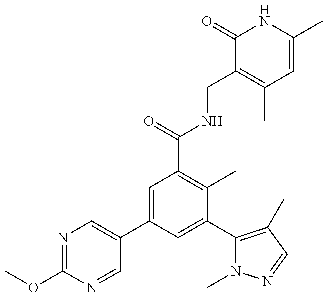 | N-[(4,6-dimethyl-2-oxo-1,2-dihydropyridin- 3-yl)methyl]-3-(1,4-dimethyl-1H-pyrazol-5- yl)-5-(2-methoxypyrimidin-5-yl)-2- methylbenzamide |
| 26 | 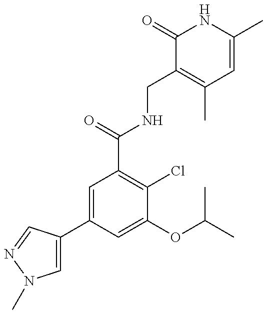 [SMILES: Cc1cc(C)c(CNC(=O)c2cc(-c3cnn(C)c3)cc(OC(C)C)c2Cl)c(=O)[nH]1] | 2-chloro-N-[(4,6-dimethyl-2-oxo-1,2- dihydropyridin-3-yl)methyl]-3-isopropoxy-5- (1-methyl-1H-pyrazol-4-yl)benzamide |
| 27 | 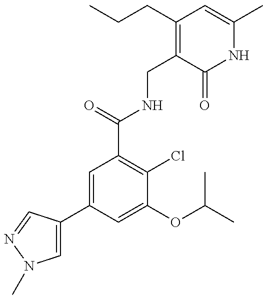 [SMILES: CCCc1cc(C)[nH]c(=O)c1CNC(=O)c1cc(-c2cnn(C)c2)cc(OC(C)C)c1Cl] | 2-chloro-3-isopropoxy-N-[(6-methyl-2-oxo- 4-propyl-1,2-dihydropyridin-3-yl)methyl]-5- (1-methyl-1H-pyrazol-4-yl)benzamide |
| 28 | 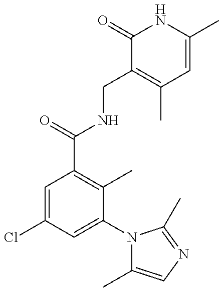 [SMILES: Cc1cc(C)c(CNC(=O)c2cc(Cl)cc(-n3c(C)cnc3C)c2C)c(=O)[nH]1] | 5-chloro-3-(2,5-dimethyl-1H-imidazol-1-yl)- N-[(4,6-dimethyl-2-oxo-1,2-dihydropyridin- 3-yl)methyl]-2-methylbenzamide |
| 29 | 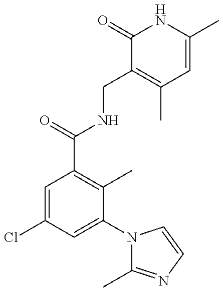 [SMILES: Cc1cc(C)c(CNC(=O)c2cc(Cl)cc(-n3ccnc3C)c2C)c(=O)[nH]1] | 5-chloro-N-[(4,6-dimethyl-2-oxo-1,2- dihydropyridin-3-yl)methyl]-2-methyl-3-(2- methyl-1H-imidazol-1-yl)benzamide |
| 30 | 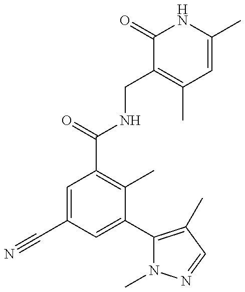 [SMILES: Cc1cc(C)c(CNC(=O)c2cc(C#N)cc(-c3c(C)cnn3C)c2C)c(=O)[nH]1] | 5-cyano-N-[(4,6-dimethyl-2-oxo-1,2- dihydropyridin-3-yl)methyl]-3-(1,4-dimethyl- 1H-pyrazol-5-yl)-2-methylbenzamide |
| 31 | 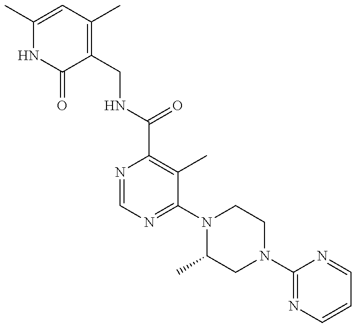 [SMILES: Cc1cc(C)c(CNC(=O)c2ncnc(N3CCN(c4ncccn4)C[C@@H]3C)c2C)c(=O)[nH]1] | (S)-N-((4,6-dimethyl-2-oxo-1,2- dihydropyridin-3-yl)methyl)-5-methyl-6-(2- methyl-4-(pyrimidin-2-yl)piperazin-1- yl)pyrimidine-4-carboxamide |
| 32 | 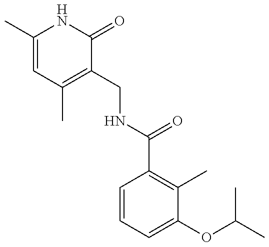 [SMILES: Cc1cc(C)c(CNC(=O)c2cccc(OC(C)C)c2C)c(=O)[nH]1] | N-((4,6-dimethyl-2-oxo-1,2-dihydropyridin- 3-yl)methyl)-3-isopropoxy-2- methylbenzamide |
| 33 | 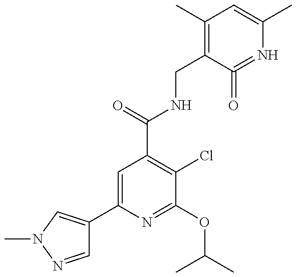 | 3-chloro-N-[(4,6-dimethyl-2-oxo-1,2- dihydropyridin-3-yl)methyl]-2-isopropoxy-6- (1-methyl-1H-pyrazol-4-yl)isonicotinamide |
| 34 | 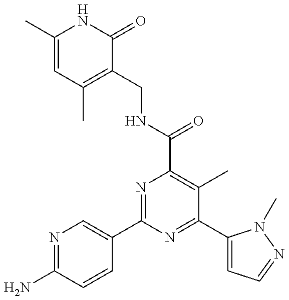 [SMILES: Cc1cc(C)c(CNC(=O)c2nc(-c3ccc(N)nc3)nc(-c3ccnn3C)c2C)c(=O)[nH]1] | 2-(6-aminopyridin-3-yl)-N-[(4,6-dimethyl-2- oxo-1,2-dihydropyridin-3-yl)methyl]-5- methyl-6-(1-methyl-1H-pyrazol-5- yl)pyrimidine-4-carboxamide |
| 35 | 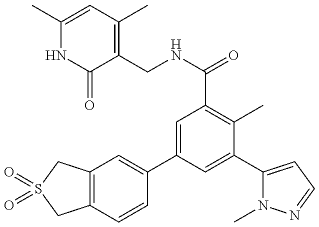 [SMILES: Cc1cc(C)c(CNC(=O)c2cc(-c3ccc4c(c3)CS(=O)(=O)C4)cc(-c3ccnn3C)c2C)c(=O)[nH]1] | N-[(4,6-dimethyl-2-oxo-1,2-dihydropyridin- 3-yl)methyl]-5-(2,2-dioxido-1,3-dihydro-2- benzothien-5-yl)-2-methyl-3-(1-methyl-1H- pyrazol-5-yl)benzamide |
| 36 | 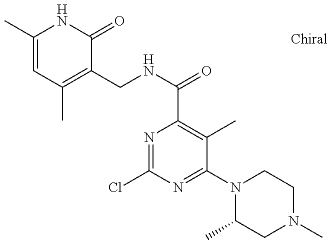 | 2-chloro-N-[(4,6-dimethyl-2-oxo-1,2- dihydropyridin-3-yl)methyl]-6-[(2S)-2,4- dimethylpiperazin-1-yl]-5-methylpyrimidine- 4-carboxamide |

The compounds in Table 2 were prepared by modification or extension of Methods A-N, which are exemplified herein for the preparation of Examples 1-36, 43, 45, 102 and 147 throughout the Examples section.

TABLE 2

| Ex. | Structure | Name | ¹H NMR/LC-MS | Method |
|---|---|---|---|---|
| 37 | 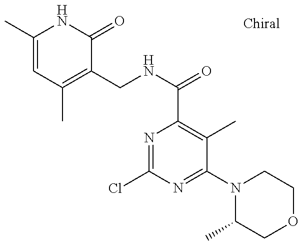 [SMILES: Cc1cc(C)c(CNC(=O)c2nc(Cl)nc(N3CCOC[C@@H]3C)c2C)c(=O)[nH]1] | 2-chloro-N- [(4,6-dimethyl- 2-oxo-1,2- dihydropyridin- 3-yl)methyl]-5- methyl-6-[(3S)- 3- methylmorpholin- 4- yl]pyrimidine- 4-carboxamide | ¹H NMR (400 MHz, DMSO-d6) δ 11.54 (s, 1H), 8.49 (s, 1H), 5.86 (s, 1H), 4.25 (d, J = 5.2 Hz, 2H), 4.05-4.07 (m, J = 6.8 Hz, 1H), 3.81-3.84 (m, J = 11.2 Hz, 1H), 3.51-3.54 (m, J = 13.2 Hz, 2H), 3.47-3.49 (m, J = 11.2 Hz, 2H), 3.36- 3.40 (m, J = 16.8 Hz, 1H), 2.18 (s, 3H), 2.16 (s, 3H), 2.11 (s, 3H), 1.21-1.23 (m, J = 6.8 Hz, 3H); MS: 406 [M + 1] | L |
| 38 | 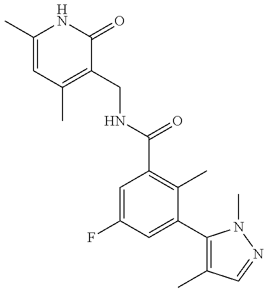 [SMILES: Cc1cc(C)c(CNC(=O)c2cc(F)cc(-c3c(C)cnn3C)c2C)c(=O)[nH]1] | N-[(4,6- dimethyl-2- oxo-1,2- dihydropyridin- 3-yl)methyl]-3- (1,4-dimethyl- 1H-pyrazol-5- yl)-5-fluoro-2- methylbenz- amide | ¹H NMR (400 MHz, DMSO- d6) δ 11.47 (s, 1H), 8.39- 8.37 (m, 1H), 7.33 (s, 1H), 7.19-7.11 (m, 2H), 5.86 (s, 1H), 4.28-4.27 (d, J = 4.8 Hz, 2H), 3.49 (s, 3H), 2.19 (s, 3H), 2.10 (s, 3H), 1.92 (s, 3H), 1.79 (s, 3H); MS: 383 [M + 1] | B |
| 39 | 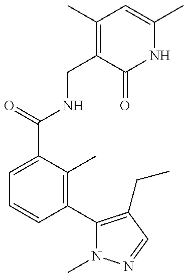 [SMILES: CCc1cnn(C)c1-c1cccc(C(=O)NCc2c(C)cc(C)[nH]c2=O)c1C] | N-[(4,6- dimethyl-2- oxo-1,2- dihydropyridin- 3-yl)methyl]-3- (4-ethyl-1- methyl-1H- pyrazol-5-yl)-2- methylbenz- amide | ¹H NMR (400 MHz, chloroform-d) δ 11.47 (br.s., 1H), 7.40 (br.s., 2H), 7.23-7.17 (m, 3H), 5.96 (s, 1H), 4.55 (d, J = 5 Hz, 2H), 3.54 (s, 3H), 2.41 (s, 3H), 2.23-2.16 (m, 5H), 2.11 (s, 3H), 1.03 (t, J = 7.4 Hz, 3H); MS: 379 [M + 1] | B |
| 40 | 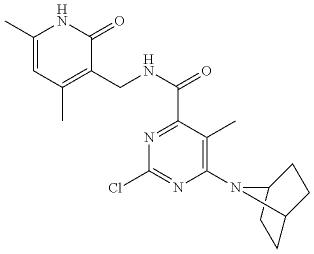 [SMILES: Cc1cc(C)c(CNC(=O)c2nc(Cl)nc(N3C4CCC3CC4)c2C)c(=O)[nH]1] | 6-(7- azabicyclo[2.2.1] hept-7-yl)-2- chloro-N-[(4,6- dimethyl-2- oxo-1,2- dihydropyridin- 3-yl)methyl]-5- methylpyrimidine- 4- carboxamide | ¹H NMR (400 MHz, DMSO-d6) δ 11.54 (s, 1H), 8.48 (s, 1H), 5.87 (s, 1H), 4.56 (s, 2H), 4.24-4.26 (m, J = 5.6 Hz, 2H), 2.20 (s, 3H), 2.19 (s, 3H), 2.11 (s, 3H), 1.71-1.73 (m, J = 7.2 Hz, 4H), 1.47-1.49 (m, J = 6.8 Hz, 4H); MS: 402 [M + 1] | L |
| 41 | 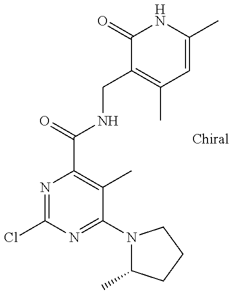 [SMILES: Cc1cc(C)c(CNC(=O)c2nc(Cl)nc(N3CCC[C@@H]3C)c2C)c(=O)[nH]1] | 2-chloro-N- [(4,6-dimethyl- 2-oxo-1,2- dihydropyridin- 3-yl)methyl]-5- methyl-6-[(2S)- 2- methylpyrrolidin- 1- yl]pyrimidine- 4-carboxamide | ¹H NMR (400 MHz, DMSO-d6) δ 11.42 (br. s., 1H), 8.31 (t, J = 5.3 Hz, 1H), 5.78 (s, 1H), 4.18-4.25 (m, 1H), 4.11-4.18 (m, 2H), 3.69 (ddd, J = 10.4, 7.7, 7.5 Hz, 1H), 3.44- 3.57 (m, 1H), 2.14 (s, 3H), 2.10 (s, 3H), 2.02 (s, 3H), 1.93-2.05 (m, 1H), 1.79- 1.91 (m, 0H), 1.56-1.70 (m, 0H), 1.37-1.52 (m, 0H), 1.07 (d, J = 6.1 Hz, 3H); MS: 390 [M + 1] | L |
| 42 | 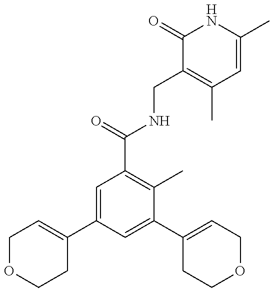 [SMILES: Cc1cc(C)c(CNC(=O)c2cc(C3=CCOCC3)cc(C3=CCOCC3)c2C)c(=O)[nH]1] | 3,5-di-3,6- dihydro-2H- pyran-4-yl-N- [(4,6-dimethyl- 2-oxo-1,2- dihydropyridin- 3-yl)methyl]-2- methylbenz- amide | ¹H NMR (400 MHz, DMSO-d6) δ 2.03 (s, 3 H) 2.10 (s, 3 H) 2.20 (s, 3 H) 3.57 (s, 2 H) 3.80 (t, J = 5.5 Hz, 2 H) 4.20 (d, J = 2.7 Hz, 2 H) 4.29 (d, J = 4.9 Hz, 2 H) 5.86 (s, 1 H) 6.24 (d, J = 1.7 Hz, 1 H) 6.32 (br. s., 1 H) 7.31 (d, J = 1.7 Hz, 1 H) 7.38 (d, J = 1.7 Hz, 1 H) 7.49 (d, (d, J = 1.7 Hz, 1 H) 8.28 (s, 1 H) 11.46 (br. s., 1 H); MS: 435 [M + 1] | C |
| 43 | 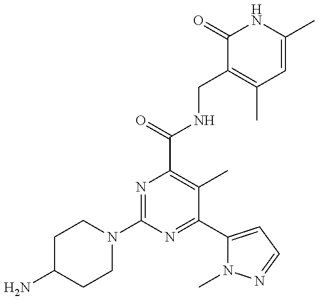 [SMILES: Cc1cc(C)c(CNC(=O)c2nc(N3CCC(N)CC3)nc(-c3ccnn3C)c2C)c(=O)[nH]1] | 2-(4- aminopiperidin- 1-yl)-N-[(4,6- dimethyl-2- oxo-1,2- dihydropyridin- 3-yl)methyl]-5- methyl-6-(1- methyl-1H- pyrazol-5- yl)pyrimidine- 4-carboxamide | MS: 451 [M + 1] | M |
| 44 | 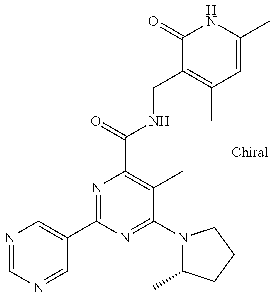 [SMILES: Cc1cc(C)c(CNC(=O)c2nc(-c3cncnc3)nc(N3CCC[C@@H]3C)c2C)c(=O)[nH]1] | N-[(4,6- dimethyl-2- oxo-1,2- dihydropyridin- 3-yl)methyl]-5- methyl-6-[(2S)- 2- methylpyrrolidin- 1-yl]-2,5′- bipyrimidine-4- carboxamide | ¹H NMR (400 MHz, DMSO-d6) δ 11.52 (br. s., 1H), 9.46 (s, 2H), 9.20 (s, 1H), 8.90 (t, J = 5.3 Hz, 1H), 5.80 (s, 1H), 4.41 (d, J = 7.1 Hz, 1H), 4.13-4.30 (m, 2H), 3.94 (q, J = 7.1 Hz, 1H), 3.69-3.81 (m, 1H), 3.54 (t, J = 10.4 Hz, 1H), 2.30 (s, 3H), 2.14 (s, 3H), 2.04-2.10 (m, 1H), 2.03 (s, 3H), 1.19 (d, J = 6.1 Hz, 3H), 1.09 (t, J = 7.1 Hz, 2H); MS: 434 [M + 1] | L |
| 45 | 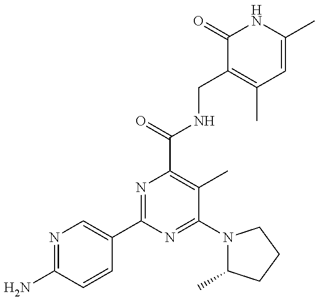 [SMILES: Cc1cc(C)c(CNC(=O)c2nc(-c3ccc(N)nc3)nc(N3CCC[C@@H]3C)c2C)c(=O)[nH]1] | 2-(6- aminopyridin- 3-yl)-N-[(4,6- dimethyl-2- oxo-1,2- dihydropyridin- 3-yl)methyl]-5- methyl-6-[(2S)- 2- methylpyrrolidin- 1- yl]pyrimidine- 4-carboxamide | ¹H NMR (400 MHz, DMSO-d6) δ 8.78 (d, J = 2.3 Hz, 1H), 7.98-8.28 (m, 0H), 6.39 (d, J = 8.6 Hz, 1H), 6.25 (s, 1H), 5.59- 5.88 (m, 1H), 4.28-4.47 (m, 1H), 4.13-4.28 (m, 2H), 3.62-3.80 (m, 1H), 3.44 (t, J = 10.0 Hz, 1H), 2.54-2.62 (m, 1H), 2.23- 2.26 (m, J = 1.5 Hz, 1H), 2.23 (s, 3H), 2.14 (s, 3H), 2.04-2.09 (m, J = 5.3 Hz, 1H), 2.03 (s, 3H), 1.75- 1.91 (m, 1H), 1.57-1.71 (m, 1H), 1.42-1.57 (m, 1H), 1.15 (d, J = 6.1 Hz, 3H); MS: 448 [M + 1] | L |
| 46 | 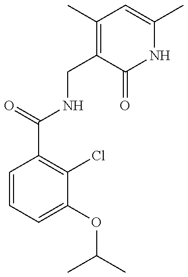 [SMILES: Cc1cc(C)c(CNC(=O)c2cccc(OC(C)C)c2Cl)c(=O)[nH]1] | 2-chloro-N- [(4,6-dimethyl- 2-oxo-1,2- dihydropyridin- 3-yl)methyl]-3- isopropoxy- benzamide | ¹H NMR (400 MHz, DMSO-d6) δ 11.44 (br. s., 1H), 8.23 (t, J = 4.80 Hz, 1H), 7.22-7.28 (m ,1H), 7.12-7.19 (m, 1H), 6.87 (dd, J = 1.26, 7.33 Hz, 1H), 5.85 (s, 1H), 4.65 (td, J = 5.97, 12.06 Hz, 1H), 4.26 (d, J = 5.05 Hz, 2H), 2.18 (s, 3H), 2.10 (s, 3H), 1.28 (d, J = 6.06 Hz, 6H); MS: 349 [M + 1] | I |
| 47 | 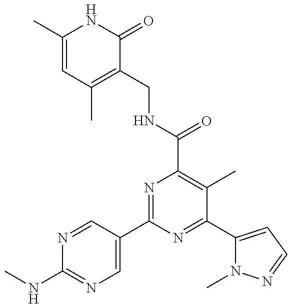 [SMILES: CNc1ncc(-c2nc(C(=O)NCc3c(C)cc(C)[nH]c3=O)c(C)c(-c3ccnn3C)n2)cn1] | N-[(4,6- dimethyl-2- oxo-1,2- dihydropyridin- 3-yl)methyl]-5- methyl-2′- (methylamino)- 6-(1-methyl- 1H-pyrazol-5- yl)-2,5′- bipyrimidine-4- carboxamide | ¹H NMR (400 MHz, methanol -d4) δ 9.27 (br. s., 1H), 7.61 (d, J = 2.0 Hz, 1H), 6.69 (d, J = 2.0 Hz, 1H), 6.11 (s, 1H), 4.55 (s, 2H), 4.44 (br. s., 2H), 4.01 (s, 3H), 3.01 (s, 3H), 2.53 (s, 3H), 2.41 (s, 3H), 2.25 (s, 3H); MS: 460 [M + 1] | L |
| 48 | 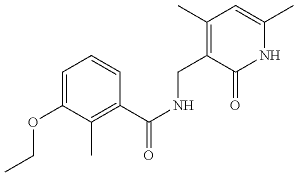 [SMILES: CCOc1cccc(C(=O)NCc2c(C)cc(C)[nH]c2=O)c1C] | N-[(4,6- dimethyl-2- oxo-1,2- dihydropyridin- 3-yl)methyl]-3- ethoxy-2- methylbenzamide | ¹H NMR (400 MHz, chloroform-d) δ 11.68 (s, 1H), 7.03-7.00 (d, J = 8.8 Hz, 1H), 6.84-6.82 (d, J = 7.6 Hz, 1H), 6.76- 6.75 (d, J = 8.4 Hz, 1H), 5.86 (s, 1H), 4.46-4.45 (d, J = 6 Hz, 2H), 3.96-3.91 (dd, J = 13.6 Hz, 2H), 2.32 (s, 3H), 2.17 (s, 3H), 2.12 (s, 3H), 1.35-1.32 (t, J = 7.2 Hz, | I |
| | | | 3H); | |
| | | | MS 315 [M + 1] | |
| 49 | 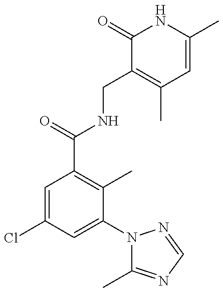 [SMILES: Cc1cc(C)c(CNC(=O)c2cc(Cl)cc(-n3ncnc3C)c2C)c(=O)[nH]1] | 5-chloro-N- [(4,6-dimethyl- 2-oxo-1,2- dihydropyridin- 3-yl)methyl]-2- methyl-3-(5- methyl-1H- 1,2,4-triazol-1- yl)benzamide | ¹H NMR (400 MHz, DMSO-d6) δ 11.48 (s, 1H), 8.50 (t, J = 4.83 Hz, 1H), 8.06 (s, 1H), 7.64 (d, J = 2.20 Hz, 1H), 7.49 (d, J = 2.20 Hz, 1H), 5.86 (s, 1H), 4.27 (d, J = 4.89 Hz, 2H), 2.25 (s, 3H), 2.19 (s, 3H), 2.11 (s, 3H), 1.86 (s, 3H); MS: 386 [M + 1] | F |
| 50 | 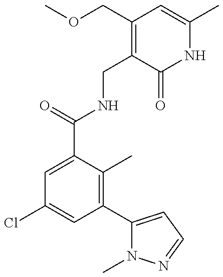 [SMILES: COCc1cc(C)[nH]c(=O)c1CNC(=O)c1cc(Cl)cc(-c2ccnn2C)c1C] | 5-chloro-N-{[4- (methoxymeth- yl)-6-methyl-2- oxo-1,2- dihydropyridin- 3-yl]methyl}-2- methyl-3-(1- methyl-1H- pyrazol-5- yl)benzamide | ¹H NMR (400 MHz, DMSO-d6) δ 2.01 (s, 3 H) 2.16 (s, 3 H) 3.32 (s, 3 H) 3.58 (s, 3 H) 4.25 (d, J = 5.05 Hz, 2 H) 4.46 (s, 2 H) 6.07 (s, 1 H) 6.27 (d, J = 1.77 Hz, 1 H) 7.37 (d, J = 0.51 Hz, 2 H) 7.50 (d, J = 1.77 Hz, 1 H) 8.45 (t, J = 5.05 Hz, 1 H) 11.64 (br. s., 1 H); MS: 415/417 [M + 1], Cl isotope pattern | B |
| 51 | 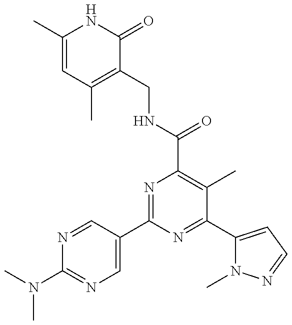 [SMILES: Cc1cc(C)c(CNC(=O)c2nc(-c3cnc(N(C)C)nc3)nc(-c3ccnn3C)c2C)c(=O)[nH]1] | 2′- (dimethylamin- o)-N-[(4,6- dimethyl-2- oxo-1,2- dihydropyridin- 3-yl)methyl]-5- methyl-6-(1- methyl-1H- pyrazol-5-yl)- 2,5′- bipyrimidine-4- carboxamide | ¹H NMR (400 Mhz, DMSO-d6) δ 9.16 (s, 1H), 9.04 (s, 1H), 7.50 (d, J = 2.0 Hz, 1H), 6.66 (d, J = 2.0 Hz, 1H), 5.82 (s, 1H), 4.28 (d, J = 5.6 Hz, 2H), 3.90 (s, 3H), 3.14 (s, 6H), 2.36 (s, 3H), 2.16 (s, 3H), 2.04 (s, 3H); MS: 474 [M + 1] | I |
| 52 | 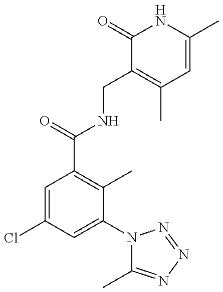 [SMILES: Cc1cc(C)c(CNC(=O)c2cc(Cl)cc(-n3nnnc3C)c2C)c(=O)[nH]1] | 5-chloro-N- [(4,6-dimethyl- 2-oxo-1,2- dihydropyriidn- 3-yl)methyl]-2- methyl-3-(5- methyl-1H- tetrazol-1- yl)benzamide | ¹H NMR (400 MHz, DMSO-d6) δ 11.49 (br. s., 1H), 8.54 (t, J = 4.83 Hz, 1H), 7.82 (d, J = 2.20 Hz, 1H), 7.59 (d, J = 2.20 Hz, 1H), 5.86 (s, 1H), 4.28 (d, J = 4.89 Hz, 2H), 2.40 (s, 3H), 2.20 (s, 3H), 2.11 (s, 3H), 1.86 (s, 3H); MS: 387 [M + 1] | F |
| 53 | 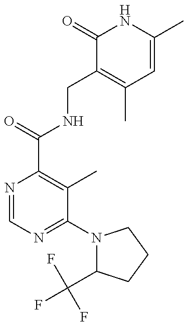 [SMILES: Cc1cc(C)c(CNC(=O)c2ncnc(N3CCCC3C(F)(F)F)c2C)c(=O)[nH]1] | N-[(4,6- dimethyl-2- oxo-1,2- dihydropyridin- 3-yl)methyl]-5- methyl-6-[2- (trifluoromethyl) pyrrolidin-1- yl]pyrimidine- 4-carboxamide | ¹H NMR (400 MHz, DMSO-d6) δ 1.76-1.87 (m, 1 H) 1.94-2.04 (m, 2 H) 2.11 (s, 3 H) 2.20 (s, 3 H) 2.25 (m, J = 8.19 Hz, 1 H) 2.30 (s, 3 H) 3.37-3.45 (m, 1 H) 3.75 (dt, J = 9.60, 7.37 Hz, 1 H) 4.22-4.33 (m, 2 H) 5.61 (quoin, J = 7.93, 7.93, 7.93, 7.93, 4.71 Hz, 1 H) 5.87 (s, 1 H) 8.49 (s, 1 H) 8.53 (t, J = 5.38 Hz, 1 H) 11.50 (br. s., 1 H); MS: 410 [M + 1] | |
| 54 | 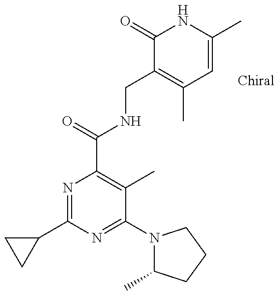 [SMILES: Cc1cc(C)c(CNC(=O)c2nc(C3CC3)nc(N3CCC[C@@H]3C)c2C)c(=O)[nH]1] | 2-cyclopropyl- N-[(4,6- dimethyl-2- oxo-1,2- dihydropyridin- 3-yl)methyl]-5- methyl-6-[(2S)- 2- methylpyrrolidin- 1- yl]pyrimidine- 4-carboxamide | ¹H NMR (400 MHz, chloroform-d) δ 12.35 (br. s., 1 H) ,8.73 (t, J = 5.68 Hz, 1 H), 5.90 (s, 1 H), 4.53 (qd, J = 13.98, 6.32 Hz, 2 H), 4.26-4.36 (m, 1 H), 3.73 (td, J = 9.98, 6.57 Hz, 1 H), 3.39 (t, J = 8.46 Hz, 1 H), 2.42 (s, 3 H), 2.37 (s, 3 H), 2.31 (s, 3 H), 2.07- 2.18 (m, 1 H), 1.87-2.02 (m, 2 H), 1.69-1.77 (m, 1 H), 1.50-1.62 (m, 1 H), 1.19 (d, J = 5.81 Hz, 3 H), 0.94-1.09 (m, 2 H), 0.81- 0.92 (m, 2 H); MS: 396 [M + 1] | H |
| 55 | 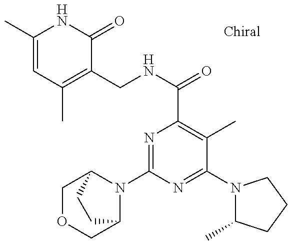 [SMILES: Cc1cc(C)c(CNC(=O)c2nc(N3[C@@H]4CC[C@H]3COC4)nc(N3CCC[C@@H]3C)c2C)c(=O)[nH]1] | N-[(4,6- dimethyl-2- oxo-1,2- dihydropyridin- 3-yl)methyl]-5- methyl-6-[(2S)- 2- methylpyrrolidin- 1-yl]-2- [(1R,5S)-3- oxa-8- azabicyclo[3.2.1] oct-8- yl]pyrimidine- 4-carboxamide | ¹H NMR (400 MHz, DMSO-d6) δ 8.64 (s, 1H), 5.86 (s, 1H), 4.47 (s, 2H), 4.19-4.25 (m, 3H), 3.46- 3.78 (m, 6H), 2.16-2.20 (m, 6H), 2.10 (s, 3H), 2.05- 2.07 (m, 1H), 1.80-1.91 (m, J = 7.8 Hz, 5H), 1.66- 1.67 (m, 1H), 1.50-1.51 (m, 1H), 1.14-1.15 (m, J = 6.0 Hz, 3H).; MS: 467 [M + 1] | M |
| 56 | 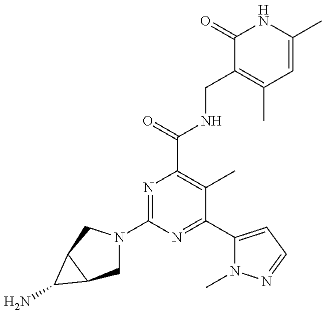 [SMILES: Cc1cc(C)c(CNC(=O)c2nc(N3C[C@@H]4[C@@H](N)[C@@H]4C3)nc(-c3ccnn3C)c2C)c(=O)[nH]1] | 2-[(1R,5S,6s)- 6-amino-3- azabicyclo[3.1.0] hex-3-yl]-N- [(4,6-dimethyl- 2-oxo-1,2- dihydropyridin- 3-yl)methyl]-5- methyl-6-(1- methyl-1H- pyrazol-5- yl)pyrimidine- 4-carboxamide | MS: 449 [M + 1] | M |
| 57 | 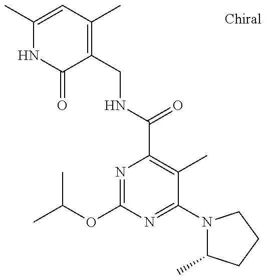 [SMILES: Cc1cc(C)c(CNC(=O)c2nc(OC(C)C)nc(N3CCC[C@@H]3C)c2C)c(=O)[nH]1] | N-[(4,6- dimethyl-2- oxo-1,2- dihydropyridin- 3-yl)methyl]-2- isopropoxy-5- methyl-6-[(2S)- 2- methylpyrrolidin- 1- yl]pyrimidine- 4-carboxamide | ¹H NMR (400 MHz, chloroform-d) δ 1.22 (d, J = 5.87 Hz, 3 H) 1.31 (dd, J = 10.51, 6.11 Hz, 6 H) 1.52-1.65 (m, 1 H) 1.76 (d, 3 H) 1.95 (ddd, J = 8.80, 5.87, 3.18 Hz, 1 H) 2.08- 2.21 (m, 1 H) 2.30 (s, 3 H) 2.34 (s, 3 H) 2.40 (s, 3 H) 3.49 (t, J = 7.95 Hz, 1 H) 3.77 (td, J = 9.66, 6.85 Hz, 1 H) 4.31-4.44 (m, 1 H) 4.44-4.62 (m, 2 H) 5.16 (dt, J = 12.29, 6.20 Hz, 1 H) 5.87 (s, 1 H) 8.61 (br. s., 1 H) 12.35 (br. s., 1 H); MS: 414 [M + 1] | M |
| 58 | 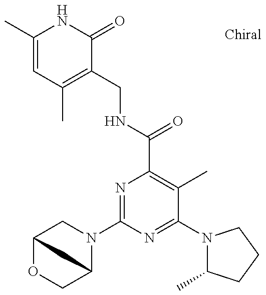 [SMILES: Cc1cc(C)c(CNC(=O)c2nc(N3C[C@@H]4C[C@H]3CO4)nc(N3CCC[C@@H]3C)c2C)c(=O)[nH]1] | N-[(4,6- dimethyl-2- oxo-1,2- dihydropyridin- 3-yl)methyl]-5- methyl-6-[(2S)- 2- methylpyrrolidin- 1-yl]-2- [(1S,4S)-2- oxa-5- azabicyclo[2.2.1] hept-5- yl]pyrimidine- 4-carboxamide | ¹H NMR (400 MHz, DMSO-d6) δ 8.67 (s, 1H), 5.86 (s, 1H), 4.81 (s, 1H), 4.60 (s, 1H), 4.23-4.28 (m, J = 5.3 Hz, 3H), 3.66-3.75 (m, 2H), 3.60-3.62 (m, 1H), 3.43-3.45 (m, 1H), 3.34- 3.38 (m, 1H), 2.53 (s, 1H), 2.19 (s, 3H), 2.06-2.13 (m, 6H), 2.04-2.05 (m, 1H), 1.79-1.87 (m, 3H), 1.65- 1.69 (m, 1H), 1.49-1.55 (m, 1H), 1.13-1.15 (m, J = 6.0 Hz, 3H).; MS: 453 [M + 1] | M |
| 59 | 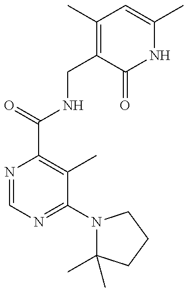 [SMILES: Cc1cc(C)c(CNC(=O)c2ncnc(N3CCCC3(C)C)c2C)c(=O)[nH]1] | N-[(4,6- dimethyl-2- oxo-1,2- dihydropyridin- 3-yl)methyl]-6- (2,2- dimethylpyrroli- din-1-yl)-5- methylpyrimidine- 4- carboxamide | ¹H NMR (400 MHz, chloroform-d) δ 1.58 (s, 7 H) 1.79-1.87 (m, 2 H) 1.87-1.98 (m, 2 H) 2.30 (s, 3 H) 2.37 (s, 3 H) 2.42 (s, 3 H) 3.67 (t, J = 6.11 Hz, 2 H) 4.52 (d, J = 5.87 Hz, 2 H) 5.90 (s, 1 H) 8.29 (s, 1 H) 8.76 (br. s., 1 H) 11.95 (br. s., 1 H); MS: 370 [M + 1] | H |
| 60 | 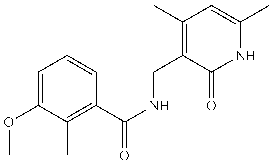 [SMILES: COc1cccc(C(=O)NCc2c(C)cc(C)[nH]c2=O)c1C] | N-[(4,6- dimethyl-2- oxo-1,2- dihydropyridin- 3-yl)methyl]-3- methoxy-2- methylbenz- amide | ¹H NMR (400 MHz, chloroform-d) δ 10.81 (s, 1H), 7.07-7.05 (d, J = 8 Hz, 1H), 7.03-6.99 (d, J = 14 Hz, 1H), 6.85-6.84 (d, J = 7.2 Hz, 1H), 6.78-6.76 (d, J = 8.4 Hz, 1H), 5.85 (s, 1H), 4.46-4.44 (d, J = 6 Hz, 2H), 3.74 (s, 3H), 2.32 (s, 3H), 2.16 (s, 3H), 2.13 (s, | I |
| | | | 3H).; | |
| | | | MS: 301 [M + 1] | |
| 61 | 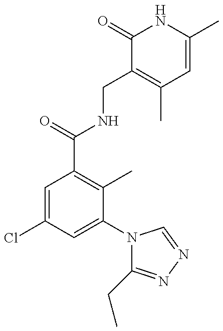 [SMILES: CCc1nncn1-c1cc(Cl)cc(C(=O)NCc2c(C)cc(C)[nH]c2=O)c1C] | 5-chloro-N- [(4,6-dimethyl- 2-oxo-1,2- dihydropyridin- 3-yl)methyl]-3- (3-ethyl-4H- 1,2,4-triazol-4- yl)-2- methylbenz- amide | ¹H NMR (600 MHz, DMSO-d6) δ 11.51 (br. s., 1H), 8.57 (s, 1H), 8.50 (t, J = 4.98 Hz, 1H), 7.67 (d, J = 2.21 Hz, 1H), 7.49 (d, J = 2.21 Hz, 1H), 5.87 (s, 1H), 4.27 (d, J = 4.98 Hz, 2H), 2.44-2.50 (m, 2H), 2.19 (s, 3H), 2.11 (s, 3H), 1.86 (s, 3H), 1.12 (t, J = 7.46 Hz, 3H); MS: 400 [M + 1] | F |
| 62 | 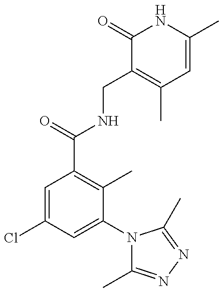 [SMILES: Cc1cc(C)c(CNC(=O)c2cc(Cl)cc(-n3c(C)nnc3C)c2C)c(=O)[nH]1] | 5-chloro-N- [(4,6-dimethyl- 2-oxo-1,2- dihydropyridin- 3-yl)methyl]-3- (3,5-dimethyl- 4H-1,2,4- triazol-4-yl)-2- methylbenz- amide | ¹H NMR (600 MHz, DMSO-d6) δ 8.51 (t, J = 4.98 Hz, 1H), 7.62 (d, J = 2.21 Hz, 1H), 7.50 (d, J = 2.21 Hz, 1H), 5.87 (s, 1H), 4.27 (d, J = 4.98 Hz, 2H), 2.20 (s, 3H), 2.11 (s, 3H), 2.06 (s, 6H), 1.80 (s, 3H); MS: 400 [M + 1] | F |
| 63 | 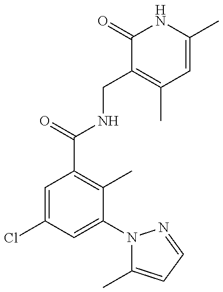 [SMILES: Cc1cc(C)c(CNC(=O)c2cc(Cl)cc(-n3nccc3C)c2C)c(=O)[nH]1] | 5-chloro-N- [(4,6-dimethyl- 2-oxo-1,2- dihydropyridin- 3-yl)methyl]-2- methyl-3-(5- methyl-1H- pyrazol-1- yl)benzamide | ¹H NMR (400 MHz, DMSO-d6) δ 11.47 (s, 1H), 8.48 (t, J = 4.93 Hz, 1H), 7.56 (d, J = 1.52 Hz, 1H), 7.47 (d, J = 2.27 Hz, 1H), 7.43 (d, J = 2.27 Hz, 1H), 6.26 (d, J = 1.01 Hz, 1H), 5.86 (s, 1H), 4.27 (d, J = 4.80 Hz, 2H), 2.19 (s, 3H), 2.11 (s, 3H), 2.07 (s, 3H), 1.81 (s, 3H); MS: 385 [M + 1] | F |
| 64 | 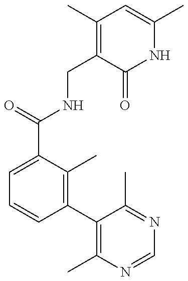 [SMILES: Cc1cc(C)c(CNC(=O)c2cccc(-c3c(C)ncnc3C)c2C)c(=O)[nH]1] | N-[(4,6- dimethyl-2- oxo-1,2- dihydropyridin- 3-yl)methyl]-3- (4,6- dimethylpyrimi- din-5-yl)-2- methylbenz- amide | ¹H NMR (400 MHz, methanol-d4) δ 8.90 (s, 1H), 7.46-7.38 (m, 2H), 7.19 (d, J = 6.8 Hz, 1H), 6.12 (s, 1H), 4.51 (s, 2H), 3.33 (s, 3H), 2.40 (s, 3H), 2.26 (s, 3H), 2.21 (s, 6H), 2.01 (s, 3H).; MS: 377 [M + 1] | B |
| 65 | 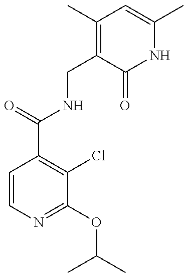 [SMILES: Cc1cc(C)c(CNC(=O)c2ccnc(OC(C)C)c2Cl)c(=O)[nH]1] | 3-chloro-N- [(4,6-dimethyl- 2-oxo-1,2- dihydropyridin- 3-yl)methyl]-2- isopropoxyisoni- cotinamide | ¹H NMR (400 MHz, DMSO-d6) δ 11.46 (br. s., 1H), 8.50 (t, J = 4.93 Hz, 1H), 8.08 (d, J = 5.05 Hz, 1H), 6.89 (d, J = 5.05 Hz, 1H), 5.86 (s, 1H), 5.30 (quin, J = 6.19 Hz, 1H), 4.27 (d, J = 5.05 Hz, 2H), 2.18 (s, 3H), 2.11 (s, 3H), 1.31 (d, J = 6.06 Hz, 6H); MS: 350 [M + 1] | I |
| 66 | 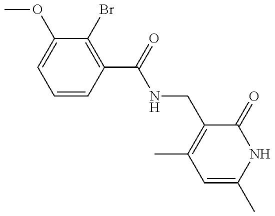 [SMILES: COc1cccc(C(=O)NCc2c(C)cc(C)[nH]c2=O)c1Br] | 2-bromo-N- [(4,6-dimethyl- 2-oxo-1,2- dihydropyridin- 3-yl)methyl]-3- methoxybenza- mide | MS: 365 [M + 1] | I |
| 67 | 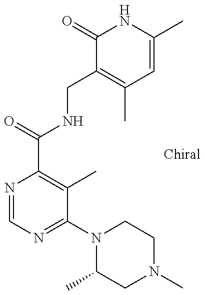 [SMILES: Cc1cc(C)c(CNC(=O)c2ncnc(N3CCN(C)C[C@@H]3C)c2C)c(=O)[nH]1] | N-[(4,6- dimethyl-2- oxo-1,2- dihydropyridin- 3-yl)methyl]-6- [(2S)-2,4- dimethylpipera- zin-1-yl]-5- methylpyrimidine- 4- carboxamide | ¹H NMR (400 MHz, DMSO-d6) δ 1.19 (d, J = 6.57 Hz, 3 H) 1.97-2.09 (m, 1 H) 2.11 (s, 3 H) 2.16 (s, 4 H) 2.20 (s, 3 H) 2.25 (s, 3 H) 2.55 (br.s., 1 H) 2.69 (d, J = 11.62 Hz, 1 H) 3.22-3.29 (m, 1 H) 3.47 (d, J = 13.39 Hz, 1 H) 3.97- 4.11 (m, 1 H) 4.27 (d, J = 5.56 Hz, 1 H) 5.86 (s, 1 H) 8.50 (s, 1 H) 8.54 (t, J = 5.56 Hz, 1 H) 11.50 (br. s., 1 H); MS: 385 [M + 1] | H |
| 68 | 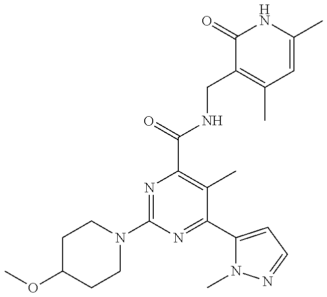 | N-[(4,6- dimethyl-2- oxo-1,2- dihydropyridin- 3-yl)methyl]-2- (4- methoxypiperi- din-1-yl)-5- methyl-6-(1- methyl-1H- pyrazol-5- yl)pyrimidine- 4-carboxamide | MS: 466 [M + 1] | M |
| 69 | 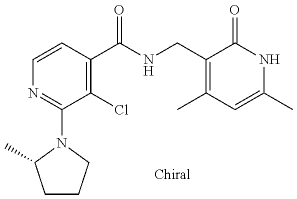 | 3-chloro-N- [(4,6-dimethyl- 2-oxo-1,2- dihydropyridin- 3-yl)methyl]-2- [(2S)-2- methylpyrrolidin- 1- yl]isonicotinamide | ¹H NMR (400 MHz, DMSO-d60 δ 1.09 (d, J = 5.81 Hz, 3 H) 1.47-1.59 (m, 1 H) 1.61-1.75 (m, 1 H) 1.85-1.95 (m, 1 H) 2.06-2.15 (m, 4 H) 2.18 (s, 3 H) 3.31 (m, J = 17.68, 2.02 Hz, 1 H) 3.82 (td, J = 9.85, 6.57 Hz, 1 H) 4.26 (d, J = 5.05 Hz, 2 H) 4.28- 4.38 (m, 1 H) 5.86 (s, 1 H) 6.65 (d, J = 4.80 Hz, 1 H) | H |
| | | | 8.05 (d, J = 4.80 Hz, 1 H) | |
| | | | 8.39 (t, J = 4.93 Hz, 1 H) | |
| | | | 11.46 (br. s., 1 H); | |
| | | | MS: 375 [M + 1] | |
| 70 | 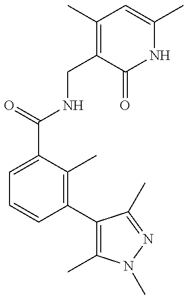 [SMILES: Cc1cc(C)c(CNC(=O)c2cccc(-c3c(C)nn(C)c3C)c2C)c(=O)[nH]1] | N-[(4,6- dimethyl-2- oxo-1,2- dihydropyridin- 3-yl)methyl]-2- methyl-3- (1,3,5- trimethyl-1H- pyrazol-4- yl)benzamide | ¹H NMR (400 MHz, methanol-d4) δ 7.32-7.25 (m, 2H), 7.15 (d, J = 7.3 Hz, 1H), 6.13 (s, 1H), 4.50 (s, 2H), 3.78 (s, 3H), 2.40 (s, 3H), 2.26 (s, 3H), 2.10 (s, 3H), 2.06 (s, 3H), 1.99 (s, 3H); MS: 379 [M + 1] | B |
| 71 | 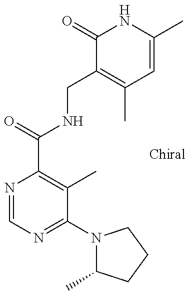 [SMILES: Cc1cc(C)c(CNC(=O)c2ncnc(N3CCC[C@@H]3C)c2C)c(=O)[nH]1] | N-[(4,6- dimethyl-2- oxo-1,2- dihydropyridin- 3-yl)methyl]-5- methyl-6-[(2S)- 2- methylpyrrolidin- 1- yl]pyrimidine- 4-carboxamide | ¹H NMR (400 MHz, chloroform-d) δ 13.26 (br. s., 1 H) 8.82 (t, J = 5.81 Hz, 1 H), 8.32 (s, 1 H), 5.90 (s, 1 H), 4.51-4.61 (m, 1 H), 4.39-4.49 (m, 1 H), 4.26- 4.38 (m, 1 H), 3.67-3.80 (m, 1 H), 3.39 (t, J = 8.21 Hz, 1 H), 2.45 (s, 3 H), 2.33 (d, J = 3.03 Hz, 6 H), 2.09-2.19 (m, 1 H), 1.86- 1.97 (m, 1 H), 1.63-1.77 (m, 1 H), 1.49-1.63 (m, 1 H), 1.18 (d, J = 6.06 Hz, 3 H); MS: 356 [M + 1] | H |
| 72 | 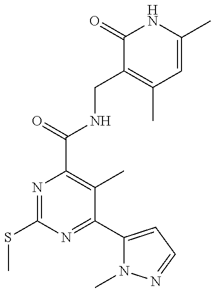 [SMILES: CSc1nc(C(=O)NCc2c(C)cc(C)[nH]c2=O)c(C)c(-c2ccnn2C)n1] | N-[(4,6- dimethyl-2- oxo-1,2- dihydropyridin- 3-yl)methyl]-5- methyl-6-(1- methyl-1H- pyrazol-5-yl)-2- (methylthio)pyri- midine-4- carboxamide | ¹H NMR (400 MHz, DMSO-d6) δ 11.56 (br. s., 1H), 8.76 (t, J = 5.14 Hz, 1H), 7.57 (d, J = 1.96 Hz, 1H), 6.70 (d, J = 1.96 Hz, 1H), 5.88 (s, 1H), 4.31 (d, J = 5.62 Hz, 2H), 3.90 (s, 3H), 2.55 (s, 3H), 2.29 (s, 3H), 2.21 (s, 3H), 2.12 (s, 3H); MS: 399 [M + 1] | M |
| 73 | 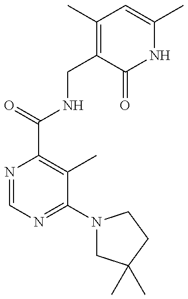 [SMILES: Cc1cc(C)c(CNC(=O)c2ncnc(N3CCC(C)(C)C3)c2C)c(=O)[nH]1] | N-[(4,6- dimethyl-2- oxo-1,2- dihydropyridin- 3-yl)methyl]-6- (3,3- dimethylpyrroli- din-1-yl)-5- methylpyrimidine- 4- carboxamide | ¹H NMR (400 MHz, methanol -d4) δ 1.12 (s, 6 H) 1.75 (t, 2 H) 2.24 (s, 3 H) 2.36 (s, 3 H) 2.37 (s, 3 H) 3.44 (s, 2 H) 3.78 (t, 2 H) 4.47 (s, 2 H) 6.09 (s, 1 H) 8.24 (s, 1 H); MS: 370 [M + 1] | H |
| 74 | 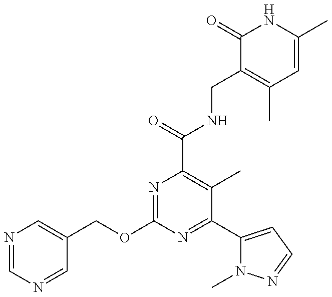 [SMILES: Cc1cc(C)c(CNC(=O)c2nc(OCc3cncnc3)nc(-c3ccnn3C)c2C)c(=O)[nH]1] | N-[(4,6- dimethyl-2- oxo-1,2- dihydropyridin- 3-yl)methyl]-5- methyl-6-(1- methyl-1H- pyrazol-5-yl)-2- (pyrimidin-5- ylmethoxy)pyri- midine-4- carboxamide | ¹H NMR (400 MHz, DMSO-d6) δ 11.54 (br. s., 1H), 9.17 (s, 1H), 8.92 (s, 2H), 8.72 (t, J = 5.14 Hz, 1H), 7.57 (d, J = 1.96 Hz, 1H), 6.69 (d, J = 1.96 Hz, 1H), 5.88 (s, 1H), 5.50 (s, 2H), 4.32 (d, J = 5.38 Hz, 2H), 3.88 (s, 3H), 2.29 (s, 3H), 2.21 (s, 3H), 2.11 (s, 3H); MS: 461 [M + 1] | M |
| 75 | 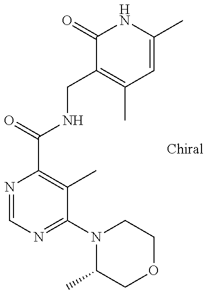 [SMILES: Cc1cc(C)c(CNC(=O)c2ncnc(N3CCOC[C@@H]3C)c2C)c(=O)[nH]1] | N-[(4,6- dimethyl-2- oxo-1,2- dihydropyridin- 3-yl)methyl]-5- methyl-6-[(3S)- 3- methylmorpholin- 4- yl]pyrimidine- 4-carboxamide | ¹H NMR (400 MHz, DMSO-d6) δ 1.16 (d, J = 6.57 Hz, 3 H) 2.11 (s, 3 H) 2.20 (s, 3 H) 2.27 (s, 3 H) 3.34-3.41 (m, 2 H) 3.51-3.60 (m, 2 H) 3.62- 3.70 (m, 1 H) 3.82 (d, J = 11.12 Hz, 1 H) 3.88- 3.99 (m, 1 H) 4.28 (d, J = 4.80 Hz, 2 H) 5.86 (s, 1 H) 8.53 (s, 1 H) 8.54-8.59 (m, 1 H) 11.50 (br. s., 1 H); MS: 372 [M + 1] | H |
| 76 | 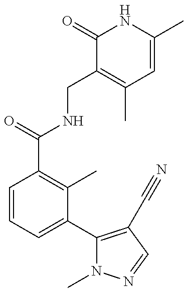 [SMILES: Cc1cc(C)c(CNC(=O)c2cccc(-c3c(C#N)cnn3C)c2C)c(=O)[nH]1] | 3-(4-cyano-1- methyl-1H- pyrazol-5-yl)- N-[(4,6- dimethyl-2- oxo-1,2- dihydropyridin- 3-yl)methyl]-2- methylbenzamide | ¹H NMR (400 MHz, DMSO-d6) δ 11.33 (br. s., 1H), 8.30 (t, J = 4.77 Hz, 1H), 8.10 (s, 1H), 7.30- 7.40 (m, 3H), 5.79 (s, 1H), 4.18-4.28 (m, 2H), 3.56 (s, 3H), 2.14 (s, 3H), 2.04 (s, 3H), 1.99 (s, 3H); MS: 376 [M + 1] | B |
| 77 | 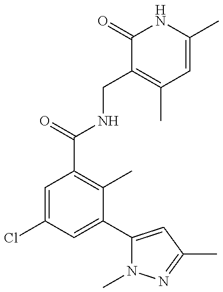 [SMILES: Cc1cc(-c2cc(Cl)cc(C(=O)NCc3c(C)cc(C)[nH]c3=O)c2C)n(C)n1] | 5-chloro-N- [(4,6-dimethyl- 2-oxo-1,2- dihydropyridin- 3-yl)methyl]-3- (1,3-dimethyl- 1H-pyrazol-5- yl)-2- methylbenz- amide | ¹H NMR (400 MHz, DMSO-d6) δ 11.47 (br. s., 1H), 8.39 (t, J = 4.9 Hz, 1H), 7.34 (s, 2H), 6.04 (s, 1H), 5.86 (s, 1H), 4.27 (d, J = 4.9 Hz, 2H), 3.49 (s, 3H), 2.19 (s, 3H), 2.17 (s, 3H), 2.11 (s, 3H), 2.01 (s, 3H); MS: 399 [M + 1] | A |
| 78 | 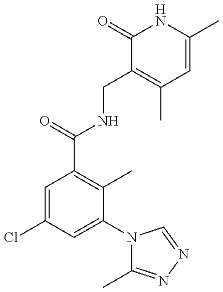 [SMILES: Cc1cc(C)c(CNC(=O)c2cc(Cl)cc(-n3cnnc3C)c2C)c(=O)[nH]1] | 5-chloro-N- [(4,6-dimethyl- 2-oxo-1,2- dihydropyridin- 3-yl)methyl]-2- methyl-3-(3- methyl-4H- 1,2,4-triazol-4- yl)benzamide | ¹H NMR (600 MHz, DMSO-d6) δ 8.57 (s, 1H), 8.49 (t, J = 5.09 Hz, 1H), 7.65 (d, J = 2.03 Hz, 1H), 7.49 (d, J = 2.03 Hz, 1H), 5.87 (s, 1H), 4.27 (d, J = 5.09 Hz, 2H), 2.19 (s, 3H), 2.16 (s, 3H), 2.11 (s, 3H), 1.87 (s, 3H); MS: 386 [M + 1] | F |
| 79 | 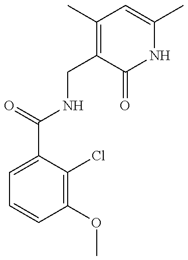 [SMILES: COc1cccc(C(=O)NCc2c(C)cc(C)[nH]c2=O)c1Cl] | 2-chloro-N- [(4,6-dimethyl- 2-oxo-1,2- dihydropyridin- 3-yl)methyl]-3- methoxybenza- mide | ¹H NMR (400 MHz, DMSO-d6) δ 11.45 (br. s., 1 H) 8.25 (t, J = 4.93 Hz, 1 H) 7.25-7.33 (m, 1 H) 7.14 (d, J = 8.08 Hz, 1 H) 6.90 (d, J = 7.58 Hz, 1 H) 5.86 (s, 1 H) 4.27 (d, J = 5.05 Hz, 2 H) 3.85 (s, 3 H) 2.18 (s, 3 H) 2.11 (s, 3 H); MS: 321 [M + 1] | I |
| 80 | 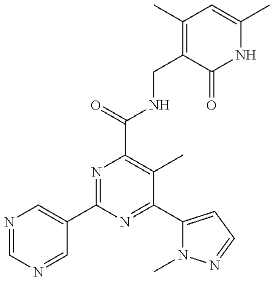 [SMILES: Cc1cc(C)c(CNC(=O)c2nc(-c3cncnc3)nc(-c3ccnn3C)c2C)c(=O)[nH]1] | N-[(4,6- dimethyl-2- oxo-1,2- dihydropyridin- 3-yl)methyl]-5- methyl-6-(1- methyl-1H- pyrazol-5-yl)- 2,5′- bipyrimidine-4- carboxamide | ¹H NMR (400 MHz, methanol -d4) δ 9.68 (s, 2H), 9.21 (s, 1H), 7.55 (d, J = 2.0 Hz, 1H), 6.66 (d, J = 2.0 Hz, 1H), 6.05 (s, 1H), 4.50 (s, 2H), 3.96 (s, 3H), 2.53 (s, 3H), 2.35 (s, 3H), 2.18 (s, 3H); MS: 431 [M + 1] | L |
| 81 | 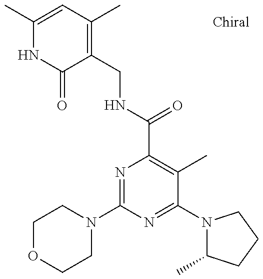 [SMILES: Cc1cc(C)c(CNC(=O)c2nc(N3CCOCC3)nc(N3CCC[C@@H]3C)c2C)c(=O)[nH]1] | N-[(4,6- dimethyl-2- oxo-1,2- dihydropyridin- 3-yl)methyl]-5- methyl-6-[(2S)- 2- methylpyrrolidin- 1-yl]-2- morpholin-4- ylpyrimidine-4- carboxamide | ¹H NMR (400 MHz, DMSO-d6) δ 1.16 (d, J = 5.87 Hz, 3 H) 1.19-1.29 (m, 1 H) 1.44-1.59 (m, 1 H) 1.67 (br. s., 1 H) 1.88 (br. s., 1 H) 2.10 (s, 4 H) 2.17 (s, 3 H) 2.20 (s, 3 H) 3.37-3.49 (m, 1 H) 3.50- 3.78 (m, 9 H) 4.09-4.44 (m, 3 H) 5.86 (s, 1 H) 8.60 (t, J = 5.26 Hz, 1 H) 11.56 (br. s., 1 H); MS: 441 [M + 1] | M |
| 82 | 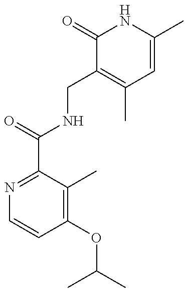 [SMILES: Cc1cc(C)c(CNC(=O)c2nccc(OC(C)C)c2C)c(=O)[nH]1] | N-[(4,6- dimethyl-2- oxo-1,2- dihydropyridin- 3-yl)methyl]-4- isopropoxy-3- methylpyridine- 2- carboxamide | ¹H NMR (400 MHz, DMSO-d6) δ 11.46 (br. s., 1H), 8.51 (t, J = 5.75 Hz, 1H), 8.24 (d, J = 5.62 Hz, 1H), 7.11 (d, J = 5.62 Hz, 1H), 5.86 (s, 1H), 4.76 (td, J = 6.01, 12.07 Hz, 1H), 4.27 (d, J = 5.87 Hz, 2H), 2.35 (s, 3H), 2.21 (s, 3H), 2.11 (s, 3H), 1.30 (d, J = 5.99 Hz, 6H); MS: 330 [M + 1] | J |
| 83 | 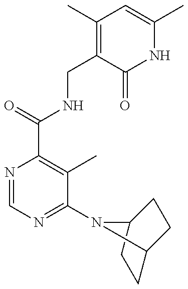 [SMILES: Cc1cc(C)c(CNC(=O)c2ncnc(N3C4CCC3CC4)c2C)c(=O)[nH]1] | 6-(7- azabicyclo[2.2.1] hept-7-yl)-N- [(4,6-dimethyl- 2-oxo-1,2- dihydropyridin- 3-yl)methyl]-5- methylpyrimidine- 4- carboxamide | ¹H NMR (400 MHz, methanol -d4) δ 1.53 (d, J = 7.09 Hz, 4 H) 1.76-1.89 (m, 4 H) 2.24 (s, 3 H) 2.35 (s, 3 H) 2.37 (s, 3 H) 4.47 (s, 2 H) 4.56 (br. s., 2 H) 6.09 (s, 1 H) 8.37 (s, 1 H); MS: 368 [M + 1] | H |
| 84 | 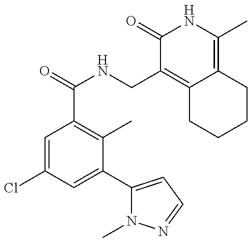 [SMILES: Cc1[nH]c(=O)c(CNC(=O)c2cc(Cl)cc(-c3ccnn3C)c2C)c2c1CCCC2] | 5-chloro-2- methyl-N-[(1- methyl-3-oxo- 2,3,5,6,7,8- hexahydroiso- quinolin-4- yl)methyl]-3-(1- methyl-1H- pyrazol-5- yl)benzamide | ¹H NMR (400 MHz, DMSO-d6) δ 1.65 (br. s., 4 H) 1.98-2.06 (s, 3 H) 2.11 (s, 3 H) 2.38 (br. s., 2 H) 2.70-2.78 (m, 2 H) 3.59 (s, 3 H) 4.31 (d, J = 4.77 Hz, 2 H) 6.28 (d, J = 1.83 Hz, 1 H) 7.37 (d, J = 2.20 Hz, 2 H) 7.51 (d, J = 1.83 Hz, 1 H) 8.36 (t, J = 4.71 Hz, 1 H) 11.50 (br. s., 1 H); MS: 425 [M + 1] | B |
| 85 | 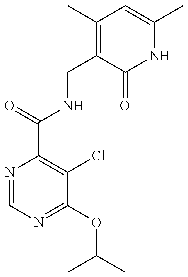 [SMILES: Cc1cc(C)c(CNC(=O)c2ncnc(OC(C)C)c2Cl)c(=O)[nH]1] | 5-chloro-N- [(4,6-dimethyl- 2-oxo-1,2- dihydropyridin- 3-yl)methyl]-6- isopropoxypyri- midine-4- carboxamide | ¹H NMR (400 MHz, chloroform-d) δ 12.92 (br. s., 1 H), 8.73 (t, J = 5.69 Hz, 1 H), 8.47 (s, 1 H), 5.95 (s, 1 H), 5.43 (m, 1 H), 4.54 (d, J = 6.11 Hz, 2 H), 2.38 (s, 3 H), 2.33 (s, 3 H), 1.41 (d, J = 6.24 Hz, 6 H); MS: 351 [M + 1] | J |
| 86 | 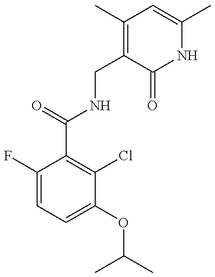 [SMILES: Cc1cc(C)c(CNC(=O)c2c(F)ccc(OC(C)C)c2Cl)c(=O)[nH]1] | 2-chloro-N- [(4,6-dimethyl- 2-oxo-1,2- dihydropyridin- 3-yl)methyl]-6- fluoro-3- isopropoxyben- zamide | ¹H NMR (700 MHz, DMSO-d6) δ 1.21-1.34 (m, 6 H) 2.05-2.14 (m, 3 H) 2.14-2.23 (m, 3 H) 4.29 (d, J = 4.84 Hz, 2 H) 4.60 (spt, J = 5.98 Hz, 1 H) 5.88 (s, 1 H) 7.13-7.21 (m, 2 H) 8.58 (br. s., 1 H) 11.48 (br. s, 1 H); MS: 367 [M + 1] | I |
| 87 | 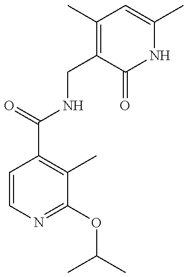 [SMILES: Cc1cc(C)c(CNC(=O)c2ccnc(OC(C)C)c2C)c(=O)[nH]1] | N-[(4,6- dimethyl-2- oxo-1,2- dihydropyridin- 3-yl)methyl]-2- isopropoxy-3- methylisonicoti- namide | ¹H NMR (600 MHz, DMSO-d6) δ 8.32 (t, J = 4.98 Hz, 1H), 7.94-7.97 (m, 1H), 6.75 (d, J = 4.98 Hz, 1H), 5.86 (s, 1H), 5.24 (td, J = 6.12, 12.37 Hz, 1H), 4.25 (d, J = 4.98 Hz, 2H), 2.18 (s, 3H), 2.10 (s, 3H), 2.03 (s, 3H), 1.28 (d, J = 6.08 Hz, 6H); MS: 330 [M + 1] | J |
| 88 | 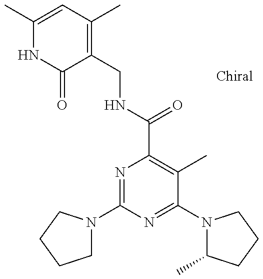 [SMILES: Cc1cc(C)c(CNC(=O)c2nc(N3CCCC3)nc(N3CCC[C@@H]3C)c2C)c(=O)[nH]1] | N-[(4,6- dimethyl-2- oxo-1,2- dihydropyridin- 3-yl)methyl]-5- methyl-6-[(2S)- 2- methylpyrrolidin- 1-yl]-2- pyrrolidin-1- ylpyrimidine-4- carboxamide | ¹H NMR (400 MHz, methanol -d4) δ 1.23 (d, J = 5.87 Hz, 3 H) 1.49-1.82 (m, 2 H) 1.86-2.04 (m ,5 H) 2.08-2.17 (m, 1 H) 2.18 (s, 3 H) 2.23 (s, 3 H) 2.37 (s, 3 H) 3.40-3.61 (m, 5 H) 3.75 (td, J = 9.72, 6.72 Hz, 1 H) 4.32-4.42 (m, 1 H) 4.45 (d, J = 3.91 Hz, 2 H) 6.08 (s, 1 H); MS: 425 [M + 1] | M |
| 89 | 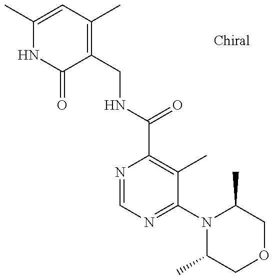 [SMILES: Cc1cc(C)c(CNC(=O)c2ncnc(N3[C@@H](C)COC[C@@H]3C)c2C)c(=O)[nH]1] | 6-[(3S,5S)-3,5- dimethylmorph- olin-4-yl]-N- [(4,6-dimethyl- 2-oxo-1,2- dihydropyridin- 3-yl)methyl]-5- methylpyrimidine- 4- carboxamide | ¹H NMR (700 MHz, DMSO-d6) δ 8.73 (1 H, s), 8.61 (1 H, t, J = 5.50 Hz), 5.87 (1 H, s), 4.29-4.34 (1 H, m), 4.24-4.28 (1 H, m), 3.80 (2H, dd, J = 11.00, 3.08 Hz), 2.32 (3 H, s), 2.21 (3 H, s), 2.11 (3 H, s), 0.86 (6 H, d, J = 6.38 Hz); MS: 386 [M + 1] | H |
| 90 | 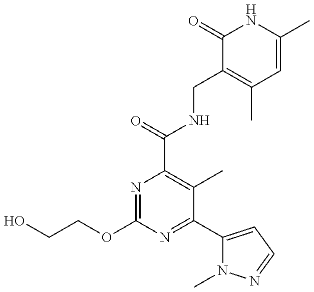 [SMILES: Cc1cc(C)c(CNC(=O)c2nc(OCCO)nc(-c3ccnn3C)c2C)c(=O)[nH]1] | N-[(4,6- dimethyl-2- oxo-1,2- dihydropyridin- 3-yl)methyl]-2- (2- hydroxyethoxy)- 5-methyl-6- (1-methyl-1H- pyrazol-5- yl)pyrimidine- 4-carboxamide | ¹H NMR (400 MHz, DMSO-d6) δ 11.53 (s, 1H), 8.67 (t, J = 5.43 Hz, 1H), 7.56 (d, J = 1.77 Hz, 1H), 6.68 (d, J = 2.02 Hz, 1H), 5.88 (s, 1H), 4.89 (t, J = 5.68 Hz, 1H), 4.26-4.38 (m, 4H), 3.92 (s, 3H), 3.72 (q, J = 5.47 Hz, 2H), 2.29 (s, 3H), 2.21 (s, 3H), 2.12 (s, 3H); MS: 413 [M + 1] | M |
| 91 | 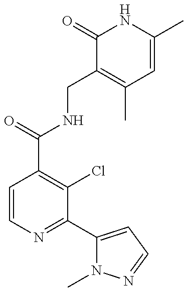 [SMILES: Cc1cc(C)c(CNC(=O)c2ccnc(-c3ccnn3C)c2Cl)c(=O)[nH]1] | 3-chloro-N- [(4,6-dimethyl- 2-oxo-1,2- dihydropyridin- 3-yl)methyl]-2- (1-methyl-1H- pyrazol-5- yl)isonicotinamide | ¹H NMR (400 MHz, DMSO-d6) δ 2.11 (s, 3 H) 2.20 (s, 3 H) 3.79 (s, 3 H) 4.30 (d, J = 4.89 Hz, 2 H) 5.87 (s, 1 H) 6.59 (d, J = 1.96 Hz, 1 H) 7.44 (d, J = 4.89 Hz, 1 H) 7.53 (d, J = 1.96 Hz, 1 H) 8.65 (d, J = 4.65 Hz, 2 H) 11.46 (br. s., 1 H); MS: 372 [M + 1] | A |
| 92 | 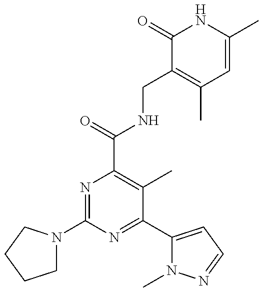 [SMILES: Cc1cc(C)c(CNC(=O)c2nc(N3CCCC3)nc(-c3ccnn3C)c2C)c(=O)[nH]1] | N-[(4,6- dimethyl-2- oxo-1,2- dihydropyridin- 3-yl)methyl]-5- methyl-6-(1- methyl-1H- pyrazol-5-yl)-2- pyrrolidin-1- ylpyrimidine-4- carboxamide | ¹H NMR (600 MHz, DMSO-d6) δ 11.49 (br. s., 1H), 8.25 (t, J = 4.98 Hz, 1H), 7.43-7.46 (m, 2H), 7.37-7.41 (m, 2H), 7.30- 7.35 (m, 1H), 7.08 (s, 1H), 6.82 (s, 1H), 5.86 (s, 1H), 5.14 (s, 2H), 4.26 (d, J = 4.98 Hz, 2H), 2.93 (s, 2H), 2.15 (s, 3H), 2.11 (s, 3H), 1.39 (s, 6H); MS: 422 [M + 1] | M |
| 93 | 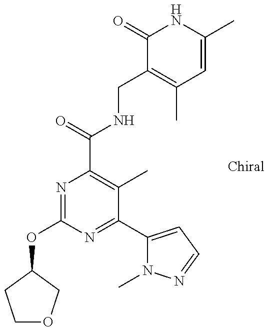 [SMILES: Cc1cc(C)c(CNC(=O)c2nc(O[C@@H]3CCOC3)nc(-c3ccnn3C)c2C)c(=O)[nH]1] | N-[(4,6- dimethyl-2- oxo-1,2- dihydropyridin- 3-yl)methyl]-5- methyl-6-(1- methyl-1H- pyrazol-5-yl)-2- [(3R)- tetrahydrofuran- 3- yloxy]pyrimidine- 4- carboxamide | ¹H NMR (400 MHz, DMSO-d6) δ 11.55 (br. s., 1H), 8.69 (t, J = 5.26 Hz, 1H), 7.56 (d, J = 1.96 Hz, 1H), 6.68 (d, J = 1.96 Hz, 1H), 5.88 (s, 1H), 5.47- 5.56 (m, 1H), 4.31 (d, J = 5.38 Hz, 2H), 3.70-3.99 (m, 7H), 2.29 (s, 3H), 2.22- 2.28 (m, 1H), 2.21 (s, 3H), 2.12 (s, 3H), 2.02-2.10 (m, 1H); MS: 439 [M + 1] | M |
| 94 | 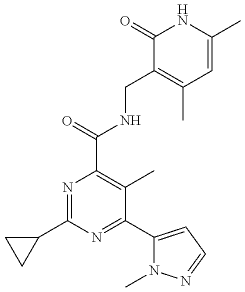 [SMILES: Cc1cc(C)c(CNC(=O)c2nc(C3CC3)nc(-c3ccnn3C)c2C)c(=O)[nH]1] | 2-cyclopropyl- N-[(4,6- dimethyl-2- oxo-1,2- dihydropyridin- 3-yl)methyl]-5- methyl-6-(1- methyl-1H- pyrazol-5- yl)pyrimidine- 4-carboxamide | ¹H NMR (400 MHz, chloroform-d) δ 12.09 (br. s., 1 H), 8.86 (t, J = 5.81 Hz, 1 H), 7.54 (d, J = 1.77 Hz, 1 H), 6.46 (d, J = 1.77 Hz, 1 H), 5.95 (s, 1 H), 4.56 (d, J = 6.06 Hz, 2 H), 3.93 (s, 3 H), 2.61 (s, 3 H), 2.40 (s, 3 H), 2.33 (s, 3 H), 2.19- 2.28 (m, 1 H), 1.09-1.17 (m, 2 H), 1.04 (dt, J = 8.02, 2.94 Hz, 2 H); MS: 393 [M + 1] | H |
| 95 | 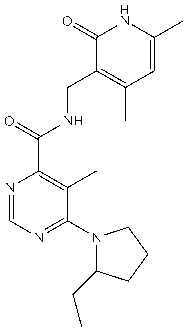 [SMILES: CCC1CCCN1c1ncnc(C(=O)NCc2c(C)cc(C)[nH]c2=O)c1C] | N-[(4,6- dimethyl-2- oxo-1,2- dihydropyridin- 3-yl)methyl]-6- (2- ethylpyrrolidin- 1-yl)-5- methylpyrimidine- 4- carboxamide | ¹H NMR (400 MHz, DMSO-d6) δ 0.82 (t, J = 7.46 Hz, 3 H) 1.26-1.39 (m, 1 H) 1.56-1.80 (m, 3 H) 1.91 (d, J = 3.06 Hz, 1H) 2.02-2.09 (m, 1 H) 2.11 (s, 3 H) 2.20 (s, 3 H) 2.28 (s, 3 H) 3.46-3.54 (m, 1 H) 3.66-3.75 (m, 1 H) 4.20-4.32 (m, 3 H) 5.86 (s, 1 H) 8.37 (s, 1 H) 8.51 (t, J = 5.20 Hz, 1 H) 11.50 (s, 1 H); MS: 370 [M + 1] | H |
| 96 | 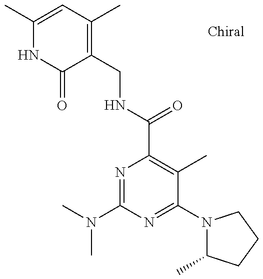 [SMILES: Cc1cc(C)c(CNC(=O)c2nc(N(C)C)nc(N3CCC[C@@H]3C)c2C)c(=O)[nH]1] | 2- (dimethyalmino)- N-[(4,6- dimethyl-2- oxo-1,2- dihydropyridin- 3-yl)methyl]-5- methyl-6-[(2S)- 2- methylpyrrolidin- 1- yl]pyrimidine- 4-carboxamide | ¹H NMR (400 MHz, methanol -d4) δ 1.23 (d, J = 5.87 Hz, 3 H) 1.52-1.81 (m, 2 H) 1.87-2.01 (m, 1 H) 2.09-2.18 (m, 1 H) 2.20 (s, 3 H) 2.23 (s, 3H) 2.37 (s, 3 H) 3.08 (s, 7 H) 3.40-3.51 (m, 1 H) 3.75 (td, J = 9.78, 6.60 Hz, 1 H) 4.36 (d, J = 8.07 Hz, 1 H) 4.45 (d, J = 7.58 Hz, 2 H) 6.08 (s, 1 H); MS: 399 [M + 1] | M |
| 97 | 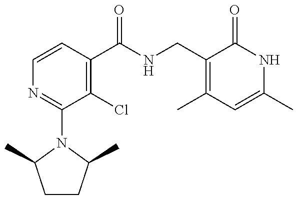 | 3-chloro-N- [(4,6-dimethyl- 2-oxo-1,2- dihydropyridin- 3-yl)methyl]-2- [(2R,5S)-2,5- dimethylpyrroli- din-1-yl]iso- nicotinamide | ¹H NMR (400 MHz, DMSO-d6) δ 1.25 (d, J = 6.24 Hz, 6 H) 1.62-1.75 (m, 2 H) 1.85-1.98 (m, 2 H) 2.11 (s, 3 H) 2.18 (s, 3 H) 4.25 (d, J = 4.77 Hz, 2 H) 4.30-4.46 (m, 2 H) 5.86 (s, 1 H) 6.57 (d, J = 4.77 Hz, 1 H) 8.02 (d, J = 4.65 Hz, 1 H) 8.37 (t, J = 4.58 Hz, 1 H) 11.41 (br. s., 1 H); MS: 389 [M + 1] | J |
| 98 | 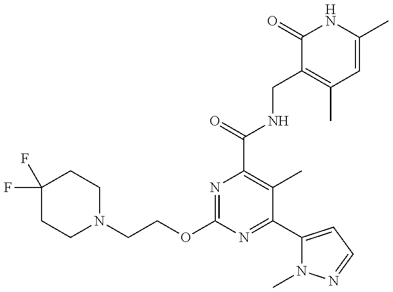 [SMILES: Cc1cc(C)c(CNC(=O)c2nc(OCCN3CCC(F)(F)CC3)nc(-c3ccnn3C)c2C)c(=O)[nH]1] | 2-[2-(4,4- difluoropiperidin- 1-yl)ethoxy]- N-[(4,6- dimethyl-2- oxo-1,2- dihydropyridin- 3-yl)methyl]-5- methyl-6-(1- methyl-1H- pyrazol-5- yl)pyrimidine- 4-carboxamide | ¹H NMR (400 MHz, DMSO-d6) δ 7.56 (d, J = 1.77 Hz, 1H), 6.67 (d, J = 1.77 Hz, 1H), 5.88 (s, 1H), 4.44 (t, J = 5.68 Hz, 2H), 4.31 (d, J = 5.56 Hz, 2H), 3.91 (s, 3H), 2.78 (t, J = 5.56 Hz, 2H), 2.59 (t, J = 5.18 Hz, 4H), 2.28 (s, 3H), 2.21 (s, 3H), 2.12 (s, 3H); MS: 516 [M + 1] | M |
| 99 | 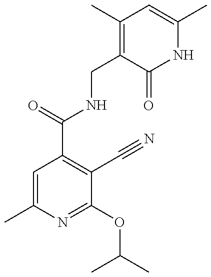 | 3-cyano-N- [(4,6-dimethyl- 2-oxo-1,2- dihydropyridin- 3-yl)methyl]-2- isopropoxy-6- methyliso- nicotinamide | ¹H NMR (600 MHz, DMSO-d6) δ 11.52 (br. s., 1H), 8.72 (t, J = 4.98 Hz, 1H), 7.06 (s, 1H), 5.88 (s, 1H), 5.36 (td, J = 6.22, 12.44 Hz, 1H), 4.28 (d, J = 4.98 Hz, 2H), 2.45 (s, 3H), 2.17 (s, 3H), 2.11 (s, 3H), 1.32 (d, J = 6.08 Hz, 6H); MS: 355 [M + 1] | J ] |
| 100 | 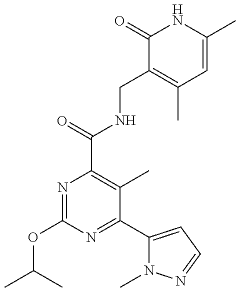 [SMILES: Cc1cc(C)c(CNC(=O)c2nc(OC(C)C)nc(-c3ccnn3C)c2C)c(=O)[nH]1] | N-[(4,6- dimethyl-2- oxo-1,2- dihydropyridin- 3-yl)methyl]-2- isopropoxy-5- methyl-6-(1- methyl-1H- pyrazol-5- yl)pyrimidine- 4-carboxamide | ¹H NMR (400 MHz, DMSO-d6) δ 11.53 (s, 1H), 8.66 (t, J = 5.43 Hz, 1H), 7.55 (d, J = 2.02 Hz, 1H), 6.66 (d, J = 2.02 Hz, 1H), 5.88 (s, 1H), 5.21 (quin, J = 6.19 Hz, 1H), 4.31 (d, J = 5.56 Hz, 2H), 3.91 (s, 3H), 2.27 (s, 3H), 2.21 (s, 3H), 2.12 (s, 3H), 1.32 (d, J = 6.06 Hz, 6H); MS: 411 [M + 1] | M |
| 101 | 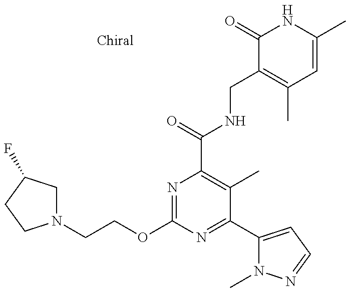 [SMILES: Cc1cc(C)c(CNC(=O)c2nc(OCCN3CC[C@H](F)C3)nc(-c3ccnn3C)c2C)c(=O)[nH]1] | N-[(4,6- dimethyl-2- oxo-1,2- dihydropyridin- 3-yl)methyl]-2- {2-[(3S)-3- fluoropyrrolidin- 1-yl]ethoxy}-5- methyl-6-(1- methyl-1H- pyrazol-5- yl)pyrimidine- 4-carboxamide | ¹H NMR (700 MHz, DMSO-d6) δ 11.52 (br. s., 1H), 8.68 (t, J = 4.95 Hz, 1H), 7.56 (s, 1H), 6.67 (s, 1H), 5.88 (s, 1H), 5.09- 5.24 (m, 1H), 4.42 (t, J = 5.61 Hz, 2H), 4.31 (d, J = 5.06 Hz, 2H), 3.91 (s, 3H), 2.77-2.91 (m, 4H), 2.62-2.71 (m, 1H), 2.38 (q, J = 7.92 Hz, 1H), 2.27 (s, 3H), 2.21 (s, 3H), 2.11 (s, 3H), 2.04-2.10 (m, 1H), 1.78-1.89 (m, 1H); MS: 484 [M + 1] | M |
| 102 | 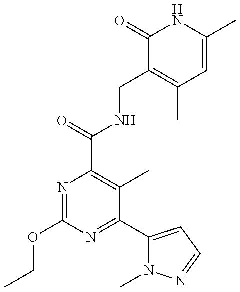 [SMILES: CCOc1nc(C(=O)NCc2c(C)cc(C)[nH]c2=O)c(C)c(-c2ccnn2C)n1] | N-[(4,6- dimethyl-2- oxo-1,2- dihydropyridin- 3-yl)methyl]-2- ethoxy-5- methyl-6-(1- methyl-1H- pyrazol-5- yl)pyrimidine- 4-carboxamide | ¹H NMR (400 MHz, DMSO-d6) δ 8.68 (t, J = 5.31 Hz, 1H), 7.56 (d, J = 2.02 Hz, 1H), 6.67 (d, J = 1.77 Hz, 1H), 5.88 (s, 1H), 4.37 (q, J = 6.99 Hz, 2H), 4.31 (d, J = 5.31 Hz, 2H), 3.91 (s, 3H), 2.28 (s, 3H), 2.21 (s, 3H), 2.12 (s, 3H), 1.34 (t, J = 7.07 Hz, 3H); MS: 397 [M + 1] | M |
| 103 | 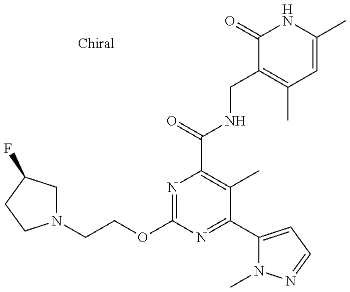 [SMILES: Cc1cc(C)c(CNC(=O)c2nc(OCCN3CC[C@@H](F)C3)nc(-c3ccnn3C)c2C)c(=O)[nH]1] | N-[(4,6- dimethyl-2- oxo-1,2- dihydropyridin- 3-yl)methyl]-2- {2-[(3R)-3- fluoropyrrolidin- 1-yl]ethoxy}-5- methyl-6-(1- methyl-1H- pyrazol-5- yl)pyrimidine- 4-carboxamide | ¹H NMR (700 MHz, DMSO-d6) δ 11.51 (br. s., 1H), 8.67 (t, J = 5.06 Hz, 1H), 7.56 (s, 1H), 6.67 (s, 1H), 5.88 (s, 1H), 5.09- 5.24 (m, 1H), 4.42 (t, J = 5.61 Hz, 2H), 4.31 (d, J = 5.28 Hz, 2H), 3.91 (s, 3H), 2.77-2.91 (m, 4H), 2.61-2.72 (m, 1H), 2.38 (q, J = 7.92 Hz, 1H), 2.27 (s, 3H), 2.21 (s, 3H), 2.11 (s, 3H), 2.05-2.09 (m, 1H), 1.78-1.89 (m, 1H); MS: 484 [M + 1] | M |
| 104 | 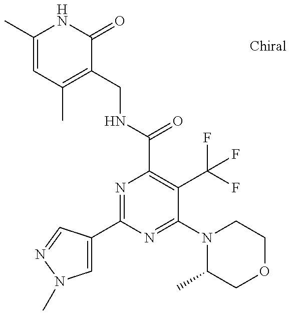 [SMILES: Cc1cc(C)c(CNC(=O)c2nc(-c3cnn(C)c3)nc(N3CCOC[C@@H]3C)c2C(F)(F)F)c(=O)[nH]1] | N-[(4,6- dimethyl-2- oxo-1,2- dihydropyridin- 3-yl)methyl]-6- [(3S)-3- methylmorpholin- 4-yl]-2-(1- methyl-1H- pyrazol-4-yl)-5- (trifluoromethyl) pyrimidine-4- carboxamide | ¹H NMR (400 MHz, DMSO-d6) δ 8.43 (t, J = 4.9 Hz, 1H), 8.18 (s, 1H), 7.79 (s, 1H), 5.78 (s, 1H), 4.63 (br. s., 1H), 4.28 (br. s., 1H), 4.16 (d, J = 4.8 Hz, 2H), 3.82 (s, 3H), 3.64 (d, J = 11.4 Hz, 1H), 3.48 (dd, J = 11.7, 2.9 Hz, 1H), 3.29- 3.39 (m, 2H), 3.05-3.19 (m, 2H), 2.09 (s, 3H), 2.03 (s, 3H), 1.15 (d, J = 6.8 Hz, 3H); MS: 506 [M + 1] | H |
| 105 | 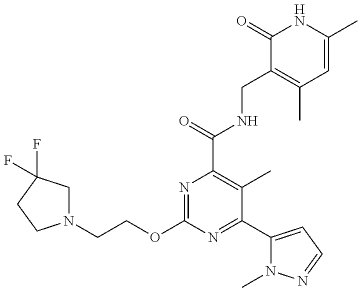 [SMILES: Cc1cc(C)c(CNC(=O)c2nc(OCCN3CCC(F)(F)C3)nc(-c3ccnn3C)c2C)c(=O)[nH]1] | 2-[2-(3,3- difluoropyrrolidin- 1-yl)ethoxy]- N-[(4,6- dimethyl-2- oxo-1,2- dihydropyridin- 3-yl)methyl]-5- methyl-6-(1- methyl-1H- pyrazol-5- yl)pyrimidine- 4-carboxamide | ¹H NMR (700 MHz, DMSO-d6) δ 11.53 (br. s., 1H), 8.68 (br. s., 1H), 7.55 (s, 1H), 6.67 (s, 1H), 5.88 (s, 1H), 4.42 (t, J = 4.84 Hz, 2H), 4.31 (d, J = 4.84 Hz, 2H), 3.91 (s, 3H), 2.96 (t, J = 13.42 Hz, 2H), 2.84 (t, J = 4.84 Hz, 2H), 2.77 (t, J = 6.71 Hz, 2H), 2.27 (s, 3H), 2.16-2.25 (m, 5H), 2.11 (s, 3H); MS: 502 [M + 1] | M |
| 106 | 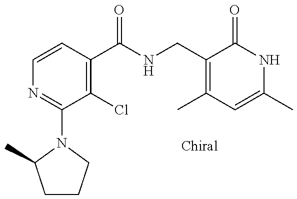 | 3-chloro-N- [(4,6-dimethyl- 2-oxo-1,2- dihydropyridin- 3-yL)methyl]-2- [(2R)-2- methylpyrrolidin- 1- yl]isonicotin- amide | ¹H NMR (400 MHz, DMSO-d6) δ 1.09 (d, J = 5.81 Hz, 3 H) 1.48-1.59 (m, 1 H) 1.60-1.76 (m, 1 H) 1.85-1.95 (m, 1 H) 2.06-2.15 (m, 4 H) 2.18 (s, 3 H) 3.31 (m, J = 18.06, 2.15 Hz, 1 H) 3.82 (td, J = 9.92, 6.69 Hz, 1 H) 4.26 (d, J = 4.80 Hz, 2 H) 4.28- 4.37 (m, 1 H) 5.86 (s, 1 H) 6.65 (d, J = 4.55 Hz, 1 H) | J |
| | | | 8.05 (d, J = 4.80 Hz, 1 H) | |
| | | | 8.39 (t, J = 4.93 Hz, 1 H) | |
| | | | 11.46 (br. s., 1 H); | |
| | | | MS: 375 [M + 1] | |
| 107 | 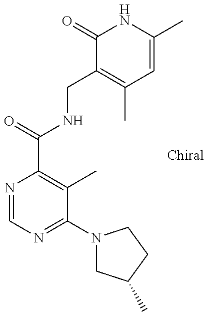 [SMILES: Cc1cc(C)c(CNC(=O)c2ncnc(N3CC[C@H](C)C3)c2C)c(=O)[nH]1] | N-[(4,6- dimethyl-2- oxo-1,2- dihydropyridin- 3-yl)methyl]-5- methyl-6-[(3S)- 3- methylpyrrolidin- 1- yl]pyrimidine- 4-carboxamide | ¹H NMR (400 MHz, DMSO-d6) δ 1.04 (d, J = 6.82 Hz, 3 H) 1.47 (dq, J = 11.87, 8.67 Hz, 1 H) 1.94-2.05 (m, 1 H) 2.11 (s, 3 H) 2.20 (s, 3 H) 2.21- 2.28 (m, 1 H) 2.34 (s, 3 H) 3.19 (dd, J = 10.36, 8.34 Hz, 1 H) 3.56-3.68 (m, 2 H) 3.71 (dd, J = 10.61, 7.07 Hz, 1 H) 4.25 (d, J = 5.31 Hz, 2 H) 5.86 (s, 1 H) 8.27 (s, 1 H) 8.42 (t, J = 5.56 Hz, 1 H) 11.49 (br. s., 1 H); MS: 356 [M + 1] | H |
| 108 | 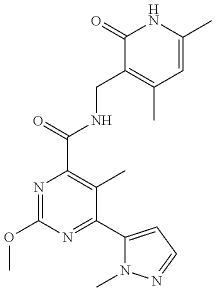 [SMILES: COc1nc(C(=O)NCc2c(C)cc(C)[nH]c2=O)c(C)c(-c2ccnn2C)n1] | N-[(4,6- dimethyl-2- oxo-1,2- dihydropyridin- 3-yl)methyl]-2- methoxy-5- methyl-6-(1- methyl-1H- pyrazol-5- yl)pyrimidine- 4-carboxamide | ¹H NMR (400 MHz, DMSO-d6) δ 8.66-8.74 (m, 1H), 7.56 (d, J = 2.02 Hz, 1H), 6.69 (d, J = 2.02 Hz, 1H), 5.88 (s, 1H), 4.31 (d, J = 4.29 Hz, 2H), 3.95 (s, 3H), 3.93 (s, 3 H), 2.28 (s, 3H), 2.21 (s, 3H), 2.12 (s, 3H); MS: 383 [M + 1] | M |
| 109 | 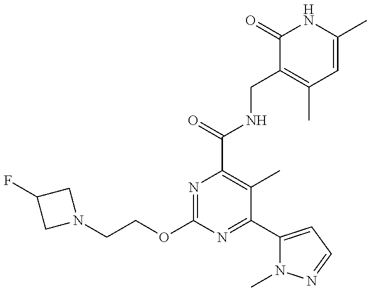 [SMILES: Cc1cc(C)c(CNC(=O)c2nc(OCCN3CC(F)C3)nc(-c3ccnn3C)c2C)c(=O)[nH]1] | N-[(4,6- dimethyl-2- oxo-1,2- dihydropyridin- 3-yl)methyl]-2- [2-(3- fluoroazetidin- 1-yl)ethoxy]-5- methyl-6-(1- methyl-1H- pyrazol-5- yl)pyrimidine- 4-carboxamide | ¹H NMR (700 MHz, DMSO-d6) δ 11.53 (br. s., 1H), 8.68 (t, J = 5.17 Hz, 1H), 7.56 (d, J = 1.76 Hz, 1H), 6.63-6.72 (m, 1H), 5.88 (s, 1H), 5.05-5.20 (m, 1H), 4.27-4.34 (m, 4H), 3.90 (s, 3H), 3.55-3.63 (m, 2H), 3.12-3.21 (m, 2H), 2.81 (t, J = 5.50 Hz, 2H), 2.27 (s, 3H), 2.21 (s, 3H), 2.11 (s, 3H); MS: 470 [M + 1] | M |
| 110 | 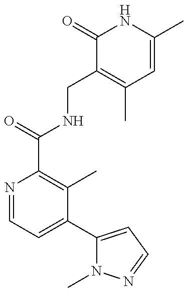 [SMILES: Cc1cc(C)c(CNC(=O)c2nccc(-c3ccnn3C)c2C)c(=O)[nH]1] | N-[(4,6- dimethyl-2- oxo-1,2- dihydropyridin- 3-yl)methyl]-3- methyl-4-(1- methyl-1H- pyrazol-5- yl)pyridine-2- carboxamide | ¹H NMR (400 MHz, DMSO-d6) δ 11.51 (s, 1H), 8.58 (t, J = 5.56 Hz, 1H), 8.49 (d, J = 4.80 Hz, 1H), 7.54 (d, J = 2.02 Hz, 1H), 7.44 (d, J = 4.80 Hz, 1H), 6.37 (d, J = 2.02 Hz, 1H), 5.87 (s, 1H), 4.32 (d, J = 5.56 Hz, 2H), 3.61 (s, 3H), 2.30 (s, 3H), 2.23 (s, 3H), 2.12 (s, 3H); MS: 352 [M + 1] | B |
| 111 | 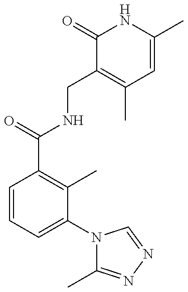 [SMILES: Cc1cc(C)c(CNC(=O)c2cccc(-n3cnnc3C)c2C)c(=O)[nH]1] | N-[(4,6- dimethyl-2- oxo-1,2- dihydropyridin- 3-yl)methyl]-2- methyl-3-(3- methyl-4H- 1,2,4-triazol-4- yl)benzamide | ¹H NMR (600 MHz, DMSO-d6) δ 8.55 (s, 1H), 8.34 (t, J = 4.58 Hz, 1H), 7.41-7.44 (m, 1H), 7.39 (d, J = 5.09 Hz, 2H), 5.87 (s, 1H), 4.28 (d, J = 5.09 Hz, 2H), 2.19 (s, 3H), 2.14 (s, 3H), 2.11 (s, 3H), 1.91 (s, 3H); MS: 352 [M + 1] | F |
| 112 | 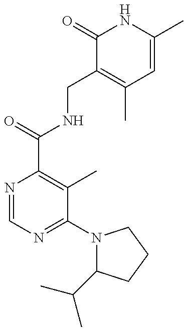 [SMILES: Cc1cc(C)c(CNC(=O)c2ncnc(N3CCCC3C(C)C)c2C)c(=O)[nH]1] | N-[(4,6- dimethyl-2- oxo-1,2- dihydropyriidn- 3-yl)methyl]-6- (2- isopropylpyrroli- din-1-yl)-5- methylpyrimidine- 4- carboxamide | ¹H NMR (400 MHz, DMSO-d6) δ 0.72 (d, J = 6.72 Hz, 3 H) 0.85 (d, J = 6.97 Hz, 3 H) 1.54-1.72 (m, 2 H) .183-1.92 (m, 2 H) 2.11 (s, 3 H) 2.13-2.19 (m, 1 H) 2.20 (s, 3 H) 2.28 (s, 3 H) 3.41-3.48 (m, 1 H) 3.53-3.63 (m, 1 H) 4.19-4.32 (m, 2 H) 4.33- 4.40 (m ,1 H) 5.86 (s, 1 H) 8.35 (s, 1 H) 8.48 (t, J = 5.62 Hz, 1 H) 11.49 (br. s., 1 H); MS: 384 [M + 1] | H |
| 113 | 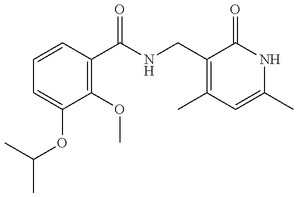 [SMILES: COc1c(OC(C)C)cccc1C(=O)NCc1c(C)cc(C)[nH]c1=O] | N-[(4,6- dimethyl-2- oxo-1,2- dihydropyridin- 3-yl)methyl]-3- isopropoxy-2- methoxy- benzamide | ¹H NMR (400 MHz, DMSO-d6) δ 1.28 (d, J = 6.06 Hz, 6 H) 2.11 (s, 3 H) 2.23 (s, 3 H) 3.76 (s, 3 H) 4.30 (d, J = 5.56 Hz, 2 H) 4.61 (spt, J = 6.02 Hz, 1 H) 5.86 (s, 1 H) 7.05-7.11 (m, 1 H) 7.16 (dd, J = 8.08, 1.26 Hz, 1 H) 7.33 (dd, J = 7.83, 1.52 Hz, 1 H) 8.66 (t, J = 5.31 Hz, 1 H) 11.55 (br. s., 1 H); | |
| | | | MS: 345 [M + 1] | |
| 114 | 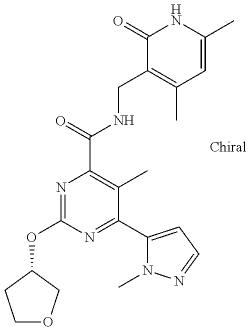 [SMILES: Cc1cc(C)c(CNC(=O)c2nc(O[C@H]3CCOC3)nc(-c3ccnn3C)c2C)c(=O)[nH]1] | N-[(4,6- dimethyl-2- oxo-1,2- dihydropyridin- 3-yl)methyl]-5- methyl-6-(1- methyl-1H- pyrazol-5-yl)-2- [(3S)- tetrahydrofuran- 3- yloxy]pyrimidine- 4- carboxamide | ¹H NMR (400 MHz, DMSO-d6) δ 11.51 (br. s., 1H), 8.70 (t, J = 5.38 Hz, 1H), 7.56 (d, J = 1.96 Hz, 1H), 6.68 (d, J = 1.96 Hz, 1H), 5.88 (s, 1H), 5.51 (dd, J = 4.52, 6.24 Hz, 1H), 4.31 (d, J = 5.38 Hz, 2H), 3.74- 3.95 (m, 7H), 2.29 (s, 3H), 2.25 (dd, J = 6.24, 13.57 Hz, 1H), 2.21 (s, 3H), 2.12 (s, 3H), 2.06 (td, J = 6.48, 13.45 Hz, 1H); MS: 439 [M + 1] | M |
| 115 | 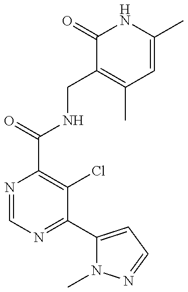 [SMILES: Cc1cc(C)c(CNC(=O)c2ncnc(-c3ccnn3C)c2Cl)c(=O)[nH]1] | 5-chloro-N- [(4,6-dimethyl- 2-oxo-1,2- dihydropyridin- 3-yl)methyl]-6- (1-methyl-1H- pyrazol-5- yl)pyrimidine- 4-carboxamide | ¹H NMR (400 MHz, chloroform-d) δ 12.43 (br. s., 1 H), 9.05 (s, 1 H), 8.65 (br. s., 1 H), 7.58 (d, J = 1.52 Hz, 1 H), 6.94 (d, J = 1.52 Hz, 1 H), 5.99 (s, 1 H), 4.59 (d, J = 5.81 Hz, 2 H), 4.06 (s, 3 H), 2.41 (s, 3 H), 2.34 (s, 3 H); MS: 373 [M + 1] | B |
| 116 | 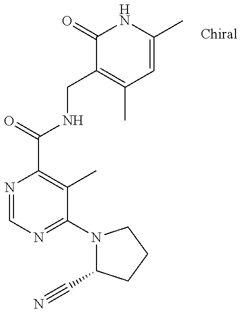 [SMILES: Cc1cc(C)c(CNC(=O)c2ncnc(N3CCC[C@@H]3C#N)c2C)c(=O)[nH]1] | 6-[(2R)-2- cyanopyrrolidin- 1-yl]-N-[(4,6- dimethyl-2- oxo-1,2- dihydropyridin- 3-yl)methyl]-5- methylpyrimidine- 4- carboxamide | ¹H NMR (400 MHz, methanol -d4) δ 9.00 (s, 1H), 6.62 (s, 1H), 5.60 (t, J = 6.7 Hz, 1H), 5.03 (s, 2H), 4.68 (br. s., 1H), 4.52 (dt, J = 9.5, 7.1 Hz, 1H), 4.34 (ddd, J = 9.6, 7.7, 5.2 Hz, 1H), 3.04 (s, 3H), 2.97 (ddd, J = 12.8, 6.6, 6.4 Hz, 1H), 2.92 (s, 3H), 2.83- 2.89 (m, 1H), 2.68-2.79 (m, 1H), 2.55-2.63 (m, 1H), 2.43 (s, 2H); MS: 367 [M + 1] | H |
| 117 | 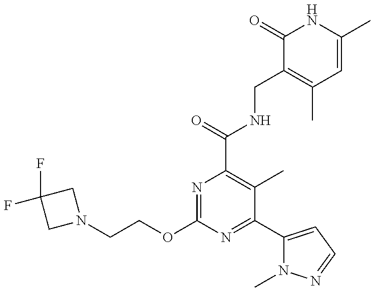 [SMILES: Cc1cc(C)c(CNC(=O)c2nc(OCCN3CC(F)(F)C3)nc(-c3ccnn3C)c2C)c(=O)[nH]1] | 2-[2-(3,3- difluoroazetidin- 1-yl)ethoxy]- N-[(4,6- dimethyl-2- oxo-1,2- dihydropyridin- 3-yl)methyl]-5- methyl-6-(1- methyl-1H- pyrazol-5- yl)pyrimidine- 4-carboxamide | ¹H NMR (700 MHz, DMSO-d6) δ 11.07 (br. s., 1H), 8.22 (t, J = 5.17 Hz, 1H), 7.07-7.12 (m, 1H), 6.21 (s, 1H), 5.42 (s, 1H), 3.87 (t, J = 5.28 Hz, 2H), 3.85 (d, J = 5.28 Hz, 2H), 3.44 (s, 3H), 3.17 (t, J = 12.43 Hz, 4H), 2.46 (t, J = 5.06 Hz, 2H), 1.81 (s, 3H), 1.75 (s, 3H), 1.65 (s, 3H); MS: 488 [M + 1] | M |
| 118 | 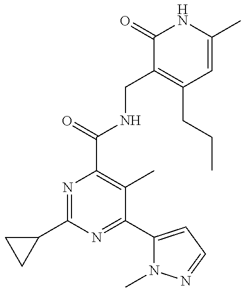 [SMILES: CCCc1cc(C)[nH]c(=O)c1CNC(=O)c1nc(C2CC2)nc(-c2ccnn2C)c1C] | 2- cyclopropyl-5- methyl-N-[(6- methyl-2-oxo- 4-propyl-1,2- dihydropyridin- 3-yl)methyl]-6- (1-methyl-1H- pyrazol-5- yl)pyrimidine- 4-carboxamide | ¹H NMR (400 MHz, chloroform-d) δ 11.82 (br. s., 1 H), 8.89 (t, J = 6.06 Hz, 1 H), 7.54 (d, J = 2.02 Hz, 1 H), 6.47 (d, J = 2.02 Hz, 1 H), 5.95 (s, 1 H), 4.57 (d, J = 6.06 Hz, 2 H), 3.93 (s, 3 H), 2.66-2.73 (m, 2 H), 2.60 (s, 3 H), 2.34 (s, 3 H), 2.19-2.28 (m, 1 H), 1.55- 1.68 (m, 2 H), 1.10-1.16 (m, 2 H), 0.97-1.07 (m, 5 H); MS: 421 [M + 1] | H |
| 119 | 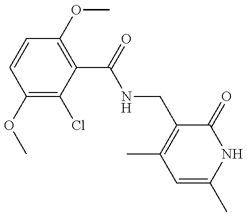 [SMILES: COc1ccc(OC)c(C(=O)NCc2c(C)cc(C)[nH]c2=O)c1Cl] | 2-chloro-N- [(4,6-dimethyl- 2-oxo-1,2- dihydropyridin- 3-yl)methyl]- 3,6- dimethoxybenz- amide | MS: 351 [M + 1] | I |
| 120 | 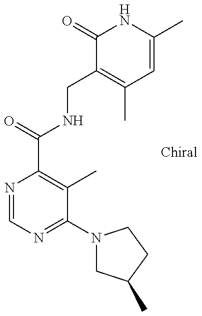 [SMILES: Cc1cc(C)c(CNC(=O)c2ncnc(N3CC[C@@H](C)C3)c2C)c(=O)[nH]1] | N-[(4,6- dimethyl-2- oxo-1,2- dihydropyridin- 3-yl)methyl]-5- methyl-6-[(3R)- 3- methylpyrrolidin- 1- yl]pyrimidine- 4-carboxamide | ¹H NMR (400 MHz, DMSO-d6) δ 1.04 (d, J = 6.57 Hz, 3 H) 1.47 (dq, J = 11.81, 8.69 Hz, 1 H) 1.89-2.06 (m, 1 H) 2.11 (s, 3 H) 2.20 (s, 3 H) 2.21- 2.27 (m, 1 H) 2.35 (s, 3 H) 3.19 (dd, J = 10.36, 8.34 Hz, 1 H) 3.54-3.67 (m, 2 H) 3.72 (dd, J = 10.48, 6.95 Hz, 1H) 4.25 (d, J = 5.31 Hz, 2 H) 5.86 (s, 1 H) 8.27 (s, 1 H) 8.43 (t, J = 5.43 Hz, 1 H); MS: 356 [M + 1] | H |
| 121 | 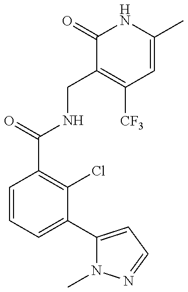 [SMILES: Cc1cc(C(F)(F)F)c(CNC(=O)c2cccc(-c3ccnn3C)c2Cl)c(=O)[nH]1] | 2-chloro-N-{[6- methyl-2-oxo- 4- (trifluoromethyl)- 1,2- dihydropyridin- 3-yl]methyl}-3- (1-methyl-1H- pyrazol-5- yl)benzamide | ¹H NMR (400 MHz, DMSO-d6) δ 12.35 (br. s., 1H), 8.69 (br. s., 1H), 7.50 (d, J = 1.8 Hz, 1H), 7.40- 7.48 (m, 3H), 6.31 (d, J = 2.0 Hz, 1H), 6.21 (s, 1H), 4.37 (d, J = 3.0 Hz, 2H), 3.62 (s, 3H), 2.22 (s, 3H); MS: 425 [M + 1] | A |
| 122 | 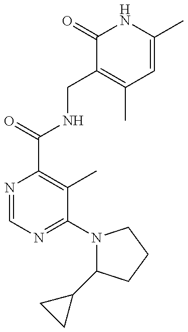 [SMILES: Cc1cc(C)c(CNC(=O)c2ncnc(N3CCCC3C3CC3)c2C)c(=O)[nH]1] | 6-(2- cyclopropylpyr- rolidin-1-yl)-N- [(4,6-dimethyl- 2-oxo-1,2- dihydropyridin- 3-yl)methyl]-5- methylpyrimidine- 4- carboxamide | ¹H NMR (400 MHz, DMSO-d6) δ 0.09-0.17 (m, 1 H) 0.24-0.37 (m, 2 H) 0.40-0.47 (m, 1 H) 0.95-1.05 (m, 1 H) 1.54- 1.71 (m, 2 H) 1.87-2.02 (m, 2 H) 2.11 (s, 3 H) 2.20 (s, 3 H) 2.28 (s, 3 H) 3.38 (ddd, J = 10.21, 7.21, 3.24 Hz, 1 H) 3.73 (td, J = 9.23, 6.72 Hz, 1 H) 4.19-4.32 (m, 2 H) 2 H) 4.35 (q, J = 6.93 Hz, 1 H) 5.86 (s, 1 H) 8.33 (s, 1 H) 8.47 (t, J = 5.62 Hz, 1 H) 1.48 (br. s., 1 H); MS: 382 [M + 1] | H |
| 123 | 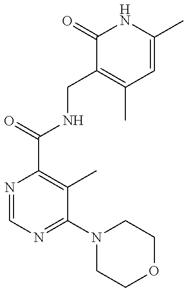 [SMILES: Cc1cc(C)c(CNC(=O)c2ncnc(N3CCOCC3)c2C)c(=O)[nH]1] | N-[(4,6- dimethyl-2- oxo-1,2- dihydropyridin- 3-yl)methyl]-5- methyl-6- morpholin-4- ylpyrimidine-4- carboxamide | ¹H NMR (400 MHz, DMSO-d6) δ 2.11 (s, 3 H) 2.20 (s, 3 H) 2.28 (s, 3 H) 3.34 (d, J = 5.05 Hz, 4 H) 3.63-3.75 (m, 4 H) 4.28 (d, J = 5.81 Hz, 2 H) 5.86 (s, 1 H) 8.54 (s, 1 H) 8.56 (t, J = 5.43 Hz, 1 H); MS: 358 [M + 1] | H |
| 124 | 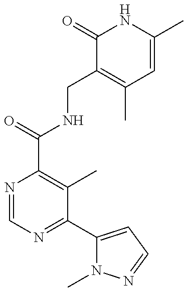 [SMILES: Cc1cc(C)c(CNC(=O)c2ncnc(-c3ccnn3C)c2C)c(=O)[nH]1] | N-[(4,6- dimethyl-2- oxo-1,2- dihydropyridin- 3-yl)methyl]-5- methyl-6-(1- methyl-1H- pyrazol-5- yl)pyrimidine- 4-carboxamide | ¹H NMR (400 MHz, chloroform-d) δ 12.30 (br. s., 1 H), 9.05 (s, 1 H), 8.96 (t, J = 5.68 Hz, 1 H), 7.57 (d, J = 2.02 Hz, 1 H), 6.51 (d, J = 2.02 Hz, 1 H), 5.98 (s, 1 H), 4.58 (d, J = 6.06 Hz, 2 H), 3.95 (s, 3 H), 2.70 (s, 3 H), 2.40 (s, 3 H), 2.37 (s, 3 H); MS: 353 [M + 1] | B |
| 125 | 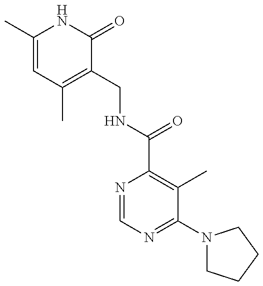 [SMILES: Cc1cc(C)c(CNC(=O)c2ncnc(N3CCCC3)c2C)c(=O)[nH]1] | N-[(4,6- dimethyl-2- oxo-1,2- dihydropyridin- 3-yl)methyl]-5- methyl-6- pyrrolidin-1- ylpyrimidine-4- carboxamide | ¹H NMR (400 MHz, DMSO-d6) δ 11.51 (s, 1H), 8.42-8.45 (m, J = 5.6 Hz, 1H), 8.27 (s, 1H), 5.86 (s, 1H), 4.24-4.25 (m, J = 5.6 Hz, 2H), 3.56-3.59 (m, J = 5.6 Hz, 4H), 2.34 (s, 3H), 2.19 (s, 3H), 2.10 (s, 3H), 1.82-1.86 (m, J = 5.6 Hz, 4H).; MS: 342 [M + 1] | H |
| 126 | 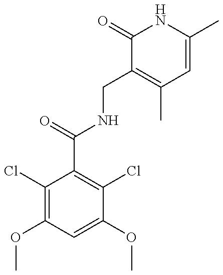 [SMILES: COc1cc(OC)c(Cl)c(C(=O)NCc2c(C)cc(C)[nH]c2=O)c1Cl] | 2,6-dichloro-N- [(4,6-dimethyl- 2-oxo-1,2- dihydropyridin- 3-yl)methyl]- 3,5- dimethoxybenz- amide | ¹H NMR (400 MHz, DMSO-d6) δ 11.47 (br. s., 1H), 8.44 (t, J = 4.9 Hz, 1H), 6.88 (s, 1H), 5.85 (s, 1H), 4.27 (d, J = 5.1 Hz, 2H), 3.90 (s, 6H), 2.19 (s, 3H), 2.10 (s, 3H); MS: 387 [M + 1] | I |
| 127 | 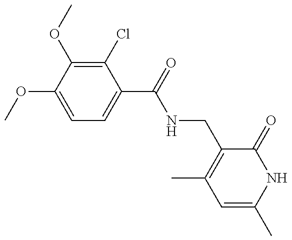 [SMILES: COc1ccc(C(=O)NCc2c(C)cc(C)[nH]c2=O)c(Cl)c1OC] | 2-chloro-N- [(4,6-dimethyl- 2-oxo-1,2- dihydropyridin- 3-yl)methyl]- 3,4- dimethoxybenz- amide | MS: 351 [M + 1] | I |
| 128 | 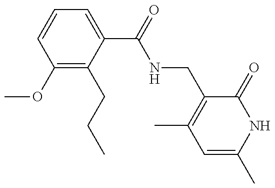 [SMILES: CCCc1c(OC)cccc1C(=O)NCc1c(C)cc(C)[nH]c1=O] | N-[(4,6- dimethyl-2- oxo-1,2- dihydropyridin- 3-yl)methyl]-3- methoxy-2- propylbenzamide | MS: 329 [M + 1] | I |
| 129 | 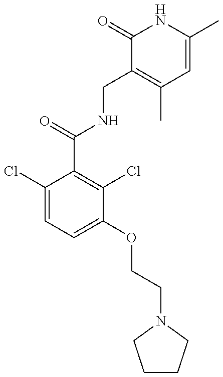 [SMILES: Cc1cc(C)c(CNC(=O)c2c(Cl)ccc(OCCN3CCCC3)c2Cl)c(=O)[nH]1] | 2,6-dichloro-N- [(4,6-dimethyl- 2-oxo-1,2- dihydropyridin- 3-yl)methyl]-3- (2-pyrrolidin-1- ylethoxy)benza- mide | ¹H NMR (400 MHz, DMSO-d6) δ 7.08 (d, J = 9.1 Hz, 1H), 6.87 (d, J = 8.8 Hz, 1H), 5.83 (s, 1H), 4.20 (s, 2H), 4.05-4.16 (m, 2H), 3.11 (br. s., 4H), 2.11 (s, 3H), 1.97 (s, 3H), 1.78 (br. s., 4H); MS: 440 [M + 1] | I |
| 130 | 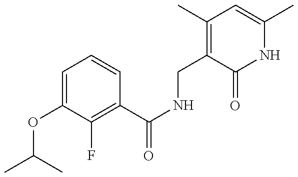 [SMILES: Cc1cc(C)c(CNC(=O)c2cccc(OC(C)C)c2F)c(=O)[nH]1] | N-[(4,6- dimethyl-2- oxo-1,2- dihydropyridin- 3-yl)methyl]-2- fluoro-3- isopropxoybenz- amide | ¹H NMR (400 MHz, DMSO) δ 11.6 (s, 1H), 8.27 (s, 1H), 7.26-7.11 (m, 3H), 5.88 (s, 1H), 4.65- 4.60 (m, 1H), 4.29-4.27 (d, J = 5.0 Hz, 2H), 2.18 (s, 3H), 2.12 (s, 3H), 1.28- 1.26 (d, J = 6.0 Hz, 6H); MS: 333 [M + 1] | I |
| 131 | 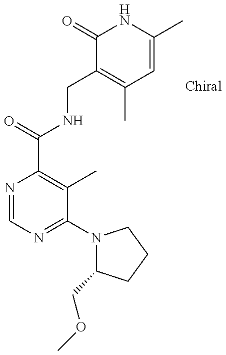 [SMILES: COC[C@H]1CCCN1c1ncnc(C(=O)NCc2c(C)cc(C)[nH]c2=O)c1C] | N-[(4,6- dimethyl-2- oxo-1,2- dihydropyridin- 3-yl)methyl]-6- [(2R)-2- (methoxymeth- yl)pyrrolidin-1- yl]-5- methylpyrimidine- 4- carboxamide | ¹H NMR (400 MHz, DMSO-d6) δ 1.64-1.85 (m, 2 H) 1.88-1.98 (m, 1 H) 1.98-2.07 (m, 1 H) 2.11 (s, 3 H) 2.20 (s, 3 H) 2.29 (s, 3 H) 3.22 (s, 3 H) 3.25 (dd, J = 9.29, 6.97 Hz, 1 H) 3.44-3.52 (m, 2 H) 3.65-3.74 (m, 1 H) 4.19- 4.33 (m, 2 H) 4.48-4.56 (m, 1 H) 5.86 (s, 1 H) 8.35 (s, 1 H) 8.45 (t, J = 5.38 Hz, 1 H) 11.49 (br. s., 1 H); MS: 386 [M + 1] | H |
| 132 | 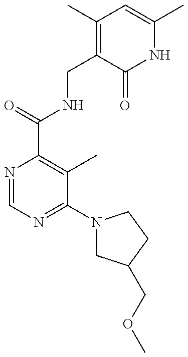 [SMILES: COCC1CCN(c2ncnc(C(=O)NCc3c(C)cc(C)[nH]c3=O)c2C)C1] | N-[(4,6- dimethyl-2- oxo-1,2- dihydropyridin- 3-yl)methyl]-6- [3- (methoxymeth- yl)pyrrolidin-1- yl]-5- methylpyrimidine- 4- carboxamide | ¹H NMR (400 MHz, methanol -d4) δ 1.74 (dq, J = 12.26, 8.30 Hz, 1 H) 2.00-2.14 (m, 1 H) 2.24 (s, 3H) 2.37 (s, 6 H) 2.53 (dt, J = 14.49, 7.06 Hz, 1 H) 3.35 (s, 3 H) 3.37-3.56 (m, 2 H) 3.66-3.85 (m, 2 H) 4.47 (s, 1 H) 6.09 (s, 1 H) 8.24 (s, 1 H); MS: 386 [M + 1] | H |
| 133 | 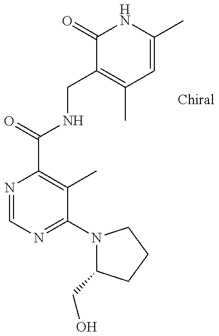 [SMILES: Cc1cc(C)c(CNC(=O)c2ncnc(N3CCC[C@@H]3CO)c2C)c(=O)[nH]1] | N-[(4,6- dimethyl-2- oxo-1,2- dihydropyridin- 3-yl)methyl]-6- [(2R)-2- (hydroxymeth- yl)pyrrolidin-1- yl]-5- methyl- pyrimidine-4- carboxamide | ¹H NMR (400 MHz, DMSO-d6) δ 1.63-1.76 (m, 1 H) 1.82-2.02 (m, 3 H) 2.11 (s, 3 H) 2.20 (s, 3 H) 2.30 (s, 3 H) 3.32-3.37 (m, 1 H) 3.45-3.56 (m ,2 H) 3.66-3.74 (m, 1 H) 4.26 (ddd, J = 23.24, 13.64, 5.56 Hz, 2 H) 4.40 (qd, J = 6.44, 3.41 Hz, 1 H) 4.65 (t, J = 5.56 Hz, 1 H) 5.86 (s, 1 H) 8.31 (s, 1 H) 8.44 (t, J = 5.56 Hz, 1 H) 11.49 (s, 1 H); MS: 372 [M + 1] | H |
| 134 | 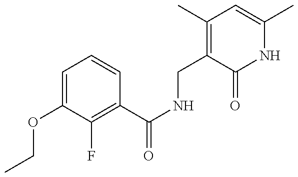 [SMILES: CCOc1cccc(C(=O)NCc2c(C)cc(C)[nH]c2=O)c1F] | N-[(4,6- dimethyl-2- oxo-1,2- dihydropyridin- 3-yl)methyl]-3- ethoxy-2- fluorobenz- amide | ¹H NMR (400 MHz, DMSO-d6) δ 11.6 (s, 1H), 8.27 (s, 1H), 7.24-7.20 (dd, J = 8x(2) Hz, 1H), 7.15-7.10 (m, 1H), 7.09-7.06 (m, 1H),, 5.87 (s, 1H), 4.29- 4.27 (d, J = 5.0 Hz, 2H), 4.10-4.08 (q, 2H), 2.17 (s, 3H), 2.10 (s, 3H), 1.36- 1.31 (t, J = 6.9x(2) 3H); MS: 319 [M + 1] | I |
| 135 | 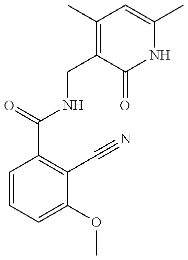 [SMILES: COc1cccc(C(=O)NCc2c(C)cc(C)[nH]c2=O)c1C#N] | 2-cyano-N- [(4,6-dimethyl- 2-oxo-1,2- dihydropyridin- 3-yl)methyl]-3- methoxybenz- amide | ¹H NMR (400 MHz, DMSO-d6) δ 11.51 (s, 1H), 8.58 (t, J = 5.56 Hz, 1H), 8.49 (d, J = 4.80 Hz, 1H), 7.54 (d, J = 2.02 Hz, 1H), 7.44 (d, J = 4.80 Hz, 1H), 6.37 (d, J = 2.02 Hz, 1H), 5.87 (s, 1H), 4.32 (d, J = 5.56 Hz, 2H), 3.61 (s, 3H), 2.30 (s, 3H), 2.23 (s, 3H), 2.12 (s, 3H); MS: 312 [M + 1] | I |
| 136 | 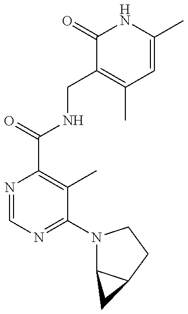 [SMILES: Cc1cc(C)c(CNC(=O)c2ncnc(N3CC[C@H]4C[C@H]43)c2C)c(=O)[nH]1] | 6-[(1R,5S)-2- azabicyclo[3.1.0] hex-2-yl]-N- [(4,6-dimethyl- 2-oxo-1,2- dihydropyridin- 3-yl)methyl]-5- methyl- pyrimidine-4- carboxamide | ¹H NMR (400 MHz, DMSO-d6) δ 0.59 (m, J = 2.20 Hz, 1 H) 0.80-0.89 (m, 1 H) 1.69-1.78 (m, 1 H) 1.83-1.93 (m, 1 H) 2.11 (s, 3 H) 2.13-2.18 (m, 1 H) 2.20 (s, 3 H) 2.44 (s, 3 H) 3.22-3.29 (m, 1 H) 3.64-3.71 (m, 1 H) 3.93-4.03 (m, 1 H) 4.26 (d, J = 5.14 Hz, 2 H) 5.86 (s, 1 H) 8.31 (s, 1 H) 8.44 (t, J = 5.01 Hz, 1 H) 11.49 (br. s., 1 H); MS: 354 [M + 1] | H |
| 137 | 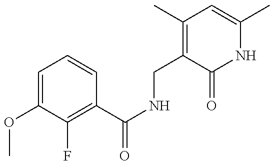 | N-[(4,6- dimethyl-2- oxo-1,2- dihydropyridin- 3-yl)methyl]-2- fluoro-3- methoxybenz- amide | ¹H NMR (400 MHz, DMSO-d6) δ 11.5 (s, 1H), 8.26 (s, 1H), 7.25-7.24 (m, J = 8x(2) Hz, 1H), 7.16-7.14 (m, 1H), 7.12-7.10 (m, 1H), 5.87 (s, 1H), 4.29-4.28 (d, J = 5.3 Hz, 2H), 3.84 (s, 3H), 2.18 (s, 3H), 2.11 (s, 3H); MS: 305 [M + 1] | I |
| 138 | 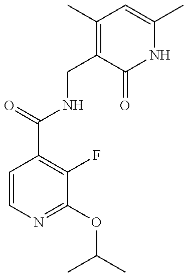 [SMILES: Cc1cc(C)c(CNC(=O)c2ccnc(OC(C)C)c2F)c(=O)[nH]1] | N-[(4,6- dimethyl-2- oxo-1,2- dihydropyridin- 3-yl)methyl]-3- fluoro-2- isopropoxyiso- nicotinamide | ¹H NMR (600 MHz, DMSO-d6) δ 8.56 (t, J = 5.09 Hz, 1H), 7.95 (d, J = 5.09 Hz, 1H), 6.98-7.00 (m, 1H), 5.87 (s, 1H), 5.30 (quin, J = 6.10 Hz, 1H), 4.28 (d, J = 5.09 Hz, 2H), 2.17 (s, 3H), 2.11 (s, 3H), 1.31 (d, J = 6.10 Hz, 6H); MS: 334 [M + 1] | J |
| 139 | 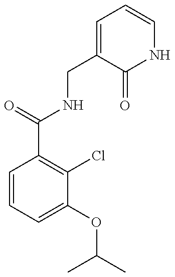 [SMILES: CC(C)Oc1cccc(C(=O)NCc2ccc[nH]c2=O)c1Cl] | 2-chloro-3- isopropoxy-N- [(2-oxo-1,2- dihydropyridin- 3- yl)methyl]benz- amide | ¹H NMR (600 MHz, DMSO-d6) δ 8.70 (t, J = 5.80 Hz, 1H), 7.39 (dd, J = 1.80, 6.77 Hz, 1H), 7.29- 7.34 (m, 2H), 7.21 (dd, J = 1.24, 8.43 Hz, 1H), 7.02 (dd, J = 1.24, 7.60 Hz, 1H), 6.21 (t, J = 6.63 Hz, 1H), 4.69 (td, J = 6.05, 11.96 Hz, 1H), 4.17 (d, J = 5.80 Hz, 2H), 1.30 (d, J = 6.08 Hz, 6H); MS: 321 [M + 1] | I |
| 140 | 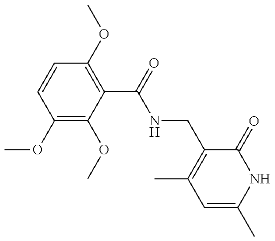 [SMILES: COc1ccc(OC)c(C(=O)NCc2c(C)cc(C)[nH]c2=O)c1OC] | N-[(4,6- dimethyl-2- oxo-1,2- dihydropyridin- 3-yl)methyl]- 2,3,6- trimethoxy- benzamide | MS: 347 [M + 1] | I |
| 141 | 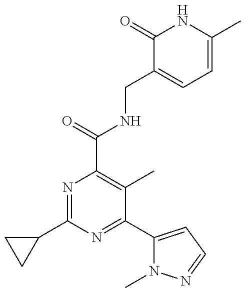 [SMILES: Cc1ccc(CNC(=O)c2nc(C3CC3)nc(-c3ccnn3C)c2C)c(=O)[nH]1] | 2-cyclopropyl- 5-methyl-N-[(6- methyl-2-oxo- 1,2- dihydropyridin- 3-yl)methyl]-6- (1-methyl-1H- pyrazol-5- yl)pyrimidine- 4-carboxamide | ¹H NMR (400 MHz, DMSO-d6) δ 11.69 (br. s., 1 H), 9.00 (t, J = 6.06 Hz, 1 H), 7.55 (d, J = 2.02 Hz, 1 H), 7.27 (d, J = 6.82 Hz, 1 H), 6.67 (d, J = 2.02 Hz, 1 H), 5.99 (d, J = 7.07 Hz, 1 H), 4.22 (d, J = 6.06 Hz, 2 H), 3.88 (s, 3 H), 2.34 (s, 3 H), 2.22-2.29 (m, 1 H), 2.16 (s, 3 H), 1.04-1.11 (m, 4 H); MS: 379 [M + 1] | H |
| 142 | 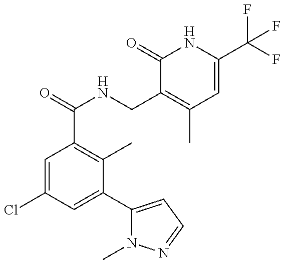 [SMILES: Cc1cc(C(F)(F)F)[nH]c(=O)c1CNC(=O)c1cc(Cl)cc(-c2ccnn2C)c1C] | 5-chloro-2- methyl-N-{[4- methyl-2-oxo- 6- (trifluoromethyl)- 1,2- dihydropyridin- 3-yl]methyl}-3- (1-methyl-1H- pyrazol-5- yl)benzamide | ¹H NMR (600 MHz, DMSO-d6) δ 2.00 (s, 3 H) 2.44 (s, 3 H) 3.58 (s, 3 H) 4.43 (d, J = 4.70 Hz, 2 H) 6.28 (d, J = 1.94 Hz, 1 H) 7.21 (br. s., 1 H), 7.39 (s, 2 H) 7.51 (d, J = 1.66 Hz, 1 H) 8.65 (t, J = 4.98 Hz, 1 H) 11.86 (br. s., 1 H); MS: 439 [M + 1] | B |
| 143 | 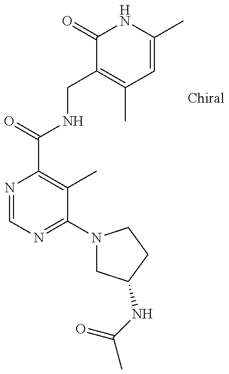 [SMILES: CC(=O)N[C@H]1CCN(c2ncnc(C(=O)NCc3c(C)cc(C)[nH]c3=O)c2C)C1] | 6-[(3S)-3- acetamidopyr- rolidin-1-yl]-N- [(4,6-dimethyl- 2-oxo-1,2- dihydropyridin- 3-yl)methyl]-5- methylpyrimi- dine-4- carboxamide | ¹H NMR (400 MHz, DMSO-d6) δ 1.73-1.86 (m, 4 H) 1.98-2.08 (m, 1 H) 2.11 (s, 3 H) 2.20 (s, 3 H) 2.35 (s, 3 H) 3.43 (dd, J = 11.12, 4.29 Hz, 1 H) 3.58-3.69 (m, 1 H) 3.69- 3.86 (m, 2 H) 4.18-4.24 (m, 1 H) 4.26 (d, J = 5.81 Hz, 2 H) 5.86 (s, 1 H) 8.09 (d, J = 6.57 Hz, 1 H) 8.31 (s, 1 H) 8.44 (t, J = 5.43 Hz, 1 H); MS: 399 [M + 1] | H |
| 144 | 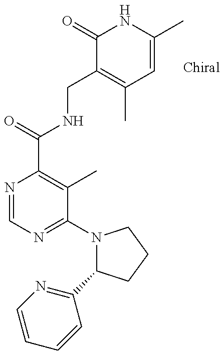 [SMILES: Cc1cc(C)c(CNC(=O)c2ncnc(N3CCC[C@@H]3c3ccccn3)c2C)c(=O)[nH]1] | N-[(4,6- dimethyl-2- oxo-1,2- dihydropyridin- 3-yl)methyl]-5- methyl-6-[(2R)- 2-pyridin-2- ylpyrrolidin-1- yl]pyrimidine- 4-carboxamide | ¹H NMR (400 MHz, DMSO-d6) δ 1.78-1.99 (m, 3 H) 2.10 (s, 3 H) 2.18 (s, 3 H) 2.31-2.41 (m, 4 H) 3.74-3.82 (m, 1 H) 3.99-4.08 (m, 1 H) 4.19- 4.29 (m, 2 H) 5.43 (t, J = 6.95 Hz, 1 H) 5.85 (s, 1 H) 7.15-7.22 (m, 2 H) 7.65 (td, J = 7.71, 1.77 Hz, 1 H) 8.17 (s, 1 H) 8.39 (t, J = 5.43 Hz, 1 H) 8.46 (m, J = 3.98, 0.79, 0.79 Hz, 1 H) 11.47 (s, 1 H); MS: 419 [M + 1] | H |
| 145 | 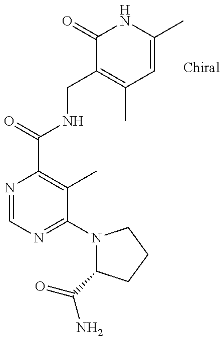 [SMILES: Cc1cc(C)c(CNC(=O)c2ncnc(N3CCC[C@@H]3C(N)=O)c2C)c(=O)[nH]1] | 6-[(2R)-2- carbamoylpyr- rolidin-1-yl]-N- [(4,6-dimethyl- 2-oxo-1,2- dihydropyridin- 3-yl)methyl]-5- methylpyrimi dine-4- carboxamide | ¹H NMR (400 MHz, DMSO-d6) δ 1.77-1.86 (m, 2 H) 1.88-1.99 (m, 1 H) 2.11 (s, 3 H) 2.13-2.18 (m, 1 H) 2.19 (s, 3 H) 2.38 (s, 3 H) 3.66-3.74 (m, 1 H) 3.82-3.91 (m, 1 H) 4.20-4.32 (m, 2 H) 4.52 (t, J = 7.03 Hz, 1 H) 5.86 (s, 1 H) 6.85 (s, 1 H) 7.30 (s, 1 H) 8.26 (s, 1 H) 8.42 (t, J = 5.50 Hz, 1 H) 11.51 (br. s., 1 H); MS: 385 [M + 1] | H |
| 146 | 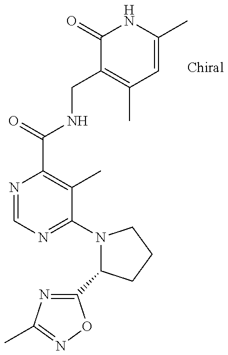 | N-[(4,6- dimethyl-2- oxo-1,2- dihydropyridin- 3-yl)methyl]-5- methyl-6-[(2R)- 2-(3-methyl- 1,2,4- oxadiazol-5- yl)pyrrolidin-1- yl]pyrimidine- 4-carboxamie | ¹H NMR (400 MHz, DMSO-d6) δ 1.89-2.08 (m, 3 H) 2.11 (s, 3 H) 2.19 (s, 3 H) 2.26 (s, 3 H) 2.32- 2.44 (m, 4 H) 3.79-3.89 (m, 1 H) 3.94-4.02 (m, 1 H) 4.26 (d, J = 5.31 Hz, 2 H) 5.48 (t, J = 6.82 Hz, 1 H) 5.86 (s, 1 H) 8.19 (s, 1 H) 8.44 (t, J = 5.31 Hz, 2 H) 11.48 (s, 1 H); MS: 424 [M + 1] | H |
| 147 | 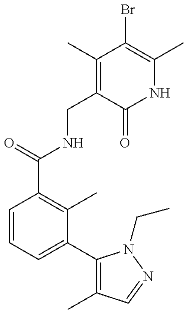 [SMILES: CCn1ncc(C)c1-c1cccc(C(=O)NCc2c(C)c(Br)c(C)[nH]c2=O)c1C] | N-[(5-bromo- 4,6-dimethyl-2- oxo-1,2- dihydropyridin- 3-yl)methyl]-3- (1-ethyl-4- methyl-1H- pyrazol-5-yl)-2- methyl- benzamide | ¹H NMR (400 MHz, DMSO-d6) δ 11.96 (br. s., 1 H), 8.34 (t, J = 4.71 Hz, 1 H), 7.27-7.41 (m, 3 H), 7.22 (dd, J = 7.09, 1.83 Hz, 1 H), 4.40 (d, J = 4.89 Hz, 2 H), 3.77-3.89 (m, 1 H), 3.65-3.77 (m, 1 H), 2.35 (s, 3 H), 2.32 (s, 3 H), 1.99 (s, 3 H), 1.79 (s, 3 H), 1.15 (t, J = 7.15 Hz, 3 H); MS: 457.9 [M + 1] | N |
| 148 | 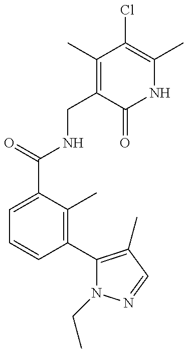 [SMILES: CCn1ncc(C)c1-c1cccc(C(=O)NCc2c(C)c(Cl)c(C)[nH]c2=O)c1C] | N-[(5-chloro- 4,6-dimethyl-2- oxo-1,2- dihydropyridin- 3-yl)methyl]-3- (1-ethyl-4- methyl-1H- pyrazol-5-yl)-2- methyl- benzamide | ¹H NMR (400 MHz, methanol -d4) δ 7.44- 7.48 (m, 1 H), 7.42 (s, 1 H), 7.39 (t, J = 7.58 Hz, 1 H), 7.27 (dd, J = 7.58, 1.47 Hz, 1 H), 4.57 (s, 2 H), 3.86-3.96 (m, 1 H), 3.75- 3.86 (m, 1 H), 2.51 (s, 3 H), 2.40 (s, 3 H), 2.09 (s, 3 H), 1.86 (s, 3 H), 1.23 (t, J = 7.21 Hz, 3 H); MS: 413.1 [M + 1] | N |
| 149 | 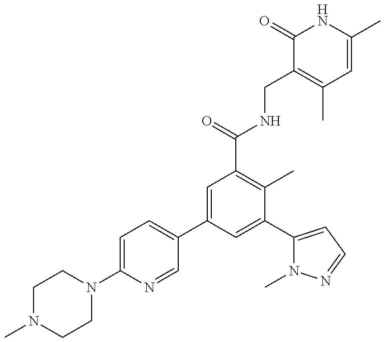 | N-[(4,6- dimethyl-2- oxo-1,2- dihydropyridin- 3-yl)methyl]-2- methyl-5-[6-(4- methylpiperazin- 1-yl)pyridin- 3-yl]-3-(1- methyl-1H- pyrazol-5- yl)benzamide | ¹H NMR (400 MHz, methanol -d4) δ 8.42 (d, J = 2.32 Hz, 1 H), 7.88 (dd, J = 8.86, 2.51 Hz, 1 H), 7.65 (d, J = 1.83 Hz, 1 H), 7.57 (d, J = 1.83 Hz, 1 H), 7.52 (d, J = 1.83 Hz, 1 H), 6.92 (d, J = 8.93 Hz, 1 H), 6.31 (d, J = 1.96 Hz, 1 H), 6.12 (s, 1 H), 4.52 (s, 2 H); MS: 526.3 [M + 1] | C |
| 150 | 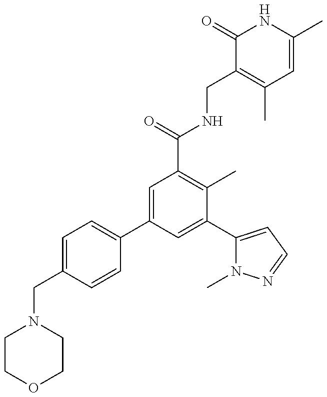 [SMILES: Cc1cc(C)c(CNC(=O)c2cc(-c3ccc(CN4CCOCC4)cc3)cc(-c3ccnn3C)c2C)c(=O)[nH]1] | N-[(4,6- dimethyl-2- oxo-1,2- dihydropyridin- 3-yl)methyl]-4- methyl-5-(1- methyl-1H- pyrazol-5-yl)- 4′-(morpholin- 4-ylmethyl)bi- phenyl-3- carboxamide | 1H NMR (400 MHz, methanol -d4) δ 7.72 (d, J = 1.71 Hz, 1 H), 7.65 (d, J = 8.07 Hz, 2 H), 7.58 (d, J = 1.71 Hz, 2 H), 7.45 (d, J = 8.07 Hz, 2 H), 6.33 (d, J = 1.96 Hz, 1 H), 6.14 (s, 1 H), 4.54 (s, 2 H), 3.72 (d, J = 4.65 Hz, 4 H), 3.68 (s, 3 H), 3.58 (s, 2 H), 2.50 (d, J = 3.91 Hz, 4 H), 2.42 (s, 3 H), 2.27 (s, 3 H), 2.17 (s, 3 H); MS: 526.2 [M + 1] | C |

### Biological Assays and Data

### Nucleosome Assay Protocol:

A. Compound Preparation
1. Prepare 10 mM stock solutions in 100% DMSO from solid material
2. Serial dilute 10 mM compound stocks either 2 or 3-fold in 100% DMSO to generate compounds for 11 point dose response

B. Reagent Preparation
1. Prepare 1× assay buffer containing 100 mM Tris pH 8.5, 4 mM DTT and 0.01% Tween-20
2. Dilute purified HeLA oligonucleosomes and recombinant histone H1 (New England Biolabs) in assay buffer to 1.67×.
3. Dilute EZH2 4 protein complex (EZH2, EED, SUZ12, RbAp48) to 3.5× in assay buffer
4. Prepare 10× ³H SAM solution in assay buffer using 0.94 μCi/well of radioactive SAM (Perkin Elmer) and sufficient non-labeled SAM (Sigma) for 1.5 μM final concentration.
5. Dilute TCA to 20% in DI water

C. Enzyme Reaction
1. Final reaction conditions are 5 nM EZH2 4-protein complex, 1.5 μM SAM, 25 μg/mL oligonucleosomes, 50 nM rH1 in a 50 μl reaction volume.
2. Add 1 μl of diluted compound to the assay plate (96-well V-bottom polypropylene plates) or 1 μl of DMSO for control wells.
3. Add 30 μl of nucleosomes to the assay plate
4. Add 14 μl of EZH2 4 protein complex to the assay plate
5. Add 5 μl of ³H SAM to start the reaction.
6. Stop the reaction after 60 minutes with the addition of 100 μl of 20% TCA
7. Transfer 150 μl of quenched reaction into a prepared filterplate (Millipore #MSIPN4B10)
8. Apply vacuum to the filterplate to filter the reaction mix through the membrane.
9. Wash the filterplate with 8×200 μl of PBS, blot dry and dry in an oven for 30 minutes
10. Add 50 μl of microscint-20 scintillation fluid (Perkin Elmer) to each well, wait 30 minutes and count on a liquid scintillation counter.

D. Data Analysis
1. IC₅₀ values were determined by fitting the data to a 4-parameter IC₅₀ equation using proprietary curve fitting software.

Preparation of HeLA Oligonucleosomes:

Reagents
Cell Pellet: 15 L HeLa S3 (Accelgen)+6 L HeLa S3 (in house)
Mnase (Worthington Biochemicals)

Equipment
SW-28 Rotor
Dounce Homogenizer/B Pestle

Buffers
Lysis: 20 mM Hepes pH 7.5, 0.25M Sucrose, 3 mM MgCl₂, 0.5% Nonidet P-40, 0.5 mM TCEP, 1 Roche Protease Tablet
B: 20 mM Hepes pH7.5, 3 mM MgCl₂, 0.5 mM EDTA, 0.5 mM TCEP, 1 Roche Protease Tablet
MSB: 20 mM Hepes pH7.5, 0.4 M NaCl, 1 mM EDTA, 5% v/v Glycerol, 0.5 mM TCEP, 0.2 mM PMSF
LSB: 20 mM Hepes pH7.5, 0.1M NaCl, 1 mM EDTA, 0.5 mM TCEP, 0.2 mM PMSF
NG: 20 mM Hepes pH7.5, 1 mM EDTA, 0.4 m NaCl, 0.2 mM PMSF, 0.5 mM TCEP
Storage: 20 mM Hepes pH7.5, 1 mM EDTA, 10% Glycerol, 0.2 mM PMSF, 0.5 mM TCEP

### Protocol

A. Nuclei
1. Resuspend ˜10 L pellet in 2×40 mL lysis using dounce homogenizer
2. Spin 3000×g 15′
3. Repeat 2 more times
4. Resuspend pellet in 2×40 mL B
5. Spin 3000×g 15′

B. Nuclei Resuspension
1. Resuspend pellet in 2×40 mL MSB. Spin 5000×g 20′
2. Resuspend pellet in 2×15 mL HSB
3. Pool and Homogenize 40 Strokes to shear DNA
4. Pellet 10000×g 20′
5. Dialyze O/N 4° C. in LSB except for Batch A which was Dialyzed LSB at 50 nM NaCl for 3 hr

C. Mnase Digestion
Test Mnase digestion (200 μl)
1. Warm to 37° C. for 5′
2. Add CaCl₂ to 3 mM and add 10U of Mnase
3. 37° C. 30′ taking 25 μl sample every 5′
4. Process reaction with 1 μL 0.5M EDTA, 40 μL H₂O, 15 μL 10% SDS, 10 μL 5M NaCl, and 100 μL phenol-chloroform vortexing after each addition
5. Spin 5′ 13 k
6. Run 5 μL of Aqueous phase on 1% agarose gel
7. Take time that yields ˜2 kb fragments
8. Selected 15′ for A & B and 20′ for C & D for scale up Added NaCl to 0.6M

D. Sucrose Gradient 1
1. Poured 6×34 mL gradient from 5 to 35% sucrose in NG using AKTA purifier in 38.5 mL pollyallomer tubes
2. Lead ˜4.0 mL on top of MN1 digest
3. Spin 26 k 16 hr 4° C.
4. Take 2 mL fractions from top
5. Run on Page Gel
6. Dialyze Fractions 7-14 0/N 4° C. in 4 L LSB except Batch D which had 2× 2 hr
7. Repeat 3×

E. Final
1. Pool all and concentrate in Amicon (somewhat cloudy)
2. Added 10% Glycerol
3. Spun 5K 15′
4. 1.8 mg/mL at 80 mL for 144 mg Total

Results for the biological examples are summarized below. Wild type (WT) EZH2% Effect at 20 μM and IC₅₀ values (μM) generated in WT EZH2 or EZH2PRC2 mutant Y641N nucleosome assays are provided in Table 3 below. A blank entry in the table indicates the data was not generated in that assay.

TABLE 3

| | % Effect in | | EZH2PCR2 |
|---|---|---|---|
| | WT EZH2 | WT EZH2 | Mutant Y641N |
| Ex | at 20 μM | IC₅₀ (μM) | IC₅₀ (μM) |
| 1 | — | 0.47 | — |
| 2 | — | 0.34 | 5.53 |
| 3 | — | 0.92 | — |
| 4 | — | 0.23 | — |
| 5 | 94.88 | 0.45 | — |
| 6 | — | 0.022 | 0.82 |
| 7 | — | 0.22 | — |
| 8 | — | 0.88 | — |
| 9 | — | 0.70 | — |
| 10 | — | 0.38 | — |
| 11 | — | 0.36 | — |
| 12 | — | 0.11 | — |
| 13 | — | 0.038 | 0.62 |
| 14 | — | 0.027 | 0.64 |
| 15 | — | 0.035 | 0.3 |
| 16 | — | 0.084 | 1.33 |
| 17 | — | 0.16 | — |
| 18 | — | 0.14 | — |
| 19 | — | 0.041 | — |
| 20 | — | 0.19 | — |
| 21 | — | 0.019 | — |
| 22 | — | 0.027 | 0.75 |
| 23 | — | 0.015 | — |
| 24 | — | 0.022 | 0.55 |
| 25 | — | 0.16 | — |
| 26 | 93.705 | 0.93 | — |
| 27 | — | 0.29 | 5.15 |
| 28 | — | 0.26 | — |
| 29 | — | 0.71 | — |
| 30 | — | 0.54 | — |
| 31 | — | 0.14 | 1.31 |
| 32 | 83.654 | 0.87 | 11.2 |
| 33 | 92.965 | 0.78 | — |
| 34 | — | 0.94 | — |
| 35 | — | 0.042 | 0.35 |
| 36 | — | 2.08 | 13 |
| 37 | — | 1.19 | 6.25 |
| 38 | — | 0.71 | — |
| 39 | — | 0.87 | — |
| 40 | — | 1.03 | — |
| 41 | — | 1.10 | 7.81 |
| 42 | — | 1.12 | — |
| 43 | — | 1.34 | — |
| 44 | — | 1.41 | — |
| 45 | — | 1.53 | — |
| 46 | 93.582 | 1.63 | — |
| 47 | — | 1.70 | — |
| 48 | — | 1.81 | 19.8 |
| 49 | — | 1.95 | — |
| 50 | — | 2.05 | 26.7 |
| 51 | — | 2.11 | — |
| 52 | — | 2.46 | — |
| 53 | — | 2.52 | — |
| 54 | — | 2.83 | — |
| 55 | — | 2.89 | — |
| 56 | — | 3.18 | — |
| 57 | — | 3.20 | — |
| 58 | — | 3.52 | — |
| 59 | — | 3.91 | — |
| 60 | — | 3.98 | 50.4 |
| 61 | — | 4.21 | — |
| 62 | — | 4.22 | — |
| 63 | — | 4.38 | — |
| 64 | — | 4.42 | — |
| 65 | 71.459 | 4.51 | — |
| 66 | 76.288 | 4.88 | — |
| 67 | — | 4.93 | 23.5 |
| 68 | — | 5.19 | — |
| 69 | — | 5.19 | — |
| 70 | — | 5.21 | — |
| 71 | — | 6.33 | — |
| 72 | — | 6.37 | — |
| 73 | — | 6.50 | — |
| 74 | — | 6.61 | — |
| 75 | — | 6.78 | — |
| 76 | — | 6.94 | — |
| 77 | 69.743 | 7.03 | — |
| 78 | — | 7.31 | — |
| 79 | 71.325 | 7.52 | 43.1 |
| 80 | — | 7.53 | — |
| 81 | — | 7.74 | — |
| 82 | 69.830 | 7.79 | — |
| 83 | — | 7.96 | — |
| 84 | — | 8.14 | — |
| 85 | 69.248 | 9.12 | — |
| 86 | 64.416 | 9.38 | — |
| 87 | — | 9.76 | — |
| 88 | — | 9.82 | 34.8 |
| 89 | — | 11.0 | 82.4 |
| 90 | — | 12.0 | — |
| 91 | — | 12.1 | — |
| 92 | — | 12.8 | — |
| 93 | — | 14.5 | — |
| 94 | — | 16.0 | — |
| 95 | — | 16.5 | — |
| 96 | — | 16.5 | — |
| 97 | — | 16.9 | — |
| 98 | — | 17.3 | — |
| 99 | — | 17.6 | — |
| 100 | — | 17.6 | — |
| 101 | — | 18.4 | — |
| 102 | — | 18.6 | — |
| 103 | — | 19.1 | — |
| 104 | — | 19.4 | — |
| 105 | — | 19.8 | — |
| 106 | — | 19.8 | 86.0 |
| 107 | — | 21.8 | — |
| 108 | — | 22.2 | — |
| 109 | — | 23.0 | — |
| 110 | — | 23.2 | — |
| 111 | — | 24.8 | — |
| 112 | — | 25.3 | — |
| 113 | 43.834 | 27.1 | — |
| 114 | — | 28.4 | — |
| 115 | — | 28.7 | — |
| 116 | — | 29.2 | — |
| 117 | — | 31.1 | — |
| 118 | — | 31.8 | — |
| 119 | 44.087 | 37.4 | — |
| 120 | — | 40.5 | — |
| 121 | — | 42.1 | — |
| 122 | — | 43.6 | — |
| 123 | — | 44.4 | — |
| 124 | — | 49.3 | — |
| 125 | — | 50.7 | — |
| 126 | 38.121 | 52.0 | — |
| 127 | 35.052 | 53.3 | — |
| 128 | 38.993 | 54.3 | — |
| 129 | 32.145 | 54.3 | — |
| 130 | — | 67.9 | — |
| 131 | — | 77.8 | — |
| 132 | — | 81.4 | — |
| 133 | — | 87.5 | — |
| 134 | — | 127.2 | — |
| 135 | 62.306 | 140.9 | >200 |
| 136 | — | 161.2 | — |
| 137 | 20.924 | 166.7 | — |
| 138 | 44.736 | 172.1 | — |
| 139 | — | >200 | — |
| 140 | — | >200 | — |
| 141 | — | >200 | — |
| 142 | — | >200 | — |
| 143 | — | >200 | — |
| 144 | — | >200 | — |
| 145 | — | >200 | — |
| 146 | — | >200 | — |
| 147 | — | 178 | >200 |
| 148 | — | >200 | — |
| 149 | — | 0.0342 | 0.198 |
| 150 | — | 0.113 | 0.679 |

All publications and patent applications cited in this specification and all references cited therein are herein incorporated by reference as if each individual publication or patent application or reference were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A compound of formula (III): 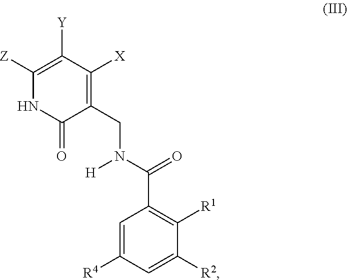
or a pharmaceutically acceptable salt thereof,
wherein:
R¹ is C₁-C₈ alkyl, C₁-C₈ alkoxy, halo, —OH, —CN or —NR⁷R⁸, where each said C₁-C₈ alkyl or C₁-C₈ alkoxy is optionally substituted by one or more R²¹;
R² is C₆-C₁₂ aryl, 5-12 membered heteroaryl or C₁-C₈ alkoxy, where said C₁-C₈ alkoxy is optionally substituted by one or more R²², and each said aryl or heteroaryl is optionally substituted by one or more R³²;
R⁴ is independently selected from the group consisting of H, C₁-C₈ alkyl, C₁-C₈ alkoxy, C₁-C₈ thioalkoxy, halo, —OH, —CN, C₃-C₈ cycloalkyl, 3-12 membered heterocyclyl, C₆-C₁₂ aryl, 5-12 membered heteroaryl, —OR¹¹ and —NR⁷R⁸, where each said C₁-C₈ alkyl, C₁-C₈ alkoxy, C₁-C₈ thioalkoxy or C₃-C₈ cycloalkyl is optionally substituted by one or more R²⁴, and each said heterocyclyl, aryl, heteroaryl or R¹¹ is optionally substituted by one or more R³⁴;
each R⁷ and R⁸ is independently H or C₁-C₈ alkyl, where said C₁-C₈ alkyl is optionally substituted by one or more R²⁷; or
R⁷ and R⁸ may be taken together with the N atom to which they are attached to form a 3-12 membered heterocyclyl or 5-12 membered heteroaryl, each optionally containing 1, 2 or 3 additional heteroatoms selected from O, N and S, wherein each said heterocyclyl or heteroaryl is optionally substituted by one or more R³⁷;
each R²¹ and R²² is independently selected from the group consisting of halo, —OH, C₁-C₄ alkoxy, —CN and —NR⁹R¹⁰;
each R²⁴ and R²⁷ is independently selected from the group consisting of halo, —OH, C₁-C₄ alkoxy, —CN, —NR⁹R¹⁰, C₃-C₈ cycloalkyl, 3-12 membered heterocyclyl, C₆-C₁₂ aryl and 5-12 membered heteroaryl, where each said cycloalkyl, heterocyclyl, aryl or heteroaryl is optionally substituted by one or more substituents independently selected from the group consisting of halo, —OH, ═O, C₁-C₄ alkyl, C₁-C₄ alkoxy, —CN, —NH₂, —NH(C₁-C₄ alkyl) and —N(C₁-C₄ alkyl)₂;
each R⁹ and R¹⁰ is independently H or C₁-C₄ alkyl; or
R⁹ and R¹⁰ may be taken together with the N atom to which they are attached to form a 3-12 membered heterocyclyl or 5-12 membered heteroaryl, each optionally containing 1, 2 or 3 additional heteroatoms selected from O, N and S, where each said heterocyclyl or heteroaryl is optionally substituted by one or more substituents independently selected from the group consisting of halo, —OH, ═O, C₁-C₄ alkyl, C₁-C₄ alkoxy, —CN, —NH₂, —NH(C₁-C₄ alkyl) and —N(C₁-C₄ alkyl)₂;
R¹¹ is selected from the group consisting of C₃-C₈ cycloalkyl, 3-12 membered heterocyclyl, C₆-C₁₂ aryl and 5-12 membered heteroaryl;
each R³², R³⁴ and R³⁷ is independently selected from the group consisting of halo, C₁-C₈ alkyl, —CN, ═O, —COR^{c}, —CO₂R^{c}, —CONR^{c}R^{d}, —OR^{c}, —SR^{c}, —SOR^{c}, —SO₂R^{c}, —SO₂NR^{c}R^{d}, —NO₂, —NR^{c}R^{d}, —NR^{c}C(O)R^{d}, —NR^{c}C(O)NR^{c}R^{d}, —NR^{c}C(O)OR^{d}—NR^{c}SO₂R^{d}, —NR^{c}SO₂NR^{c}R^{d}, —OC(O)R^{c}, —OC(O)NR^{c}R^{d}, C₃-C₈ cycloalkyl, 3-12 membered heterocyclyl, C₆-C₁₂ aryl and 5-12 membered heteroaryl;
each R^{c} and R^{d} is independently selected from the group consisting of H, C₁-C₈ alkyl, C₃-C₈ cycloalkyl, 3-12 membered heterocyclyl, C₆-C₁₂ aryl and 5-12 membered heteroaryl; or
R^{c} and R^{d} may be taken together with the N atom to which they are attached to form a 3-12 membered heterocyclyl or 5-12 membered heteroaryl ring, each optionally containing 1, 2 or 3 additional heteroatoms selected from O, N and S;
wherein each said alkyl, cycloalkyl, heterocyclyl, aryl or heteroaryl in R³², R³⁴, R³⁷, R^{c} and R^{d} is optionally substituted by 1 to 3 substituents independently selected from the group consisting of halo, —OH, ═O, C₁-C₄ alkyl, C₁-C₄ alkoxy, C₁-C₆ haloalkyl, C₁-C₆ hydroxyalkyl, C₁-C₄ alkoxy-C₁-C₆ alkyl, —CN, —NH₂, —NH(C₁-C₄ alkyl) and —N(C₁-C₄ alkyl)₂;
X and Z are independently selected from the group consisting of H, C₁-C₈ alkyl, C₂-C₈ alkenyl, C₂-C₈ alkynyl, C₃-C₈ cycloalkyl, 3-12 membered heterocyclyl, C₆-C₁₂ aryl, 5-12 membered heteroaryl, halo, CN, —COR^{a}, —CO₂R^{a}, —CONR^{a}R^{b}, —SR^{a}, —SOR^{a}, —SO₂R^{a}, —SO₂NR^{a}R^{b}, —NO₂, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}—OR^{a}, —OC(O)R^{a} or —OC(O)NR^{a}R^{b};
wherein each said C₁-C₈ alkyl, C₂-C₈ alkenyl, C₂-C₈ alkynyl, C₃-C₈ cycloalkyl, 3-12 membered heterocyclyl, C₆-C₁₂ aryl, or 5-12 membered heteroaryl group is optionally substituted by one or more substituents independently selected from the group consisting of halo, —CN, —COR^{a}, —CO₂R^{a}, —CONR^{a}R^{b}, —SR^{a}, —SOR^{a}, —SO₂R^{a}, —SO₂NR^{a}R^{b}, —NO₂, —NR^{a}R^{b}, —NR^{a}C(O)R^{b}, —NR^{a}C(O)NR^{a}R^{b}, —NR^{a}C(O)OR^{a}—NR^{a}SO₂R^{b}, —NR^{a}SO₂NR^{a}R^{b}, —OR^{a}, —OC(O)R^{a}, —OC(O)NR^{a}R^{b}, C₃-C₈ cycloalkyl, 3-12 membered heterocyclyl, C₈-C₁₂ aryl, and 5-12 membered heteroaryl;
each R^{a} and R^{b} is independently H, C₁-C₈ alkyl, C₂-C₈ alkenyl, C₂-C₈ alkynyl, C₃-C₈ cycloalkyl, 3-12 membered heterocyclyl, C₆-C₁₂ aryl or 5-12 membered heteroaryl, where each said alkyl, alkenyl, alkynyl, cycloalkyl, heterocyclyl, aryl and heteroaryl is optionally substituted by one or more substituents independently selected from the group consisting of halo, C₁-C₄ alkyl, —OR″, —NR″₂, —CO₂R″, —CONR″₂, —SO₂R″ and —SO₂NR″₂, where each R″ is independently H or C₁-C₄ alkyl; or
R^{a} and R^{b} may be taken together with the N atom to which they are attached to form a 3-12 membered heterocyclyl or 5-12 membered heteroaryl, each optionally containing 1, 2 or 3 additional heteroatoms selected from O, N and S, wherein said heterocyclyl or heteroaryl is optionally substituted by one or more substituents independently selected from the group consisting of halo, —OH, ═O, C₁-C₄ alkyl, C₁-C₄ alkoxy, C₁-C₆ haloalkyl, C₁-C₆ hydroxyalkyl, C₁-C₄ alkoxy-C₁-C₆ alkyl, —CN, —NH₂, —NH(C₁-C₄ alkyl) and —N(C₁-C₄ alkyl)₂; and
Y is H, halo, —OH or C₁-C₄ alkoxy.

2. The compound or salt of claim 1, wherein R² is 5-12 membered heteroaryl optionally substituted by 1 to 3 R³².

3. The compound or salt of claim 2, wherein said 5-12 membered heteroaryl is selected from the group consisting of pyrazolyl, imidazolyl, triazolyl and pyrrolyl, where said 5-12 membered heteroaryl is optionally substituted by 1 to 3 R³².

4. The compound or salt of claim 1, wherein each R³² is independently selected from the group consisting of halo, C₁-C₄ alkyl, —OR^{c}, —SR^{c}, —SO₂R^{c} and —NR^{c}R^{d}; and each R^{c} and R^{d} is independently H or C₁-C₄ alkyl; or R^{c} and R^{d} in —NR^{c}R^{d} may be taken together with the N atom to which they are attached to form a 4-6 membered heterocyclyl optionally containing 1 additional heteroatom selected from O, N and S, where said 4-6 membered heterocyclyl is optionally substituted by 1 to 3 substituents independently selected from the group consisting of halo, —OH, ═O, C₁-C₄ alkyl, C₁-C₄ alkoxy, C₁-C₆ haloalkyl, C₁-C₆ hydroxyalkyl, C₁-C₄ alkoxy-C₁-C₆ alkyl, —CN, —NH₂, —NH(C₁-C₄ alkyl) and —N(C₁-C₄ alkyl)₂.

5. The compound or salt of claim 1, wherein R² is C₁-C₈ alkoxy optionally substituted by 1 to 3 R²².

6. The compound or salt of claim 1, wherein R¹ is C₁-C₄ alkyl or halo.

7. The compound or salt of claim 1, wherein R⁴ is H, halo, —CN or 5-12 membered heteroaryl, where said 5-12 membered heteroaryl is optionally substituted by 1 to 3 R³⁴.

8. The compound or salt of claim 7, wherein R⁴ is 5-12 membered heteroaryl selected from the group consisting of pyridyl, pyrimidinyl, pyrazinyl, pyrazolyl, imidazolyl, triazolyl and pyrrolyl, where said 5-12 membered heteroaryl is optionally substituted by 1 to 3 R³⁴.

9. The compound or salt of claim 7, wherein each R³⁴ is independently selected from the group consisting of halo, C₁-C₄ alkyl, —OR^{c}, —SR^{c}, —SO₂R^{c} and —NR^{c}R^{d}; and each R^{c} and R^{d} is independently H or C₁-C₄ alkyl; or R^{c} and R^{d} in —NR^{c}R^{d} may be taken together with the N atom to which they are attached to form a 4-6 membered heterocyclyl optionally containing 1 additional heteroatom selected from O, N and S, where said 4-6 membered heterocyclyl is optionally substituted by 1 to 3 substituents independently selected from the group consisting of halo, —OH, ═O, C₁-C₄ alkyl, C₁-C₄ alkoxy, C₁-C₆ haloalkyl, C₁-C₆ hydroxyalkyl, C₁-C₄ alkoxy-C₁-C₆ alkyl, —CN, —NH₂, —NH(C₁-C₄ alkyl) and —N(C₁-C₄ alkyl)₂.

10. The compound or salt of claim 1, wherein X and Z are independently C₁-C₄ alkyl, and Y is H.

11. A pharmaceutical composition comprising a compound of claim 1, or a pharmaceutically acceptable salt thereof, and a pharmaceutically acceptable carrier or excipient.

12. A method for the treatment of abnormal cell growth in a subject, comprising administering to the subject a therapeutically effective amount of a compound of claim 1, or a pharmaceutically acceptable salt thereof.

13. The method of claim 12, wherein the abnormal cell growth is cancer.

14. The method of claim 12, wherein the subject is human.

15. (canceled)
